(19) United States
(12) Patent Application Publication
(10) Pub. No.: US 2024/0252671 A1
(43) Pub. Date: Aug. 1, 2024

(54) NEODEGRADER-ANTI-CD33 ANTIBODY CONJUGATES

(71) Applicant: Orum Therapeutics, Inc., Yuseong-gu, Daejeon (KR)

(72) Inventors: Nathan FISHKIN, Cambridge, MA (US); Peter U. PARK, Cambridge, MA (US); Chen BAI, Cambridge, MA (US)

(21) Appl. No.: 18/566,805

(22) PCT Filed: Jun. 2, 2022
(86) PCT No.: PCT/IB2022/055168

§ 371 Date: Dec. 4, 2023

Related U.S. Application Data
(60) Provisional application No. 63/202,272, filed on Jun. 3, 2021.
(60) Provisional application No. 63/282,588, filed on Nov. 23, 2021.

(51) Int. Cl. A61K 47/68; A61P 35/00

(52) U.S. Cl. CPC A61K 47/6849; A61K 47/6803; A61K 47/6889; A61P 35/00

(57) ABSTRACT

The present disclosure provides neoDegraders conjugated to anti-CD33 antibodies. Also provided are compositions comprising the conjugates. The compounds and compositions are useful for treating a disease or condition, e.g., cancer, in a subject in need thereof.

### REFERENCE TO SEQUENCE LISTING SUBMITTED ELECTRONICALLY

The content of the electronically submitted sequence listing in ASCII text file (Name: 4547_017PC02_Seglisting_ST25; Size: 42, 724 bytes; and Date of Creation: May 31, 2022) filed with the application is incorporated herein by reference in its entirety.

### FIELD

The present disclosure provides neoDegrader conjugates, wherein the neoDegrader is conjugated to an anti-CD33 antibody or antigen-binding portion thereof. Also provided are compositions comprising the conjugates. The conjugates and compositions are useful for treating cancer in a subject in need thereof.

### BACKGROUND

Protein degradation has been validated as a therapeutic strategy by the effectiveness of immunomodulatory imide drugs. These compounds have the ability to bind to cereblon (CRBN) and promote recruitment and ubiquitination of substrate proteins mediated by CRL4^{CRBN} E3 ubiquitin ligase. It is thought that immunomodulatory imides act as “molecular glues,” filling the binding interface as a hydrophobic patch that reprograms protein interactions between the ligase and neosubstrates.

Despite the excitement for these compounds as novel treatments for cancer, thus far they have been limited to use in hematologic malignancies such as multiple myeloma and myelodysplastic syndrome (MDS). Expanding the library of compounds that can function by degrading other oncoproteins, many of which have been considered ‘undruggable,’ is an active area of drug development. Thus there is a continuing need for new compounds that can target these alternative oncoproteins and treat a wide array of cancers.

### SUMMARY

Treatment of patients with acute myeloid leukemia (AML) with small-molecule GSPT1 degraders has been shown to drive clinical responses, but has been associated with severe adverse events (AE). Patients with AML frequently have high levels of CD33 on the cancer cells—as supported by the clinical approval of a CD33-targeting ADC, Mylotarg, for the treatment of AML. The present invention is based on the discovery that combining a GSPT1 degrading payload molecule with a CD33 targeting antibody can improve both the clinical efficacy and the tolerability of a GSPT1 degrader.

In a first aspect, the present disclosure provides a conjugate of formula (I):

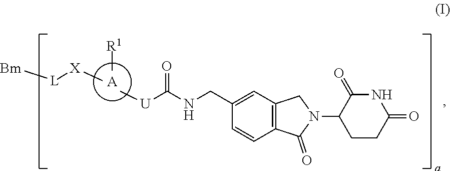

or a pharmaceutically acceptable salt thereof, wherein:
a is an integer from 1 to 10;
A is phenyl or a C₄-C₁₀cycloalkyl ring;
U is selected from NH and CF₂;
R¹ is independently selected from hydrogen and halo;
X is selected from —NR²—, ═C(CH₃)—, -Q-(CH₂)ₙ—, and -Q(CH₂)ₘQ′(CH₂)ₙ—; wherein
   Q and Q′ are each independently O, S, or N(R²);
   v is 1 or 2;
   each R² is independently hydrogen or C₁-C₆alkyl;
   n is an integer from 1 to 6; and
   m is an integer from 2 to 6;
wherein the left side of each group is attached to L and the right side is attached to A; provided that when X is NH or -Q-(CH₂)ₙ—, R¹ is halo;
L is a cleavable linker or non-cleavable linker; and
Bm is an anti-CD33 antibody or antigen-binding portion thereof.

In some aspects, the anti-CD33 antibody or antigen-binding portion thereof comprises a heavy chain variable region (VH) complementarity determining region (CDR) 1 (VH-CDR1) comprising the amino acid sequence as set forth in SEQ ID NO: 1, a VH-CDR2 comprising the amino acid as sequence set forth in SEQ ID NO: 2, a VH-CDR3 comprising the amino acid sequence as set forth in SEQ ID NO: 3, a light chain variable region (VL) CDR1 (VL-CDR1) comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7. In some aspects, the anti-CD33 antibody or antigen-binding portion thereof comprises a VH comprising the amino acid sequence set forth in SEQ ID NO:4 and a VL comprising the amino acid sequence as set forth in SEQ ID NO:8. In some aspects, the anti-CD33 antibody or antigen-binding portion thereof comprises a constant region, wherein the constant region comprises at least one amino acid different from Gemtuzumab. In some aspects, the anti-CD33 antibody or antigen-binding portion thereof is an IgG1 antibody or antigen-binding portion thereof. In some aspects, the anti-CD33 antibody or antigen-binding portion thereof comprises alanine at amino acid 297 corresponding to the constant region. In some aspects, the anti-CD33 antibody comprises a heavy chain as set forth in SEQ ID NO: 9 and a light chain as set forth in SEQ ID NO: 10.

In some aspects, the anti-CD33 antibody or antigen-binding portion thereof comprises a VH-CDR1 comprising the amino acid sequence as set forth in SEQ ID NO: 19, a VH-CDR2 comprising the amino acid as sequence set forth in SEQ ID NO: 20, a VH-CDR3 comprising the amino acid sequence as set forth in SEQ ID NO: 21, a VL-CDR1 comprising the amino acid sequence as set forth in SEQ ID NO: 22, a VL-CDR2 comprising the amino acid sequence as set forth in SEQ ID NO: 23, and a VL-CDR3 comprising the amino acid sequence as set forth in SEQ ID NO: 24. In some aspects, the anti-CD33 antibody or antigen-binding portion thereof comprises a VH comprising the amino acid sequence set forth in SEQ ID NO: 27 and a VL comprising the amino acid sequence as set forth in SEQ ID NO:28. In some aspects, the anti-CD33 antibody or antigen-binding portion thereof is an IgG1 antibody or antigen-binding portion thereof. In some aspects, the anti-CD33 antibody comprises a heavy chain as set forth in SEQ ID NO: 25 and a light chain as set forth in SEQ ID NO: 26.

In some aspects, a is an integer from 2 to 8.

In some aspects, L is a non-cleavable linker. In some aspects, L is selected from the group consisting of

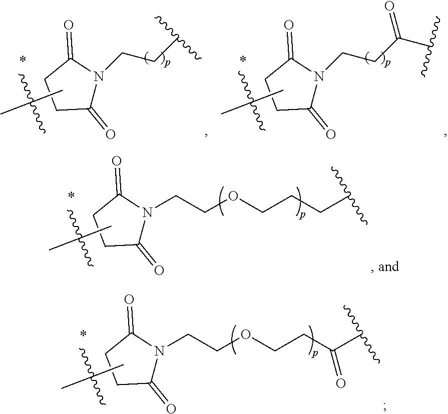

wherein:
p is an integer from 1 to 10;
[symbol] is the point of attachment to X; and
[symbol] is the point of attachment to the anti-CD33 antibody or antigen-binding portion thereof.

In some aspects, L is

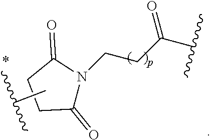

In some aspects, p is 5.

In some aspects, L is a cleavable linker. In some aspects, the cleavable linker is cleavable by a protease. In some aspects, L is selected from the group consisting of

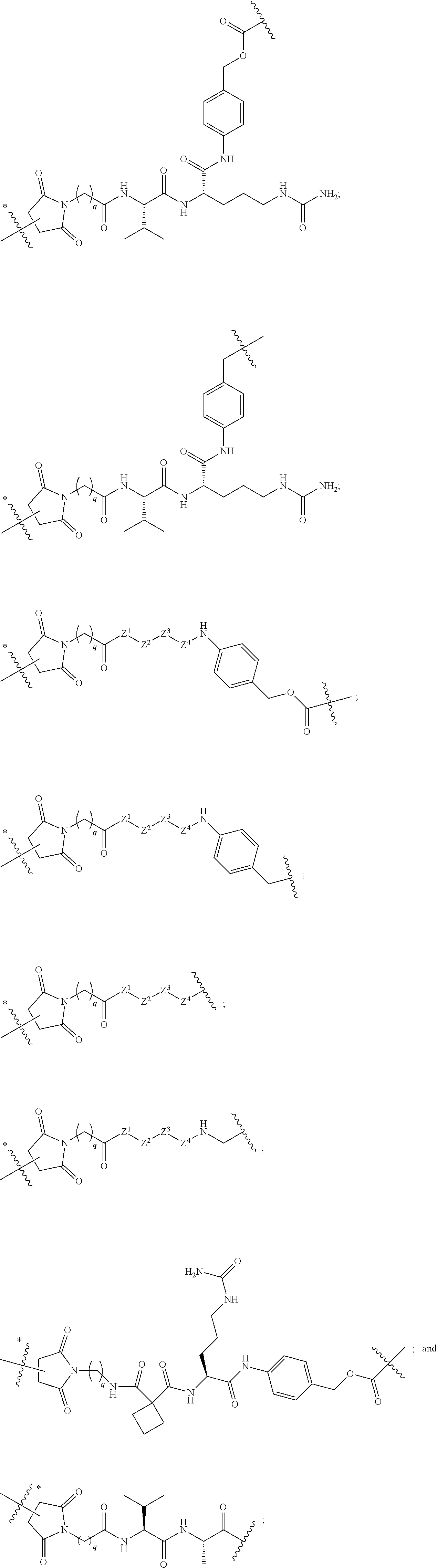

wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues;
[symbol] is the point of attachment to X; and
[symbol] is the point of attachment to the anti-CD33 antibody or antigen-binding portion thereof.

In some aspects, Z¹, Z², Z³, and Z⁴ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

In some aspects, Z¹ is absent or glycine; Z² is absent or selected from the group consisting of L-glutamine, D-glutamine, L-glutamic acid, D-glutamic acid, L-aspartic acid, D-aspartic acid, L-alanine, D-alanine, and glycine; Z³ is selected from the group consisting of L-valine, D-valine, L-alanine, D-alanine, L-phenylalanine, D-phenylalanine, and glycine; and Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

In some aspects, L is

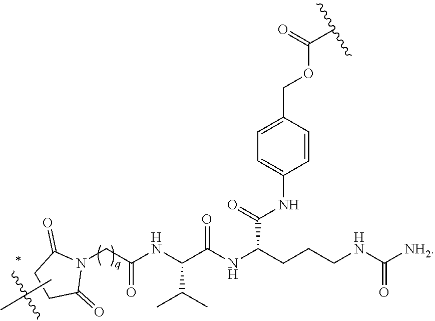

In some aspects, q is 5.

In some aspects, L is a bioreducible linker. In some aspects, L is selected from the group consisting of

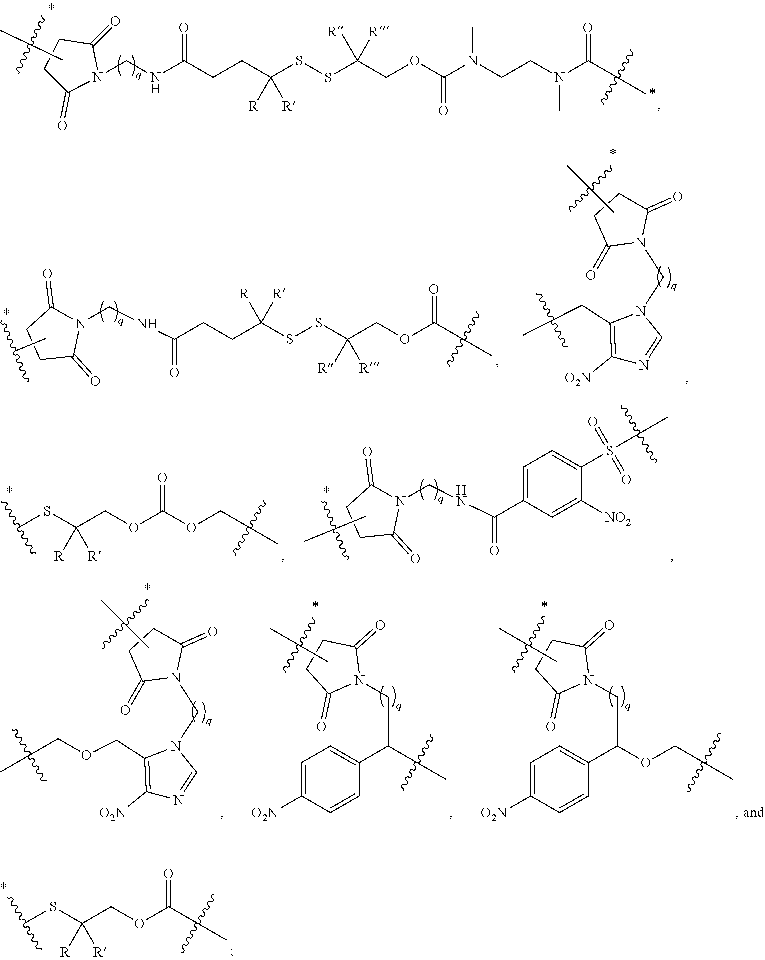

wherein:
q is an integer from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;

[symbol] is the point of attachment to X; and
[symbol] is the point of attachment to the anti-CD33 antibody or antigen-binding portion thereof.

In some aspects, L is an acid cleavable linker. In some aspects, L is selected from the group consisting of

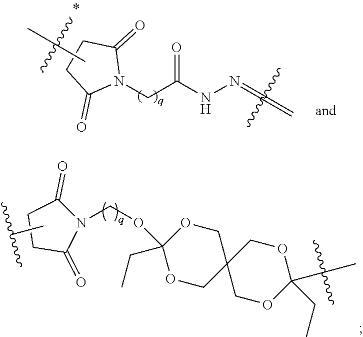

wherein:
q is an integer from 2 to 10;
[symbol] is the point of attachment to X; and
[symbol] is the point of attachment to the anti-CD33 antibody or antigen-binding portion thereof.

In some aspects, L is a click-to-release linker. In some aspects, L is selected from

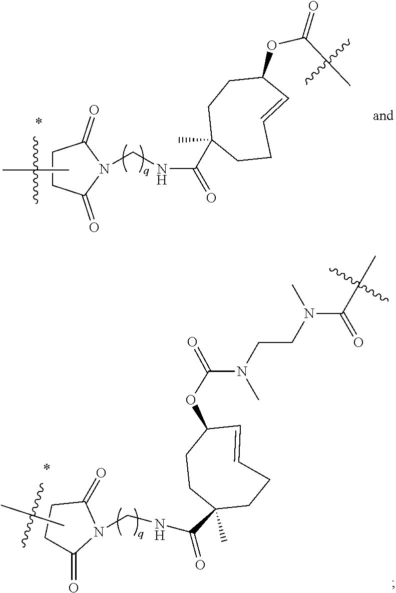

wherein:
q is an integer from 2 to 10;
[symbol] is the point of attachment to X; and
[symbol] is the point of attachment to the anti-CD33 antibody or antigen-binding portion thereof.

In some aspects, L is a pyrophosphatase cleavable linker. In some aspects, L is

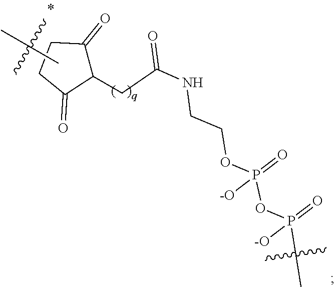

wherein:
q is an integer from 2 to 10;
[symbol] is the point of attachment to X; and
[symbol] is the point of attachment to the anti-CD33 antibody or antigen-binding portion thereof.

In some aspects, L is a beta-glucuronidase cleavable linker. In some aspects, L is selected from

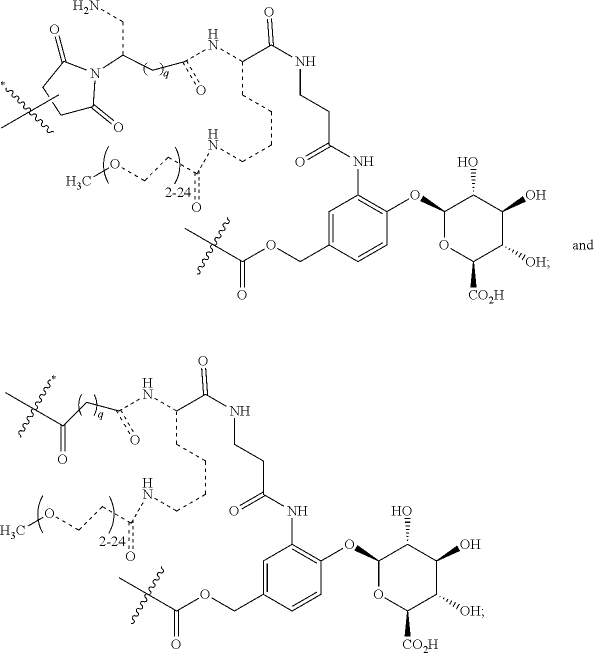

wherein:
q is an integer from 2 to 10;
---- is absent or a bond;
[symbol] is the point of attachment to X; and
[symbol] is the point of attachment to the anti-CD33 antibody or antigen-binding portion thereof.

In certain aspects, the present disclosure provides a conjugate of formula (I), wherein:
A is phenyl;
U is NH;
R¹ is halo; and
X is —N(R²)ᵥ(CH₂)ₘO(CH₂)ₙ—; wherein:
   v is 1;
   m and n are 2; and
   R² is methyl.

In certain aspects, the present disclosure provides a conjugate of formula (I), wherein:
A is phenyl;
U is NH;
R¹ is halo; and
X is —N(R²)ᵥ(CH₂)ₘO(CH₂)ₙ—; wherein:
   v is 2;
   m and n are 2; and
   each R² is methyl.

In certain aspects, the present disclosure provides a conjugate of formula (I), wherein:
A is phenyl;
U is NH;
R¹ is halo; and
X is —O(CH₂)ₙ—; wherein:
   n is 2.

In certain aspects, the present disclosure provides a conjugate of formula (I), wherein:
A is phenyl;
U is NH;
R¹ is halo; and
X is —S(CH₂)ₙ—; wherein:
   n is 2.

In certain aspects, the present disclosure provides a conjugate of formula (I), wherein:
A is phenyl;
U is NH;
R¹ is hydrogen; and
X is —NR²—; wherein:
   R² is methyl.

In certain aspects, the present disclosure provides a conjugate of formula (I), wherein:
A is phenyl;
U is NH;
R¹ is halo; and
X is —NR²—; wherein:
   R² is hydrogen.

In certain aspects, the present disclosure provides a conjugate of formula (I), wherein:
A is phenyl;
U is NH;
R¹ is hydrogen; and
X is —C(CH₃)═.

In certain aspects, the present disclosure provides a conjugate of formula (I), wherein:
A is a C₄-C₁₀cycloalkyl ring;
U is NH;
R¹ is hydrogen; and
X is —N(R²)(CH₂)ₘO(CH₂)ₙ—; wherein:
   n is 1;
   m is 2; and
   R² is methyl.

In certain aspects, the present disclosure provides a conjugate of formula (V):

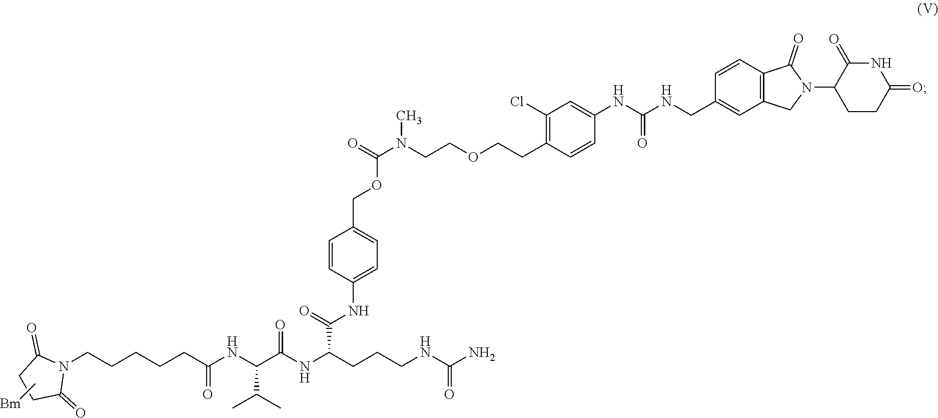

or a pharmaceutically acceptable salt thereof, wherein Bm is an anti-CD33 antibody or antigen-binding portion thereof. The anti-CD33 antibody or antigen-binding portion thereof can comprise e.g., (i) aVH-CDR1 comprising the amino acid sequence as set forth in SEQ ID NO: 1, a VH-CDR2 comprising the amino acids sequence as set forth in SEQ ID NO: 2, a VH-CDR3 comprising the amino acid sequence as set forth in SEQ ID NO: 3, a VL-CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7, (ii) a VH comprising the amino acid sequence as set forth in SEQ ID NO:4 and a VL comprising the amino acid sequence as set forth in SEQ ID NO:8, (iii) a heavy chain as set forth in SEQ ID NO: 9, and a light chain as set forth in SEQ ID NO: 10, (iv) aVH-CDR1 comprising the amino acid sequence as set forth in SEQ ID NO: 19, a VH-CDR2 comprising the amino acid as sequence set forth in SEQ ID NO: 20, a VH-CDR3 comprising the amino acid sequence as set forth in SEQ ID NO: 21, a VL-CDR1 comprising the amino acid sequence as set forth in SEQ ID NO: 22, a VL-CDR2 comprising the amino acid sequence as set forth in SEQ ID NO: 23, and a VL-CDR3 comprising the amino acid sequence as set forth in SEQ ID NO: 24, (v) a VH comprising the amino acid sequence as set forth in SEQ ID NO: 27 and a VL comprising the amino acid sequence as set forth in SEQ ID NO: 28, or (vi) a heavy chain as set forth in SEQ ID NO: 25, and a light chain as set forth in SEQ ID NO: 26.

In certain aspects, the present disclosure provides a conjugate of formula (VI)

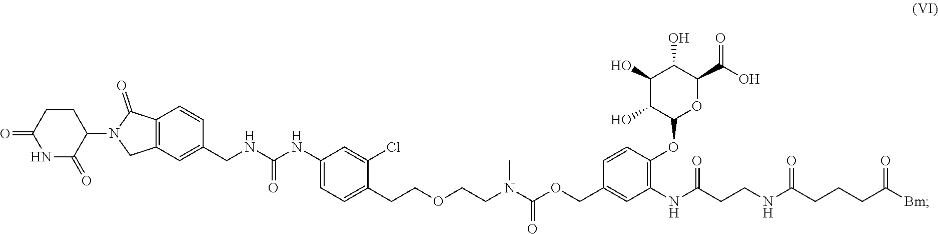

or a pharmaceutically acceptable salt thereof, wherein Bm is an anti-CD33 or antigen-binding portion thereof. The anti-CD33 antibody or antigen-binding portion thereof can comprise e.g., (i) aVH-CDR1 comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a VL-CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7, (ii) a VH comprising the amino acid sequence as set forth in SEQ ID NO: 4 and a VL comprising the amino acid sequence as set forth in SEQ ID NO: 8, (iii) a heavy chain as set forth in SEQ ID NO: 9, and a light chain as set forth in SEQ ID NO: 10, (iv) aVH-CDR1 comprising the amino acid sequence as set forth in SEQ ID NO: 19, a VH-CDR2 comprising the amino acid sequence as set forth in SEQ ID NO: 20, a VH-CDR3 comprising the amino acid sequence as set forth in SEQ ID NO: 21, a VL-CDR1 comprising the amino acid sequence as set forth in SEQ ID NO: 22, a VL-CDR2 comprising the amino acid sequence as set forth in SEQ ID NO: 23, and a VL-CDR3 comprising the amino acid sequence as set forth in SEQ ID NO: 24, (v) a VH comprising the amino acid sequence as set forth in SEQ ID NO: 27 and a VL comprising the amino acid sequence as set forth in SEQ ID NO: 28, or (vi) a heavy chain as set forth in SEQ ID NO: 25, and a light chain as set forth in SEQ ID NO: 26.

In certain aspects, the present disclosure provides a conjugate of formula (VI):

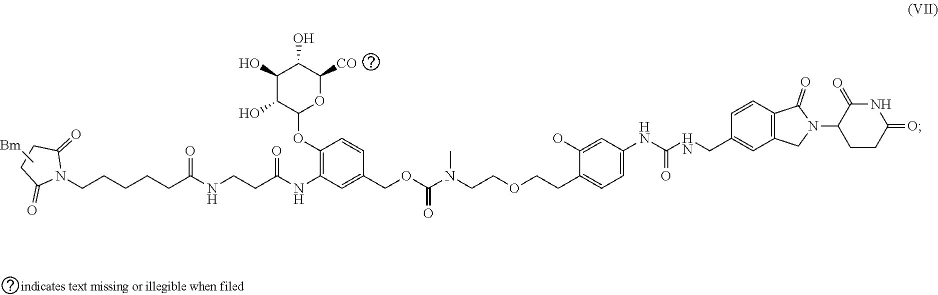

or a pharmaceutically acceptable salt thereof, wherein Bm is an anti-CD33 antibody or antigen-binding portion thereof. The anti-CD33 antibody or antigen-binding portion thereof can comprise e.g., (i) aVH-CDR1 comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a VL-CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7, (ii) a VH comprising the amino acid sequence as set forth in SEQ ID NO:4 and a VL comprising the amino acid sequence as set forth in SEQ ID NO: 8, (iii) a heavy chain as set forth in SEQ ID NO: 9, and a light chain as set forth in SEQ ID NO: 10, (iv) aVH-CDR1 comprising the amino acid sequence as set forth in SEQ ID NO: 19, a VH-CDR2 comprising the amino acid sequence as set forth in SEQ ID NO: 20, a VH-CDR3 comprising the amino acid sequence as set forth in SEQ ID NO: 21, a VL-CDR1 comprising the amino acid sequence as set forth in SEQ ID NO: 22, a VL-CDR2 comprising the amino acid sequence as set forth in SEQ ID NO: 23, and a VL-CDR3 comprising the amino acid sequence as set forth in SEQ ID NO: 24, (v) a VH comprising the amino acid sequence as set forth in SEQ ID NO: 27 and a VL comprising the amino acid sequence as set forth in SEQ ID NO: 28, or (vi) a heavy chain as set forth in SEQ ID NO: 25, and a light chain as set forth in SEQ ID NO: 26.

In certain aspects, the present disclosure provides a pharmaceutical composition comprising a conjugate or compound provided here, or a pharmaceutically acceptable salt thereof, and one or more pharmaceutically acceptable carriers.

In certain aspects, the present disclosure provides a method of treating cancer or myelodysplastic syndrome in a subject in need thereof, the method comprising administering to the subject a pharmaceutically acceptable amount of a conjugate or composition described above, or a pharmaceutically acceptable salt thereof. In some aspects, the cancer is a hematological/blood cancer. In some aspects, the cancer is a multiple myeloma, leukemia, malignant lymphoma, Hodgkin's disease, or chronic myeloproliferative disease. In some aspects, the cancer is acute myeloid leukemia or lymphoma. In some aspects, the cancer is acute myeloid leukemia. In some aspects, the cancer is resistant or refractory to Mylotarg.

In some aspects, the method further comprises administering to the subject a pharmaceutically acceptable amount of an additional agent prior to, after, or simultaneously with the conjugate, or a pharmaceutically acceptable salt thereof. In some aspects, the additional agent is a cytotoxic agent or an immune response modifier. In some aspects, the immune response modifier is a checkpoint inhibitor. In some aspects, the checkpoint inhibitor comprises a PD-1 inhibitor, a PD-L1 inhibitor, a CTLA-4 inhibitor, a TIM3 inhibitor, and/or a LAG-3 inhibitor.

In certain aspects, the present disclosure provides a method of preparing a conjugate of formula (I), or a pharmaceutically acceptable salt thereof, the process comprising reacting an anti-CD33 antibody or antigen-binding portion thereof with a compound of formula (I-1):

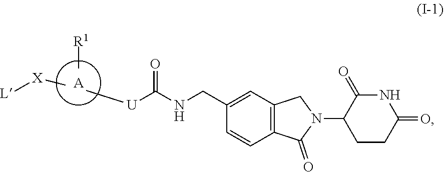

or a pharmaceutically acceptable salt thereof, wherein:
a is an integer from 1 to 10;
A is phenyl or a C₄-C₁₀cycloalkyl ring;
R¹ is independently selected from hydrogen and halo;
U is selected from NH and CF₂;
X is selected from —N(R²)ᵥ—, ═C(CH₃)—, -Q-(CH₂)ₙ—, and -Q(CH₂)ₘQ′(CH₂)ₙ—; wherein
   v is 1 or2;
   Q and Q′ are each independently O, S, or NR²;
   each R² is independently hydrogen or C₁-C₆alkyl;
   n is an integer from 1 to 6; and
   m is an integer from 2 to 6;
wherein the left side of each group is attached to L′ and the right side is attached to A;
provided that when X is NH or -Q-(CH₂)ₙ—, R¹ is halo;
L′ is a cleavable or non-cleavable linker precursor that conjugates to the anti-CD33 antibody or antigen-binding portion thereof. In some aspects, the anti-CD33 antibody or antigen-binding portion thereof comprises a VH-CDR1 comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a VL-CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7. In some aspects, the anti-CD33 antibody or antigen-binding portion thereof comprises a heavy chain variable region as set forth in SEQ ID NO: 4, and a light chain variable region as set forth in SEQ ID NO: 8. In some aspects, the anti-CD33 antibody or antigen-binding portion thereof comprises a constant region, wherein the constant region comprises at least one amino acid different from Gemtuzumab. In some aspects, the anti-CD33 antibody or antigen-binding portion thereof is an IgG1 antibody or antigen-binding portion thereof. In some aspects, the anti-CD33 antibody or antigen-binding portion thereof comprises alanine at amino acid 297 corresponding to the constant region. In some aspects, the anti-CD33 antibody comprises a heavy chain as set forth in SEQ ID NO: 9 and a light chain as set forth in SEQ ID NO: 10. In some aspects, the anti-CD33 antibody or antigen-binding portion thereof comprises a VH-CDR1 comprising the amino acid sequence as set forth in SEQ ID NO: 19, a VH-CDR2 comprising the amino acid sequence as set forth in SEQ ID NO: 20, a VH-CDR3 comprising the amino acid sequence as set forth in SEQ ID NO: 21, a VL-CDR1 comprising the amino acid sequence as set forth in SEQ ID NO: 22, a VL-CDR2 comprising the amino acid sequence as set forth in SEQ ID NO: 23, and a VL-CDR3 comprising the amino acid sequence as set forth in SEQ ID NO: 24. In some aspects, the anti-CD33 antibody or antigen-binding portion thereof comprises a VH comprising the amino acid sequence set forth in SEQ ID NO: 27 and a VL comprising the amino acid sequence as set forth in SEQ ID NO: 28. In some aspects, the anti-CD33 antibody or antigen-binding portion thereof is an IgG1 antibody or antigen-binding portion thereof. In some aspects, the anti-CD33 antibody comprises a heavy chain as set forth in SEQ ID NO: 25 and a light chain as set forth in SEQ ID NO: 26.

In some aspects, the method further comprises reducing the anti-CD33 antibody or antigen-binding portion thereof prior to reacting with the compound of formula (I-1).

In some aspects, a is an integer from 2 to 8. In some aspects, L′ is a non-cleavable linker precursor, a cleavable linker precursor, a bioreducible linker precursor, an acid cleavable linker precursor, a click-to-release linker precursor, a pyrophosphatase cleavable linker precursor, a beta-glucoronidase cleavable linker precursor, or any combination thereof.

### BRIEF DESCRIPTION OF THE FIGURES

FIG. 1 depicts in vivo activity of representative neoDegrader conjugates against MV411 (CD33+) tumors. The X axis shows the day after dosing. The Y axis shows the tumor volume (mm³) after dosing with vehicle, 3 mg/kg CD33AB—Compound (Ia), 2.83 mg/kg CD33AB-Compound (Ie), 2.99 mg/kg CD33AB-Compound (1h), 0.1 mg/kg Mylotarg, 50 mg/kg Venetoclax, and 5 mg/kg CC-90009.

FIG. 2 depicts the in vitro activity of huMy9-6 (AB1)—Compound (Ia) in CD-33 positive and CD33-negative malignancies.

FIG. 3 depicts in vivo activity of AB1 based conjugates against MV4-11 (CD33+) tumors. The X axis shows the day after dosing. The Y axis shows the tumor volume (mm³) after dosing with vehicle, AB1—Compound (Ia), AB1—Compound (Ii), AB1—Compound (Id), AB1-Compound (Ij), AB1—Compound (Ie), AB1—Compound (Ik), Mylotarg, Gemtuzumab-Compound I(a), and CC-90009.

FIG. 4 depicts the stability of Gemtuzumab and CD33AB conjugates.

FIG. 5 depicts in vivo activity of Gemtuzumab based conjugates against MV4-11 (CD33+) tumors. The X axis shows the day after dosing. The Y axis shows the tumor volume (mm³) after dosing with vehicle, 3 mg/kg Gemtuzumab—Compound (Ia), 5 mg/kg Gemtuzumab—Compound (Ia), 3 mg/kg CD33AB-Compound (Ia) 5 mg/kg CD33AB-Compound (Ia), 3 mg/kg Gemtuzumab IgG1 LALA-Compound (Ia), 5 mg/kg Gemtuzumab IgG1 LALA-Compound (Ia), 5 mg/kg Gemtuzumab-Compound (Ie), 25 mg/kg Venetoclax, and 50 mg/kg Ventoclax.

FIGS. 6A and 6B depict the in vitro activity of the AB1—Compound (Ia) conjugate against Mylotarg-insensitive AML cells (AML-193 (FIG. 6A) and Kasumi-6 (FIG. 6B)). The X axis shows concentration, and the Y axis shows the percent viability of the cell line after treatment.

### DETAILED DESCRIPTION

The present disclosure is directed to a conjugate of formula (I):

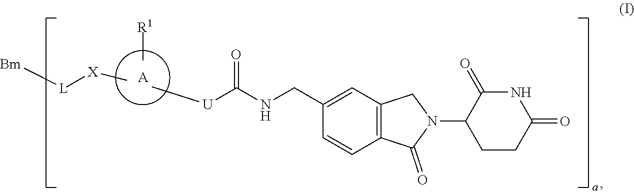

or a pharmaceutically acceptable salt thereof, wherein:
a is an integer from 1 to 10;
A is phenyl or a C₄-C₁₀cycloalkyl ring;
R¹ is independently selected from hydrogen and halo;
U is selected from NH and CF₂;
X is selected from —N(R²)ᵥ—, ═C(CH₃)—, -Q-(CH₂)ₙ—, and -Q(CH₂)ₘQ′(CH₂)ₙ—; wherein
   Q and Q′ are each independently O, S, or N(R²)ᵥ;
   v is 1 or 2;
   each R² is independently hydrogen or C₁-C₆alkyl;
   n is an integer from 1 to 6;
   m is an integer from 2 to 6;
wherein the left side of each group is attached to L and the right side is attached to A;
provided that when X is NH or -Q-(CH₂)ₙ—, R¹ is halo;
L is a cleavable linker or non-cleavable linker; and
Bm is an anti-CD33 antibody or antigen-binding portion thereof, e.g., an anti-CD33 antibody or antigen-binding portion thereof a VH-CDR1 comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a light chain variable region (VL) CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7; an anti-CD33 antibody or antigen-binding portion thereof comprising a VH comprising the amino acid sequence as set forth in SEQ ID NO: 4 and a VL comprising the amino acid sequence as set forth in SEQ ID NO: 8; or an anti-CD33 antibody comprising a heavy chain as set forth in SEQ ID NO: 9 and a light chain as set forth in SEQ ID NO: 10.

The present disclosure also provides the compound above that is fused to the anti-CD33 antibody or antigen-binding portion thereof, the composition comprising the compound or the conjugate, or the method of using or making the compound or the conjugate.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “DAR,” as used herein, refers to the drug antibody ratio of the conjugate, which is the average number of neoDegrader-linker complexes linked to each antibody. In certain aspects, the DAR of the conjugates described herein is from 1 to 10. In some aspects, the DAR of the conjugates described herein is from 1 to 8. In some aspects, the DAR of the conjugates described herein is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.

The term “antibody,” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “single domain antibody,” also known as a nanobody, is an antibody fragment consisting of a single monomeric variable antibody domain with a molecular weight of from about 12 kDa to about 15 kDa. Single body antibodies can be based on heavy chain variable domains or light chains. Examples of single domain antibodies include, but are not limited to, V_{H}H fragments and V_{NAR} fragments.

“Antibody fragments” comprise a portion of an intact antibody, generally the antigen binding or variable region thereof. Examples of antibody fragments include Fab, Fab′, F(ab′).sub.2, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

An “intact antibody” is one which comprises an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method, or may be made by recombinant DNA methods. The “monoclonal antibodies” may also be isolated from phage antibody libraries.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g., Old World Monkey, Ape etc.) and human constant region sequences.

Various methods have been employed to produce monoclonal antibodies (MAbs). Hybridoma technology, which refers to a cloned cell line that produces a single type of antibody, uses the cells of various species, including mice (murine), hamsters, rats, and humans. Another method to prepare MAbs uses genetic engineering including recombinant DNA techniques. Monoclonal antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen. While useful for diagnostics and short-term therapies, murine antibodies cannot be administered to people long-term without increasing the risk of a deleterious immunogenic response. This response, called Human Anti-Mouse Antibody (HAMA), occurs when a human immune system recognizes the murine antibody as foreign and attacks it. A HAMA response can cause toxic shock or even death.

Chimeric and humanized antibodies reduce the likelihood of a HAMA response by minimizing the nonhuman portions of administered antibodies. Furthermore, chimeric and humanized antibodies can have the additional benefit of activating secondary human immune responses, such as antibody dependent cellular cytotoxicity.

The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule is an antibody-drug conjugate.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. Conservation of sequence can apply to the entire length of an polynucleotide or polypeptide or can apply to a portion, region or feature thereof.

As used herein, the terms “linking” and “conjugating” are used interchangeably and each refer to the covalent or non-covalent attachment of two or more moieties comprising a neoDegrader and an anti-CD33 antibody or antigen-binding portion thereof. In some aspects the linking or conjugating can comprise a linker.

The term “amino acid sequence variant” refers to polypeptides having amino acid sequences that differ to some extent from a native sequence polypeptide. Ordinarily, amino acid sequence variants will possess at least about 70% sequence identity with at least one receptor binding domain of a native antibody or with at least one ligand binding domain of a native receptor, and typically, they will be at least about 80%, more typically, at least about 90% homologous by sequence with such receptor or ligand binding domains. The amino acid sequence variants possess substitutions, deletions, and/or insertions at certain positions within the amino acid sequence of the native amino acid sequence. Amino acids are designated by the conventional names, one-letter and three-letter codes.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay may be performed.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a .beta.-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies. The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al supra) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains which enables the scFv to form the desired structure for antigen binding.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain aspects, the antibody will be purified (1) to greater than 95% by weight of antibody as determined by the Lowry method, or more than 99% by weight, (2) to a degree sufficient to obtain at least 15 residues of N-terminal or internal amino acid sequence by use of a gas phase protein sequencer, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the term “T cell” and “T lymphocytes” are interchangeable and refer to any lymphocytes produced or processed by the thymus gland. In some aspects, a T cell is a CD4⁺ T cell. In some aspects, a T cell is a CD8+ T cell. In some aspects, a T cell is a NKT cell.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent (e.g., neoDegrader or neoDegrader conjugate disclosed herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the composition can, for example, (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of the neoDegrader or neoDegrader conjugate clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the drug promotes cancer regression to the point of eliminating the cancer.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### II. “NeoDegraders”

The present disclosure provides neoDegraders of formula (II):

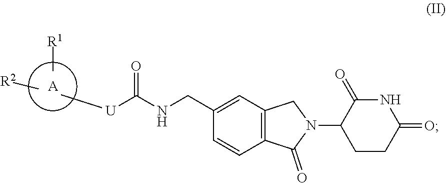

or pharmaceutically acceptable salts thereof, wherein:
A is phenyl or a C₄-C₁₀cycloalkyl ring;
U is selected from NH and CF₂;
R¹ is independently selected from hydrogen and halo;
R² is selected from —C(O)R³, —N(R⁴)₂, —(CH₂)ₙOH, —(CH₂)ₙSH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
   R³ is hydrogen or C₁-C₆alkyl;
   each R⁴ is independently hydrogen or C₁-C₆alkyl;
   Q′ is O, S, or NR⁴;
   n is 1-6; and
   m is 2-5;
provided that when R² is NH₂, —(CH₂)ₙNH₂, or —(CH₂)ₙOH then R¹ is halo.

In certain aspects, the present disclosure provides compounds of formula (II), or pharmaceutically acceptable salts thereof, wherein:
A is a phenyl ring or a C₄-C₁₀cycloalkyl ring;
U is NH;
R¹ is selected from hydrogen and halo;
R² is selected from —(CH₂)ₙQ′(CH₂)ₘN(R⁴)₂, —(CH₂)ₙOH, —(CH₂)ₙSH, —N(R⁴)₂, and —C(O)R³; wherein:
m is 2;
n is 2;
Q′ is —O—;
R³ is methyl; and
each R⁴ is independently selected from hydrogen and methyl; provided that when R² is NH₂ or —(CH₂)ₙOH, then R¹ is halo.

As used herein, the term “C₁-C₆alkoxy,” as used herein, refers to a C₁-C₆alkyl group attached to the parent molecular moiety through an oxygen atom.

As used herein, the term “C₁-C₆alkoxyC₁-C₆alkyl” refers to a C₁-C₆alkoxy group attached to the parent molecular moiety through a C₁-C₆alkyl group.

As used herein, the term “C₁-C₆alkyl” refers to a group derived from a straight or branched chain saturated hydrocarbon containing from one to six carbon atoms.

As used herein, the term “C₄-C₁₀cycloalkyl” refers to a a saturated monocyclic, hydrocarbon ring system having four to ten carbon atoms and zero heteroatoms. Representative examples of cycloalkyl groups include, but are not limited to, cyclobutyl, cyclopentyl, and cyclohexyl. The cycloalkyl groups containing between seven and ten atoms may be monocyclic or fused, spirocyclic, or bridged bicyclic structures.

As used herein, the term “halo” refers to F, Cl, Br, or I.

In some aspects, the neoDegrader of formula (II) is a compound selected from the group consisting of:

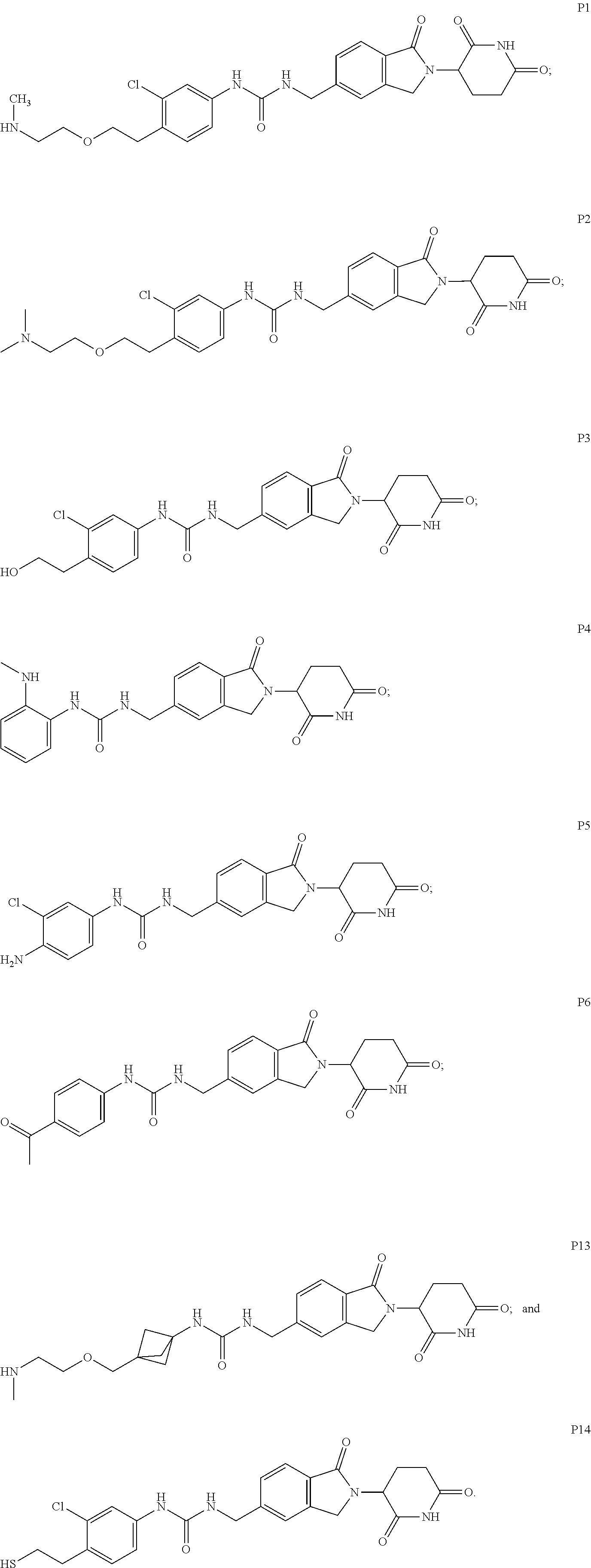

In some aspects, the neoDegrader of formula (II) is

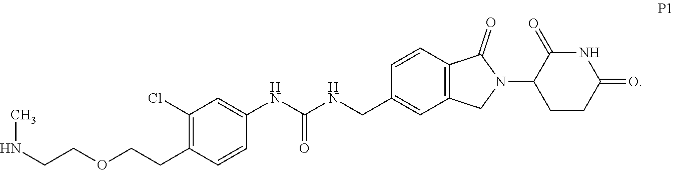 [SMILES: CNCCOCCc1ccc(NC(=O)NCc2ccc3c(c2)CN(C2CCC(=O)NC2=O)C3=O)cc1Cl]

In some aspects, the neoDegrader of formula (II) is

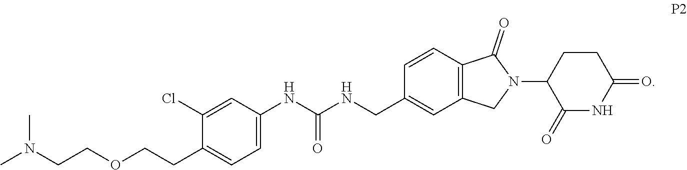 [SMILES: CN(C)CCOCCc1ccc(NC(=O)NCc2ccc3c(c2)CN(C2CCC(=O)NC2=O)C3=O)cc1Cl]

In some aspects, the neoDegrader of formula (II) is

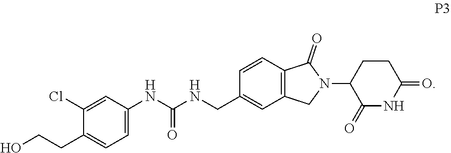 [SMILES: O=C1CCC(N2Cc3cc(CNC(=O)Nc4ccc(CCO)c(Cl)c4)ccc3C2=O)C(=O)N1]

In some aspects, the neoDegrader of formula (II) is

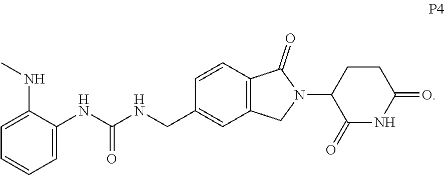 [SMILES: CNc1ccccc1NC(=O)NCc1ccc2c(c1)CN(C1CCC(=O)NC1=O)C2=O]

In some aspects, the neoDegrader of formula (II) is

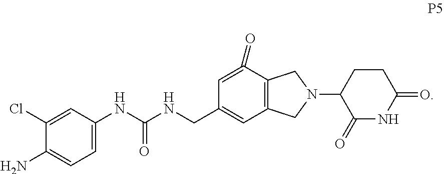

In some aspects, the neoDegrader of formula (II) is

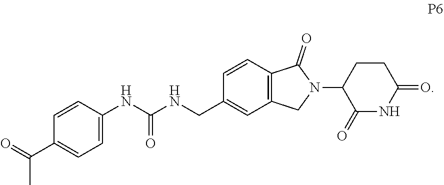 [SMILES: CC(=O)c1ccc(NC(=O)NCc2ccc3c(c2)CN(C2CCC(=O)NC2=O)C3=O)cc1]

In some aspects, the neoDegrader of formula (II) is

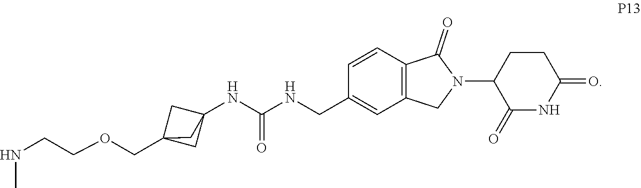 [SMILES: CNCCOCC12CC(NC(=O)NCc3ccc4c(c3)CN(C3CCC(=O)NC3=O)C4=O)(C1)C2]

In some aspects, the neoDegrader of formula (II) is

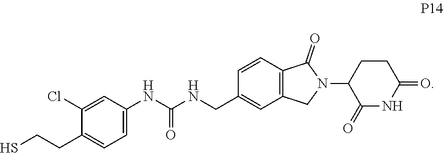 [SMILES: O=C1CCC(N2Cc3cc(CNC(=O)Nc4ccc(CCS)c(Cl)c4)ccc3C2=O)C(=O)N1]

In some aspects, the present disclosure provides neoDegraders of formula (II), or pharmaceutically acceptable salts thereof, wherein A is phenyl; U is NH; R¹ is halo; and R² is —(CH₂)ₙQ′(CH₂)ₘN(R⁴)₂, wherein m and n are 2, Q′ is O, one R⁴ is hydrogen and the other is methyl.

In some aspects, the present disclosure provides neoDegraders of formula (II), wherein A is phenyl; U is NH; R¹ is halo; and R² is —(CH₂)ₙQ′(CH₂)ₘN(R⁴)₂, wherein m and n are 2, Q′ is O, and each R⁴ is methyl.

In some aspects, the present disclosure provides neoDegraders of formula (II), wherein A is phenyl; U is NH; R¹ is halo; and R² is —(CH₂)ₙOH, wherein n is 2.

In some aspects, the present disclosure provides neoDegraders of formula (II), wherein A is phenyl; U is NH; R¹ is halo; and R² is —(CH₂)ₙSH, wherein n is 2.

In some aspects, the present disclosure provides neoDegraders of formula (II), wherein A is phenyl; U is NH; R¹ is hydrogen; and R² is —N(R⁴)₂, wherein one R⁴ is hydrogen and the other is methyl.

In some aspects, the present disclosure provides neoDegraders of formula (II), wherein A is phenyl; U is NH; R¹ is halo; and R² is —N(R⁴)₂, wherein each R⁴ is hydrogen. In some aspects, the present disclosure provides neoDegraders of formula (II), wherein A is phenyl; R¹ is hydrogen; and R²—C(O)R³, wherein R³ is methyl.

In some aspects, the present disclosure provides neoDegraders of formula (II), wherein A is a C₄-C₁₀cycloalkyl ring; U is NH; R¹ is hydrogen; and R² is —(CH₂)ₙQ′(CH₂)ₘN(R⁴)₂, wherein m and n are 2, Q′ is O, one R⁴ is hydrogen and the other is methyl.

In some aspects, a neoDegrader is a molecule that forms a ternary complex with an E3 ubiquitin ligase which is capable of targeting a protein for degradation.

### III. NeoDegrader Conjugates

The present disclosure provides conjugates of one or more neoDegraders disclosed herein and an anti-CD33 antibody or antigen-binding portion thereof disclosed herein. These conjugates can degrade proteins by binding to cereblon (CRBN), promoting recruitment and ubiquitination of substrate proteins mediated by CRL4^{CRBN} E3 ubiquitin ligase. These agents act as “molecular glues,” filling the binding interface as a hydrophobic patch that reprograms protein interactions between the ligase and neosubstrates.

In some aspects, the present disclosure provides a compound of formula (I),

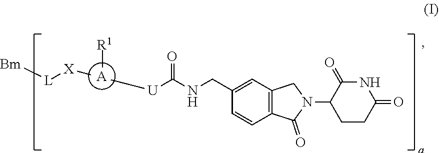

or a pharmaceutically acceptable salt thereof, wherein:
a is an integer from 1 to 10;
A is phenyl or a C₄-C₁₀cycloalkyl ring;
R¹ is selected from hydrogen and halo;
U is selected from NH and CF₂;
X is selected from —NR²—, ═C(CH₃)—, -Q-(CH₂)ₙ—, and -Q(CH₂)ₘQ′(CH₂)ₙ—; wherein:
   Q and Q′ are each independently O, S, or NR²;
   R² is hydrogen or C₁-C₆alkyl;
   n is an integer from 1 to 6;
   m is an integer from 2 to 6;
wherein the left side of each group is attached to L and the right side is attached to A;
provided that when X is NH or -Q-(CH₂)ₙ—, R¹ is halo;
L is a cleavable linker or non-cleavable linker; and
Bm is an anti-CD33 antibody or antigen-binding portion thereof disclosed herein.

In some aspects, U is NH.

In some aspects, the neoDegrader conjugate described herein has in vitro anti-proliferative activity against a tumor cell line. In some aspects, the neoDegrader conjugate comprising a neoDegrader and an anti-CD33 antibody or antigen-binding portion thereof disclosed herein has in vitro anti-proliferative activity at least about 50%, at least about 60%, at least about 70%, at least about 80%, at least about 90%, at least about 95%, or at least about 100% higher than the neoDegrader alone or the anti-CD33 antibody or antigen-binding portion thereof disclosed herein alone. In some aspects, the neoDegrader conjugate comprising a neoDegrader and an anti-CD33 antibody or antigen-binding portion thereof disclosed herein has in vitro anti-proliferative activity at least about 2 fold, at least about 3 fold, at least about 4 fold, at least about 5 fold, at least about 6 fold, at least about 7 fold, at least about 8 fold, at least about 9 fold, at least about 10 fold higher than the neoDegrader alone or the anti-CD33 antibody or antigen-binding portion thereof disclosed herein alone.

In some aspects, the neoDegrader conjugates described herein have in vitro anti-proliferative activity against a Daudi lymphoma cell line, e.g., higher anti-proliferative activity against a Daudi lymphoma cell line, compared to the neoDegrader alone or the anti-CD33 antibody or antigen-binding portion thereof alone. In some aspects the neoDegrader conjugates described herein have in vitro anti-proliferative activity against the HL-60 acute myeloid leukemia cell line, e.g., higher anti-proliferative activity against a HL-60 acute myeloid leukemia cell line, compared to the neoDegrader alone or the anti-CD33 antibody or antigen-binding portion thereof alone. In some aspects, the neoDegrader conjugates described herein have in vitro anti-proliferative activity against a Ramos non-Hodgkins lymphoma cell line, e.g., higher anti-proliferative activity against a Ramos non-Hodgkins lymphoma cell line, compared to the neoDegrader alone or the anti-CD33 antibody or antigen-binding portion thereof alone. In some aspects the neoDegrader conjugates described herein is capable of maintaining their anti-proliferative activity in the presence of human serum. The neoDegrader conjugates described herein can be used in the treatment of cancers.

In some aspects, an antibody neoDegrader conjugate (AnDC) is a conjugate of one or more neoDegraders disclosed herein and an anti-CD33 antibody or antigen-binding portion thereof disclosed herein.

### III.A. Linker

The neoDegrader of the present disclosure can be linked to the anti-CD33 antibody or antigen-binding portion thereof via a linker. As used herein, the term “linker” refers to any chemical moiety capable of connecting the anti-CD33 antibody or antigen-binding portion thereof (Bm) to group X within the compounds of formula (I).

In certain aspects, the linker can contain a heterobifunctional group. In the present disclosure, the term “heterobifunctional group” refers to a chemical moiety that connects the linker of which it is a part to the anti-CD33 antibody or antigen-binding portion thereof. Heterobifunctional groups are characterized as having different reactive groups at either end of the chemical moiety. Attachment to “Bm,” can be accomplished through chemical or enzymatic conjugation, or a combination of both. Chemical conjugation involves the controlled reaction of accessible amino acid residues on the surface of the anti-CD33 antibody or antigen-binding portion thereof with a reaction handle on the heterobifunctional group. Examples of chemical conjugation include, but are not limited to, lysine amide coupling, cysteine coupling, and coupling via a non-natural amino acid incorporated by genetic engineering, wherein non-natural amino acid residues with a desired reaction handle are installed onto “Bm.” In enzymatic conjugation, an enzyme mediates the coupling of the linker with an accessible amino residue on the anti-CD33 antibody or antigen-binding portion thereof. Examples of enzymatic conjugation include, but are not limited to, transpeptidation using sortase, transpeptidation using microbial transglutaminase, and N-glycan engineering. Chemical conjugation and enzymatic conjugation may also be used sequentially. For example, enzymatic conjugation can also be used for installing unique reaction handles on “Bm” to be utilized in subsequent chemical conjugation.

In some aspects, the heterobifunctional group is selected from:

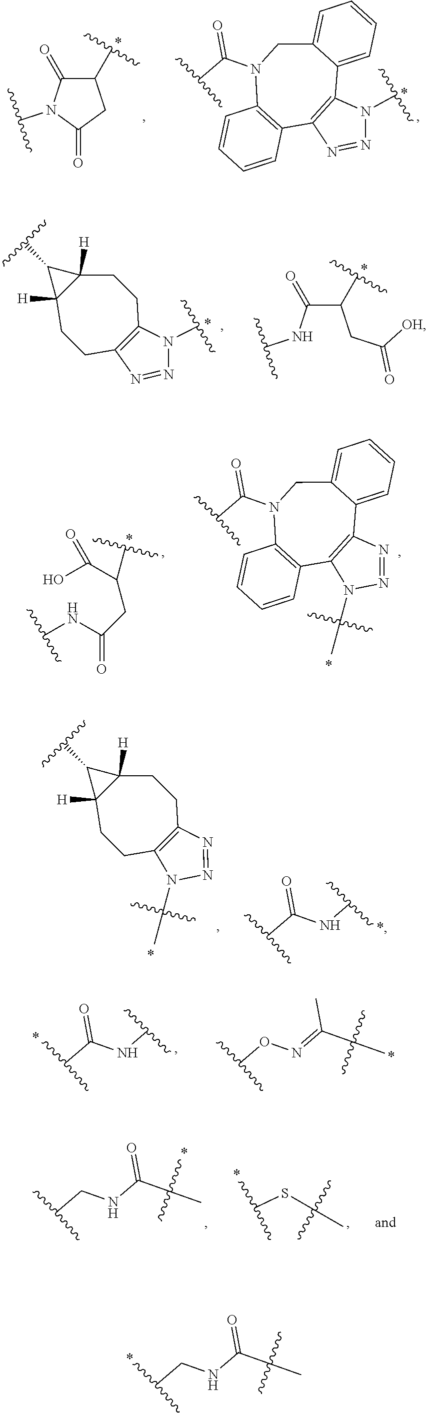

wherein
[symbol] is the point of attachment to the remaining portion of the linker; and
[symbol] the point of attachment to Bm.

In certain aspects, linker “L” is non-cleavable. As used here, the term “non-cleavable linker” is any chemical moiety that is capable of linking the anti-CD33 antibody or antigen-binding portion thereof to the neoDegrader in a stable, covalent manner and does not fall under the categories defined herein as “cleavable linkers”. Thus, non-cleavable linkers are substantially resistant to acid-induced cleavage, light-induced cleavage, bioreductive cleavage, peptidase-induced cleavage, esterase-induced cleavage, and disulfide bond cleavage. “Substantially resistant to cleavage” means that the chemical bond in the linker or adjoining the linker in at least 80%, preferably at least 85%, more preferably at least 90%, even more preferably at least 95%, and most preferably at least 99% of the antibody neoDegrader conjugate population remains non-cleavable by an acid, a photolabile-cleaving agent, a bioreductive agent, a peptidase, an esterase, or a chemical or a physiological compound that cleaves the chemical bond (for example, a disulfide bond) in a cleavable linker, for within a few hours to several days of treatment with any of the agents described above. In certain aspects the linker is not susceptible to acid-induced cleavage, photo-induced cleavage, bioreductive cleavage, enzymatic cleavage, or the like, at conditions under which the neoDegrader and/or anti-CD33 antibody or antigen-binding portion thereof can remain active. ADC catabolites generated from non-cleavable linkers contain a residual amino acid from the antibody. These catabolites can exert unique and unexpected properties in the target cells to which they are delivered.

A person of ordinary skill in the art would readily distinguish non-cleavable from cleavable linkers.

Examples of non-cleavable linkers include, but are not limited to, SMCC (succinimidyl 4-(N-maleimidomethyl)cyclohexane-1-carboxylate) linkers, succinimide thioether linkers, and linkers such as:

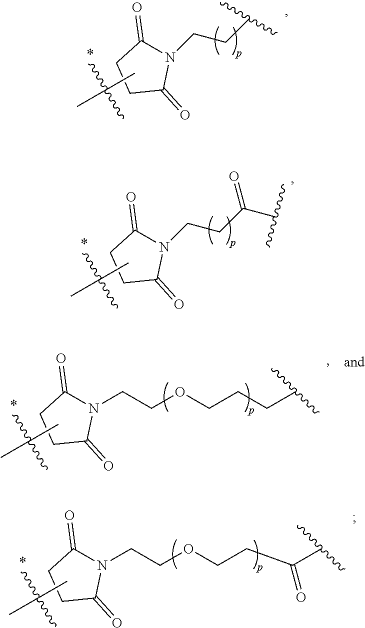

wherein:
p is an integer from 1 to 10;
[symbol] is the point of attachment to X; and
[symbol] is the point of attachment to the anti-CD33 antibody or antigen-binding portion thereof.

In some aspects, the linker is:

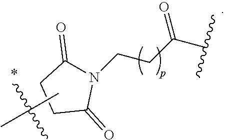

In some aspects, p is 5.

In certain aspects the linker can be cleavable. In some aspects, the linker can be susceptible to acid-induced cleavage, photo-induced cleavage, bioreductive cleavage, enzymatic cleavage, or the like, at conditions under which the neoDegrader and/or anti-CD33 antibody or antigen-binding portion thereof can remain active.

In some aspects, the cleavable linker can be cleaved enzymatically. In some aspects, the cleavable linker can be cleaved by a protease, peptidase, esterase, beta-gluroronidase, glycosidase, phosphodiesterase, phosphatase, pyrophosphatase, or lipase.

In some aspects, the cleavable linker can be cleaved by a protease. Examples of proteases include, but are not limited to, cathepsin B, VAGP tetrapeptide, and the like.

In certain aspects, the cleavable linker contains a peptide. In some aspects, the peptide is the site of cleavage of the linker, thereby facilitating release of the drug upon exposure to intracellular proteases, such as lysosomal enzymes. Peptides can be designed and optimized for enzymatic cleavage by a particular enzyme, for example, a tumor-associated protease, cathepsin B, C and D, or a plasmin protease. Examples of peptides having two amino acids include, but are not limited to, alanine-alanine (ala-ala), valine-alanine (val-ala), valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Examples of peptides having three amino acids include, but are not limited to, glycine-valine-citrulline (gly-val-cit), aspartic acid-valine-citrulline (asp-val-cit), alanine-alanine-asparagine (ala-ala-asn), alanine-phenylalanine-lysine (ala-phe-lys), glycine-glycine-phenylalanine (gly-gly-phe), and glycine-glycine-glycine (gly-gly-gly). Examples of peptides having four amino acids include, but are not limited to, glycine-glycine-valine-citrulline (gly-gly-val-cit) and glycine-glycine-phenylalanine-glycine (gly-gly-phe-gly). The amino acid combinations above can also be present in the reverse order (i.e., cit-val).

The peptides of the present disclosure can comprise L- or D-isomers of amino acid residues. The term “naturally-occurring amino acid” refers to Ala, Asp, Asx, Cit, Cys, Glu, Phe, Glx, Gly, His, Ile, Lys, Leu, Met, Asn, Pro, Gln, Arg, Ser, Thr, Val, Trp, and Tyr. “D-” designates an amino acid having the “D” (dextrorotary) configuration, as opposed to the configuration in the naturally occurring (“L-”) amino acids. The amino acids described herein can be purchased commercially (Sigma Chemical Co., Advanced Chemtech) or synthesized using methods known in the art.

In certain aspects, the linker (“L”) is a protease cleavable linker selected from

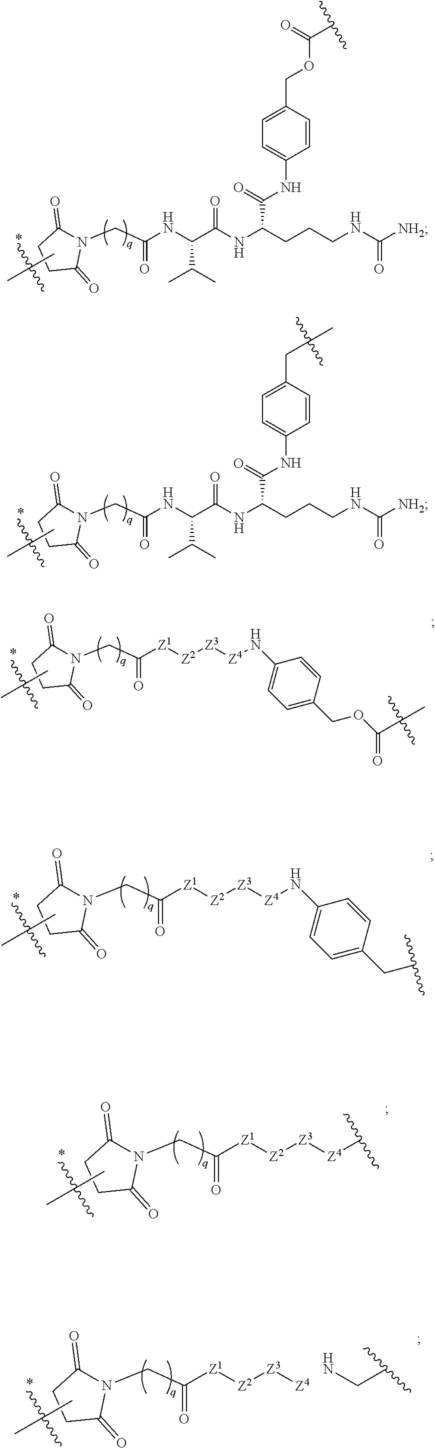

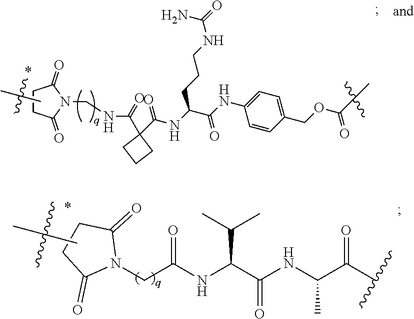

wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues;
[symbol] is the point of attachment to X; and
[symbol] is the point of attachment to the anti-CD33 antibody or antigen-binding portion thereof.

In certain aspects, Z¹, Z², Z³, and Z⁴ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

In some aspects, Z¹ is absent or glycine; Z² is absent or selected from L-glutamine, D-glutamine, L-glutamic acid, D-glutamic acid, L-aspartic acid, D-aspartic acid, L-alanine, D-alanine, and glycine; Z³ is selected from L-valine, D-valine, L-alanine, D-alanine, L-phenylalanine, D-phenylalanine, and glycine; and Z⁴ is selected from L-alanine, D-alanine, L-citrulline, D-citrulline, L-asparagine, D-asparagine, L-lysine, D-lysine, L-phenylalamine, D-phenylalanine, and glycine.

In some aspects, L is

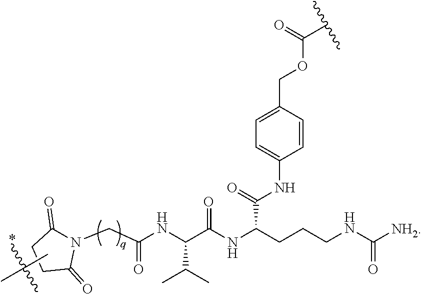

In some aspects, q is 5.

In certain aspects, L is a pyrophosphatase cleavable linker.

In some aspects, L is a pyrophosphatase cleavable linker which is:

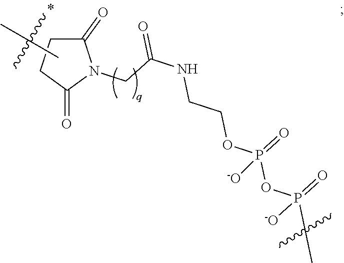

wherein:
q is an integer from 2 to 10;
[symbol] is the point of attachment to X; and
[symbol] is the point of attachment to the anti-CD33 antibody or antigen-binding portion thereof.

In certain aspects, L is a beta-glucoronidase cleavable linker.

In some aspects, L is a beta-glucoronidase cleavable linker selected from:

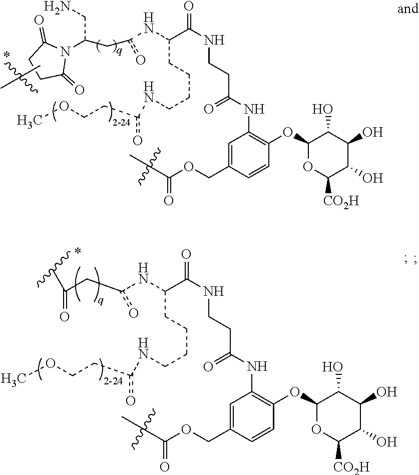

wherein:
q is an integer from 2 to 10;
---- is absent or a bond;
[symbol] is the point of attachment to X; and
[symbol] is the point of attachment to the anti-CD33 antibody or antigen-binding portion thereof.

In some aspects, the linker is bioreducible. Bioreducible linkers take advantage of the difference in reduction potential in the intracellular compartment versus plasma. Reduced glutathione presented in tumor cells' cytoplasma is up to 1000-fold higher than that present in normal cells' cytoplasma, and the tumor cells also contain enzymes which can contribute to reduction in cellular compartments. The linkers keep conjugates intact during systemic circulation, and are selectively cleaved by the high intracellular concentration of glutathione, releasing the active drugs at the tumor sites from the non-toxic prodrugs.

In some aspects, L is a bioreducible linker selected from:

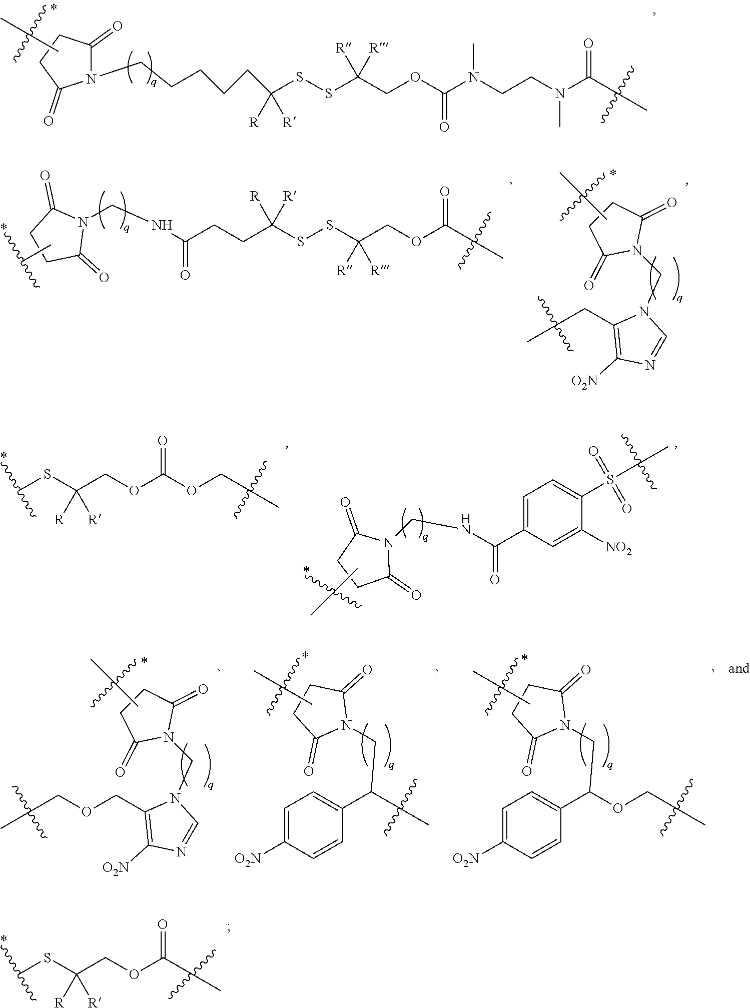

wherein:
q is an integer from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;
[symbol] is the point of attachment to X; and
[symbol] is the point of attachment to the anti-CD33 antibody or antigen-binding portion thereof.

In certain aspects, the linker is acid cleavable. Acid-cleavable linkers are specifically designed to remain stable at the neutral pH of blood circulation, but undergo hydrolysis and release the cytotoxic drug in the acidic environment of the cellular compartments.

In some aspects, L is an acid cleavable linker selected from

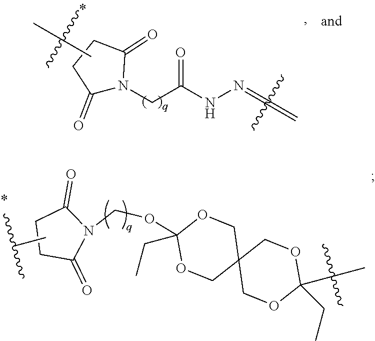

wherein:
q is an integer from 2 to 10;
[symbol] is the point of attachment to X; and
[symbol] is the point of attachment to the anti-CD33 antibody or antigen-binding portion thereof.

In certain aspects, L is wherein L is a click-to-release linker, where release of the neoDegrader is chemically triggered by a tetrazine or related compound.

In some aspects, L is a click-to-release linker selected from

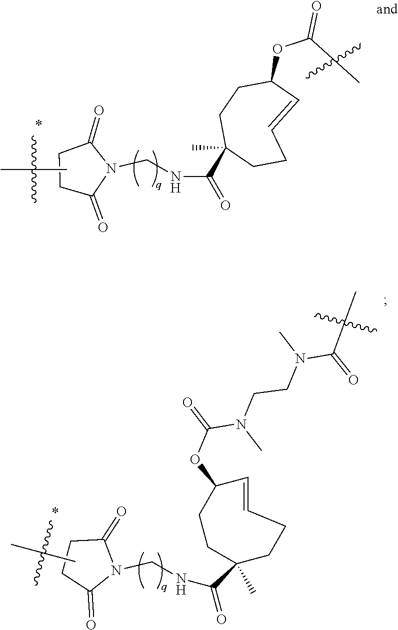

wherein:
q is an integer from 2 to 10;
[symbol] is the point of attachment to X; and
[symbol] is the point of attachment to the anti-CD33 antibody or antigen-binding portion thereof.

### III.B. Anti-CD33 Antibody

The present disclosure provides neoDegraders conjugated to an anti-CD33 antibody or antigen-binding portion thereof.

CD33 is a transmembrane receptor expressed on both myeloid and lymphoid cells. It binds sialic acid, and therefore, is a member of the sialic acid-binding immunoglobulin-type lectin (SIGLEC) family. CD33 plays a role in mediating cell-cell interactions and in maintaining immune cells in a resting state. Upon binding, the immunoreceptor tyrosine-based inhibition motif (ITIM) of CD33, present on the cytosolic portion of the protein, is phosphorylated and acts as a docking site for Src homology 2 (SH2) domain-containing proteins like SHP phosphatases. This can result in a cascade that inhibits phagocytosis in the cell. Structurally, the extracellular portion of CD33 contains two immunoglobulin domains, and the intracellular portion contains the ITIM. Synonyms of CD33 include, but are not limited to, sialic acid binding Ig-like lectin 3, SIGLEC3, SIGLEC-3, gp67, and p67.

The canonical amino acid sequence for human CD33 and known isoforms are shown in Table 1 (UniProtKB—P20138; SEQ ID NOs: 13-18).

TABLE 1
Human CD33 Amino Acid Sequences

| | |
|---|---|
| Canonical | MPLLLLLPLLWAGALAMDPNFWLQVQESVTVQEGLCVLVPCTFFHPIPYYDKNSPVHGYWFRE |
| CD33 | GAIISRDSPVATNKLDQEVQEETQGRFRLLGDPSRNNCSLSIVDARRRDNGSYFFRMERGSTK |
| | YSYKSPQLSVHVTDLTHRPKILIPGTLEPGHSKNLTCSVSWACEQGTPPIFSWLSAAPTSLGP |
| | RTTHSSVLIITPRPQDHGTNLTCQVKFAGAGVTTERTIQLNVTYVPQNPTTGIFPGDGSGKQE |
| | TRAGVVHGAIGGAGVTALLALCLCLIFFIVKTHRRKAARTAVGRNDTHPTTGSASPKHQKKSK |
| | LHGPTETSSCSGAAPTVEMDEELHYASLNFHGMNPSKDTSTEYSEVRTQ |
| | (SEQ ID NO: 13) |
| CD33 Human | MPLLLLLPLLWADLTHRPKILIPGTLEPGHSKNLTCSVSWACEQGTPPIFSWLSAAPTSLGPR |
| Isoform 2 | TTHSSVLIITPRPQDHGTNLTCQVKFAGAGVTTERTIQLNVTYVPQNPTTGIFPGDGSGKQET |
| | RAGVVHGAIGGAGVTALLALCLCLIFFIVKTHRRKAARTAVGRNDTHPTTGSASPKHQKKSKL |
| | HGPTETSSCSGAAPTVEMDEELHYASLNFHGMNPSKDTSTEYSEVRTQ (SEQ ID NO: 14) |
| CD33 Human | MPLLLLLPLLWAGALAMDPNFWLQVQESVTVQEGLCVLVPCTFFHPIPYYDKNSPVHGYWFRE |
| Isoform 3 | GAIISRDSPVATNKLDQEVQEETQGRFRLLGDPSRNNCSLSIVDARRRDNGSYFFRMERGSTK |
| | YSYKSPQLSVHVTDLTHRPKILIPGTLEPGHSKNLTCSVSWACEQGTPPIFSWLSAAPTSLGP |
| | RTTHSSVLIITPRPQDHGTNLTCQVKFAGAGVTTERTIQLNVTYVPQNPTTGIFPGDGSGKQE |
| | TRAGVVHGAIGGAGVTALLALCLCLIFFIVKTHRRKAARTAVGRNDTHPTTGSASPVR (SEQ |
| | ID NO: 15) |
| CD33 Human | MDLGEAATRARPAVISPGVNSCAQKSTSEWKDFRHGVRMSQMALKEALEAASSDMPLLLLLPL |
| Isoform X1 | LWAGALAMDPNFWLQVQESVTVQEGLCVLVPCTFFHPIPYYDKNSPVHGYWFREGAIISRDSP |
| | VATNKLDQEVQEETQGRFRLLGDPSRNNCSLSIVDARRRDNGSYFFRMERGSTKYSYKSPQLS |
| | VHVTDLTHRPKILIPGTLEPGHSKNLTCSVSWACEQGTPPIFSWLSAAPTSLGPRTTHSSVLI |
| | ITPRPQDHGTNLTCQVKFAGAGVTTERTIQLNVTYVPQNPTTGIFPGDGSGKQETRAGVVHGA |
| | IGGAGVTALLALCLCLIFFIVKTHRRKAARTAVGRNDTHPTTGSASPKHQKKSKLHGPTETSS |
| | CSGAAPTVEMDEELHYASLNFHGMNPSKDTSTEYSEVRTQ (SEQ ID NO: 16) |
| CD33 Human | MDLGEAATRARPAVISPGVNSCAQKSTSEWKDFRHGVRMSQMALKEALEAASSDMPLLLLLPL |
| Isoform X2 | LWAGALAMDPNFWLQVQESVTVQEGLCVLVPCTFFHPIPYYDKNSPVHGYWFREGAIISRDSP |
| | VATNKLDQEVQEETQGRFRLLGDPSRNNCSLSIVDARRRDNGSYFFRMERGSTKYSYKSPQLS |
| | VHVTDLTHRPKILIPGTLEPGHSKNLTCSVSWACEQGTPPIFSWLSAAPTSLGPRTTHSSVLI |
| | ITPRPQDHGTNLTCQVKFAGAGVTTERTIQLNVTWKQETRAGVVHGAIGGAGVTALLALCLCL |
| | IFFIVKTHRRKAARTAVGRNDTHPTTGSASPKHQKKSKLHGPTETSSCSGAAPTVEMDEELHY |
| | ASLNFHGMNPSKDTSTEYSEVRTQ (SEQ ID NO: 17) |
| CD33 Human | MDLGEAATRARPAVISPGVNSCAQKSTSEWKDFRHGVRMSQMALKEALEAASSDMPLLLLLPL |
| Isoform X4 | LWADLTHRPKILIPGTLEPGHSKNLTCSVSWACEQGTPPIFSWLSAAPTSLGPRTTHSSVLII |
| | TPRPQDHGTNLTCQVKFAGAGVTTERTIQLNVTYVPQNPTTGIFPGDGSGKQETRAGVVHGAI |
| | GGAGVTALLALCLCLIFFIVKTHRRKAARTAVGRNDTHPTTGSASPKHQKKSKLHGPTETSSC |
| | SGAAPTVEMDEELHYASLNFHGMNPSKDTSTEYSEVRTQ (SEQ ID NO: 18) |

CD33 is expressed in approximately 90% of acute myeloid leukemia (AML) cases and has demonstrated utility as a target of therapeutic antibodies. High CD33 expression on AML blasts has been reported approximately three decades ago. CD33 was detected on blasts of 8590 of patients presenting with AML as well as on normal myeloid progenitors and myelocytes. CD33 is restricted to hematopoietic cells, but absent on normal hematopoietic stem cells, making it an ideal target for AML therapy.

Anti-CD33 antibodies for the conjugates of the present disclosure are capable of specifically binding to CD33. In some aspects, anti-CD33 antibodies described herein bind to human CD33 with high affinity, for example, with a K_{D} of 10⁻⁶ M or less, 10⁻⁷ M or less, 10⁻⁸ M or less, 10⁻⁹ M or less, 10⁻¹⁰ M or less, 10⁻¹¹ M or less, 10⁻¹²M or less, 10⁻¹²M to 10⁻⁷ M, 10⁻¹¹M to 10⁻⁷M, 10⁻¹⁰ M to 10⁻⁷ M, or 10⁻⁹ M to 10⁻⁷ M.

In some aspects, the anti-CD33 antibody comprises a heavy chain and a light chain, wherein the heavy chain comprises a heavy chain variable region (VH) and the light chain comprises a light chain variable region (VL); wherein the VH comprises a VH complementarity determining region (CDR) 1 (VH-CDR1), a VH-CDR2, and a VH-CDR3 and the VL comprises a VL-CDR1, a VL-CDR2, and a VL-CDR3; wherein the VH-CDR3 comprises an amino acid sequence having at least about 90%, at least about 95%, at least about 96%, at least about 97%, at least about 98%, or at least about 99% sequence identity to SEQ ID NO: 3. In some aspects, the anti-CD33 antibody comprises a VH-CDR2 comprising an amino acid sequence with at least about 90%, at least about 95%, at least about 96%, at least about 97%, at least about 98%, or at least about 99% sequence identity to an amino acid sequence of SEQ ID NO: 2. In some aspects, the anti-CD33 antibody comprises a VH-CDR1 comprising an amino acid sequence with at least about 90%, at least about 95%, at least about 96%, at least about 97%, at least about 98%, or at least about 99% sequence identity to an amino acid sequence of SEQ ID NO: 1. In some aspects, the anti-CD33 antibody comprises a VL-CDR1 comprising an amino acid sequence with at least about 90%, at least about 95%, at least about 96%, at least about 97%, at least about 98%, or at least about 99% sequence identity to an amino acid sequence of SEQ ID NO: 5. In some aspects, the anti-CD33 antibody comprises a VL-CDR2 comprising an amino acid sequence with at least about 90%, at least about 95%, at least about 96%, at least about 97%, at least about 98%, or at least about 99% sequence identity to an amino acid sequence of SEQ ID NO: 6. In some aspects, the anti-CD33 antibody comprises a VL-CDR3 comprising an amino acid sequence with at least about 90%, at least about 95%, at least about 96%, at least about 97%, at least about 98%, or at least about 99% sequence identity to an amino acid sequence of SEQ ID NO: 7. In some aspects, the CDRs comprises the sequences shown in Table 2 below.

TABLE 2
CD33AB and Gemtuzumab CDR Sequences and Variable Region Sequences

| | |
|---|---|
| VH-CDR1 | DSNIH |
| (SEQ ID NO: | |
| 1) | |
| VH-CDR2 | YIYPYNGGTDYNQKFKN |
| (SEQ ID NO: | |
| 2) | |
| VH-CDR3 | GNPWLAY |
| (SEQ ID NO: | |
| 3) | |
| VH (SEQ ID | EVQLVQSGAEVKKPGSSVKVSCKASGYTITDSNIHWVRQAPGQSLEWI |
| NO: 4) | GYIYPYNGGTDYNQKFKNRATLTVDNPTNTAYMELSSLRSEDTAFYY |
| | CVNGNPWLAYWGQGTLVTVSS |
| VL-CDR1 | RASESLDNYGIRFLT |
| (SEQ ID NO: | |
| 5) | |
| VL-CDR2 | AASNQGSG |
| (SEQ ID NO: | |
| 6) | |
| VL-CDR3 | QQTKEVPWS |
| (SEQ ID NO: | |
| 7) | |
| VL (SEQ ID | DIQLTQSPSTLSASVGDRVTITCRASESLDNYGIRFLTWFQQKPGKAPK |
| NO: 8) | LLMYAASNQGSGVPSRFSGSGSGTEFTLTISSLQPDDFATYYCQQTKEV |
| | PWSFGQGTKVEVK |

In some aspects, the anti-CD33 antibody heavy chain variable region comprises an amino acid sequence with at least about 80%, at least about 85%, at least about 90%, at least about 95% at least about 96%, at least about 97% at least about 98%, or at least about 99% sequence identity to an amino acid sequence of SEQ ID NO: 4. In some aspects, the anti-CD33 antibody light chain variable region comprises an amino acid sequence with at least about 80%, at least about 85%, at least about 90%, at least about 95%, at least about 96%, at least about 970%, at least about 98%, or at least about 99% sequence identity to an amino acid sequence of SEQ ID NO: 8.

In some aspects, the anti-CD33 antibody comprises a heavy chain variable region comprising a sequence with at least about 80%, at least about 85%, at least about 90%, at least about 95% at least about 96%, at least about 97% at least about 98%, or at least about 99% sequence identity to an amino acid sequence of SEQ ID NO: 4, and a light chain variable region comprising a sequence with at least about 80%, at least about 85%, at least about 90%, at least about 95%, at least about 96%, at least about 97%, at least about 98%, or at least about 99% sequence identity to an amino acid sequence of SEQ ID NO: 8.

In some aspects, the anti-CD33 antibody heavy chain comprises an amino acid sequence having at least about 80%, at least about 85%, at least about 90%, at least about 95%, at least about 96%, at least about 97%, at least about 98%, or at least about 99% sequence identity to SEQ ID NO: 9 or SEQ ID NO: 11. In some aspects, the anti-CD33 antibody comprises a light chain comprising an amino acid sequence having at least about 80%, at least about 85%, at least about 90%, at least about 95%, at least about 96%, at least about 97%, at least about 98%, or at least about 99% sequence identity to SEQ ID NO: 10 or SEQ ID NO: 12.

TABLE 3
Anti-CD33 Antibody Amino Acid Sequences.

| | |
|---|---|
| CD33AB | EVQLVQSGAEVKKPGSSVKVSCKASGYTITDSNIHWVRQAPGQSLEWIGYIY |
| Heavy Chain | PYNGGTDYNQKFKNRATLTVDNPTNTAYMELSSLRSEDTAFYYCVNGNPWL |
| (SEQ ID NO: | AYWGQGTLVTVSSASTKGPSVFPLAPSSKSTSGGTAALGCLVKDYFPEPVTVS |
| 9) | WNSGALTSGVHTFPAVLQSSGLYSLSSVVTVPSSSLGTQTYICNVNHKPSNTK |
| | VDKKVEPKSCDKTHTCPPCPAPELLGGPSVFLFPPKPKDTLMISRTPEVTCVV |
| | VDVSHEDPEVKFNWYVDGVEVHNAKTKPREEQYASTYRVVSVLTVLHQDW |
| | LNGKEYKCKVSNKALPAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |
| CD33AB | DIQLTQSPSTLSASVGDRVTITCRASESLDNYGIRFLTWFQQKPGKAPKLLMY |
| Light Chain | AASNQGSGVPSRFSGSGSGTEFTLTISSLQPDDFATYYCQQTKEVPWSFGQGT |
| (SEQ ID NO: | KVEVKRTVAAPSVFIFPPSDEQLKSGTASVVCLLNNFYPREAKVQWKVDNAL |
| 10) | QSGNSQESVTEQDSKDSTYSLSSTLTLSKADYEKHKVYACEVTHQGLSSPVT |
| | KSFNRGEC |
| Gemtuzumab | EVQLVQSGAEVKKPGSSVKVSCKASGYTITDSNIHWVRQAPGQSLEWIGYIY |
| Heavy Chain | PYNGGTDYNQKFKNRATLTVDNPTNTAYMELSSLRSEDTAFYYCVNGNPWL |
| (SEQ ID NO: | AYWGQGTLVTVSSASTKGPSVFPLAPCSRSTSESTAALGCLVKDYFPEPVTVS |
| 11) | WNSGALTSGVHTFPAVLQSSGLYSLSSVVTVPSSSLGTKTYTCNVDHKPSNT |
| | KVDKRVESKYGPPCPPCPAPEFLGGPSVFLFPPKPKDTLMISRTPEVTCVVVD |
| | VSQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQEG |
| | NVFSCSVMHEALHNHYTQKSLSLSLGK |
| Gemtuzumab | DIQLTQSPSTLSASVGDRVTITCRASESLDNYGIRFLTWFQQKPGKAPKLLMY |
| Light Chain | AASNQGSGVPSRFSGSGSGTEFTLTISSLQPDDFATYYCQQTKEVPWSFGQGT |
| SEQ ID NO: | KVEVKRTVAAPSVFIFPPSDEQLKSGTASVVCLLNNFYPREAKVQWKVDNAL |
| 12) | QSGNSQESVTEQDSKDSTYSLSSTLTLSKADYEKHKVYACEVTHQGLSSPVT |
| | KSFNRGEC |

In some aspects, the anti-CD33 antibody comprises a heavy chain comprising an amino acid sequence having at least about 800%, at least about 85%, at least about 90%, at least about 95%, at least about 96%, at least about 97%, at least about 98%, or at least about 990% sequence identity to SEQ ID NO: 9 and a light chain comprising an amino acid sequence having at least about 80%, at least about 85%, at least about 90%, at least about 95% at least about 96%, at least about 97%, at least about 98%, or at least about 99% sequence identity to SEQ ID NO: 10.

In some aspects, the anti-CD33 antibody comprises a heavy chain comprising an amino acid sequence having at least about 80%, at least about 85%, at least about 90%, at least about 95%, at least about 96%, at least about 97%, at least about 98%, or at least about 99% sequence identity to SEQ ID NO: 11 and a light chain comprising an amino acid sequence having at least about 80%, at least about 85%, at least about 90%, at least about 95%, at least about 96%, at least about 97%, at least about 98%, or at least about 99% sequence identity to SEQ ID NO: 12.

In some aspects, the anti-CD33 antibody is disclosed in U.S. Pat. Nos. 5,585,089, 5,693,762, each of which are expressly incorporated herein by reference.

In some aspects, the anti-CD33 antibody comprises a heavy chain and a light chain, wherein the heavy chain comprises a heavy chain variable region (VH) and the light chain comprises a light chain variable region (VL); wherein the VH comprises a VH complementarity determining region (CDR) 1 (VH-CDR1), a VH-CDR2, and a VH-CDR3 and the VL comprises a VL-CDR1, a VL-CDR2, and a VL-CDR3; wherein the VH-CDR3 comprises an amino acid sequence having at least about 90%, at least about 95%, at least about 96%, at least about 97%, at least about 98%, or at least about 99% sequence identity to SEQ ID NO: 21. In some aspects, the anti-CD33 antibody comprises a VH-CDR2 comprising an amino acid sequence with at least about 90%, at least about 95%, at least about 96%, at least about 97%, at least about 98%, or at least about 99% sequence identity to an amino acid sequence of SEQ ID NO: 20. In some aspects, the anti-CD33 antibody comprises a VH-CDR1 comprising an amino acid sequence with at least about 90%, at least about 95%, at least about 96%, at least about 97%, at least about 98%, or at least about 99% sequence identity to an amino acid sequence of SEQ ID NO: 19. In some aspects, the anti-CD33 antibody comprises a VL-CDR1 comprising an amino acid sequence with at least about 90%, at least about 95%, at least about 96%, at least about 97%, at least about 98%, or at least about 99% sequence identity to an amino acid sequence of SEQ ID NO: 22. In some aspects, the anti-CD33 antibody comprises a VL-CDR2 comprising an amino acid sequence with at least about 90%, at least about 95%, at least about 96%, at least about 97%, at least about 98%, or at least about 99% sequence identity to an amino acid sequence of SEQ ID NO: 23. In some aspects, the anti-CD33 antibody comprises a VL-CDR3 comprising an amino acid sequence with at least about 90%, at least about 95%, at least about 96%, at least about 97%, at least about 98%, or at least about 99% sequence identity to an amino acid sequence of SEQ ID NO: 24. In some aspects, the CDRs comprises the sequences shown in Table 4 below.

TABLE 4
huMy9-6 and AB1 CDR Sequences and Variable Region Sequences

| | |
|---|---|
| VH-CDR1 | SYYIH |
| (SEQ ID NO: | |
| 19) | |
| VH-CDR2 | VIYPGNDDISYNQKFQG |
| (SEQ ID NO: | |
| 20) | |
| VH-CDR3 | EVRLRYFDV |
| (SEQ ID NO: | |
| 21) | |
| VH (SEQ ID | QVQLQQPGAEVVKPGASVKMSCKASGYTFTSYYIHWIKQTPGQGLEW |
| NO: 27) | VGVIYPGNDDISYNQKFQGKATLTADKSSTTAYMQLSSLTSEDSAVYY |
| | CAREVRLRYFDVWGQGTTVTVSS |
| VL-CDR1 | KSSQSVFFSSSQKNYLA |
| (SEQ ID NO: | |
| 22) | |
| VL-CDR2 | WASTRES |
| (SEQ ID NO: | |
| 23) | |
| VL-CDR3 | HQYLSSRT |
| (SEQ ID NO: | |
| 24) | |
| VL (SEQ ID | EIVLTQSPGSLAVSPGERVTMSCKSSQSVFFSSSQKNYLAWYQQIPGQS |
| NO: 28) | PRLLIYWASTRESGVPDRFTGSGSGTDFTLTISSVQPEDLAIYYCHQYLS |
| | SRTFGQGTKLEIK |

In some aspects, the anti-CD33 antibody heavy chain variable region comprises an amino acid sequence with at least about 80%, at least about 85%, at least about 90%, at least about 95% at least about 96%, at least about 97% at least about 98%, or at least about 99% sequence identity to an amino acid sequence of SEQ ID NO: 27. In some aspects, the anti-CD33 antibody light chain variable region comprises an amino acid sequence with at least about 80%, at least about 85%, at least about 90%, at least about 95%, at least about 96%, at least about 97%, at least about 98%, or at least about 99% sequence identity to an amino acid sequence of SEQ ID NO: 28.

In some aspects, the anti-CD33 antibody comprises a heavy chain variable region comprising a sequence with at least about 80%, at least about 85%, at least about 90%, at least about 95% at least about 96%, at least about 97% at least about 98%, or at least about 9900 sequence identity to an amino acid sequence of SEQ ID NO: 27, and a light chain variable region comprising a sequence with at least about 80%, at least about 85%, at least about 90%, at least about 95%, at least about 96%, at least about 97%, at least about 98%, or at least about 9900 sequence identity to an amino acid sequence of SEQ ID NO: 28.

In some aspects, the anti-CD33 antibody heavy chain comprises an amino acid sequence having at least about 80%, at least about 85%, at least about 90%, at least about 95%, at least about 96%, at least about 97%, at least about 98%, or at least about 99% sequence identity to SEQ ID NO: 25. In some aspects, the anti-CD33 antibody comprises a light chain comprising an amino acid sequence having at least about 80%, at least about 85%, at least about 90%, at least about 95%, at least about 96%, at least about 97%, at least about 98%, or at least about 99% sequence identity to 26.

TABLE 5
huMy9-6-IgG4-S228P (″“B1”) Anti-CD33 Antibody Amino Acid Sequences.

| | |
|---|---|
| Heavy Chain | QVQLQQPGAEVVKPGASVKMSCKASGYTFTSYYIHWIKQTPGQGLEWVGVI |
| (SEQ ID NO: | YPGNDDISYNQKFQGKATLTADKSSTTAYMQLSSLTSEDSAVYYCAREVRLR |
| 25) | YFDVWGQGTTVTVSSASTKGPSVFPLAPCSRSTSESTAALGCLVKDYFPEPVT |
| | VSWNSGALTSGVHTFPAVLQSSGLYSLSSVVTVPSSSLGTKTYTCNVDHKPSN |
| | TKVDKRVESKYGPPCPPCPAPEFLGGPSVFLFPPKPKDTLMISRTPEVTCVVVD |
| | VSQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQEG |
| | NVFSCSVMHEALHNHYTQKSLSLSLGK |
| Light Chain | EIVLTQSPGSLAVSPGERVTMSCKSSQSVFFSSSQKNYLAWYQQIPGQSPRLLI |
| (SEQ ID NO: | YWASTRESGVPDRFTGSGSGTDFTLTISSVQPEDLAIYYCHQYLSSRTFGQGT |
| 26) | KLEIKRTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGLSSPVT |
| | KSFNRGEC |

In some aspects, the anti-CD33 antibody comprises a heavy chain comprising an amino acid sequence having at least about 80%, at least about 85%, at least about 90%, at least about 95%, at least about 96%, at least about 97%, at least about 98%, or at least about 99% sequence identity to SEQ ID NO: 25 and a light chain comprising an amino acid sequence having at least about 80%, at least about 85%, at least about 90%, at least about 95%, at least about 96%, at least about 97%, at least about 98%, or at least about 99% sequence identity to SEQ ID NO: 26. The term “CD33AB” comprises a heavy chain as set forth in SEQ ID NO: 25 and a light chain as set forth in SEQ ID NO: 26.

Anti-CD33 antibodies include analogs and derivatives that are either modified, i.e., by the covalent attachment of any type of molecule as long as such covalent attachment permits the antibody to retain its antigen binding immunospecificity. For example, but not by way of limitation, the derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The anti-CD33 antibodies in neoDegrader conjugates can include antibodies having modifications (e.g., substitutions, deletions or additions) in amino acid residues that interact with Fc receptors. In particular, antibodies include antibodies having modifications in amino acid residues identified as involved in the interaction between the anti-Fc domain and the FcRn receptor. Antibodies immunospecific for a cancer cell antigen can be obtained commercially, for example, from Genentech (San Francisco, Calif.) or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, the literature publications, or by routine cloning and sequencing.

In certain aspects, the antibody of the neoDegrader conjugates can be a monoclonal antibody, e.g. a murine monoclonal antibody, a chimeric antibody, or a humanized antibody. In some aspects, the antibody can be an antibody fragment, e.g. a Fab fragment.

### IV. Compositions and Methods of Using

The conjugates and/or compounds described herein can be in the form of pharmaceutically or pharmaceutically acceptable salts. In some aspects, such salts are derived from inorganic or organic acids or bases.

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

Pharmaceutical compositions comprising the neoDegrader conjugates described herein may also comprise suitable carriers, excipients, and auxiliaries that may differ depending on the mode of administration.

In some aspects, the pharmaceutical compositions can be formulated as a suitable parenteral dosage form. Said formulations can be prepared by various methods known in the art. The pharmaceutical compositions can be administered directly into the bloodstream, into muscle, or directly into an organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle injectors, needle-free injectors, and infusion techniques.

Parenteral compositions are typically aqueous solutions which may contain excipients such as salts, carbohydrates and buffering agents. However, the composition may also be formulated a sterile non-aqueous solution or as a dried form to be used in conjunction with a suitable vehicle such as sterile pyrogen-free water.

The preparation of parenteral compositions under sterile conditions, for example, by lyophilization, can be readily accomplished using standard techniques known well to those of skill in the art.

Compositions for parenteral administration can be formulated to be immediate and/or modified release. Modified release formulations include delayed-, sustained-, pulsed-, controlled-, targeted, and programmed release. Thus, the compositions can be formulated as a solid, semi-solid, or thixotropic liquid for administration as an implanted depot providing modified release of the active agent.

The parenteral formulations can be admixed with other suitable pharmaceutically acceptable excipients used in parenteral dosage forms such as, but not limited to, preservatives.

In another aspect, the pharmaceutical compositions can be formulated as suitable oral dosage forms such as tablets, capsules, powders, pellets, suspensions, solutions, emulsions, and the like. Other suitable carriers can be present such as disintegrants, diluents, chelating agents, binders, glidants, lubricants, fillers, bulking agents, anti-adherants, and the like.

Oral dosage formulations may also contain other suitable pharmaceutical excipients such as sweeteners, vehicle/wetting agents, coloring agents, flavoring agents, preservatives, viscosity enhancing/thickening agents, and the like.

The neoDegrader or neoDegrader conjugates described herein can be used to treat various cancers. Certain conjugates of the present disclosure can be superior in terms of efficacy expression, pharmacokinetics (e.g., absorption, distribution, metabolism, excretion), solubility (e.g., water solubility), interaction with other medicaments (e.g., drug-metabolizing enzyme inhibitory action), safety (e.g., acute toxicity, chronic toxicity, genetic toxicity, reproductive toxicity, cardiotoxicity, carcinogenicity, central toxicity) and/or stability (e.g., chemical stability, stability to an enzyme), and can be useful as a medicament.

The neoDegrader or neoDegrader conjugates of the present disclosure can be used as medicaments such as an agents for the prophylaxis or treatment of diseases, for example, cancers e.g., colorectal cancers (e.g., colorectal cancer, rectal cancer, anus cancer, familial colorectal cancer, hereditary nonpolyposis colorectal cancer, gastrointestinal stromal tumor), lung cancers (e.g., non-small-cell lung cancer, small-cell lung cancer, malignant mesothelioma), mesothelioma, pancreatic cancers (e.g., pancreatic ductal carcinoma, pancreatic endocrine tumor), pharynx cancer, larynx cancer, esophageal cancer, stomach/gastric cancers (e.g., papillary adenocarcinoma, mucinous adenocarcinoma, adenosquamous carcinoma), duodenal cancer, small intestinal cancer, breast cancers (e.g., invasive ductal carcinoma, non-invasive ductal carcinoma, inflammatory breast cancer), ovarian cancers (e.g., ovarian epithelial cancer, extragonadal germ cell tumor, ovarian germ cell tumor, ovarian low-malignant potential tumor), testis tumor, prostate cancers (e.g., hormone-dependent prostate cancer, non-hormone dependent prostate cancer, castration-resistant prostate cancer), liver cancers (e.g., hepatocellular cancer, primary liver cancer, extrahepatic bile duct cancer), thyroid cancers (e.g., medullary thyroid carcinoma), renal cancers (e.g., renal cell cancers (e.g., clear cell renal cell cancer), transitional cell cancer of renal pelvis and ureter), uterine cancers (e.g., cervical cancer, uterine body cancer, uterus sarcoma), gestational choriocarcinoma, brain tumors (e.g., medulloblastoma, glioma, pineal astrocytic tumors, pilocytic astrocytoma, diffuse astrocytoma, anaplastic astrocytoma, pituitary adenoma), retinoblastoma, skin cancers (e.g., basalioma, malignant melanoma), sarcomas (e.g., rhabdomyosarcoma, leiomyosarcoma, soft tissue sarcoma, spindle cell sarcoma), malignant bone tumor, bladder cancer, hematological/blood cancers (e.g., multiple myeloma, leukemias (e.g., acute myelogenous leukemia), malignant lymphoma, Hodgkin's disease, chronic myeloproliferative disease), cancer of unknown primary; a cancer growth inhibitor; a cancer metastasis inhibitor; an apoptosis promoter; an agent for the treatment of precancerous lesions (e.g., myelodysplastic syndromes); and the like.

In certain aspects, neoDegraders or neoDegrader conjugates of the present disclosure can be used as a medicament for breast cancer, gastric cancer, ovarian cancer, uterine cancer, lung cancer, pancreatic cancer, liver cancer, lymphoma, or hematological cancers.

Furthermore, neoDegrader or neoDegrader conjugates of the present disclosure can be used concurrently with a non-drug therapy. To be precise, the conjugates can be combined with a non-drug therapy such as (1) surgery, (2) hypertensive chemotherapy using angiotensin II etc., (3) gene therapy, (4) thermotherapy, (5) cryotherapy, (6) laser cauterization and (7) radiotherapy.

For example, by using a neoDegrader or neoDegrader conjugate of the present disclosure before or after the above-mentioned surgery and the like, effects such as prevention of emergence of resistance, prolongation of Progression-Free Survival, prolongation of Disease-Free Survival, suppression of cancer metastasis or recurrence, prolongation of life and the like may be afforded.

In addition, it is possible to combine a treatment with a neoDegrader or neoDegrader conjugates of the present disclosure with a supportive therapy: (i) administration of antibiotic (e.g., β-lactam type such as pansporin and the like, macrolide type such as clarithromycin and the like) for the complication with various infectious diseases, (ii) administration of high-calorie transfusion, amino acid preparation or general vitamin preparation for the improvement of malnutrition, (iii) administration of morphine for pain mitigation, (iv) administration of a pharmaceutical agent for ameliorating side effects such as nausea, vomiting, anorexia, diarrhea, leucopenia, thrombocytopenia, decreased hemoglobin concentration, hair loss, hepatopathy, renopathy, DIC, fever and the like and (v) administration of a pharmaceutical agent for suppressing multiple drug resistance of cancer and the like.

In some aspects, the neoDegrader or neoDegrader conjugate of the disclosure can be used in combination with a standard of care therapy, e.g., one or more therapeutic agents (e.g., anti-cancer agents and/or immunomodulating agents). Accordingly, in certain aspects, a method of treating a tumor disclosed herein comprises administering the neoDegrader or neoDegrader conjugate of the disclosure in combination with one or more additional therapeutic agents. In some aspects, the neoDegrader or neoDegrader conjugate of the disclosure can be used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. A comprehensive and non-limiting list of combination treatment is disclosed in detail in the Combination Treatments section of this application.

In some aspects, the neoDegrader or neoDegrader conjugate of the disclosure is administered to the subject prior to or after the administration of the additional therapeutic agent. In other aspects, the neoDegrader or neoDegrader conjugate of the disclosure is administered to the subject concurrently with the additional therapeutic agent. In certain aspects, the neoDegrader or neoDegrader conjugate of the disclosure and the additional therapeutic agent can be administered concurrently as a single composition in a pharmaceutically acceptable carrier. In other aspects, the neoDegrader or neoDegrader conjugate of the disclosure and the additional therapeutic agent are administered concurrently as separate compositions.

In some aspects, a subject that can be treated with the neoDegrader or neoDegrader conjugate of the present disclosure is a nonhuman animal such as a rat or a mouse. In some aspects, the subject that can be treated is a human.

### V. Methods of Preparing NeoDegraders and Compositions

The present disclosure provides a method of preparing the neoDegrader conjugates, the process comprising reacting an anti-CD33 antibody or antigen-binding portion thereof with a compound of formula (I-1):

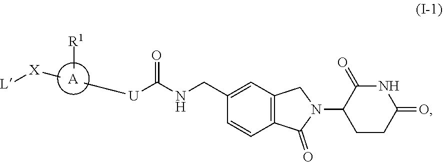

or a pharmaceutically acceptable salt thereof, wherein:
A is phenyl or a C₄-C₁₀cycloalkyl ring;
R¹ is independently selected from hydrogen and halo;
U is selected from NH and CF₂;
X is selected from —NR²—, ═C(CH₃)—, -Q-(CH₂)ₙ—, and -Q(CH₂)ₘQ′(CH₂)ₙ—; wherein
   Q and Q′ are each independently O, S, or NR²;
   R² is hydrogen or C₁-C₆alkyl;
   n is an integer from 1 to 6;
   m is an integer from 2 to 6; and
wherein the left side of each group is attached to L′ and the right side is attached to A;
provided that when X is NH or -Q-(CH₂)ₙ—, R¹ is halo;
L′ is a cleavable or non-cleavable linker precursor that conjugates to the anti-CD33 antibody or antigen-binding portion thereof.

As described herein, the linker precursor contain a heterobifunctional group that connects to the anti-CD33 antibody or antigen-binding portion thereof.

In some aspects, L′ is a non-cleavable linker precursor. In some aspects, L′ is selected from the group consisting of

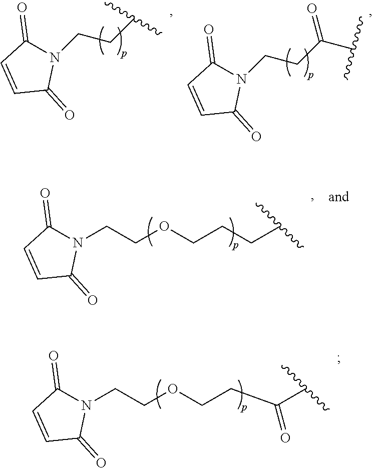

wherein:
p is an integer from 1 to 10; and
[symbol] is the point of attachment to X.

In some aspects, L′ is

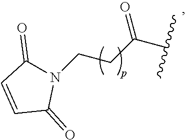

In some aspects, p is 5.

In certain aspects, L′ is a cleavable linker precursor.

In some aspects, the linker precursor is cleavable by a protease. In some aspects, the linker precursor is selected from the group consisting of

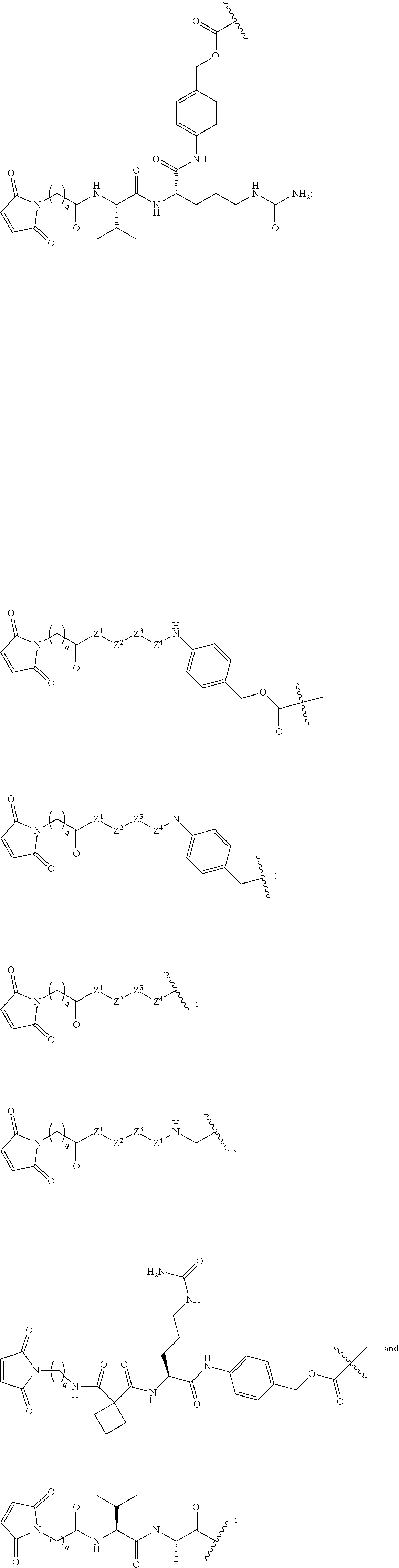

wherein:
q is an integer from 2 to 10;
Z¹, Z², Z³, and Z⁴ are each independently absent or a naturally-occurring amino acid residue in the L- or D-configuration, provided that at least two of Z¹, Z², Z³, and Z⁴ are amino acid residues; and
[symbol] is the point of attachment to X.

In some aspects, Z¹, Z², Z³, and Z⁴ are independently absent selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine, provided that at least two of Z¹, Z², Z³, and Z⁴ are amino acid residues.

In some aspects, Z¹ is absent or glycine; Z² is absent or selected from the group consisting of L-glutamine, D-glutamine, L-glutamic acid, D-glutamic acid, L-aspartic acid, D-aspartic acid, L-alanine, D-alanine, and glycine; Z³ is selected from the group consisting of L-valine, D-valine, L-alanine, D-alanine, L-phenylalanine, D-phenylalanine, and glycine; and Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

In some aspects, L′ is

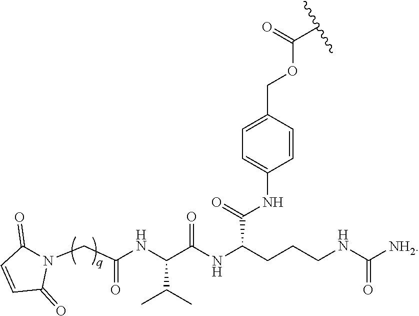

In some aspects, q is 5.

In some aspects, L′ is a bioreducible linker precursor. In some aspects, the bioreducible linker precursor is selected from the group consisting of

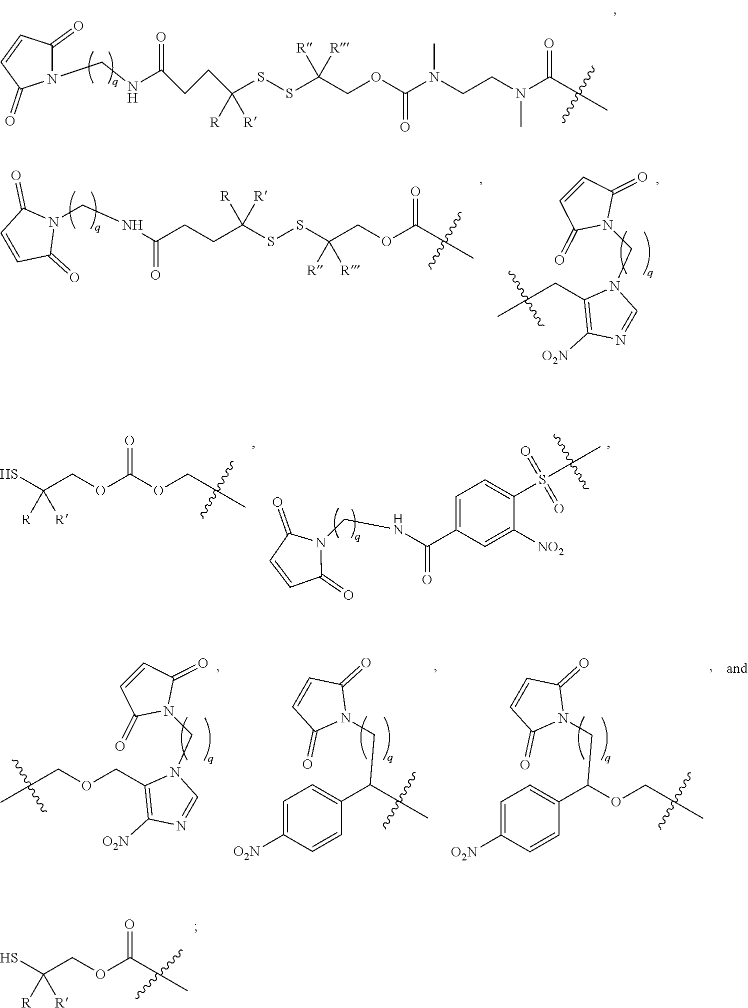

wherein:
q is an integer from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring; and
[symbol] is the point of attachment to X.

In certain aspects, L′ is an acid cleavable linker precursor. In some aspects, L′ is selected from the group consisting of

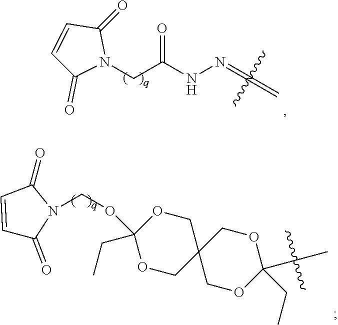

wherein:
q is an integer from 2 to 10; and
[symbol] is the point of attachment to X.

In certain aspects, L′ is a click-to-release linker precursor. In some aspects, L′ is selected from

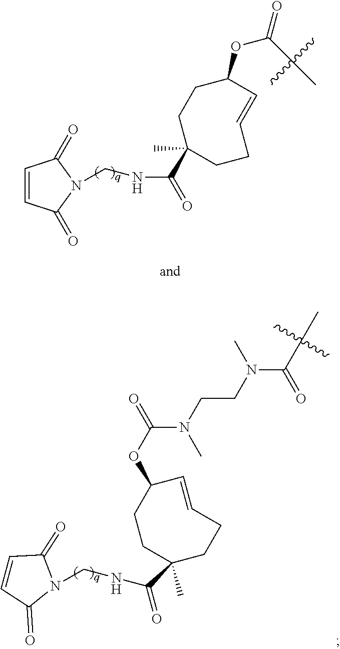

wherein:
q is an integer from 2 to 10; and
[symbol] is the point of attachment to X.

In certain aspects, L′ is a pyrophosphatase cleavable linker precursor. In some aspects, L′ is

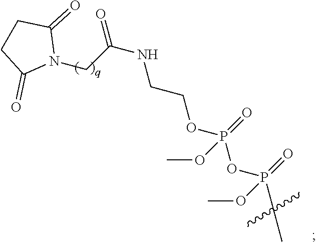

wherein:
q is an integer from 2 to 10;
[symbol] is the point of attachment to X.

In certain aspects, L′ is a beta-glucoronidase cleavable linker precursor. In some aspects, L′ is selected from

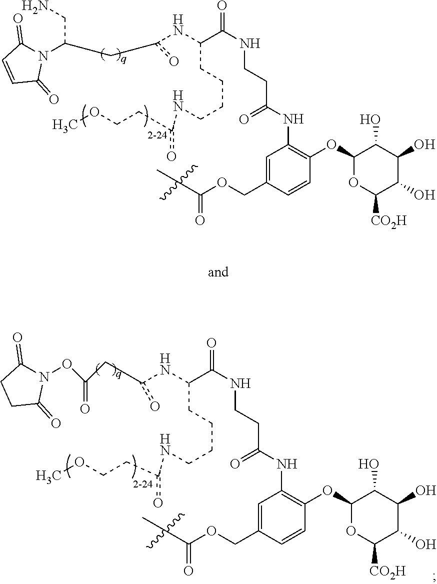

wherein:
   q is an integer from 2 to 10;
   ---- is absent or a bond; and
   [symbol] is the point of attachment to X.

In some aspects, the compound of formula (I-1) is selected from

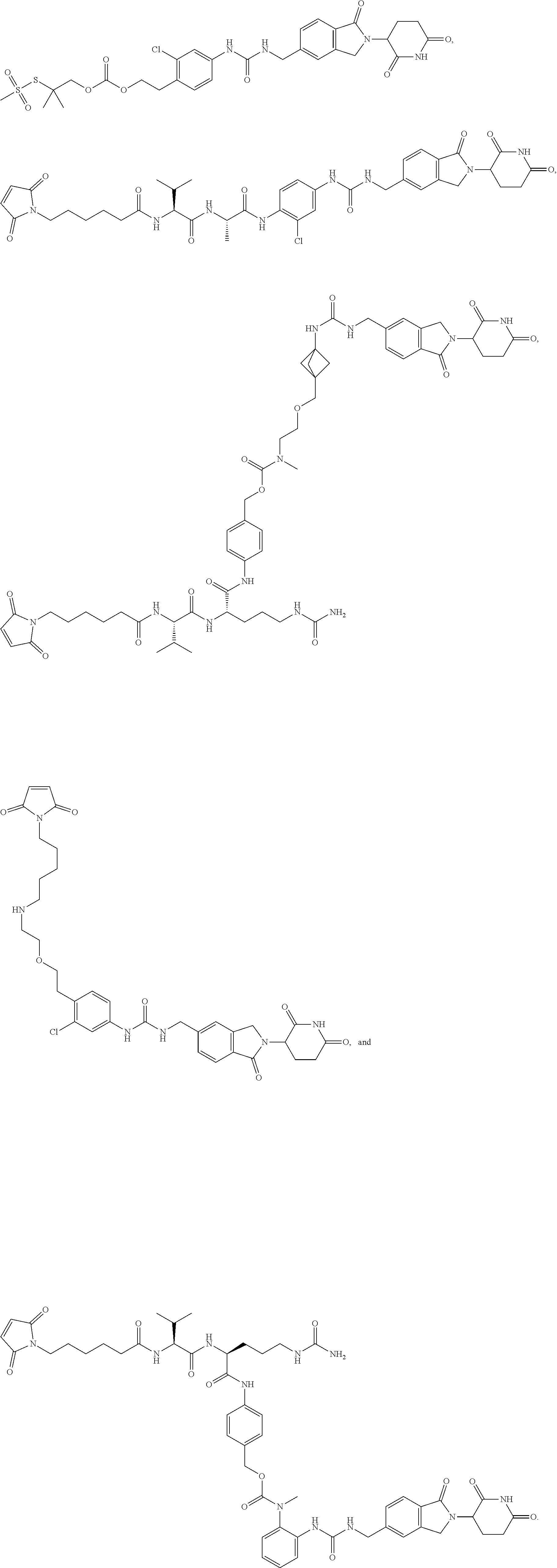

In some aspects, the anti-CD33 antibody or antigen-binding portion thereof is pre-treated before it is reacted with the compound of formula (I-1). In certain aspects, the compound of formula (I-1) is reacted with an anti-CD33 antibody or antigen-binding portion thereof. In aspects the anti-CD33 antibody or antigen-binding portion thereof can be pretreated to reduce interchain disulfides prior to reaction with the compound of formula (I-1).

### EXAMPLES

### General Synthetic Methods and Intermediates

The compounds of the present disclosure can be prepared by one of ordinary skill in the art in light of the present disclosure and knowledge in the art, and/or by reference to the schemes shown below and the synthetic examples. Exemplary synthetic routes are set forth in Schemes below and in Examples. It should be understood that the variables, (for example “R” groups) appearing in the following schemes and examples are to be read independently from those appearing elsewhere in the application. One of ordinary skill in the art would readily understand how the schemes and examples shown below illustrate the preparation of the compounds described herein.

Abbreviations used in the schemes generally follow conventions used in the art. Chemical abbreviations used in the specification and examples are defined as follows: “TIHF” for tetrahydrofuran; “DMF” for N,N-dimethylformamide; “Me” for methyl; “Bu” for butyl; “FA” for formic acid; “PE” for petrolium ether; “MeOH” for methanol; “EtOH” for ethanol; “DCM” for dichlormethane; “BOC” or “Boc” “TFA” for trifluoroacetic acid; “DMSO” for dimethylsulfoxide; “EtOAc” for ethyl acetate; “OAc” for acetate; “dppf” for 1,1′-bis(diphenylphosphino)ferrocene; “dba” for dibenzylideneacetone; “CDI” for 1,1′-carbonyldiimidazole; “TBAF” for tetrabutylammonium fluoride; “TBSCl” for tert-butyldimethylsilyl chloride; “Et₂O” for diethyl ether; “ACN” for acetonitrile; “h” for hours; “min” for minutes; “rt” for room temperature or retention time (context will dictate); “aq.” for aqueous, “sat.” for saturated; “min” for minutes; “HOBt” for 1-hydroxybenzotriazole hydrate; “HATU” for 1-[bis(dimethylamino)methylene]-1H-1,2,3-triazolo[4,5-b]pyridinium 3-oxid hexafluorophosphate or N-[(dimethylamino)-1H-1,2,3-triazolo-[4,5-b]pyridin-1-ylmethylene]-N-methylmethanaminium hexafluorophosphate N-oxide; “DIEA” and “iPrNEt₂” for diisopropylethylamine; “Et₃N” and “TEA” for triethyl amine.

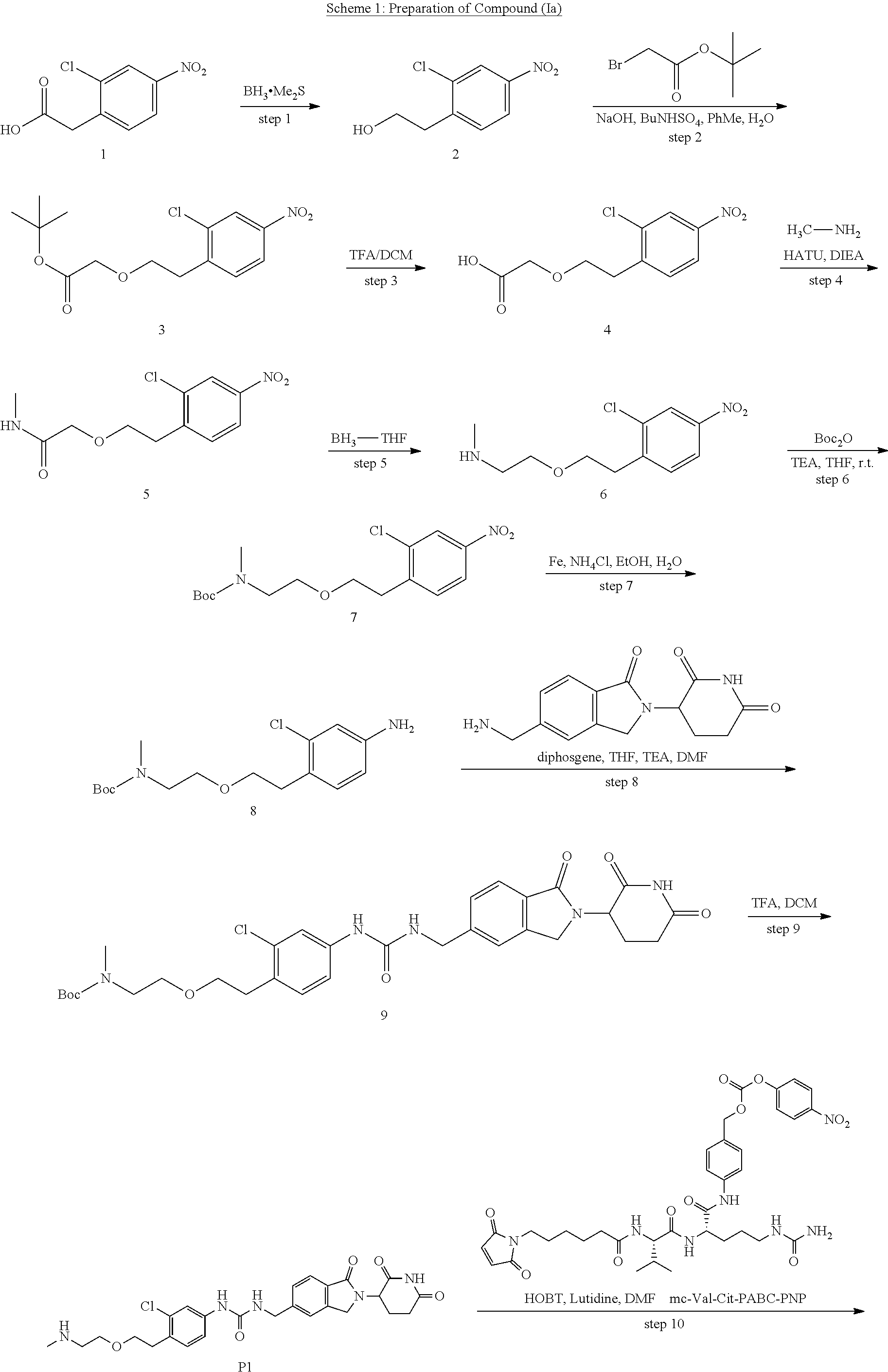

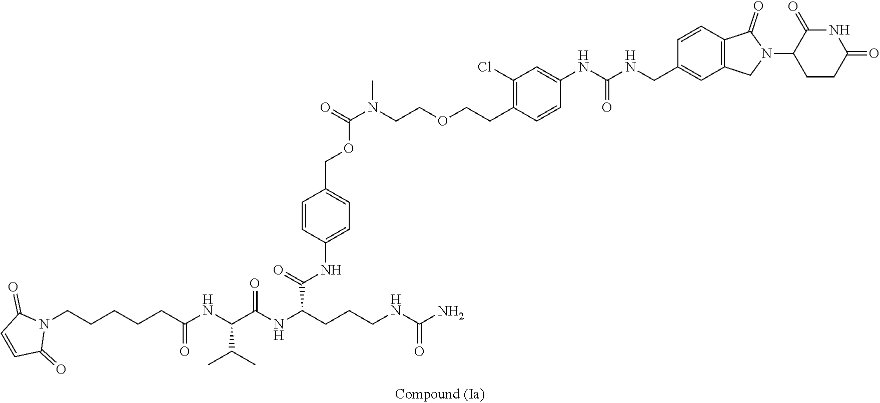 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

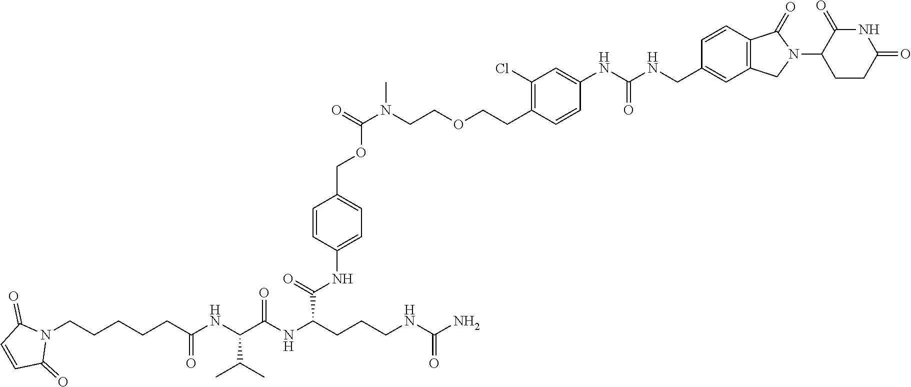 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

### Example 1: Synthesis of Compound (Ia)

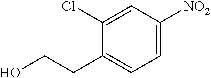 [SMILES: O=[N+]([O-])c1ccc(CCO)c(Cl)c1]

### Step 1: Synthesis of Compound 2

To a stirred solution of 2-chloro-4-nitrophenyl)acetic acid (Compound 1, 5.00 g, 23.19 mmol, 1.00 equiv) in THE (75.00 mL) was added BH₃-Me₂S (10M in THF) (5.80 mL, 58.0 mmol, 2.50 equiv) dropwise at 0° C. under nitrogen atmosphere. The resulting mixture was stirred for 2 h at 70° C. under nitrogen atmosphere. The mixture was cooled down to room temperature. The resulting mixture was concentrated under reduced pressure. The residue was purified by silica gel column chromatography (PE:EtOAc=1:1) to afford 2-(2-chloro-4-nitrophenyl)ethanol (3 g, 64%) as a yellow solid. ¹H NMR (400 MHz, CDCl₃) δ 8.26 (d, J=4.0 Hz, 1H), 8.10-8.05 (m, 1H), 7.50 (d, J=8.0 Hz, 1H), 3.99-3.91 (m, 2H), 3.16-3.09 (m, 2H).

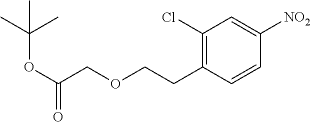 [SMILES: CC(C)(C)OC(=O)COCCc1ccc([N+](=O)[O-])cc1Cl]

### Step 2: Synthesis of Compound 3

To a stirred solution of 2-(2-chloro-4-nitrophenyl)ethanol (Compound 2, 5.00 g, 24.800 mmol, 1.00 equiv) and tert-butyl 2-bromoacetate (29.0 mL, 148.28 mmol, 8.00 equiv) in toluene (150.00 mL) was added Bu₄NHSO₄ (6.74 g, 19.84 mmol, 0.80 equiv). To the above mixture was added NaOH (5M in H₂O) (500.00 mL) dropwise over 40 min at 0° C. The resulting mixture was stirred for additional 2 h at 25° C. The resulting mixture was extracted with EtOAc (3×500 mL). The combined organic layers were washed with brine (400 mL) and dried over anhydrous Na₂SO₄. After filtration, the filtrate was concentrated under reduced pressure. The residue was purified by silica gel column chromatography (PE:EtOAc=4:1) to afford tert-butyl 2-[2-(2-chloro-4-nitrophenyl)ethoxy]acetate (8 g, 65%) as a yellow oil. ¹H NMR (400 MHz, CDCl₃) δ 8.23 (d, J=4.0 Hz, 1H), 8.10-8.04 (m, 1H), 7.60 (d, J=8.0 Hz, 1H), 4.09 (s, 2H), 3.83-3.80 (m, 2H), 3.17-3.14 (m, 2H), 1.45 (s, 9H).

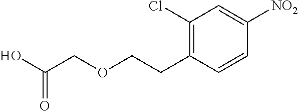 [SMILES: O=C(O)COCCc1ccc([N+](=O)[O-])cc1Cl]

### Step 3: Synthesis of Compound 4

To a stirred solution of tert-butyl 2-[2-(2-chloro-4-nitrophenyl)ethoxy]acetate (Compound 3, 8.00 g, 16.14 mmol, 1.00 equiv, 63.7%) in DCM (80.00 mL) was added TFA (16.00 mL) dropwise at room temperature. The resulting mixture was stirred for 1 h at room temperature. The resulting mixture was concentrated under vacuum. The resulting mixture was diluted with water (500 mL). The mixture was extracted with EtOAc (3×500 mL). The combined organic layers were washed with brine (200 mL), dried over anhydrous Na2SO4. After filtration, the filtrate was concentrated under reduced pressure. This resulted in [2-(2-chloro-4-nitrophenyl)ethoxy]acetic acid (6.5 g, crude) as yellow oil. LCMS (ESI): 517 (2M−H)−

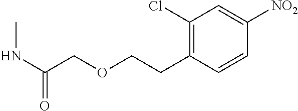 [SMILES: CNC(=O)COCCc1ccc([N+](=O)[O-])cc1Cl]

### Step 4: Synthesis of Compound 5

To a stirred solution of [2-(2-chloro-4-nitrophenyl)ethoxy]acetic acid (Compound 4, 6.30 g, 21.84 mmol, 1.00 equiv, 90%) and HATU (12.46 g, 32.76 mmol, 1.50 equiv) in DMF (65.00 mL) was added CH₃NH₂·HCl (1.77 g, 26.21 mmol, 1.20 equiv) and DIEA (15.20 g, 117.8 mmol, 4.00 equiv) dropwise at room temperature. The resulting mixture was stirred for 2 h at room temperature. The resulting mixture was diluted with water. The resulting mixture was extracted with EtOAc (2×100 mL). The combined organic layers were washed with brine (50 mL), dried over anhydrous Na₂SO₄. After filtration, the filtrate was concentrated under reduced pressure. The residue was purified by silica gel column chromatography (DCM:MeOH=10:1) to afford 2-[2-(2-chloro-4-nitrophenyl)ethoxy]-N-methylacetamide (10 g, purity: 50%, yield: 84%) as yellow oil. LCMS (ESI): 273.28 (M+H)⁺

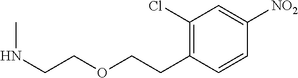 [SMILES: CNCCOCCc1ccc([N+](=O)[O-])cc1Cl]

### Step 5: Synthesis of Compound 6

To a stirred solution of 2-[2-(2-chloro-4-nitrophenyl)ethoxy]-N-methylacetamide (Compound 5, 3.3 g, 12.10 mmol, 1.00 equiv) in THE (35.00 mL) was added BH₃-THF (1M in THF) (12.10 mL, 12.10 mmol, 1.00 equiv) dropwise at room temperature under nitrogen atmosphere. The resulting mixture was stirred for 2 h at 70° C. under nitrogen atmosphere. The reaction was quenched with MeOH. The residue was acidified to pH 6 with 1N HCl. The resulting mixture was extracted with EtOAc (20 mL). The aqueous phase was basified to pH 8 with saturated NaHCO₃ (sat., aq.). The resulting mixture was extracted with EtOAc (3×100 mL), washed with brine (50 mL) and dried over anhydrous Na₂SO₄. After filtration, the filtrate was concentrated under reduced pressure. This resulted in [2-[2-(2-chloro-4-nitrophenyl)ethoxy]ethyl](methyl)amine (2.5 g, 80%) as yellow oil. LCMS (ESI): 259.26 (M+H)⁺

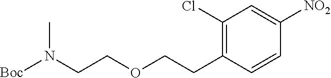 [SMILES: CN(CCOCCc1ccc([N+](=O)[O-])cc1Cl)C(=O)OC(C)(C)C]

### Step 6. Synthesis of Compound 7

To a stirred solution of [2-[2-(2-chloro-4-nitrophenyl)ethoxy]ethyl](methyl)amine (Compound 6, 2.50 g, 9.69 mmol, 1.00 equiv) and Boc₂O (2.53 g, 11.6 mmol, 1.20 equiv) in THE (40 mL) was added TEA (1.17 g, 11.6 mmol, 1.20 equiv) dropwise at 25° C. The mixture was stirred at 25° C. for 2 h. The resulting mixture was concentrated under vacuum. The residue was purified by silica gel column chromatography (DCM:MeOH=5:1) to afford tert-butyl N-[2-[2-(2-chloro-4-nitrophenyl)ethoxy]ethyl]-N-methylcarbamate (1.70 g, 50%) as yellow oil. LCMS (ESI): 359.36 (M+H)⁺

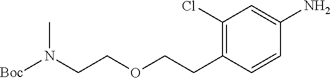 [SMILES: CN(CCOCCc1ccc(N)cc1Cl)C(=O)OC(C)(C)C]

### Step 7: Synthesis of Compound 8

To a stirred solution of tert-butyl N-[2-[2-(2-chloro-4-nitrophenyl)ethoxy]ethyl]-N-methylcarbamate (Compound 7, 1.70 g, 4.74 mmol, 1.00 equiv) and NH₄Cl (750 mg, 14.2 mmol, 3.00 equiv) in EtOH (85 mL) and H₂O (17 mL) was added Fe (1.3 g, 23.7 mmol, 5.00 equiv) at 25° C. The mixture was stirred at 80° C. for 2 h. The mixture was cooled down to room temperature. The resulting mixture was filtered, and the filter cake was washed with EtOH (3×50 mL). The filtrate was concentrated under reduced pressure. The residue was purified by silica gel column chromatography (PE:EtOAc=4:1) to afford tert-butyl N-[2-[2-(4-amino-2-chlorophenyl)ethoxy]ethyl]-N-methylcarbamate (900 mg, 58%) as yellow oil. LCMS (ESI): 329.33 (M+H)⁺

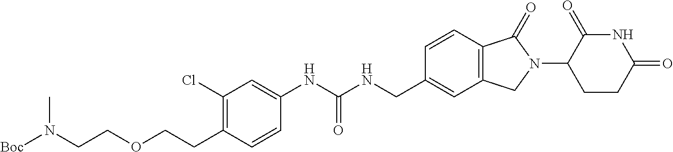 [SMILES: CN(CCOCCc1ccc(NC(=O)NCc2ccc3c(c2)CN(C2CCC(=O)NC2=O)C3=O)cc1Cl)C(=O)OC(C)(C)C]

### Step 8: Synthesis of Compound 9

To a stirred solution of tert-butyl N-[2-[2-(4-amino-2-chlorophenyl)ethoxy]ethyl]-N-methylcarbamate (Compound 8, 500 mg, 1.52 mmol, 1.00 equiv) in THE (10 mL) was added diphosgene (601 mg, 3.04 mmol, 2.00 equiv) dropwise at 25° C. The mixture was stirred at 25° C. for 1 h. The resulting mixture was concentrated under vacuum and re-dissolved in DMF (5 mL). To a stirred mixture of 3-[5-(aminomethyl)-1-oxo-3H-isoindol-2-yl]piperidine-2,6-dione (INT1, prepared as described below, 499 mg, 1.82 mmol, 1.20 equiv) and TEA (1.56 g, 15.45 mmol, 10.00 equiv) in DMF (20 mL) was added the solution mentioned above dropwise at 25° C. The mixture was stirred at 25° C. for 1 h. The resulting mixture was diluted with 40 mL of ice water. The resulting mixture was extracted with EtOAc (3×40 mL). The combined organic layers were washed with brine (5×40 mL) and dried over anhydrous Na₂SO₄. After filtration, the filtrate was concentrated under reduced pressure. The residue was purified by silica gel column chromatography (DCM:MeOH=10:1) to afford tert-butyl (2-(2-chloro-4-(3-((2-(2,6-dioxopiperidin-3-yl)-1-oxoisoindolin-5-yl)methyl)ureido)phenethoxy)ethyl)(methyl)carbamate (670 mg, 70%) as a white solid. LCMS: (ESI): 628.63 (M+H)⁺

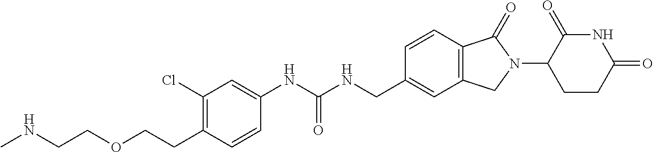 [SMILES: CNCCOCCc1ccc(NC(=O)NCc2ccc3c(c2)CN(C2CCC(=O)NC2=O)C3=O)cc1Cl]

Step 9: Synthesis of neoDegrader P1

To a stirred solution of tert-butyl N-[2-(2-[2-chloro-4-[([[2-(2,6-dioxopiperidin-3-yl)-1-oxo-3H-isoindol-5-yl]methyl]carbamoyl)amino]phenyl]ethoxy)ethyl]-N-methylcarbamate (Compound 9, 670 mg, 1.07 mmol, 1 eq) in DCM (10 mL) was added TFA (2.5 mL) dropwise at 0° C. The mixture was stirred at 25° C. for 1 h. The resulting mixture was concentrated under vacuum. The crude product was purified by Prep-HPLC with the following conditions: Column, SunFire C18 OBD Prep Column, 100 m, 19×250 mm; mobile phase, water (0.05% TFA) and ACN (5% Phase B up to 60% in 30 min); Detector, UV 220 nm. The collected fraction was lyophilized to give 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (500 mg, 89%) as a white solid. LCMS (ESI): 528.53 (M+H)⁺. ¹H NMR (400 MHz, Methanol-d₄) δ 7.77 (d, J=8.0 Hz, 1H), 7.57-7.53 (m, 2H), 7.49 (d, J=8.0 Hz, 1H), 7.21 (d, J=4.0 Hz, 2H), 5.19-5.1 (m, 1H), 4.55-4.41 (m, 4H), 3.75-3.67 (m, 4H), 3.21-3.15 (m, 2H), 3.03-3.96 (m, 2H), 2.96-2.84 (m, 1H), 2.83-2.73 (m, 2H), 2.69 (s, 3H), 2.55-2.42 (m, 1H), 2.21-2.12 (m, 1H).

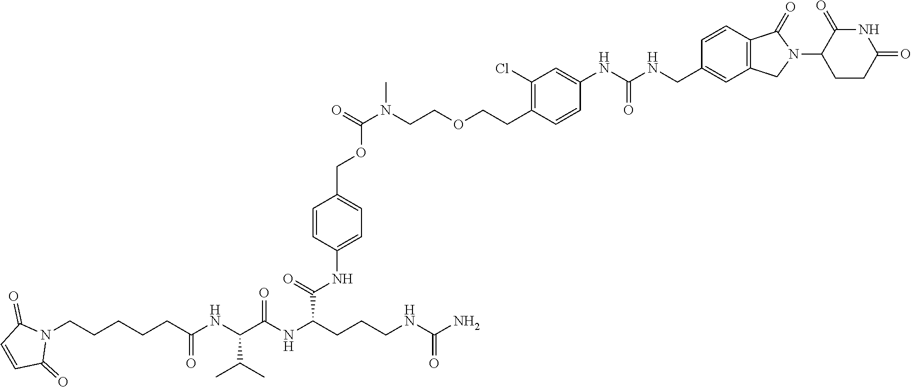 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

### Step 10: Synthesis of Compound (Ia)

To a stirred mixture of 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (neoDegrader P1, 200 mg, 0.38 mmol, 1.00 equiv) and lutidine (81 mg, 0.76 mmol, 2.00 equiv) in DMF (10 mL) were added HOBT (26 mg, 0.19 mmol, 0.50 equiv) and [4-[(2S)-5-(carbamoylamino)-2-[(2S)-2-[6-(2,5-dioxopyrrol-1-yl)hexanamido]-3-methylbutanamido]pentanamido]phenyl]methyl 4-nitrophenyl carbonate (279 mg, 0.38 mmol, 1.00 equiv) in portions at room temperature. The reaction mixture was stirred for 12 hours at 40 degrees C. under nitrogen atmosphere. After the reaction was cooled down to room temperature, the reaction was quenched with water (30 mL). The resulting mixture was extracted with DCM (3×30 mL). The combined organic layers were washed with water (2×30 mL), brine (30 mL), dried over Na₂SO₄. After filtration, the filtrate was concentrated to dryness under vacuum. The residue was purified by reverse phase column (C18, mobile phase A: 0.1% FA in water, B: ACN). The collected fraction was concentrated to dryness under vacuum. The crude product (60 mg) was purified by Prep-HPLC with the following conditions (Column: Xselect CSH OBD Column 30×150 mm 5 um, n; Mobile Phase A:Water(0.1% FA), Mobile Phase B:ACN; Flow rate: 60 mL/min; Gradient: 33 B to 50 B in 7 min; 220 nm; RT1:5.27 min). The collected fraction was lyophilized to afford [4-[(2S)-5-(carbamoylamino)-2-[(2S)-2-[6-(2,5-dioxopyrrol-1-yl)hexanamido]-3-methylbutanamido]pentanamido]phenyl]methyl N-[2-(2-[2-chloro-4-[([[2-(2,6-dioxopiperidin-3-yl)-1-oxo-3H-isoindol-5-yl]methyl]carbamoyl)amino]phenyl]ethoxy)ethyl]-N-methylcarbamate (23.8 mg, 5%) as a white solid. LCMS (ESI): 1126.11 (M+H)⁺. ¹H NMR (400 MHz, DMSO-d₆) δ 10.99 (s, 1H), 10.00 (s, 1H), 8.88 (s, 1H), 8.12-8.08 (m, 1H), 7.85-7.81 (m, 2H), 7.70-7.67 (m, 2H), 7.60-7.58 (m, 1H), 7.51 (s, 1H), 7.47-7.44 (m, 1H), 7.28-7.25 (m, 2H), 7.18-7.12 (m, 2H), 7.00 (s, 2H), 6.90 (br s, 1H), 5.97-5.95 (m, 1H), 5.42 (s, 2H), 5.12-5.05 (m, 1H), 4.98 (s, 2H), 4.42-4.32 (m, 4H), 4.18-4.15 (m, 1H), 3.56-3.40 (m, 4H), 3.37-3.36 (m, 3H), 3.05-2.90 (m, 3H), 2.89-2.85 (m, 5H), 2.72-2.55 (m, 2H), 2.40-2.33 (m, 2H), 2.25-2.15 (m, 2H), 2.00-1.87 (m, 2H), 1.74-1.57 (m, 2H), 1.50-1.42 (m, 5H), 1.22-1.10 (m, 3H), 0.85-0.80 (m, 6H).

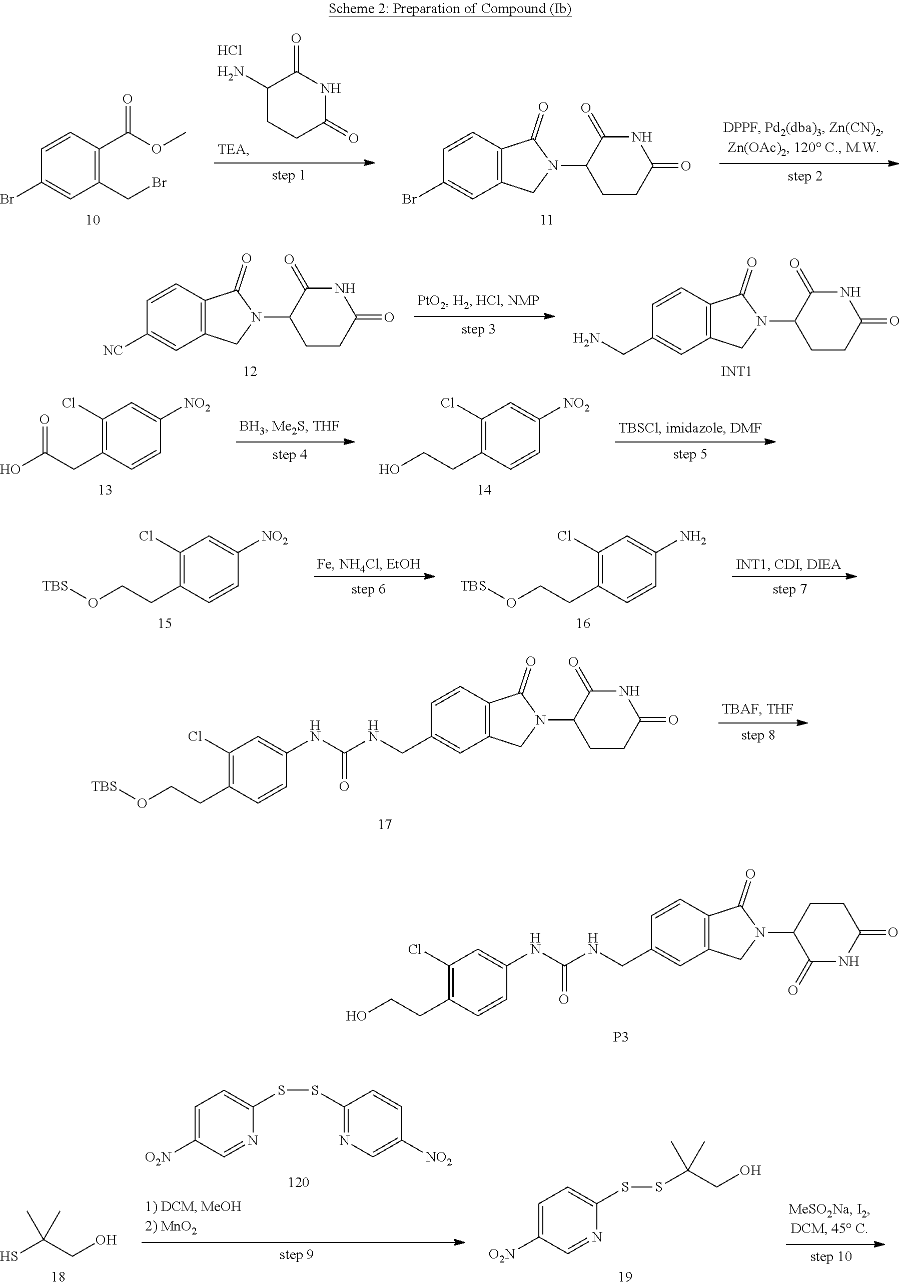

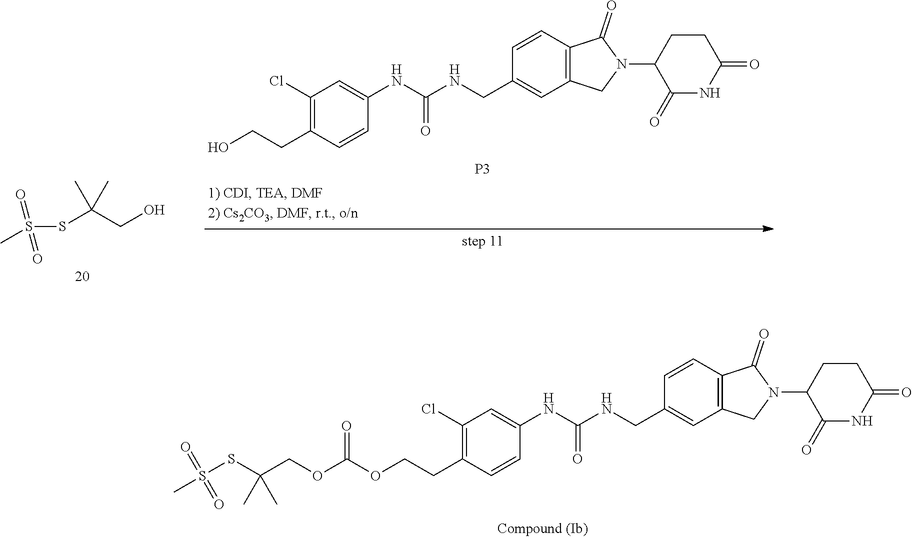

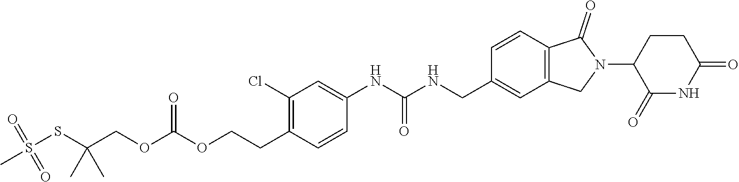 [SMILES: CC(C)(COC(=O)OCCc1ccc(NC(=O)NCc2ccc3c(c2)CN(C2CCC(=O)NC2=O)C3=O)cc1Cl)SS(C)(=O)=O]

### Example 2: Synthesis of Compound (Ib)

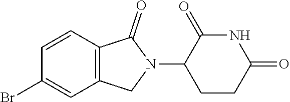 [SMILES: O=C1CCC(N2Cc3cc(Br)ccc3C2=O)C(=O)N1]

### Step 1: Synthesis of Compound 11

To a stirred mixture of methyl 4-bromo-2-(bromomethyl)benzoate (Compound 10, 20.0 g, 64.8 mmol, 1.00 equiv) and 3-aminopiperidine-2,6-dione hydrochloride (10.64 g, 83.0 mmol, 1.28 equiv) in DMF (80 ml) was added TEA (22.4 mL, 162.2 mmol, 2.50 equiv) dropwise at 25° C. under nitrogen atmosphere. The mixture was stirred at 25° C. for 16 h. This was followed by addition of H2O (60 mL), AcOH (23 mL) and Et2O (60 mL) in sequence at 25° C. The mixture was stirred at 25° C. for 2 h. The precipitated solids were collected by filtration and washed with Et2O (60 mL). This resulted in 3-(5-bromo-1-oxo-3H-isoindol-2-yl)piperidine-2,6-dione (9.0 g, 42%) as a light blue solid. LCMS (ESI): 323.32 (M+H)⁺

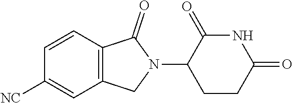 [SMILES: N#Cc1ccc2c(c1)CN(C1CCC(=O)NC1=O)C2=O]

### Step 2: Synthesis of Compound 12

To a stirred mixture of 3-(5-bromo-1-oxo-3H-isoindol-2-yl)piperidine-2,6-dione (Compound 11, 1.00 g, 3.09 mmol, 1.00 equiv) and dppf (51 mg, 0.093 mmol, 0.03 equiv) in DMF (8 mL) were added Zn(OAc)₂ (170 mg, 0.928 mmol, 0.30 equiv), Zn(CN)₂ (545 mg, 4.64 mmol, 1.50 equiv) and Pd₂(dba)₃ (28 mg, 0.031 mmol, 0.01 equiv) at 25 degrees C. under nitrogen atmosphere. The final reaction mixture was irradiated with microwave radiation for 2 h at 120° C. The mixture was cooled down to room temperature and filtered. The filter cake was washed with MeOH (3×30 mL). The filtrate was concentrated under reduced pressure. The residue was subjected to flash chromatography (silica gel, 80 g, DCM:MeOH=10:1) to give the desired product 2-(2,6-dioxopiperidin-3-yl)-1-oxo-3H-isoindole-5-carbonitrile (400 mg, 47%) as a brown solid. LCMS (ESI): 270 (M+H)⁺

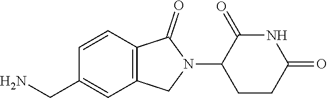 [SMILES: NCc1ccc2c(c1)CN(C1CCC(=O)NC1=O)C2=O]

### Step 3: Synthesis of INT1

To a stirred mixture of 2-(2,6-dioxopiperidin-3-yl)-1-oxo-3H-isoindole-5-carbonitrile (Compound 12, 3.0 g, 11.14 mmol, 1.00 equiv) and HCl (12M) (3.6 mL) in MeOH (25 mL) was added PtO₂ (1.25 g, 5.5 mmol, 0.49 equiv) at 25° C. The mixture was hydrogenated at room temperature for 16 h under hydrogen atmosphere using a hydrogen balloon. The resulting mixture was filtered, and the filter cake was washed with MeOH (2×30 mL). The filtrate was concentrated under reduced pressure. The resulting solid was washed with DCM:MeOH (3:1) (3×30 mL) and dried. This resulted in 3-[5-(aminomethyl)-1-oxo-3H-isoindol-2-yl]piperidine-2,6-dione (2.5 g, 80%) as grey solid. LCMS (ESI): 274 (M+H)⁺. ¹H NMR (400 MHz, DMSO-d₆) δ 11.02 (s, 1H), 8.15 (s, 1H), 7.98 (d, J=8.4 Hz, 1H), 7.89 (d, J=8.4 Hz, 1H), 5.16-5.11 (m, 1H), 4.52 (d, J=17.2 Hz, 1H), 4.40 (d, J=17.2 Hz, 1H), 2.96-2.90 (m, 1H), 2.60-2.54 (m, 1H), 2.43-2.34 (m, 1H), 2.06-1.96 (m, 1H)

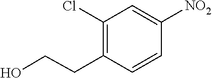 [SMILES: O=[N+]([O-])c1ccc(CCO)c(Cl)c1]

### Step 4: Synthesis of Compound 14

To a stirred solution of (2-chloro-4-nitrophenyl)acetic acid (Compound 13, 5.00 g, 22.50 mmol, 1.00 equiv) in THE (75 mL) was added BH₃-Me₂S (10M in THF) (5.60 mL, 56 mmol, 2.50 equiv) dropwise at 0° C. under nitrogen atmosphere. The mixture was stirred at 70° C. for 2 h. The resulting mixture was concentrated under vacuum. The residue was applied onto silica gel column and eluted with PE/EtOAc (5:1) to afford 2-(2-chloro-4-nitrophenyl)ethanol (4.44 g, 88%) as a yellow solid. ¹H NMR (400 MHz, CDCl₃) δ 8.26 (d, J=4.0 Hz, 1H), 8.10-8.05 (m, 1H), 7.50 (d, J=8.0 Hz, 1H), 3.99-3.91 (m, 2H), 3.16-3.09 (m, 2H)

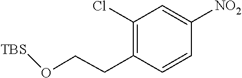

### Step 5: Synthesis of Compound 15

To a stirred mixture of 2-(2-chloro-4-nitrophenyl)ethanol (Compound 14, 4.44 g, 22.02 mmol, 1.00 equiv) and imidazole (4.50 g, 66.06 mmol, 3.00 equiv) in DMF (50.00 mL) was added TBSCl (6.97 g, 46.25 mmol, 2.10 equiv) at 25° C. The mixture was stirred at 25° C. for 16 h. The resulting mixture was diluted with water (100 mL). The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was applied onto silica gel column and eluted with PE/EtOAc (10:1) to afford tert-butyl[2-(2-chloro-4-nitrophenyl)ethoxy]dimethylsilane (6.6 g, 90%) as a colorless oil. ¹H NMR (400 MHz, CDCl₃) δ 8.24 (s, 1H), 8.06-8.04 (m, 1H), 7.46 (d, J=8.4 Hz, 1H), 3.89-3.86 (m, 2H), 3.06-0.04 (m, 2H), 0.85 (s, 9H), 0.04 (s, 6H).

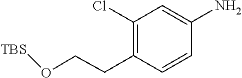

### Step 6: Synthesis of Compound 16

To a mixture of tert-butyl[2-(2-chloro-4-nitrophenyl)ethoxy]dimethylsilane (Compound 15, 5.70 g, 18.05 mmol, 1.00 equiv) and Fe (10.08 g, 180.45 mmol, 10.00 equiv) in EtOH (110 mL)/water (55 mL) was added NH₄Cl (9.65 g, 180.45 mmol, 10 equiv). The mixture was stirred at 80° C. for 2 h. The mixture was cooled down to room temperature. The resulting mixture was filtered, and the filter cake was washed with EtOH (3×50 mL). The filtrate was concentrated under reduced pressure. The residue was diluted with water (100 mL) and extracted with EtOAc (50 mL×3). The combined organic layer was dried over anhydrous sodium sulfate and evaporated to dryness in vacuo to give 4-[2-[(tert-butyldimethylsilyl)oxy]ethyl]-3-chloroaniline (5.2 g, crude) as a pale brown oil. LCMS (ESI): 286.29 (M+H)⁺

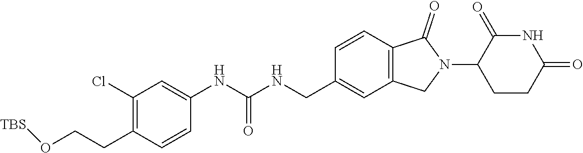

### Step 7: Synthesis of Compound 17

To a solution of 4-[2-[(tert-butyldimethylsilyl)oxy]ethyl]-3-chloroaniline (Compound 16, 200.00 mg, 0.70 mmol, 1.00 equiv) and TEA (141 mg, 1.40 mmol, 2.00 equiv) in DMF (3 mL) was added CDI (113 mg, 0.70 mmol, 1.00 equiv) in DMF (1 mL) dropwise under nitrogen dropwise at 0 degrees C. The resulting mixture was stirred at 25° C. for 1 hour. Then the above solution and TEA (141 mg, 1.40 mmol) were added dropwise into solution of 3-[5-(aminomethyl)-1-oxo-3H-isoindol-2-yl]piperidine-2,6-dione (INT1, 192 mg, 0.70 mmol, 1.00 equiv) in DMF (2 mL). The same reaction was repeated for twice. The resulting mixture was stirred at 25° C. for 1 hour. The reaction was diluted with water (20 mL), extracted with EtOAc (20 mL×3). The combined organic layer was washed with water, brine, dried over anhydrous sodium sulfate and evaporated to dryness in vacuum. The residue was purified with silica gel column (DCM:MeOH=10:1) to give 1-(4-[2-[(tert-butyldimethylsilyl)oxy]ethyl]-3-chlorophenyl)-3-[[2-(2,6-dioxopiperidin-3-yl)-1-oxo-3H-isoindol-5-yl]methyl]urea (170 mg, 21%) as a white solid. LCMS (ESI): 585.59 (M+H)⁺

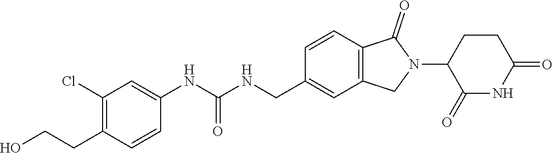 [SMILES: O=C1CCC(N2Cc3cc(CNC(=O)Nc4ccc(CCO)c(Cl)c4)ccc3C2=O)C(=O)N1]

Step 8: Synthesis of neoDegrader P3

To a solution of 1-(4-[2-[(tert-butyldimethylsilyl)oxy]ethyl]-3-chlorophenyl)-3-[[2-(2,6-dioxopiperidin-3-yl)-1-oxo-3H-isoindol-5-yl]methyl]urea (Compound 17, 170.00 mg, 0.29 mmol, 1.00 equiv) in THE (2.00 mL) was added TBAF (1 N in THF, 0.58 mL, 0.58 mmol, 2.00 equiv) at 0° C. The resulting mixture was stirred at 25 degrees C. for 8 hours. The reaction was purified with Prep-TLC (DCM:MeOH=10:1) to give 147 mg of the crude 1-(3-chloro-4-(2-hydroxyethyl)phenyl)-3-((2-(2,6-dioxopiperidin-3-yl)-1-oxoisoindolin-5-yl)methyl)urea as a white solid. LCMS (ESI): 471.47 (M+H)⁺

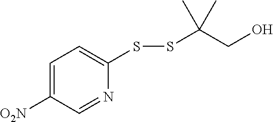 [SMILES: CC(C)(CO)SSc1ccc([N+](=O)[O-])cn1]

### Step 9: Synthesis of Compound 19

2-Methyl-2-sulfanylpropan-1-ol (Compound 18, 1.4 g, 13.2 mmol, 1.00 equiv) and 5-nitro-2-[(5-nitropyridin-2-yl)disulfanyl]pyridine (Compound 120, 2.05 g, 6.67 mmol, 0.50 equiv) were added into a mixture of solvent of dichloromethane (3.50 mL) and MeOH (3.50 mL). The resulting mixture was stirred at 15° C. Then manganese dioxide (2.29 g, 26.2 mmol, 2 equiv) was added in portions. The resulting mixture was stirred at 15° C. for 15 min. LCMS traces showed the reaction was completed. The reaction was evaporated to dryness and the residue was purified by reverse flash chromatography with the following conditions: column, C18 silica gel; mobile phase, ACN in water (0.1% NH₄HCO₃), 10% to 100% gradient in 30 min; detector, UV 254 nm. The collected fraction was concentrated to dryness under vacuum to afford 2-methyl-2-[(5-nitropyridin-2-yl)disulfanyl]propan-1-ol (2.2 g 58%) as a yellow solid. LCMS (ESI): 261 (M+H)⁺.

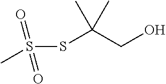

### Step 10: Synthesis of Compound 20

To a solution of 2-methyl-2-[(5-nitropyridin-2-yl)disulfanyl]propan-1-ol (Compound 20, 1.0 g, 3.84 mmol, 1.00 equiv) in anhydrous DCM (30 mL) was added MeSO₂Na (1.57 g, 15.4 mmol, 4.00 equiv) and iodine (1.95 g, 7.68 mmol, 2.00 equiv) in portions. The reaction mixture was stirred at 45° C. for 24 h. The mixture was concentrated, and the residue was purified by column chromatography on silica gel (TLC: PE:EA=3:1, Rf=0.60; 0-35% EtOAc in petroleum ether) to afford 2-(methanesulfonylsulfanyl)-2-methylpropan-1-ol (80 mg, 10%) as a yellow oil. ¹H NMR (400 MHz, CD₃Cl): δ 3.50 (s, 2H), 3.33 (s, 3H), 2.16 (br s, 1H), 1.47 (s, 6H).

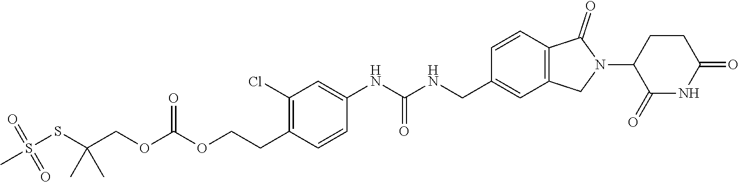 [SMILES: CC(C)(COC(=O)OCCc1ccc(NC(=O)NCc2ccc3c(c2)CN(C2CCC(=O)NC2=O)C3=O)cc1Cl)SS(C)(=O)=O]

### Step 11: Synthesis of Compound (Ib)

To a solution of 1-[3-chloro-4-(2-hydroxyethyl)phenyl]-3-[[2-(2,6-dioxopiperidin-3-yl)-1-oxo-3H-isoindol-5-yl]methyl]urea (neoDegrader P3, 200.00 mg, 0.42 mmol, 1.00 equiv) and TEA (129 mg, 1.26 mmol, 3.00 equiv) in DMF (4 mL) was added a solution of CDI (138 mg, 0.84 mmol, 2.00 equiv) in DMF (1 mL). The reaction mixture was stirred at room temperature for 2 hours. The reaction was diluted with water (50 mL) and extracted with EtOAc (20 mL×3). The combined organic layer was washed with water (20 mL×3), brine (20 mL), dried over sodium sulfate and evaporated to dryness in vacuum to give the crude product (2-[2-chloro-4-[([[2-(2,6-dioxopiperidin-3-yl)-1-oxo-3H-isoindol-5-yl]methyl]carbamoyl)amino]phenyl]ethyl imidazole-1-carboxylate, 200 mg) as pale yellow solid. To a solution of the crude product (100.00 mg, 0.18 mmol, 1.00 equiv) and Cs₂CO₃ (115 mg, 0.35 mmol, 2.00 equiv) in DMF (8 mL) was added 2-(methanesulfonylsulfanyl)-2-methylpropan-1-ol (Compound 20, 59 mg, 0.32 mmol, 1.80 equiv) in DMF (2 mL) dropwise at room temperature. The reaction was stirred at 15° C. for 22 hours. The reaction was diluted with EtOAc (50 ml) and ice-cooled water (100 mL). The organic layer was separated out. The water phase was extracted with EtOAc (30 mL×3). The combined organic layer was washed with brine (30 mL×3), dried over anhydrous sodium sulfate and evaporated to dryness in vacuum to give the crude product (150 mg) as a yellow solid. The crude product was purified with Prep-HPLC (Column: Xselect CSH OBD Column 30×150 mm 5 um; Mobile Phase A:Water (0.1% FA), Mobile Phase B:ACN; Flow rate: 60 mL/min; Gradient: 38 B to 58 B in 7 min; 220 nm; RT1:5.12 min). The collected fraction was lyophilized to give 1-[3-chloro-4-[2-([[2-(methanesulfonylsulfanyl)-2-methylpropoxy]carbonyl]-oxy)ethyl]phenyl]-3-[[2-(2,6-dioxopiperidin-3-yl)-1-oxo-3H-isoindol-5-yl]methyl]urea (15.7 mg, 11%) as a white solid. LCMS (ESI): 681.68 (M+H)⁺. ¹H NMR (400 MHz, DMSO-d₆) δ 10.99 (s, 1H), 8.86 (s, 1H), 7.70 (d, J=2.4 Hz, 1H), 7.51 (s, 1H), 7.44 (d, J=8.0 Hz, 1H), 7.24-7.17 (m, 1H), 6.87-6.84 (m, 1H), 5.76 (s, 2H), 5.13-5.11 (m, 1H), 4.42-4.40 (m, 2H), 4.32-4.28 (m, 4H), 3.54 (s, 3H), 3.00-2.87 (m, 3H), 2.62-2.58 (m, 1H), 2.44-2.34 (m, 1H), 2.01-1.95 (m, 1H), 1.45 (s, 6H).

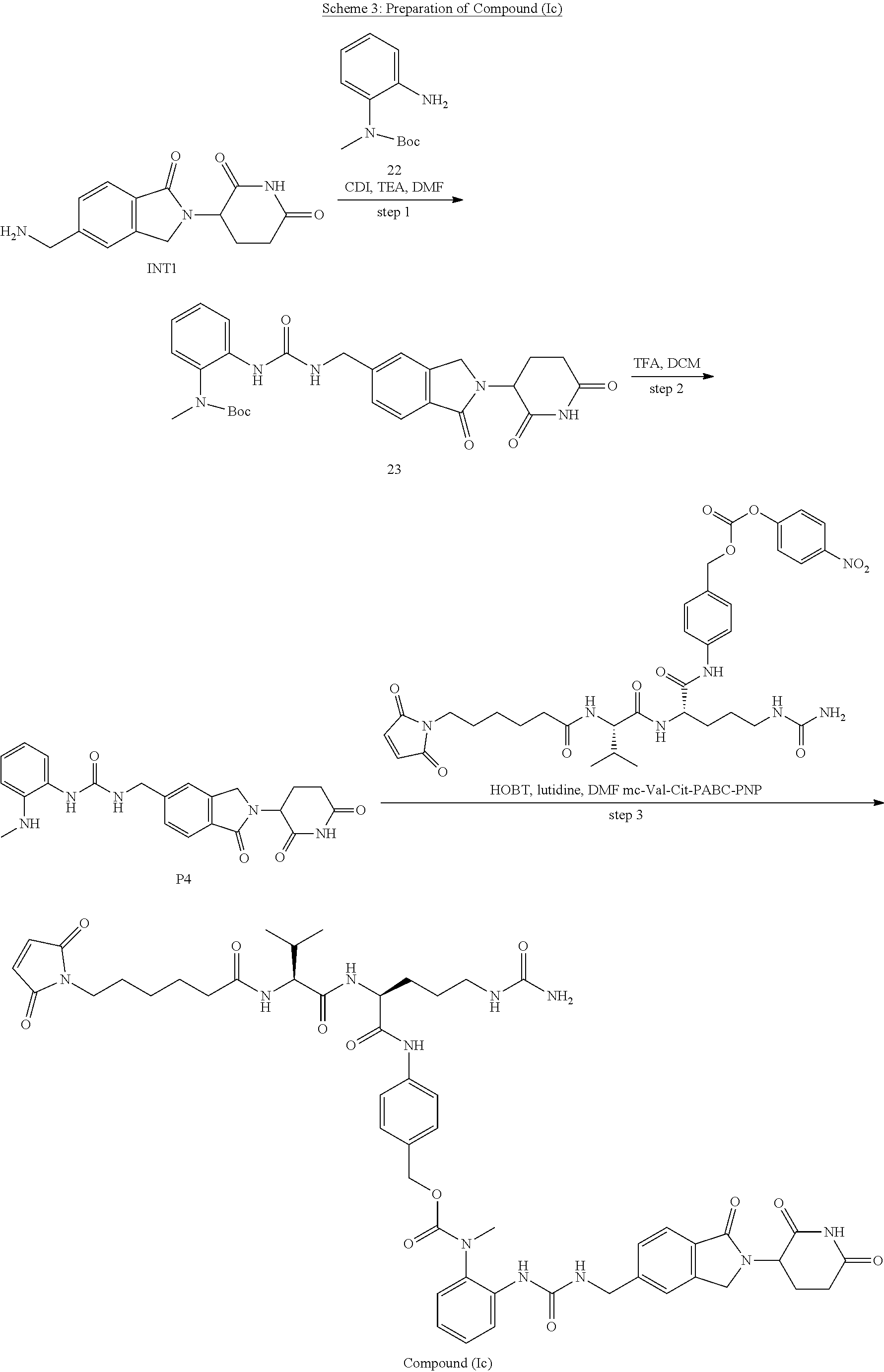

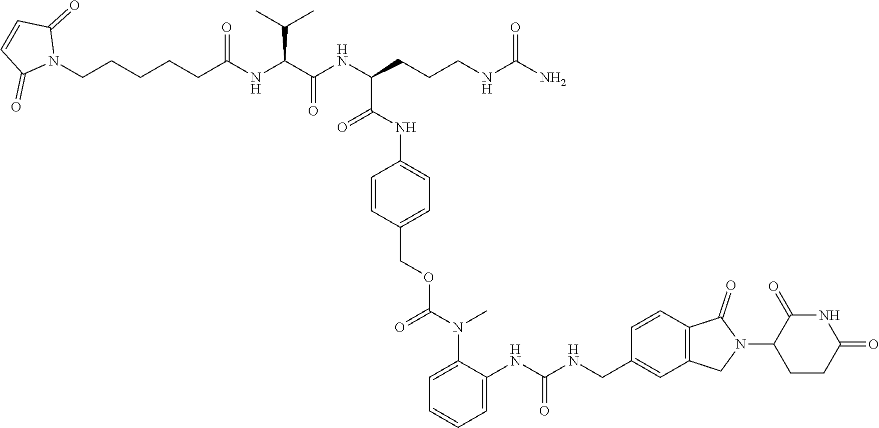 [SMILES: CC(C)[C@H](NC(=O)CCCCCN1C(=O)C=CC1=O)C(=O)N[C@@H](CCCNC(N)=O)C(=O)Nc1ccc(COC(=O)N(C)c2ccccc2NC(=O)NCc2ccc3c(c2)CN(C2CCC(=O)NC2=O)C3=O)cc1]

### Example 3: Synthesis of Compound (Ic)

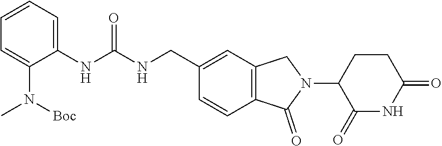 [SMILES: CN(C(=O)OC(C)(C)C)c1ccccc1NC(=O)NCc1ccc2c(c1)CN(C1CCC(=O)NC1=O)C2=O]

### Step 1: Synthesis of Compound 23

To a stirred solution of tert-butyl (2-aminophenyl)(methyl)carbamate (Compound 22, 300 mg, 1.35 mmol, 1.00 equiv) in DMF (20 mL) was added CDI (218 mg, 1.35 mmol, 1.00 equiv) and TEA (68 mg, 1.35 mmol, 1.00 equiv) dropwise at 0° C. under nitrogen atmosphere. The mixture was stirred at 0° C. for 2 h. To the above mixture was added 3-[5-(aminomethyl)-1-oxo-3H-isoindol-2-yl]piperidine-2,6-dione (INT1, 368 mg, 1.35 mmol, 1.00 equiv) in portions. The resulting mixture was stirred for overnight at 75° C. Then the reaction mixture was cooled down to room temperature. The resulting mixture was quenched with water (30 mL) and extracted with DCM (3×30 mL). The combined organic layers were washed with brine (30 mL), dried over anhydrous Na₂SO₄, filtered and concentrated under reduced pressure. The residue was purified by silica gel column chromatography (DCM/MeOH=10:1) to afford tert-butyl N-[2-[([[2-(2,6-dioxopiperidin-3-yl)-1-oxo-3H-isoindol-5-yl]methyl]carbamoyl)amino]phenyl]-N-methylcarbamate (300 mg, 42%) as white solid. LCMS (ESI): 522 (M+H)⁺

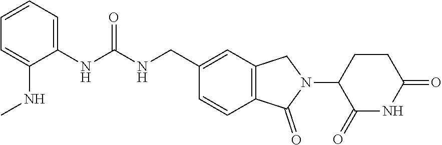 [SMILES: CNc1ccccc1NC(=O)NCc1ccc2c(c1)CN(C1CCC(=O)NC1=O)C2=O]

Step 2. Synthesis of neoDegrader P4

To a stirred solution tert-butyl N-[2-[([[2-(2,6-dioxopiperidin-3-yl)-1-oxo-3H-isoindol-5-yl]methyl]carbamoyl)amino]phenyl]-N-methylcarbamate (Compound 23, 300 mg, 1.00 equiv) in DCM (20 mL) was added TFA (5 mL) at 0° C. The mixture was stirred at 0° C. for 2 h. The resulting mixture was concentrated under vacuum. The crude product was purified by reverse phase with the following conditions (C18, Mobile Phase A: Water (0.1% FA), Mobile Phase B: ACN; Flow rate: 60 mL/min). The collected fraction was concentrated under vacuum to afford 3-[[2-(2,6-dioxopiperidin-3-yl)-1-oxo-3H-isoindol-5-yl]methyl]-1-[2-(methylamino)phenyl]urea (210 mg, 87%) as a white solid. LCMS (ESI): 422 (M+H)⁺. ¹H NMR (300 MHz, DMSO-d₆) δ 10.99 (s, 1H), 7.69 (d, J=7.8 Hz, 1H), 7.60 (s, 1H), 7.53 (s, 1H), 7.45 (d, J=8.4 Hz, 1H), 7.26-7.24 (m, 1H), 6.99-6.93 (m, 1H), 6.76-6.72 (m, 1H), 6.60-6.55 (m, 2H), 5.14-5.08 (m, 1H), 5.00-4.85 (br s, 1H), 4.48-4.28 (m, 4H), 2.92-2.82 (m, 1H), 2.70 (s, 3H), 2.62-2.57 (m, 1H), 2.49-2.41 (m, 1H), 2.02-1.95 (m, 1H).

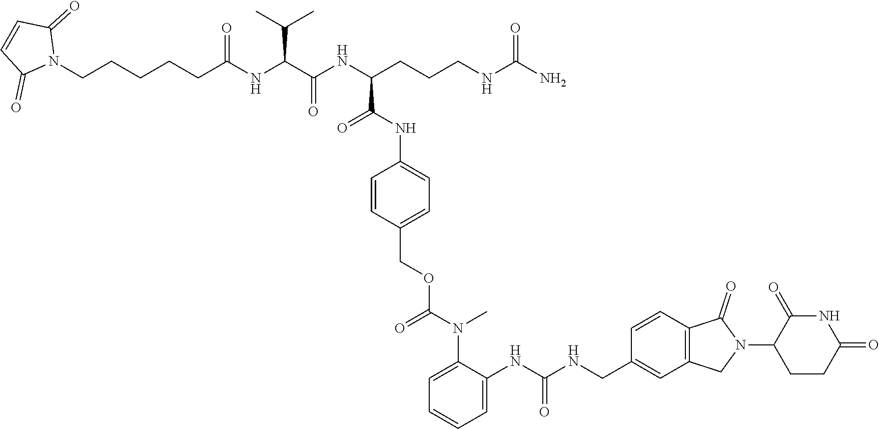 [SMILES: CC(C)[C@H](NC(=O)CCCCCN1C(=O)C=CC1=O)C(=O)N[C@@H](CCCNC(N)=O)C(=O)Nc1ccc(COC(=O)N(C)c2ccccc2NC(=O)NCc2ccc3c(c2)CN(C2CCC(=O)NC2=O)C3=O)cc1]

### Step 3. Synthesis of Compound (Ic)

To a stirred mixture of 3-[[2-(2,6-dioxopiperidin-3-yl)-1-oxo-3H-isoindol-5-yl]methyl]-1-[2-(methylamino)phenyl]urea (P4, 150.00 mg, 0.36 mmol, 1.00 equiv), 2,6-lutidine (76 mg, 0.71 mmol, 2.00 equiv) and HOBT (96 mg, 0.71 mmol, 2.00 equiv) in DMF (3.00 mL) was added [4-[(2S)-5-(carbamoylamino)-2-[(2S)-2-[6-(2,5-dioxopyrrol-1-yl)hexanamido]-3-methylbutanamido]pentanamido]phenyl]methyl 4-nitrophenyl carbonate (394 mg, 0.53 mmol, 1.50 equiv) at room temperature under nitrogen atmosphere. The reaction mixture was purified by reverse flash chromatography with the following conditions: column, C18 silica gel; mobile phase, Mobile Phase A: water (0.1% FA), Mobile Phase B: ACN;) to afford crude product (60 mg) as a white solid. The crude product (60 mg) was purified by Prep-HPLC with the following conditions (Column: Xselect CSH OBD Column 30×150 mm 5 um, n; Mobile Phase A:Water (0.1% FA), Mobile Phase B:ACN; Flow rate: 60 mL/min; Gradient: 24 B to 44 B in 7 min; 220 nm; RT1:6.33; RT2:). The collected fraction was lyophilized to afford [4-[(2S)-5-(carbamoylamino)-2-[(2S)-2-[6-(2,5-dioxopyrrol-1-yl)hexanamido]-3-methylbutanamido]pentanamido]phenyl]methyl N-[2-[([[2-(2,6-dioxopiperidin-3-yl)-1-oxo-3H-isoindol-5-yl]methyl]carbamoyl)amino]phenyl]-N-methylcarbamate (18.1 mg, 5%) as a white solid. LCMS (ESI): 1020 (M+H)⁺. ¹H NMR (400 MHz, DMSO-d₆) δ 10.99 (s, 1H), 9.96 (s, 1H), 8.19-8.06 (m, 3H), 7.79 (d, J=8.8 Hz, 1H), 7.70 (d, J=8.0 Hz, 1H), 7.53-7.41 (m, 5H), 7.20-7.05 (m, 4H), 7.00 (s, 2H), 6.95-6.90 (m, 1H), 5.95 (br s, 1H), 5.41 (s, 2H), 5.18-4.89 (m, 3H), 4.44-4.20 (m, 5H), 4.19-4.17 (m, 1H), 3.09 (s, 3H), 3.07-2.85 (m, 3H), 2.22-2.02 (m, 2H), 2.00-1.85 (m, 2H), 1.71-1.25 (m, 10H), 1.20-1.12 (m, 3H), 0.84-0.80 (m, 6H)

Scheme 4 shows how Compound (Id) was prepared from neoDegrader P1.

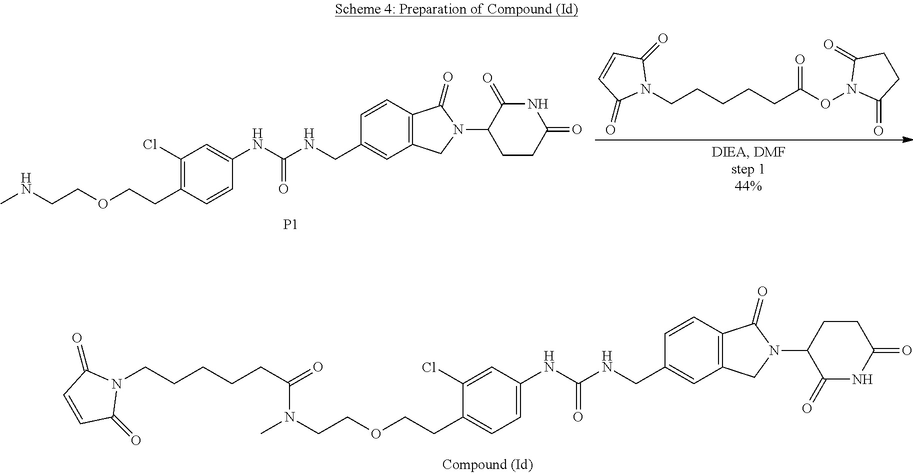

### Synthesis of Compound (Id)

To a stirred mixture of 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (P1, 40.00 mg, 0.076 mmol, 1.00 equiv) and 2,5-dioxopyrrolidin-1-yl 6-(2,5-dioxopyrrol-1-yl)hexanoate (25.00 mg, 0.081 mmol, 1.07 equiv) in DMF (2.00 mL) was added DIEA (20.00 mg, 0.16 mmol, 2.04 equiv) dropwise at room temperature. The resulting mixture was stirred for 3 h at room temperature under nitrogen atmosphere. The resulting mixture was quenched with water (30 mL), and extracted with DCM (3×30 mL). The combined organic layers were washed with water (30 mL), brine (30 mL), dried over Na₂SO₄. After filtration, the filtrate was concentrated to dryness under vacuum. The residue was purified by the following condition: Column: SunFire C18 OBD Prep Column, 100 um, 19 mm×250 mm; Mobile Phase A: water (0.05% TFA), Mobile Phase B: ACN; Flow rate: 25 mL/min; Gradient: 25 B to 55 B in 8.5 min; 220 nm; RT1:8 min; The collected fraction was lyophilized to afford N-[2-(2-[2-chloro-4-[([[2-(2,6-dioxopiperidin-3-yl)-1-oxo-3H-isoindol-5-yl]methyl]-carbamoyl)amino]phenyl]ethoxy)ethyl]-6-(2,5-dioxopyrrol-1-yl)-N-methylhexanamide (Compound (Id), 24 mg, 43%) as a white solid. LCMS: (ES, m/s): 721,723 (M+H)⁺; ¹H NMR (400 MHz, DMSO-d₆) δ 10.99 (s, 1H), 8.78 (s, 1H), 7.70-7.66 (m, 2H), 7.51 (s, 1H), 7.41 (d, J=9.6 Hz, 1H), 7.18-7.16 (m, 2H), 7.00 (d, J=5.6 Hz, 2H), 6.85-6.80 (m, 1H), 5.12-5.05 (m, 1H), 4.42-4.33 (m, 5H), 3.39-3.36 (m, 3H), 2.91-2.76 (m, 7H), 2.68-2.52 (m, 1H), 2.48-2.35 (m, 1H), 2.33-2.20 (m, 3H), 2.05-1.95 (m, 1H), 1.48-1.44 (m, 5H), 1.28-1.12 (m, 3H).

Schemes 5A and 5B show how to prepare a complex of neoDegrader P1 with an alternative tripeptide linker.

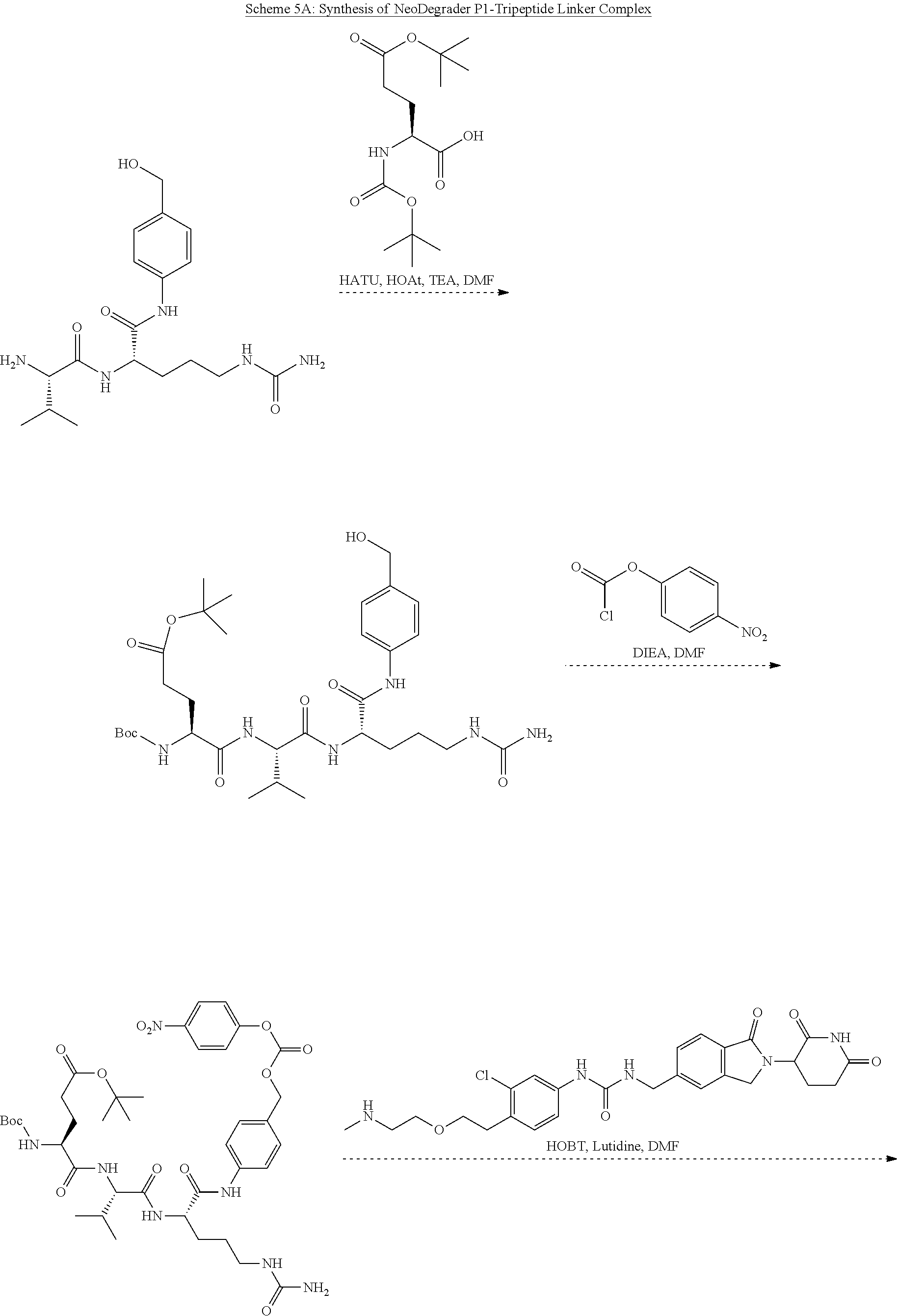 [SMILES: CC(C)[C@H](N)C(=O)N[C@@H](CCCNC(N)=O)C(=O)Nc1ccc(CO)cc1 ; CC(C)(C)OC(=O)CC[C@H](NC(=O)OC(C)(C)C)C(=O)O ; CC(C)[C@H](NC(=O)[C@H](CCC(=O)OC(C)(C)C)NC(=O)OC(C)(C)C)C(=O)N[C@@H](CCCNC(N)=O)C(=O)Nc1ccc(CO)cc1 ; O=C(Cl)Oc1ccc([N+](=O)[O-])cc1 ; CC(C)[C@H](NC(=O)[C@H](CCC(=O)OC(C)(C)C)NC(=O)OC(C)(C)C)C(=O)N[C@@H](CCCNC(N)=O)C(=O)Nc1ccc(COC(=O)Oc2ccc([N+](=O)[O-])cc2)cc1 ; CNCCOCCc1ccc(NC(=O)NCc2ccc3c(c2)CN(C2CCC(=O)NC2=O)C3=O)cc1Cl]

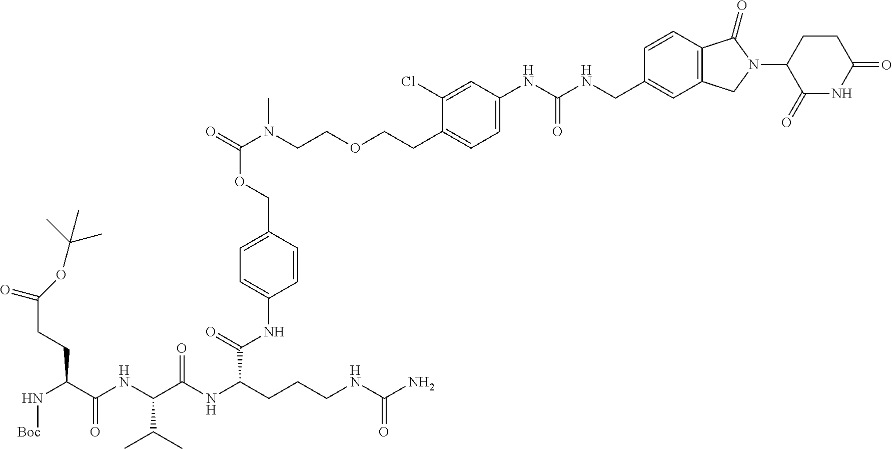 [SMILES: CC(C)[C@H](NC(=O)[C@H](CCC(=O)OC(C)(C)C)NC(=O)OC(C)(C)C)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

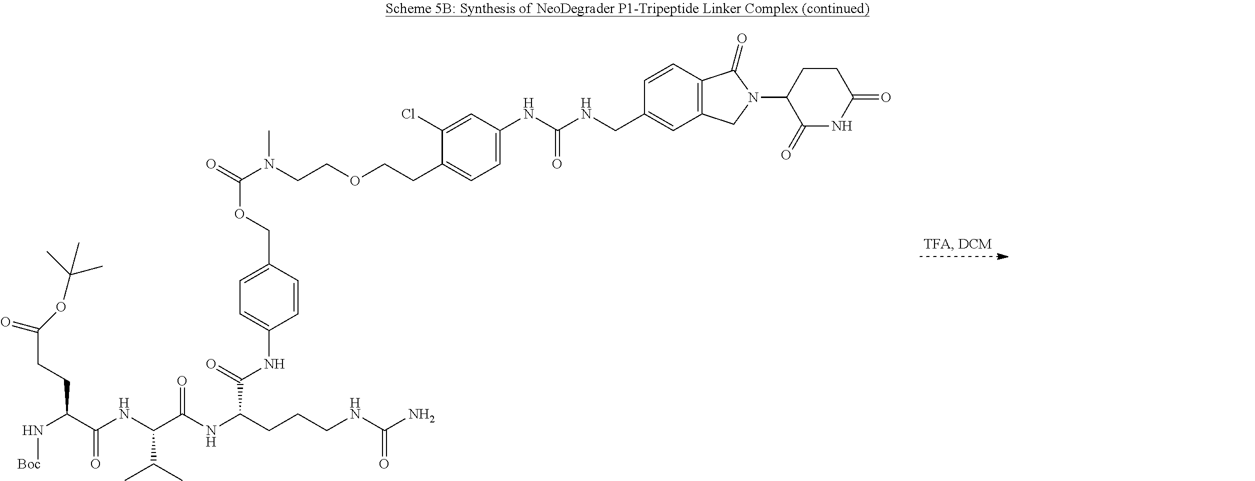 [SMILES: CC(C)[C@H](NC(=O)[C@H](CCC(=O)OC(C)(C)C)NC(=O)OC(C)(C)C)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

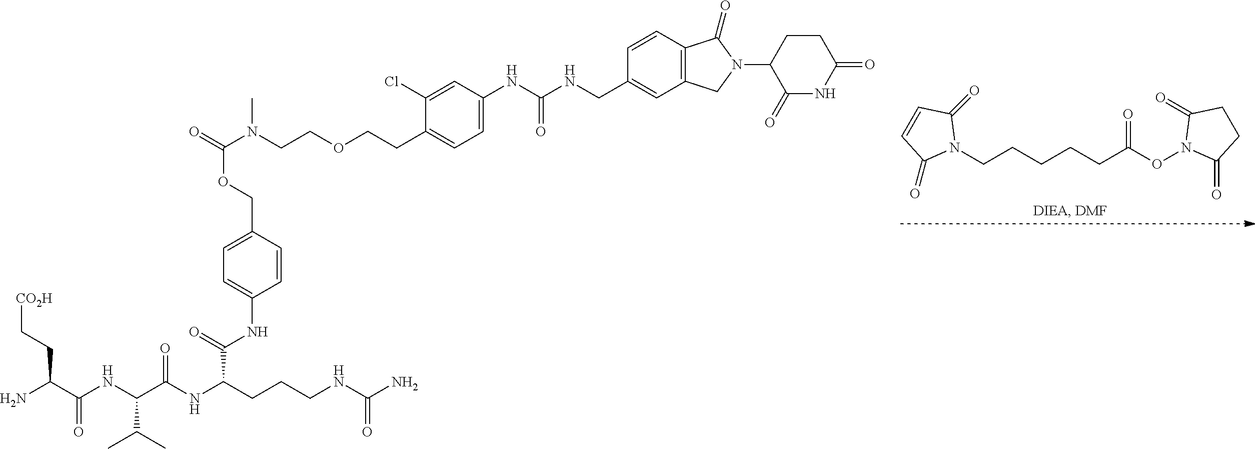 [SMILES: CC(C)[C@H](NC(=O)[C@@H](N)CCC(=O)O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1 ; O=C(CCCCCN1C(=O)C=CC1=O)ON1C(=O)CCC1=O]

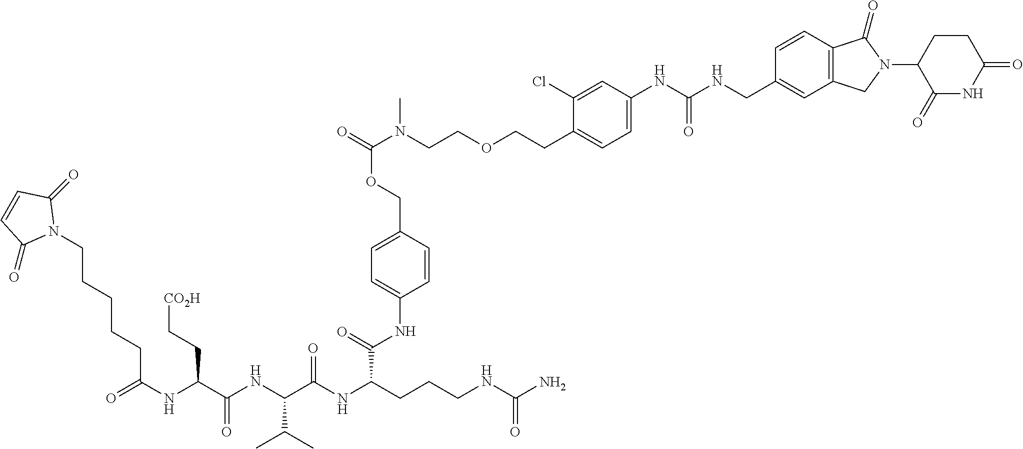 [SMILES: CC(C)[C@H](NC(=O)[C@H](CCC(=O)O)NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

Schemes 6A and 6B show how to prepare a complex of neoDegrader P1 with a β-glucuronide linker.

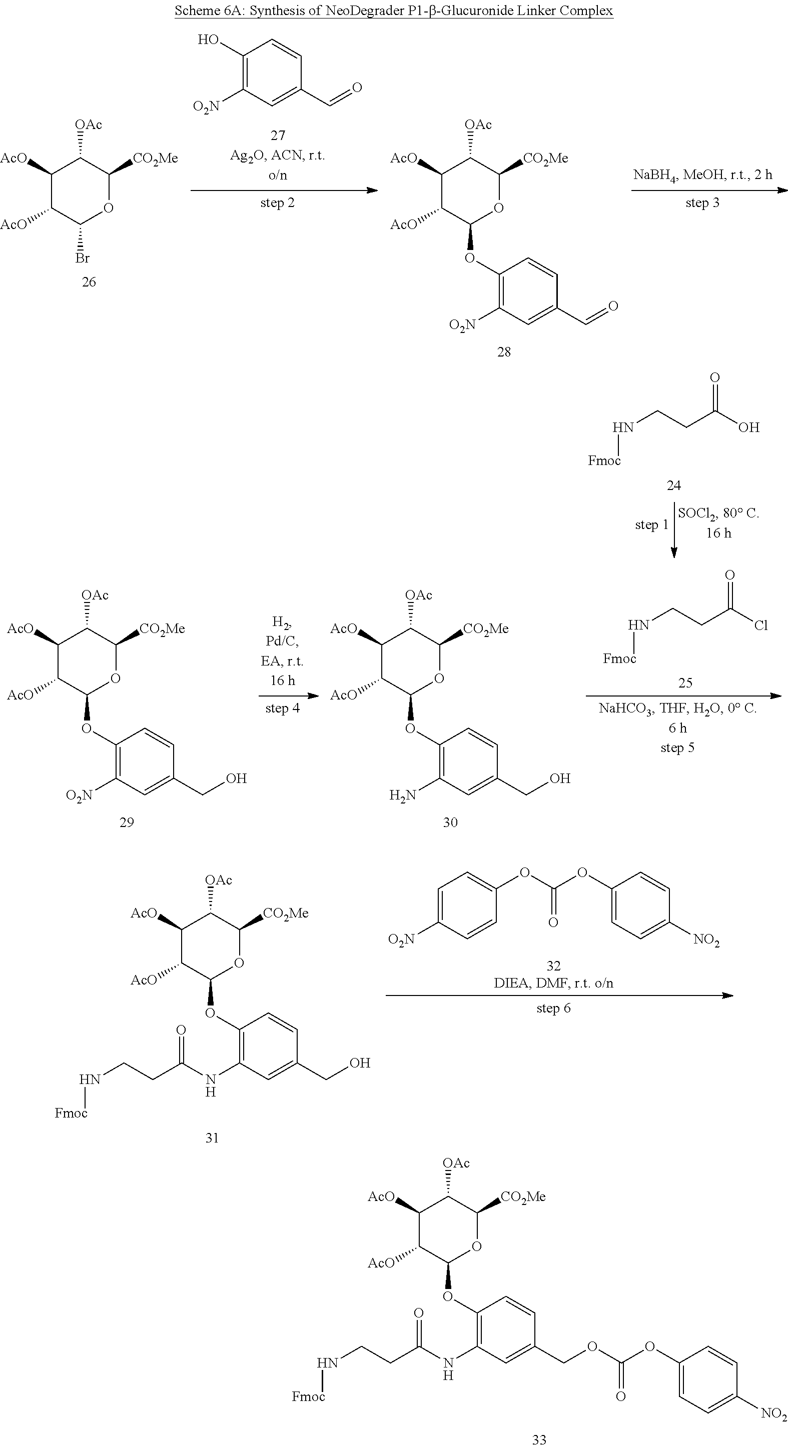

### Step 1. Synthesis of Compound 25

To a stirred mixture of 3-[[(9H-fluoren-9-ylmethoxy)carbonyl]amino]-propanoic acid (Compound 24, 5.00 g, 16.06 mmol, 1.00 equiv) in SOCl₂ (25 mL) at room temperature. The resulting mixture was stirred 16 h at 80° C. The desired product could be detected by LCMS (derivative with MeOH MS=326). LCMS indicated the reaction was completed. The resulting mixture was concentrated under vacuum to afford 9H-fluoren-9-ylmethyl N-(3-chloro-3-oxopropyl)carbamate (Compound 25, 7.5 g, crude) as a yellow oil. The crude product was used in the next step directly without further purification. ¹H-NMR analysis indicated it was the desired product (derivative with MeOH). ¹H-NMR (300 MHz, CDCl₃) δ 7.81-7.77 (m, 2H), 7.63-7.59 (m, 2H), 7.46-7.40 (m, 2H), 7.40-7.31 (m, 2H), 5.33 (s, 1H), 4.42 (d, J=3.0 Hz, 2H), 4.24 (t, J=6.0 Hz, 1H), 3.74-3.67 (m, 3H), 3.50 (d, J=3.0 Hz, 2H), 2.59 (t, J=6.0 Hz, 2H).

### Step 2. Synthesis of Compound 28

To a stirred solution of 4-formyl-2-nitrophenol (Compound 27, 4.21 g, 25.19 mmol, 1.00 equiv) and Ag₂O (7.00 g, 30.20 mmol, 1.20 equiv) in ACN (100 mL, 190.24 mmol, 75.00 equiv) were added Compound 26 (10.00 g, 25.17 mmol, 1.00 equiv) in portions at room temperature under N₂ atmosphere. The resulting mixture was stirred for overnight at room temperature under N₂ atmosphere. LCMS indicated the reaction was completed. The resulting mixture was filtered, the filter cake was washed with DCM (50 ml×3). The filtrate was concentrated under reduced pressure. The residue was purified by silica gel column chromatography, eluted with PE/EA (PE:EA=1:2) to afford methyl (2S,3S,4S,5R,6S)-3,4,5-tris(acetyloxy)-6-(4-formyl-2-nitrophenoxy)oxane-2-carboxylate (Compound 28, 10.5 g, 86%) as a white solid. ¹H-NMR analysis indicated it was the desired product. LCMS (ES, m/z): 484 [M+1]⁺. ¹H-NMR (300 MHz, CDCl₃) δ 10.00 (s, 1H), 8.34 (s, 1H), 8.13-8.09 (m, 1H), 7.52 (d, J=3.0 Hz, 1H), 5.47-5.29 (m, 4H), 4.37-4.35 (m, 1H), 3.75-3.73 (m, 3H), 2.17-2.06 (m, 9H).

### Step 3. Synthesis of Compound 29

To a stirred solution of methyl (2S,3 S,4S,5R,6S)-3,4,5-tris(acetyloxy)-6-(4-formyl-2-nitrophenoxy)oxane-2-carboxylate (Compound 28, 6.00 g, 12.41 mmol, 1.00 equiv) in MeOH (50 mL) were added NaBH₄ (0.47 g, 12.42 mmol, 1.00 equiv) in portions at RT under N₂ atmosphere. The resulting mixture was stirred for 2 h at room temperature under N₂ atmosphere. LCMS indicated the reaction was completed. The reaction was quenched with water at room temperature. The resulting was dried by Na₂SO₄. The resulting mixture was filtered, the filter cake was washed with DCM. The resulting mixture was concentrated under vacuum to afford methyl (2S,3S,4S,5R,6S)-3,4,5-tris(acetyloxy)-6-[4-(hydroxymethyl)-2-nitrophenoxy]oxane-2-carboxylate (Compound 29, 5.5 g, 91%) as a solid. LCMS (ES, m/z): 486 [M+H]+.

### Step 4. Synthesis of Compound 30

To a stirred mixture of methyl (2S,3S,4S,5R,6S)-3,4,5-tris(acetyloxy)-6-[4-(hydroxymethyl)-2-nitrophenoxy]oxane-2-carboxylate (Compound 29, 5.50 g, 11.33 mmol, 1.00 equiv) in EA (60 mL) were added Pd/C (1.10 g, 10%) in portions at room temperature. The resulting mixture was stirred for 16 h at room temperature under H2 atmosphere. LCMS indicated the reaction was completed. The resulting mixture was filtered, the filter cake was washed with DCM and MeOH, The filtrate was concentrated under vacuum to afford methyl (2S,3 S,4S,5R,6S)-3,4,5-tris(acetyloxy)-6-[2-amino-4-(hydroxymethyl)phenoxy]oxane-2-carboxylate (Compound 30, 4.0 g, 77%) as a solid. The crude product was used in the next step directly without further purification. LCMS (ES, m/z): 456[M+H]⁺.

### Step 5. Synthesis of Compound 31

To a stirred solution of methyl (2S,3S,4S,5R,6S)-3,4,5-tris(acetyloxy)-6-[2-amino-4-(hydroxymethyl)phenoxy]oxane-2-carboxylate (Compound 30, 1.00 g, 2.19 mmol, 1.00 equiv) and NaHCO₃ (0.20 g, 2.40 mmol, 1.1 equiv) in THE (10 mL) was added Compound 25 (0.87 g, 2.62 mmol, 1.20 equiv) in portions at 0° C. under N₂ atmosphere. The resulting mixture was stirred for 6 h at 0° C. under N₂ atmosphere. LCMS indicated the reaction was completed. The reaction was quenched with water at room temperature. The resulting mixture was extracted with DCM. The combined organic layers were concentrated under reduced pressure. The residue was purified by silica gel column chromatography, eluted with PE/EA (EA=100%) to afford methyl (2S,3S,4S,5R,6S)-3,4,5-tris(acetyloxy)-6-[2-(3-[[(9H-fluoren-9-ylmethoxy)carbonyl]amino]-propanamido)-4-(hydroxymethyl)phenoxy]oxane-2-carboxylate (Compound 31, 1.1 g, 66%) as a light yellow solid. LCMS (ES, m/z): 749 [M+H]⁺.

### Step 6. Synthesis Compound 33

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-(hydroxymethyl)phenoxy]oxane-2-carboxylate (Compound 31, 1.50 g, 2.00 mmol, 1.00 equiv) and bis(4-nitrophenyl) carbonate (Compound 32, 0.68 g, 2.24 mmol, 1.12 equiv) in DMF (15 mL) was added DIEA (0.52 g, 4.01 mmol, 2.00 equiv) in portions at 0° C. under N₂ atmosphere. The resulting mixture was stirred overnight at room temperature under nitrogen atmosphere. LCMS indicated the reaction was completed. The reaction mixture was purified by reverse flash chromatography with the following conditions: column, C18 silica gel; mobile phase, ACN in water (0.1% FA), 10% to 90% gradient in 40 min; detector, UV 254 nm. The collected fraction was concentrated to dryness in vacuum to afford methyl (2S,3S,4S,5R,6S)-3,4,5-tris(acetyloxy)-6-[2-(3-[[(9H-fluoren-9-ylmethoxy)carbonyl]amino]propanamido)-4-[[(4-nitrophenoxycarbonyl)oxy]methyl]phenoxy]oxane-2-carboxylate (Compound 33, 1.4 g, 48%) as a yellow solid. LCMS (ES, m/z): 914 [M+H]⁺.

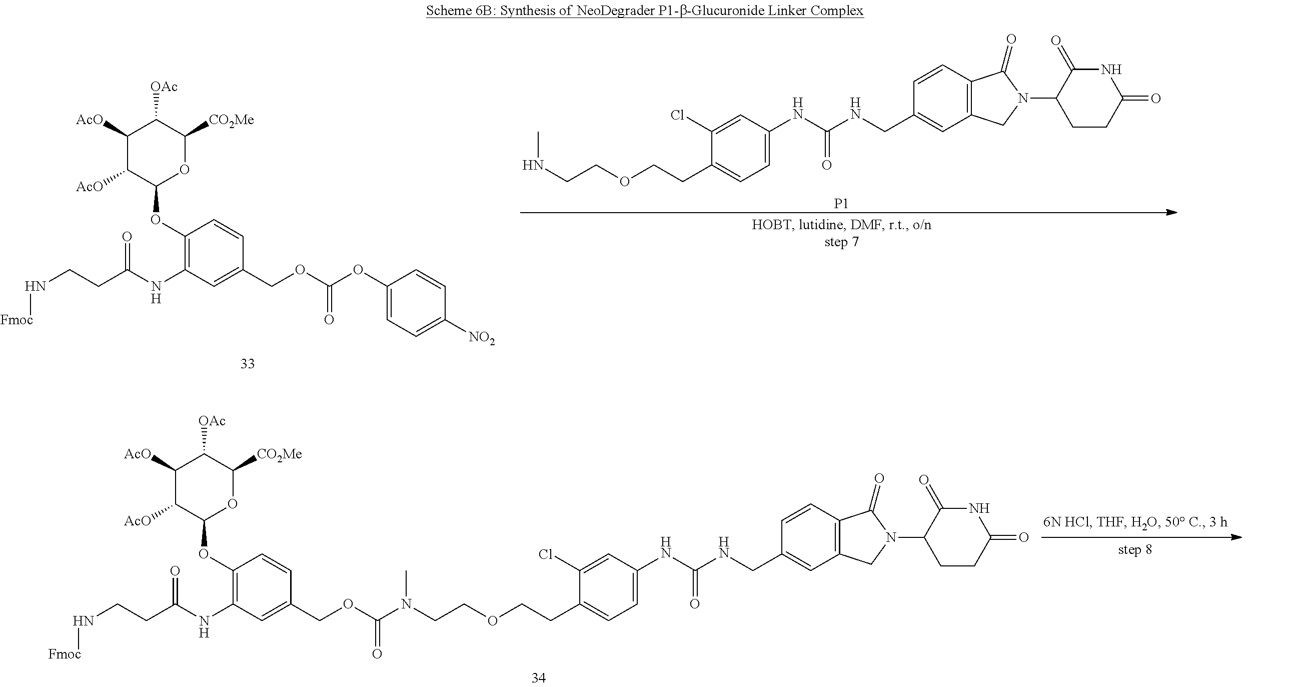

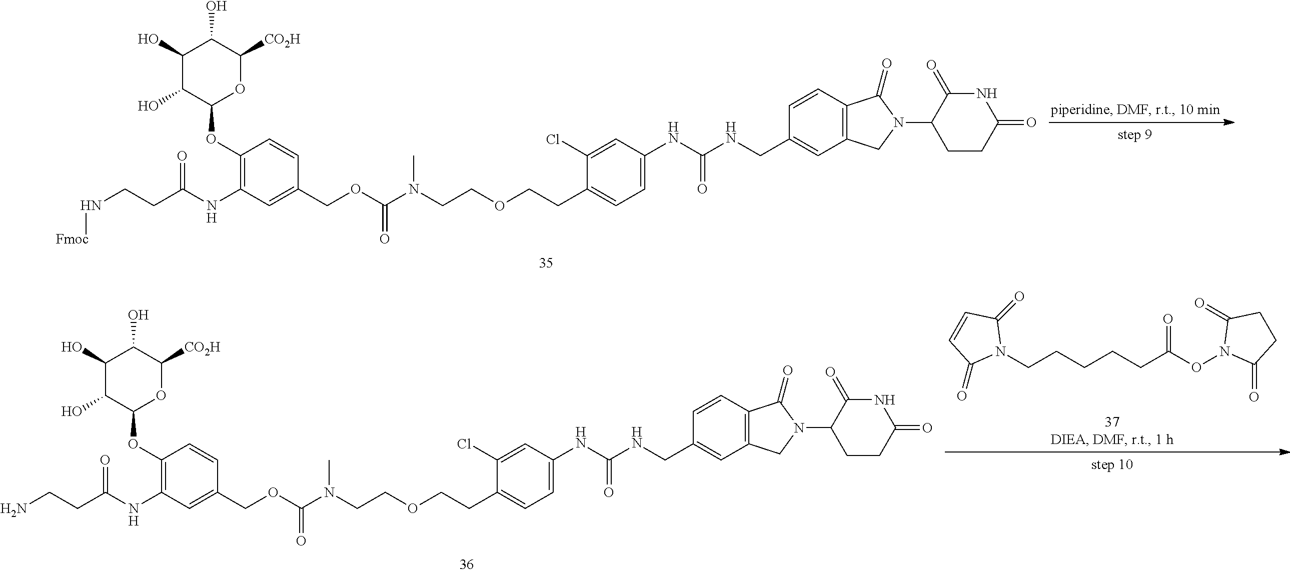

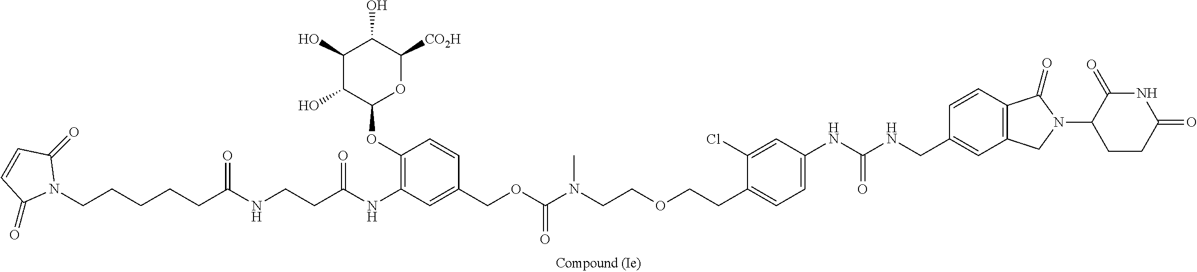 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(NC(=O)CCNC(=O)CCCCCN2C(=O)C=CC2=O)c1]

### Step 7. Synthesis Compound 34

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-[[(4-nitrophenoxycarbonyl)oxy]methyl]phenoxy]oxane-2-carboxylate (Compound 33, 1.00 g, 1.09 mmol, 1.00 equiv) and 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (neoDegrader P1, 0.58 g, 1.09 mmol, 1.00 equiv) in DMF (10 mL) were added HOBT (1.18 g, 8.72 mmol, 8.00 equiv) and 2,4-dimethylpyridine (1.07 g, 8.72 mmol, 8.00 equiv) in portions at room temperature under N₂ atmosphere. The resulting mixture was stirred for 16 h at room temperature under N₂ atmosphere. LCMS indicated the reaction was completed. The resulting mixture was used further purification. The residue was purified by reverse flash chromatography with the following conditions: column, C18 silica gel; mobile phase, ACN in water (0.1% FA), 10% to 80% gradient in 40 min; detector, UV 254 nm. The collected fraction was concentrated under vacuum to afford methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 34, 800 mg, 56%) as a solid. LCMS (ES, m/z): 1302[M+H]⁺.

### Step 8. Synthesis Compound 35

To a stirred mixture of methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 34, 800.00 mg, 0.61 mmol, 1.00 equiv) in THE (80 mL) was added HCl (6N, 80 mL) in portions at room temperature under N₂ atmosphere. The resulting mixture was stirred for 3 h at degrees 50° C. under nitrogen atmosphere. LCMS indicated the reaction was completed. The resulting mixture was concentrated under vacuum. The residue was purified by reverse flash chromatography with the following conditions: column, C18 silica gel; mobile phase, ACN in water (0.1% FA), 0% to 80% gradient in 40 min; detector, UV 254 nm. The collected fraction was lyophilized to afford (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid (Compound 35, 230 mg, 32%) as a white solid. LCMS (ES, m/z): 1162[M+H]⁺.

### Step 9. Synthesis of Compound 36

To a stirred solution of (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid (Compound 35, 230 mg, 0.2 mmol, 1.00 equiv) in DMF (2 mL) was added piperidine (0.4 mL) in portions at room temperature under nitrogen atmosphere. The resulting mixture was stirred for 10 min at room temperature under nitrogen atmosphere. LCMS indicated the reaction was completed. The resulting mixture was used directly further purification by Prep HPLC with the following conditions (Column: XSelect CSH Prep C18 OBD Column, 19×250 mm, 5 um; Mobile Phase A: water (0.05% TFA), Mobile Phase B: ACN; Flow rate: 25 mL/min; Gradient: 20 B to 40 B in 7 min; 220 nm; RT 1:5.78 min) to afford (2S,3S,4S,5R,6S)-6-[2-(3-aminopropanamido)-4-[([[2-(2-[2-chloro-4-[([[2-(2,6-dioxopiperidin-3-yl)-1-oxo-3H-isoindol-5-yl]methyl]carbamoyl)amino]phenyl]-ethoxy)ethyl](methyl)carbamoyl]oxy)methyl]phenoxy]-3,4,5-trihydroxyoxane-2-carboxylic acid (Compound 36.35 mg, 18%) as a white solid. LCMS (ES, m/z): 940[M+H]+.

### Step 10. Synthesis of Compound (Ie)

To a stirred solution of (2S,3S,4S,5R,6S)-6-[2-(3-aminopropanamido)-4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]phenoxy]-3,4,5-trihydroxyoxane-2-carboxylic acid (Compound 36, 30 mg, 0.03 mmol, 1.00 equiv) in DMF (3 mL) were added DIEA (13 mg, 0.10 mmol, 3.00 equiv) and Compound 37 (30 mg, 0.10 mmol, 3.00 equiv) in portions at room temperature under nitrogen atmosphere. The resulting mixture was stirred for 1 h at room temperature under nitrogen atmosphere. LCMS indicated the reaction was completed. The resulting mixture was purified by Prep-HPLC with the following conditions (Column: Xselect CSH OBD Column 30×150 mm 5 um, Mobile Phase A:water (0.1% FA), Mobile Phase B:ACN; Flow rate: 60 mL/min; Gradient: 21 B to 36 B in 10 min; 220 nm; RT 1:11.15 min). The collected fraction was lyophilized to afford (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl]-(methyl)carbamoyl]oxy)methyl]-2-[3-[6-(2,5-dioxopyrrol-1-yl)hexanamido]propanamido]phenoxy]-3,4,5-trihydroxyoxane-2-carboxylic acid (Compound (Je), 10.5 mg, 28%) as a white a solid. LCMS (ES, m/z): 1133[M+H]⁺. ¹H-NMR (300 MHz, DMSO-d₆) δ 10.9 (s, 1H), 9.13 (s, 1H), 8.16 (s, 1H), 7.92-7.68 (m, 4H), 7.52 (s, 1H), 7.44 (d, J=3.0 Hz, 1H), 7.18-6.99 (m, 7H), 5.76 (s, 1H), 5.20-5.10 (m, 2H), 4.98 (br s, 2H), 4.76-4.74 (m, 1H), 4.42-4.33 (m, 4H), 3.65 (br s, 1H), 3.58-3.54 (m, 5H), 3.35 (d, J=6 Hz, 2H), 2.90-2.83 (m, 7H), 2.57-2.55 (m, 3H), 2.45-2.30 (m, 1H), 2.02-1.98 (m, 4H), 1.48-1.42 (m, 5H), 1.40-1.20 (m, 3H).

Scheme 7 shows how to prepare a complex of neoDegrader P6 with a hydrazine linker.

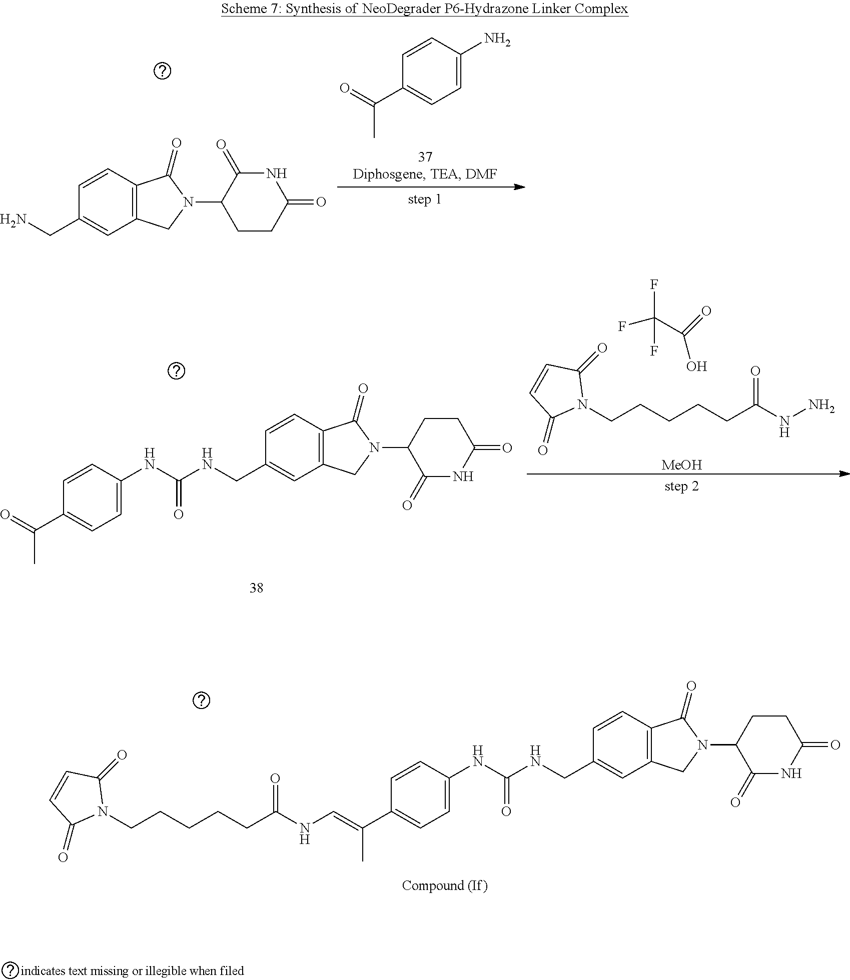

### Step 1. Synthesis of Compound 38

To a stirred solution of 4-aminoacetophenone (Compound 37, 100 mg, 0.73 mmol, 1.00 equiv) in THF (2.00 mL) were added diphosgene (0.40 mL) dropwise at room temperature. The resulting mixture was stirred for 30 min at 0′° C. The resulting mixture was concentrated under vacuum. The resulting solid was re-dissolved in DMF (1.50 mL). To the stirred solution was 3-[5-(aminomethyl)-1-oxo-3H-isoindol-2-yl]piperidine-2,6-dione (INT1, 200 mg, 0.73 mmol, 1.00 equiv) in DMF (3.00 mL) and TEA (0.50 mL) dropwise at room temperature. The resulting mixture was stirred for 1 h at 0′° C. LCMS indicated the reaction was completed. The mixture was added water (5 mL) and extracted with CH₂Cl₂ (3×10 mL). The organic layer was concentrated under vacuum. The residue was purified by reverse flash chromatography with the following conditions: column. C18 silica gel; mobile phase. ACN in water (0.05% TFA), 10% to 50% gradient in 35 min; detector, UV 254 nm. The collection fraction was concentrated to dryness to afford 1-(4-acetylphenyl)-3-[[2-(2,6-dioxopiperidin-3-yl)-1-oxo-3H-isoindol-5-yl]methyl]urea (Compound 38, 80 mg, 25%) as a light yellow solid. LCMS: (ES.m/z): 435[M+1]⁺.

### Step 2. Synthesis of Compound (If)

The mixture of 1-(4-acetylphenyl)-3-[[2-(2,6-dioxopiperidin-3-yl)-1-oxo-3H-isoindol-5-yl]methyl]urea (Compound 38, 80.00 mg, 0.18 mmol, 1.00 equiv) and 6-(2,5-dioxopyrrol-1-yl)hexanehydrazide; trifluoroacetic acid (75 mg, 1.20 equiv) in methanol (5.00 mL) was stirred for overnight at 50 degrees C. The mixture was cooled down to room temperature. LCMS indicated the reaction was completed. The precipitated solids were collected by filtration and washed with MeOH (2×5 mL). The crude solid was purified by reverse flash chromatography with the following conditions: C18 column; mobile phase, ACN in water (0.1% FA), 10% to 50% gradient in 30 min; detector, UV 254 nm. The collected fraction was extracted with DCM (3×5 mL) and concentrated under vacuum. This resulted in 3-[[2-(2,6-dioxopiperidin-3-yl)-1-oxo-3H-isoindol-5-yl]methyl]-1-[4-[(1E)-1-[[6-(2,5-dioxopyrrol-1-yl)hexanamido]imino]ethyl]phenyl]urea (Compound (If), 4.4 mg, 3.7%) as an off-white solid. LCMS: (ES.m/z): 642[M+1]⁺. ¹H NMR (400 MHz, DMSO-d₆) δ 10.99 (s, 1H), 10.26-10.15 (m, 1H), 8.82 (s, 1H), 7.69-7.62 (m, 3H), 7.52-7.43 (m, 4H), 7.01-6.99 (m, 2H), 5.13-5.09 (m, 1H), 4.42-4.33 (m, 4H), 2.98-2.82 (m, 1H), 2.62-2.58 (m, 2H), 2.20-2.12 (m, 2H), 1.58-1.51 (m, 6H), 1.26-1.09 (m, 6H)

Scheme 8 shows how to prepare a complex of neoDegrader P2 with a quaternary amine linker.

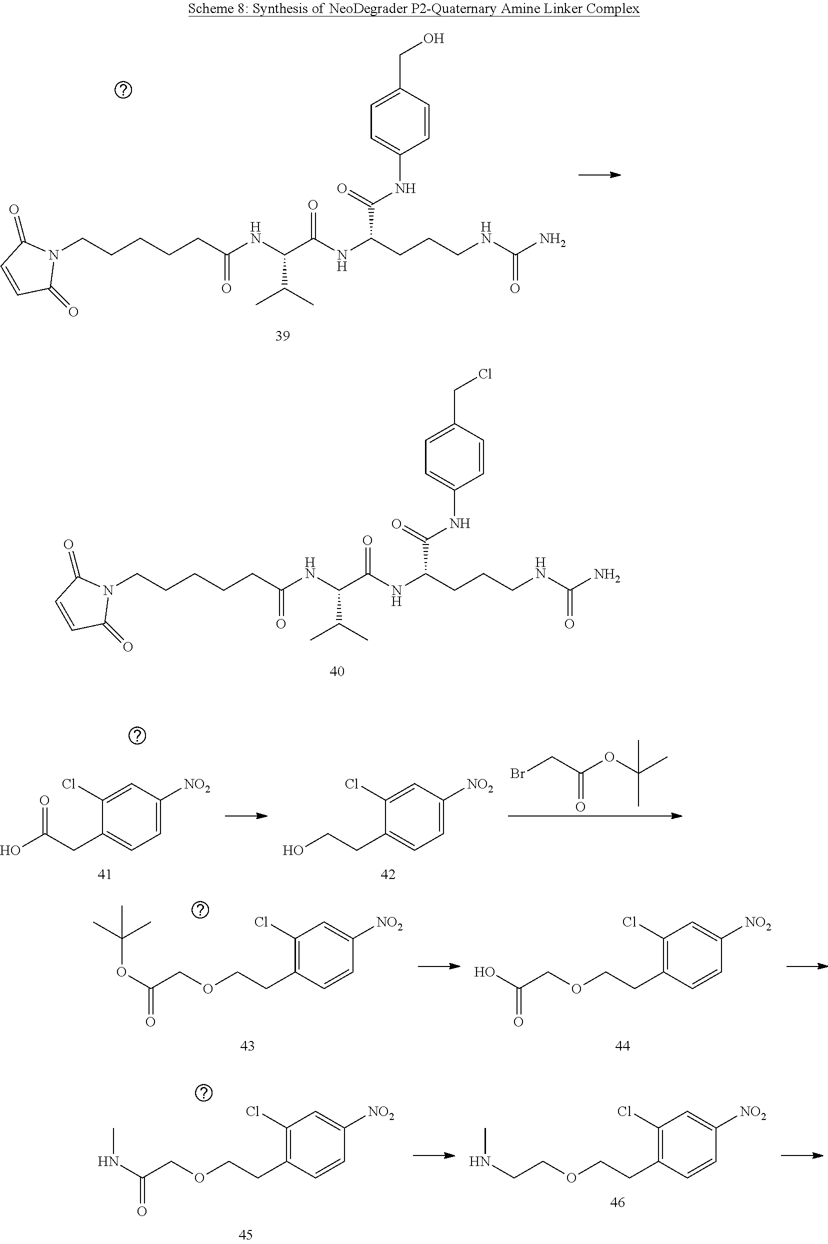

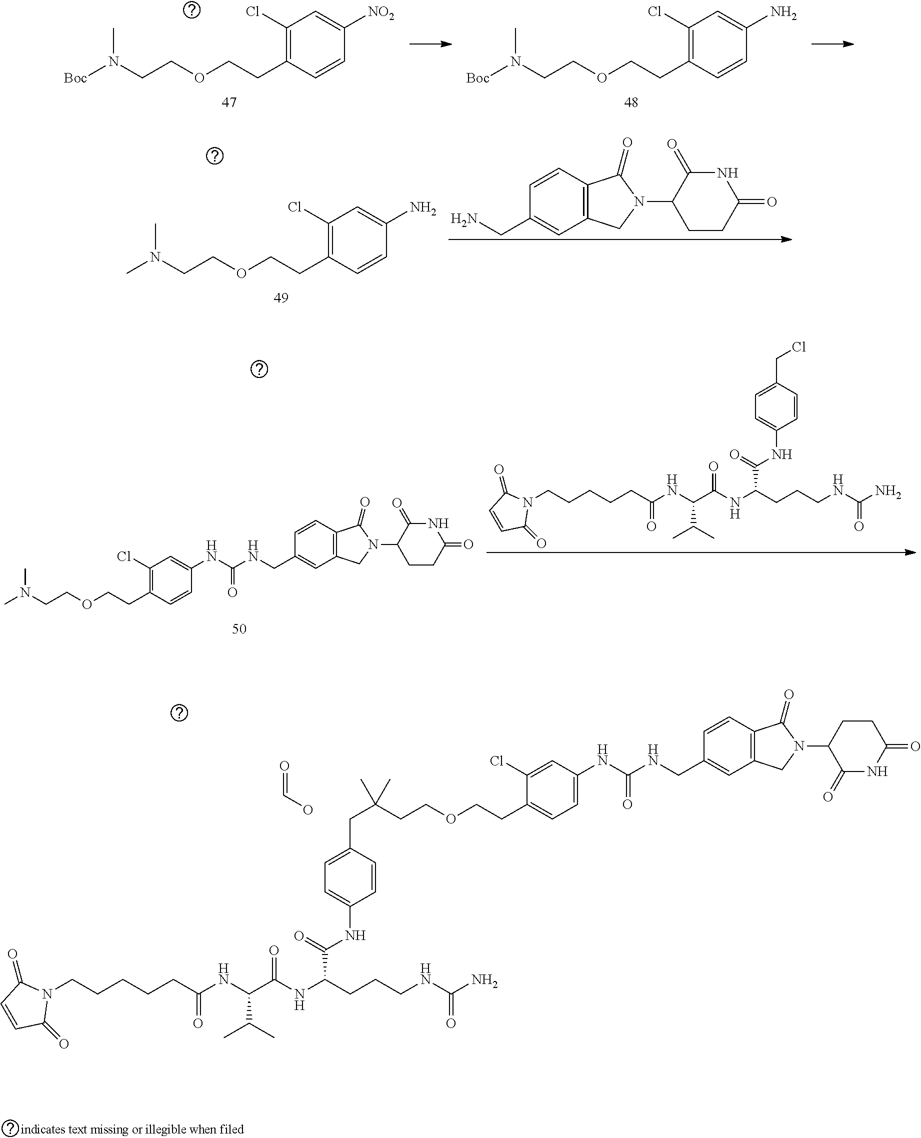

### Step 1. Synthesis of Compound 40

To a stirred solution of N-[(1S)-1-[[(1S)-4-(carbamoylamino)-1-[[4-(hydroxymethyl)phenyl]carbamoyl]butyl]carbamoyl]-2-methylpropyl]-6-(2,5-dioxopyrrol-1-yl)hexanamide (Compound 39, 100 mg, 0.18 mmol, 1.00 equiv) in DMF (2 mL) was added SOC12 (20 mg, 0.18 mmol, 1 equiv) in DCM (2 mL) dropwise under N₂ at 0 TC. The resulting mixture was stirred at 0° C. for 1 h. LCMS indicated the reaction was completed. The reaction mixture was diluted with ice-cooled water (20 mL), extracted with DCM (10 mL*3), the combined organic layer was washed with water (10 mL), brine (10 mL), dried over anhydrous sodium sulfate and concentrated to dryness under vacuum to give N-[(1S)-1-[[(1S)-4-(carbamoylamino)-1-[[4-(chloromethyl)phenyl]-carbamoyl]butyl]carbamoyl]-2-methylpropyl]-6-(2,5-dioxopyrrol-1-yl)hexanamide (Compound 40, 80 mg, 53%) of the product as a white solid. LCMS (ES, m/z): 591,593 [M+H]⁺

### Step 2. Synthesis of Compound 42

To a stirred mixture of (2-chloro-4-nitrophenyl)acetic acid (Compound 41, 8.60 g, 39.9 mmol, 1.00 equiv) in THE (130 mL) was added BH₃-Me₂S (10.00 mL, 105.4 mmol, 2.64 equiv) dropwise at 0° C. The resulting mixture was stirred for 4 h at 70° C. under nitrogen atmosphere. TLC (PE:EA=1:2) indicated the reaction was completed. The mixture was allowed to cool down to room temperature. The resulting mixture was concentrated under vacuum. The residue was purified by silica gel column chromatography, eluted with PE/EtOAc (1:1) to afford 2-(2-chloro-4-nitrophenyl)ethanol (Compound 42, 7.7 g, 96%) as a yellow solid. ¹H NMR (400 MHz, CDCl₃) δ 8.27 (d, J=4.0 Hz, 1H), 8.11-8.07 (m, 1H), 7.53 (d, J=8.0 Hz, 1H), 3.99 (t, J=8.0 Hz, 2H), 3.15 (t, J=8.0 Hz, 2H).

### Step 3. Synthesis of Compound 43

To a stirred mixture of 2-(2-chloro-4-nitrophenyl)ethanol, (Compound 42, 7.70 g, 38.2 mmol, 1.00 equiv) and tert-butyl 2-bromoacetate (57.74 g, 296.0 mmol, 7.75 equiv) in toluene (70 mL) was added Bu₄NHSO₄ (10.37 g, 30.6 mmol, 0.80 equiv) in portions at 0° C. To the above mixture was added NaOH (15.00 g, 375.0 mmol, 9.82 equiv) in H₂O (90 mL) dropwise over 30 h at 0° C. The resulting mixture was stirred for additional 4 h at room temperature. TLC (PE:EA=3:1) indicated the reaction was completed The resulting mixture was extracted with EtOAc (3×200 mL). The combined organic layers were washed with brine (200 mL), dried over anhydrous Na₂SO₄. After filtration, the filtrate was concentrated under reduced pressure. The residue was purified by silica gel column chromatography, eluted with PE/EtOAc (5:1) to afford tert-butyl 2-[2-(2-chloro-4-nitrophenyl)ethoxy]acetate (Compound 43, 12.2 g, 91%) as a yellow oil. ¹H NMR (300 MHz, CDCl₃) δ 8.20 (d, J=4.0 Hz, 1H), 8.07-8.03 (m, 1H), 7.61 (d, J=8.1 Hz, 1H), 4.11 (s, 2H), 3.83 (t, J=8.1 Hz, 2H), 3.16 (t, J=8.1 Hz, 2H), 1.45 (s, 9H).

### Step 4. Synthesis of Compound 44

To a stirred mixture of tert-butyl 2-[2-(2-chloro-4-nitrophenyl)ethoxy]acetate (Compound 43, 12.20 g, 38.6 mmol, 1.00 equiv) in DCM (120 mL) was added TFA (20 mL) dropwise at 0° C. The resulting mixture was stirred for 4 h at room temperature. LCMS indicated the reaction was completed. The resulting mixture was concentrated under reduced pressure. This resulted in [2-(2-chloro-4-nitrophenyl)ethoxy]acetic acid (Compound 44, 8.4 g, 83%) as a yellow solid. LCMS: (ES, m/s): 517 (2M−H)^{- 1}H NMR (400 MHz, DMSO-d₆) δ 12.64 (s, 1H), 8.20 (d, J=4.0 Hz, 1H), 8.11-8.08 (m, 1H), 7.72 (d, J=8.0 Hz, 1H), 4.06 (s, 2H), 3.74 (t, J=8.0 Hz, 2H), 3.06 (t, J=8.0 Hz, 2H).

### Step 5. Synthesis of Compound 45

To a stirred mixture of [2-(2-chloro-4-nitrophenyl)ethoxy]acetic acid, (Compound 44, 8.40 g, 32.35 mmol, 1.00 equiv) and HATU (19.19 g, 50.47 mmol, 1.56 equiv) in DMF (80 mL) were added CH₃NH₂·HCl (2.69 g, 39.79 mmol, 1.23 equiv) and DIEA (17.31 g, 133.93 mmol, 4.14 equiv) at 0° C. under nitrogen atmosphere. The resulting mixture was stirred for 4 h at room temperature under nitrogen atmosphere. LCMS indicated the reaction was completed. The reaction was quenched with water/ice. The resulting mixture was extracted with DCM (3×50 mL). The combined organic layers were washed with brine (50 mL), dried over anhydrous Na₂SO₄. After filtration, the filtrate was concentrated under reduced pressure. The residue was purified by silica gel column chromatography, eluted with (DCM:MeOH=10:1) to afford 2-[2-(2-chloro-4-nitrophenyl)ethoxy]-N-methylacetamide (Compound 45, 7.2 g, 81%) as a yellow oil. LCMS: (ES, m/s): 273,275 (M+H)⁺

### Step 6. Synthesis of Compound 46

To a stirred mixture of 2-[2-(2-chloro-4-nitrophenyl)ethoxy]-N-methylacetamide (Compound 45, 7.20 g, 26.40 mmol, 1.00 equiv) in THE (70 mL) was added BH₃-THF (10M in THF, 52.0 mL, 520.0 mmol, 20 equiv) dropwise at room temperature. The resulting mixture was stirred for 4 h at 70° C. LCMS indicated the reaction was completed. The mixture was allowed to cool down to room temperature. The reaction was quenched with MeOH. The residue was acidified to pH 6 with 1N HCl. The resulting mixture was extracted with EtOAc (20 mL). The aqueous phase was basified to pH 8 with saturated NaHCO₃ (sat., aq.). The resulting mixture was extracted with EtOAc (3×100 mL), washed with brine (50 mL) and dried over anhydrous Na₂SO₄. After filtration, the filtrate was concentrated under reduced pressure. The residue was purified by silica gel column chromatography, eluted with (DCM:MeOH=8:1) to afford [2-[2-(2-chloro-4-nitrophenyl)ethoxy]ethyl](methyl)amine (Compound 46, 5.4 g, 79%) as a yellow solid. LCMS: (ES, m/s): 259,261 (M+H)⁺; ¹H NMR (400 MHz, DMSO-d₆) δ 8.26 (d, J=4.0 Hz, 1H), 8.15-8.12 (m, 1H), 7.73 (d, J=8.0 Hz, 1H), 3.72 (t, J=8.0 Hz, 2H), 3.61 (t, J=8.0 Hz, 2H), 3.10 (t, J=8.0 Hz, 2H), 2.87 (t, J=8.0 Hz, 2H), 2.40 (s, 3H).

### Step 7. Synthesis of Compound 47

To a stirred mixture of [2-[2-(2-chloro-4-nitrophenyl)ethoxy]ethyl](methyl)amine (Compound 46, 4.00 g, 15.46 mmol, 1.00 equiv) and Boc₂O (3.80 g, 17.41 mmol, 1.13 equiv) in THF (20.00 mL) was added NaHCO₃ (4.00 g, 47.61 mmol, 3.08 equiv) in H₂O (20.00 mL) dropwise at room temperature. The resulting mixture was stirred for overnight at room temperature. LCMS indicated the reaction was completed. The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with (DCM:MeOH=12:1) to afford tert-butyl N-[2-[2-(2-chloro-4-nitrophenyl)ethoxy]ethyl]-N-methylcarbamate (Compound 47, 4.8 g, 77%) as a yellow solid.

LCMS: (ES, m/s): 359,361 (M+H)⁺; ¹H NMR (400 MHz, DMSO-d₆) δ 8.24 (d, J=4.0 Hz, 1H), 8.13-8.10 (m, 1H), 7.67 (d, J=8.0 Hz, 1H), 4.05-4.00 (m, 1H), 3.69 (t, J=8.0 Hz, 2H), 3.50 (t, J=8.0 Hz, 2H), 3.28 (t, J=8.0 Hz, 2H), 3.07 (t, J=8.0 Hz, 2H), 2.75 (s, 3H), 1.36 (s, 9H).

### Step 8. Synthesis of Compound 48

To a stirred mixture of tert-butyl N-[2-[2-(2-chloro-4-nitrophenyl)ethoxy]ethyl]-N-methylcarbamate, (Compound 47, 5.60 g, 15.6 mmol, 1.00 equiv) in EtOH (112.00 mL) were added NH₄Cl (2.50 g, 46.74 mmol, 2.99 equiv) in H₂O (12.00 mL) and Fe (4.40 g, 78.79 mmol, 5.05 equiv) at room temperature. The resulting mixture was stirred for 3 h at 80° C. LCMS indicated the reaction was completed. The mixture was allowed to cool down to room temperature. The resulting mixture was concentrated under reduced pressure. The resulting mixture was extracted with DCM (3×30 mL). The combined organic layers were washed with brine (30 mL), dried over anhydrous Na₂SO₄. After filtration, the filtrate was concentrated under reduced pressure. The residue was purified by silica gel column chromatography, eluted with (DCM:MeOH=10:1) to afford tert-butyl N-[2-[2-(4-amino-2-chlorophenyl)ethoxy]ethyl]-N-methylcarbamate (Compound 48, 4.2 g, 81%) as a yellow oil. LCMS: (ES, m/s): 329,331 (M+H); ¹H NMR (400 MHz, DMSO-d₆) δ 6.96 (d, J=8.0 Hz, 1H), 6.59 (d, J=4.0 Hz, 1H), 6.46-6.43 (m, 1H), 5.18 (br s, 2H), 3.50-3.45 (m, 4H), 3.29-3.26 (m, 2H), 2.75-2.71 (m, 5H), 1.38 (s, 9H).

### Step 9. Synthesis of Compound 49

To a solution of tert-butyl N-[2-[2-(4-amino-2-chlorophenyl)ethoxy]ethyl]-N-methylcarbamate (Compound 48, 100 mg, 0.30 mmol, 1.00 equiv) in THE (3 mL) was added LiAlH₄ (92 mg, 2.43 mmol, 8.00 equiv) in THE (2 mL) at 0° C. under nitrogen atmosphere. The resulting mixture was stirred at room temperature for 16 hours. The five reactions were run in parallel. LCMS indicated the reaction was completed. Then the reaction was quenched with 1N NaOH (10 mL), filtered, concentrated to dryness under vacuum and then the residue was purified by reverse flash chromatography with the following conditions: column, C18 silica gel; mobile phase, ACN in water (0.1% FA), 0% to 60% gradient in 30 min; detector, UV 254 nm. The collected fraction was concentrated to dryness to give 3-chloro-4-[2-[2-(dimethylamino)ethoxy]ethyl]aniline, 49 (180 mg, 44%) as a yellow oil. LCMS (ES, m/z): 243,245 [M+H]⁺

### Step 10. Synthesis of Compound 50

To a solution of 3-chloro-4-[2-[2-(dimethylamino)ethoxy]ethyl]aniline (Compound 49, 140 mg, 0.58 mmol, 1.00 equiv) in THE (9 mL) was added diphosgene (137 mg, 0.69 mmol, 1.20 equiv) at 0° C. under nitrogen atmosphere. The resulting mixture was stirred at 0° C. for 1 hour. Then the reaction solution was concentrated to dryness under vacuum. The residue was re-dissolved in DMF (2 mL) and then added into a solution of 3-[5-(aminomethyl)-1-oxo-3H-isoindol-2-yl]piperidine-2,6-dione (158 mg, 0.58 mmol, 1.00 equiv) and TEA (117 mg, 1.15 mmol, 2.00 equiv) in DMF (4 mL) dropwise under nitrogen atmosphere. The resulting mixture was stirred at room temperature for 16 h. LCMS indicated the reaction was completed. The reaction mixture was diluted with methanol and the resulting solution was purified by reverse flash chromatography with the following conditions: column, C18 silica gel; mobile phase, ACN in water (0.1% FA), 0% to 50% gradient in 30 min; detector, UV 254 nm to give 100 mg of the product as a colorless solid. The crude product was purified by Prep-HPLC with the following conditions: column: XBridge Shield RP18 OBD Column, 19×250 mm, 10 um; Mobile Phase A: Water (0.1% FA), Mobile Phase B: ACN; Flow rate: 25 mL/min; Gradient: 14% to 32% in 7 min; 220 nm; RT1: 5.25 min. The collected fraction was lyophilized to give 1-(3-chloro-4-[2-[2-(dimethylamino)ethoxy]ethyl]phenyl)-3-[[2-(2,6-dioxopiperidin-3-yl)-1-oxo-3H-isoindol-5-yl]methyl]urea (Compound 50, 60 mg, 18%) as a colorless solid. LCMS (ES, m/z): 542,544 [M+H]+

### Step 11. Synthesis of Compound (Ig)

To a solution of N-[(1S)-1-[[(1S)-4-(carbamoylamino)-1-[[4-(chloromethyl)phenyl]-carbamoyl]butyl]carbamoyl]-2-methylpropyl]-6-(2,5-dioxopyrrol-1-yl)hexanamide, (Compound 40, 66 mg, 0.11 mmol, 1.00 equiv), 1-(3-chloro-4-[2-[2-(dimethylamino)ethoxy]ethyl]phenyl)-3-[[2-(2,6-dioxopiperidin-3-yl)-1-oxo-3H-isoindol-5-yl]methyl]urea (Compound 50, 60 mg, 0.11 mmol, 1.00 equiv) and DIEA (29 mg, 0.22 mmol, 2.00 equiv) in DMF (1 mL) was added TBAI (4 mg, 0.01 mmol, 0.10 equiv) at room temperature in air. The resulting mixture was stirred at room temperature for 16 hours. LCMS traces showed the reaction was completed. The resulting mixture was purified by reverse phase column chromatography with the following conditions: column, C18 silica gel; mobile phase, ACN in water (0.05% TFA), 5% to 45% gradient in 40 min; detector, UV 254 nm to give 90 mg of the crude product as a yellow oil. Then the crude product was re-purified by the following condition: Column: Xselect CSH OBD Column 30*150 mm 5 um, n; Mobile Phase A: Water(0.1% FA), Mobile Phase B: ACN; Flow rate: 60 mL/min; Gradient: 15 B to 35 B in 7 min; 220 nm; RT1:6.00 min to afford ([4-[(2S)-5-(carbamoylamino)-2-[(2S)-2-[6-(2,5-dioxopyrrol-1-yl)hexanamido]-3-methylbutanamido]pentanamido]phenyl]methyl)[2-(2-[2-chloro-4-[([[2-(2,6-dioxopiperidin-3-yl)-1-oxo-3H-isoindol-5-yl]methyl]carbamoyl)amino]phenyl]ethoxy)ethyl]dimethylazanium, Compound (Ig) (19 mg, 14.8%) as a white solid. LCMS (ES, m/z): 1096 [M-FA]⁺, 549 [1/2(M-FA)]⁺; ¹H NMR (400 MHz, CD₃OD) δ 8.48 (s, 1H), 7.77-7.72 (m, 3H), 7.55-7.47 (m, 3H), 7.37-7.35 (d, J=8.4 Hz, 2H), 7.18-7.14 (m, 2H), 6.77 (s, 2H), 5.17-5.13 (q, J=8, 4 Hz, 1H), 4.51-4.46 (m, 5H), 4.35 (s, 2H), 4.12 (d, J=8.0 Hz, 1H), 3.90 (s, 2H), 3.79 (t, J=5.6 Hz, 2H), 3.45 (t, J=7.2 Hz, 4H), 3.22-3.15 (m, 1H), 3.11-3.05 (m, 1H), 3.00 (t, J=6.0 Hz, 2H), 2.92 (s, 6H), 2.89-2.84 (m, 1H), 2.81-2.73 (m, 1H), 2.54-2.43 (m, 1H), 2.27 (t, J=7.2 Hz, 2H), 2.21-2.12 (m, 1H), 2.10-2.02 (m, 1H), 1.95-1.82 (m, 1H), 1.78-1.69 (m, 1H), 1.64-1.59 (m, 7H), 1.32-1.25 (m, 2H), 0.98-0.96 (m, 6H).

Schemes 9A and 9B show how to prepare a complex of neoDegrader P13 with a peptide-containing linker.

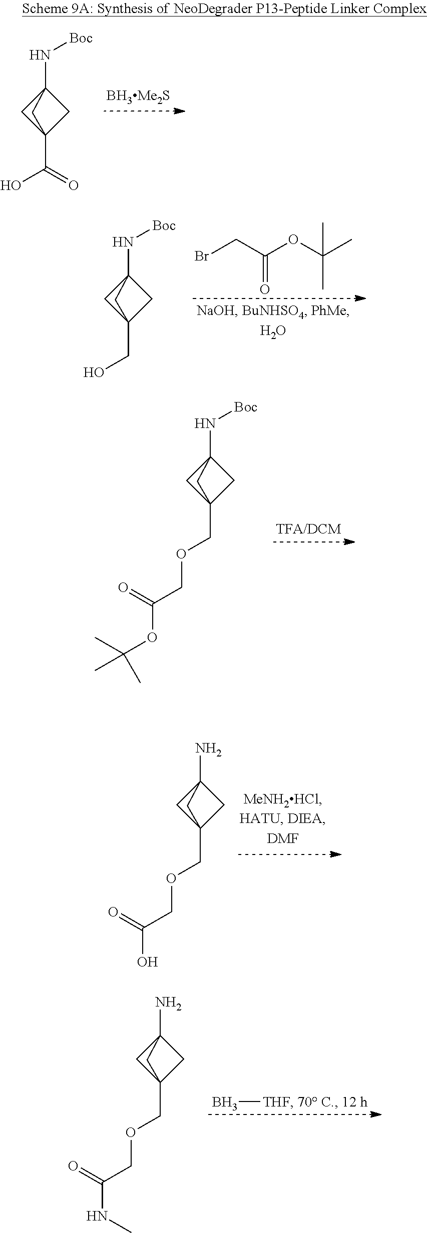

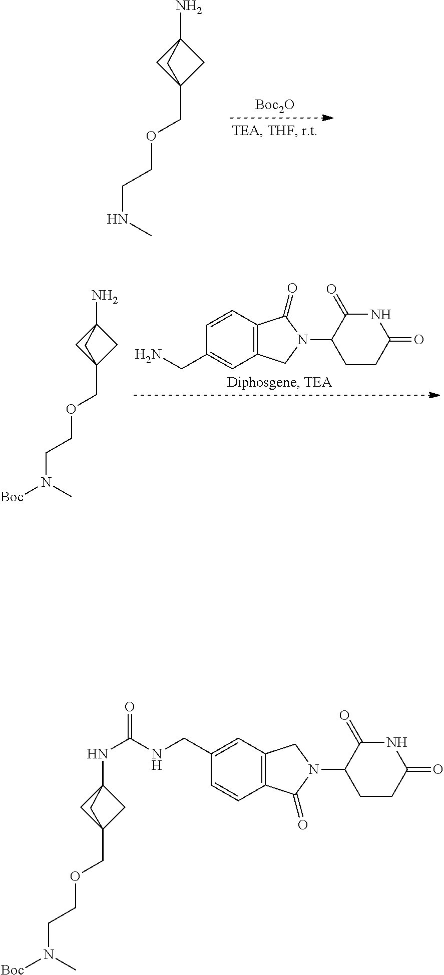

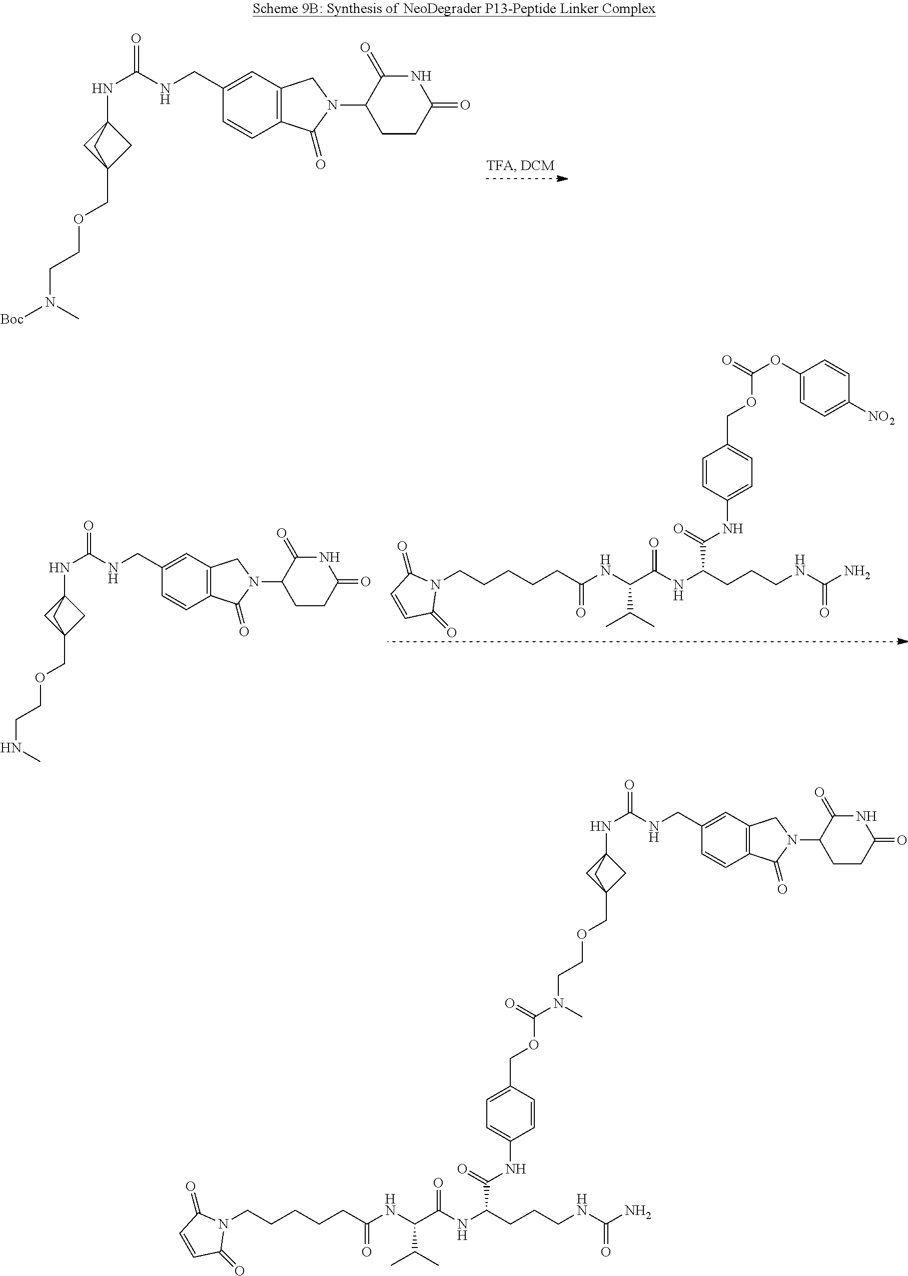

Scheme 10 shows the synthesis of compounds of formula (Ih).

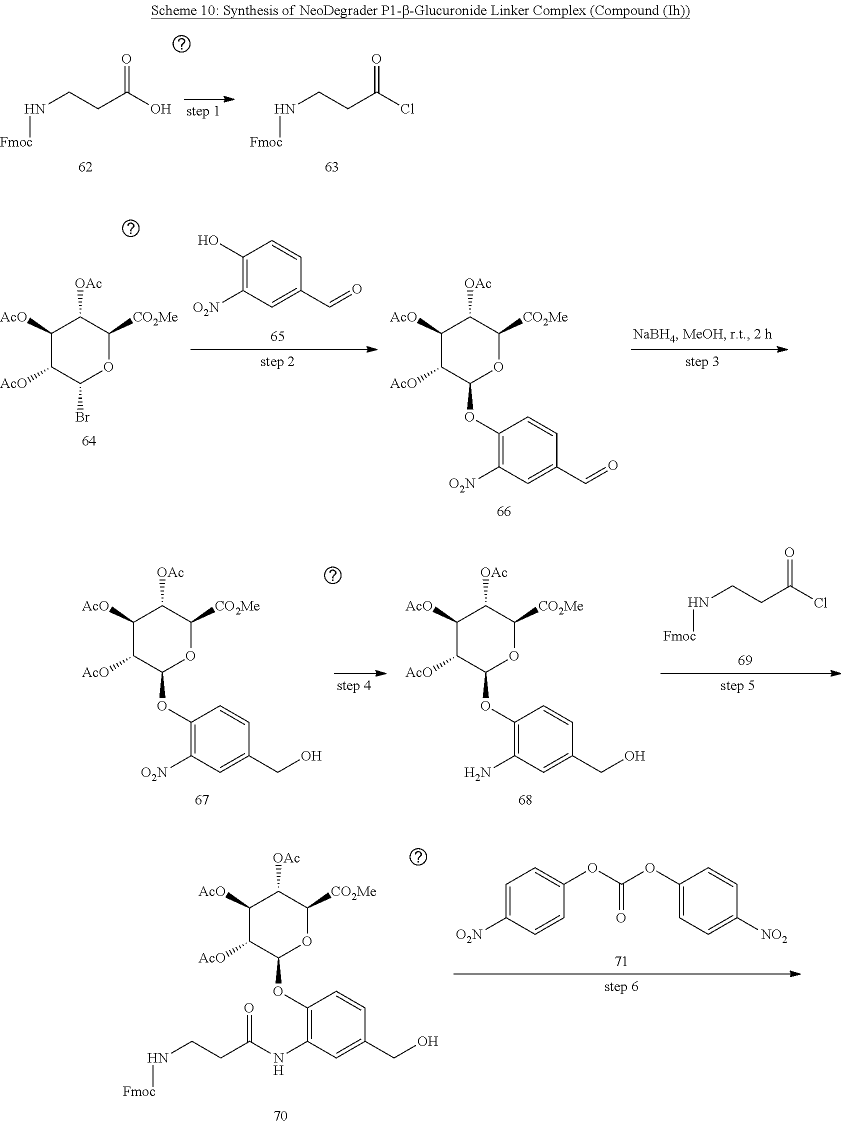

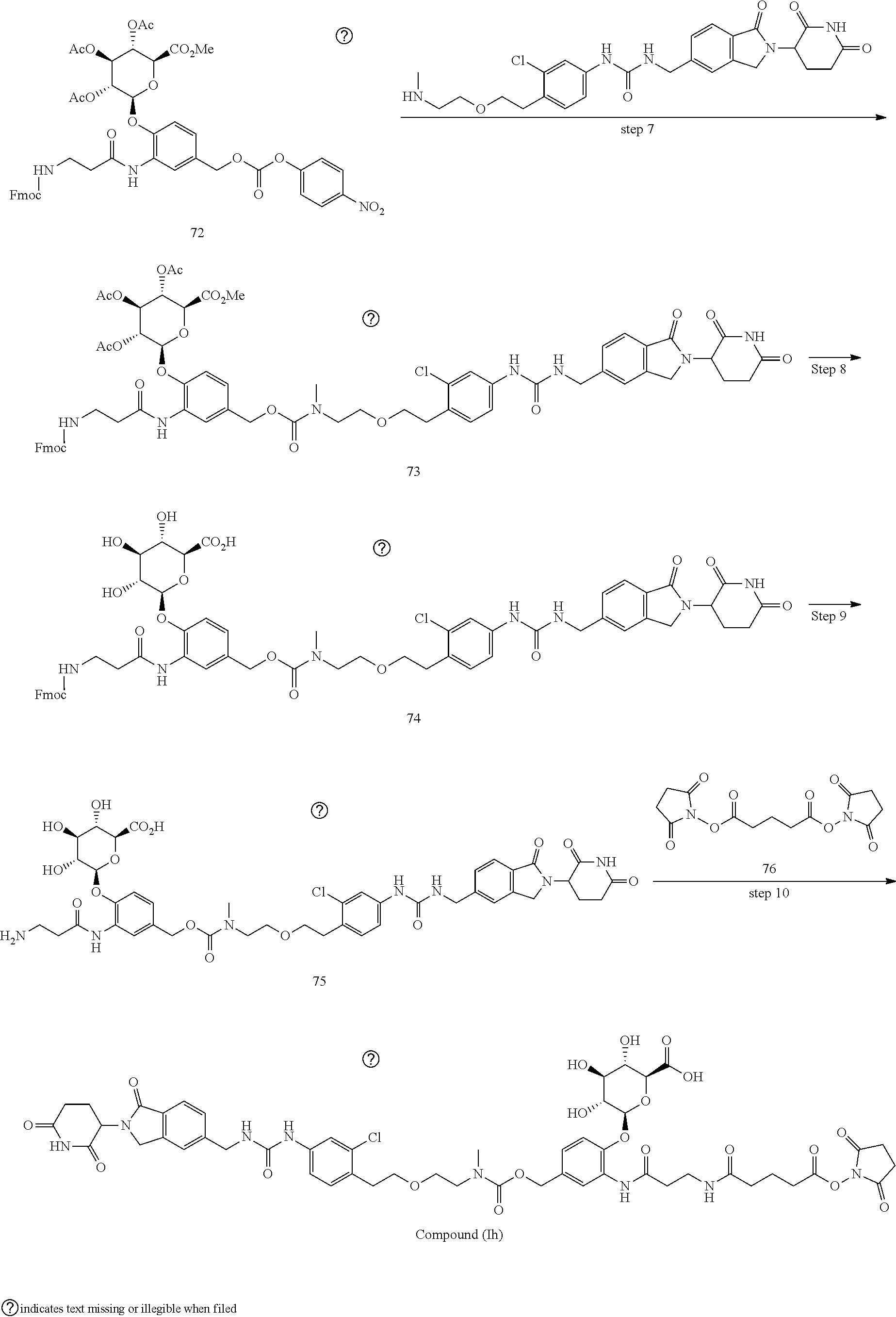

### Step 1. Synthesis of Compound 63

To a stirred mixture of 3-[[(9H-fluoren-9-ylmethoxy)carbonyl]amino]propanoic acid, (Compound 62, 5.00 g, 16.06 mmol, 1.00 equiv), SOCl₂ (25 mL) was added at room temperature. The resulting mixture was stirred 16 h at 80° C. Desired product could be detected by LCMS (derivative with MeOH MS=326). LCMS indicated the reaction was completed. The resulting mixture was concentrated under vacuum to afford 9H-fluoren-9-ylmethyl N-(3-chloro-3-oxopropyl)carbamate (Compound 63, 7.5 g, crude) as a yellow oil. The crude product was used directly in the next step without further purification. ¹H NMR analysis indicated it was the desired product (derivative with MeOH). ¹H-NMR (300 MHz, CDCl₃) δ 7.81-7.77 (m, 2H), 7.63-7.59 (m, 2H), 7.46-7.40 (m, 2H), 7.40-7.31 (m, 2H), 5.33 (s, 1H), 4.42 (d, J=3.0 Hz, 2H), 4.24 (t, J=6.0 Hz, 1H), 3.74-3.67 (m, 3H), 3.50 (d, J=3.0 Hz, 2H), 2.59 (t, J=6.0 Hz, 2H).

### Step 2. Synthesis of Compound 66

To a stirred solution of 4-formyl-2-nitrophenol (Compound 65, 4.21 g, 25.19 mmol, 1.00 equiv) and Ag₂O (7.00 g, 30.20 mmol, 1.20 equiv) in ACN (100 mL, 190.24 mmol, 75.00 equiv) were added methyl (2S,3S,4S,5R,6R)-3,4,5-tris(acetyloxy)-6-bromooxane-2-carboxylate (Compound 64, 10.00 g, 25.17 mmol, 1.00 equiv) in portions at room temperature under N₂ atmosphere. The resulting mixture was stirred for overnight at room temperature under N₂ atmosphere. LCMS indicated the reaction was completed. The resulting mixture was filtered, the filter cake was washed with DCM (50 mL×3). The filtrate was concentrated under reduced pressure. The residue was purified by silica gel column chromatography, eluted with PE/EA (PE:EA=1:2) to afford methyl (2S,3S,4S,5R,6S)-3,4,5-tris(acetyloxy)-6-(4-formyl-2-nitrophenoxy)oxane-2-carboxylate (Compound 66, 10.5 g, 86%) as a white solid. ¹H-NMR analysis indicated it was the desired product. LCMS (ES, m/z): 484 [M+1]⁺. ¹H-NMR (300 MHz, CDCl₃) δ 10.00 (s, 1H), 8.34 (s, 1H), 8.13-8.09 (m, 1H), 7.52 (d, J=3.0 Hz, 1H), 5.47-5.29 (m, 4H), 4.37-4.35 (m, 1H), 3.75-3.73 (m, 3H), 2.17-2.06 (m, 9H).

### Step 3. Synthesis of Compound 67

To a stirred solution of methyl (2S,3 S,4S,5R,6S)-3,4,5-tris(acetyloxy)-6-(4-formyl-2-nitrophenoxy)oxane-2-carboxylate (Compound 66, 6.00 g, 12.41 mmol, 1.00 equiv) in MeOH (50 mL) was added NaBH₄ (0.47 g, 12.42 mmol, 1.00 equiv) in portions at room temperature under N₂ atmosphere. The resulting mixture was stirred for 2 h at room temperature under N₂ atmosphere. LCMS indicated the reaction was completed. The reaction was quenched with water at room temperature. The resulting was dried by Na₂SO₄. The resulting mixture was filtered, the filter cake was washed with DCM. The resulting mixture was concentrated under vacuum to afford methyl (2S,3S,4S,5R,6S)-3,4,5-tris(acetyloxy)-6-[4-(hydroxymethyl)-2-nitrophenoxy]oxane-2-carboxylate, (Compound 67, 5.5 g, 91%) as a solid. LCMS (ES, m/z): 486 [M+H]⁺.

### Step 4. Synthesis of Compound 68

To a stirred mixture of methyl (2S,3S,4S,5R,6S)-3,4,5-tris(acetyloxy)-6-[4-(hydroxymethyl)-2-nitrophenoxy]oxane-2-carboxylate (Compound 67, 5.50 g, 11.33 mmol, 1.00 equiv) in EA (60 mL) were added Pd/C (1.10 g, 10%) in portions at room temperature. The resulting mixture was stirred for 16 h at room temperature under H2 atmosphere. LCMS indicated the reaction was completed. The resulting mixture was filtered, the filter cake was washed with DCM and MeOH, The filtrate was concentrated under vacuum to afford methyl (2S,3 S,4S,5R,6S)-3,4,5-tris(acetyloxy)-6-[2-amino-4-(hydroxymethyl)phenoxy]oxane-2-carboxylate (Compound 68, 4.0 g, 77%) as a solid. The crude product was used in the next step directly without further purification. LCMS (ES, m/z): 456[M+H]⁺.

### Step 5. Synthesis of Compound 70

To a stirred solution of methyl (2S,3S,4S,5R,6S)-3,4,5-tris(acetyloxy)-6-[2-amino-4-(hydroxymethyl)phenoxy]oxane-2-carboxylate (Compound 68, 1.00 g, 2.19 mmol, 1.00 equiv) and NaHCO₃ (0.20 g, 2.40 mmol, 1.1 equiv) in THE (10 mL) were added 9H-fluoren-9-ylmethyl N-(3-chloro-3-oxopropyl)carbamate (Compound 69, 0.87 g, 2.62 mmol, 1.20 equiv) in portions at 0° C. under N₂ atmosphere. The resulting mixture was stirred for 6 h at 0° C. under N₂ atmosphere. LCMS indicated the reaction was completed. The reaction was quenched with water at room temperature. The resulting mixture was extracted with DCM. The combined organic layers were concentrated under reduced pressure. The residue was purified by silica gel column chromatography, eluted with PE/EA (EA=100%) to afford methyl (2S,3S,4S,5R,6S)-3,4,5-tris(acetyloxy)-6-[2-(3-[[(9H-fluoren-9-ylmethoxy)carbonyl]amino]propanamido)-4-(hydroxymethyl)phenoxy]oxane-2-carboxylate (Compound 70, 1.1 g, 66%) as a light yellow solid. LCMS (ES, m/z): 749 [M+H]⁺.

### Step 6. Synthesis of Compound 72

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-(hydroxymethyl)phenoxy]oxane-2-carboxylate (Compound 70, 1.50 g, 2.00 mmol, 1.00 equiv) and bis(4-nitrophenyl) carbonate (Compound 71, 0.68 g, 2.24 mmol, 1.12 equiv) in DMF (15 mL) were added DIEA (0.52 g, 4.01 mmol, 2.00 equiv) in portions at 0° C. under N₂ atmosphere. The resulting mixture was stirred for overnight at room temperature under nitrogen atmosphere. LCMS indicated the reaction was completed. The reaction mixture was purified by reverse flash chromatography with the following conditions: column, C18 silica gel; mobile phase, ACN in water (0.1% FA), 10% to 90% gradient in 40 min; detector, UV 254 nm. The collected fraction was concentrated to dryness in vacuum to afford methyl (2S,3S,4S,5R,6S)-3,4,5-tris(acetyloxy)-6-[2-(3-[[(9H-fluoren-9-ylmethoxy)carbonyl]-amino]propanamido)-4-[[(4-nitrophenoxycarbonyl)oxy]methyl]phenoxy]oxane-2-carboxylate (Compound 72, 1.4 g, 48%) as a yellow solid. LCMS (ES, m/z): 914 [M+H]⁺.

### Step 7. Synthesis of Compound 73

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-[[(4-nitrophenoxycarbonyl)oxy]methyl]phenoxy]oxane-2-carboxylate (Compound 72, 1.00 g, 1.09 mmol, 1.00 equiv) and 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (neoDegrader P1, 0.58 g, 1.09 mmol, 1.00 equiv) in DMF (10 mL) were added HOBT (1.18 g, 8.72 mmol, 8.00 equiv) and 2,4-dimethylpyridine (1.07 g, 8.72 mmol, 8.00 equiv) in portions at room temperature under N₂ atmosphere. The resulting mixture was stirred for 16 h at room temperature under N₂ atmosphere. LCMS indicated the reaction was completed. The resulting mixture was used further purification. The residue was purified by reverse flash chromatography with the following conditions: column, C18 silica gel; mobile phase, ACN in water (0.1% FA), 10% to 80% gradient in 40 min; detector, UV 254 nm. The collected fraction was concentrated under vacuum to afford methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 73 (800 mg, 56%) as a solid. LCMS (ES, m/z): 1302[M+H]⁺.

### Step 8. Synthesis of Compound 74

To a stirred mixture of methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 73 (800.00 mg, 0.61 mmol, 1.00 equiv) in THE (80 mL) were added HCl (6N, 80 mL) in portions at room temperature under N₂ atmosphere. The resulting mixture was stirred for 3 h at degrees 50° C. under nitrogen atmosphere. LCMS indicated the reaction was completed. The resulting mixture was concentrated under vacuum. The residue was purified by reverse flash chromatography with the following conditions: column, C18 silica gel; mobile phase, ACN in water (0.1% FA), 0% to 80% gradient in 40 min; detector, UV 254 nm. The collected fraction was lyophilized to afford (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid (Compound 74, 230 mg, 32%) as a white solid. LCMS (ES, m/z): 1162[M+H]⁺.

### Step 9. Synthesis of Compound 75

To a stirred solution of (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid, 74 (230 mg, 0.2 mmol, 1.00 equiv) in DMF (2 mL) were added piperidine (0.4 mL) in portions at room temperature under nitrogen atmosphere. The resulting mixture was stirred for 10 min at room temperature under nitrogen atmosphere. LCMS indicated the reaction was completed. The resulting mixture was used directly further purification by Prep-HPLC with the following conditions (Column: XSelect CSH Prep C18 OBD Column, 19×250 mm, 5 um; Mobile Phase A:water (0.05% TFA), Mobile Phase B:ACN; Flow rate: 25 mL/min; Gradient: 20 B to 40 B in 7 min; 220 nm; RT1:5.78 min) to afford (2S,3S,4S,5R,6S)-6-[2-(3-aminopropanamido)-4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]phenoxy]-3,4,5-trihydroxyoxane-2-carboxylic acid (Compound 75, 35 mg, 18%) as a white solid. LCMS (ES, m/z): 940[M+H]⁺.

### Step 10. Synthesis of Compound (Ih)

To a stirred solution of (2S,3S,4S,5R,6S)-6-[2-(3-aminopropanamido)-4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]phenoxy]-3,4,5-trihydroxyoxane-2-carboxylic acid (Compound 75, 110 mg, 0.12 mmol, 1.00 equiv) and bis(2,5-dioxopyrrolidin-1-yl) pentanedioate (Compound 76, 46 mg, 0.14 mmol, 1.2 equiv) in DMF (2.0 mL) was added DIEA (30 mg, 0.23 mmol, 2.0 equiv) in portions at room temperature under nitrogen atmosphere. The resulting mixture was stirred for 1 h at room temperature under nitrogen atmosphere. LCMS indicated the reaction was completed. The reaction mixture was purified by Prep-HPLC with the following conditions (Column: Kinetex EVO prep C18, 30*150, Sum; Mobile Phase A: Water(0.05% TFA), Mobile Phase B: ACN; Flow rate: 60 mL/min; Gradient: 21% B to 41% B in 7 min, 41% B; Wave Length: 254 nm; RT1(min): 5.8. The collected fraction was lyophilized to afford (2S,3 S,4S,5R,6S)-6-{4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]-2-(3-{5-[(2,5-dioxopyrrolidin-1-yl)oxy]-5-oxopentanamido}propanamido)phenoxy}-3,4,5-trihydroxyoxane-2-carboxylic acid (Compound (Ih), 48 mg, 34% as a white solid. LCMS (ES, m/z): 1151 [M+H]⁺, 1173 [M+Na]⁺. ¹H-NMR (300 MHz, DMSO-d₆): 12.80 (br s, 1H), 10.98 (s, 1H), 9.08 (s, 1H), 8.79 (s, 1H), 8.18 (s, 1H), 7.96 (s, 1H), 7.68-7.66 (m, 2H), 7.51 (s, 1H), 7.44 (d, J=8.1 Hz, 1H), 7.25-7.00 (m, 4H), 6.82-6.80 (m, 1H), 5.86 (s, 1H), 5.39-5.30 (m, 2H), 5.14-5.07 (m, 1H), 4.97 (s, 2H), 4.84 (d, J=7.2 Hz, 1H), 4.47-4.27 (m, 4H), 3.90 (d, J=9.6 Hz, 1H), 3.56-3.48 (m, 4H), 3.45-3.36 (m, 6H), 2.95-2.80 (m, 8H), 2.75-2.65 (m, 3H), 2.62-2.55 (m, 2H), 2.49-2.35 (m, 1H), 2.21-2.16 (m, 2H), 2.01-1.95 (m, 1H), 1.85-1.80 (m, 2H).

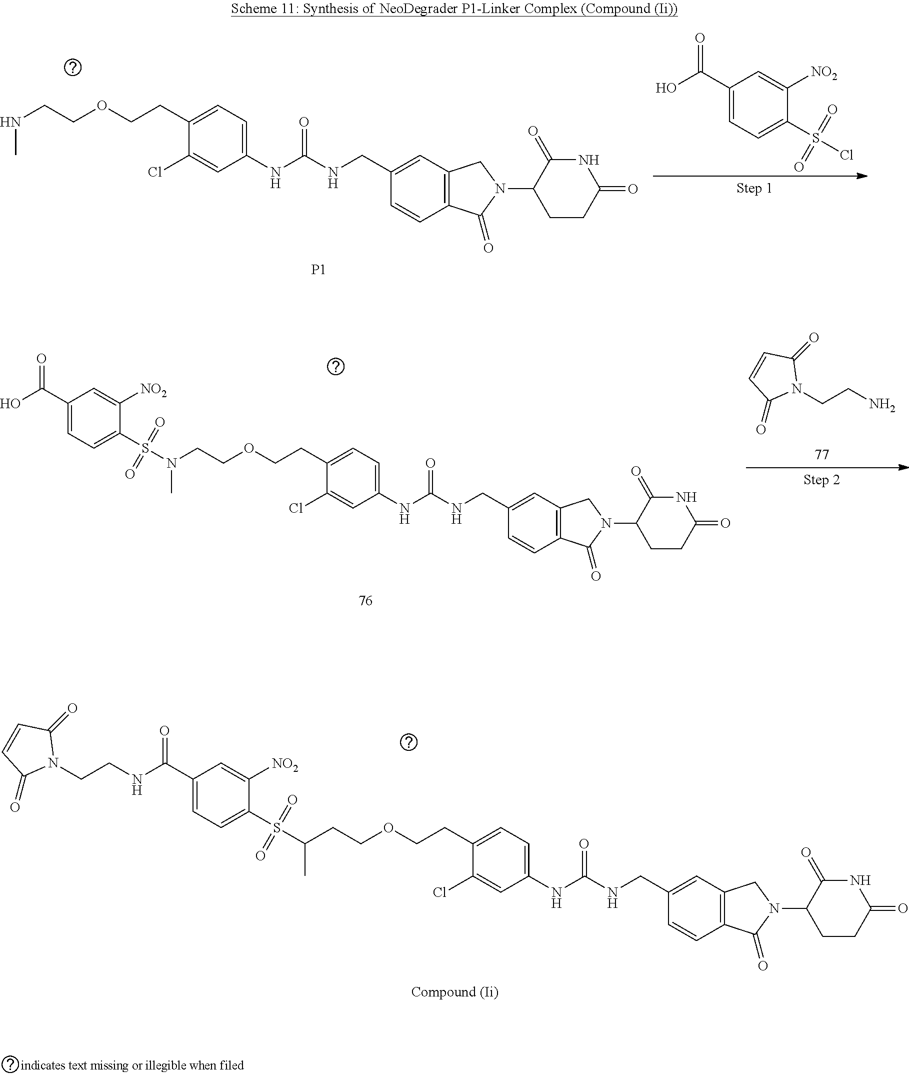 [SMILES: CNCCOCCc1ccc(NC(=O)NCc2ccc3c(c2)CN(C2CCC(=O)NC2=O)C3=O)cc1Cl ; O=C(O)c1ccc(S(=O)(=O)Cl)c([N+](=O)[O-])c1 ; "P1" ; CN(CCOCCc1ccc(NC(=O)NCc2ccc3c(c2)CN(C2CCC(=O)NC2=O)C3=O)cc1Cl)S(=O)(=O)c1ccc(C(=O)O)cc1[N+](=O)[O-] ; NCCN1C(=O)C=CC1=O ; CC(CCOCCc1ccc(NC(=O)NCc2ccc3c(c2)CN(C2CCC(=O)NC2=O)C3=O)cc1Cl)S(=O)(=O)c1ccc(C(=O)NCCN2C(=O)C=CC2=O)cc1[N+](=O)[O-]]

### Step 1. Synthesis of Compound 76

To a stirred solution of 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (Compound P1, 180 mg, 0.34 mmol, 1.00 equiv) in DMF (8 mL) were added TEA (104 mg, 1.02 mmol, 3.0 equiv) and 4-(chlorosulfonyl)-3-nitrobenzoic acid (181 mg, 0.68 mmol, 2.00 equiv) in portions at 0° C. under nitrogen atmosphere. The resulting mixture was stirred for 4 h at 0° C. under nitrogen atmosphere. LCMS indicated the reaction was completed. The resulting mixture was used further purification. The residue was purified by reverse flash chromatography with the following conditions: column, C18 silica gel; mobile phase, ACN in water (0.1% FA), 10% to 60% gradient in 10 min; detector, UV 254 nm. The mixture was lyophilized to afford 4-[[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)sulfamoyl]-3-nitrobenzoic acid, (Compound 76, 70 mg, 27%) as a light yellow solid. LCMS (ES, m/z): 757 [M+1]⁺.

### Step 2 Synthesis of Compound (Ii)

To a stirred mixture of 4-[[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)sulfamoyl]-3-nitrobenzoic acid, (Compound 76, 60 mg, 0.08 mmol, 1.00 equiv) in DMF (6 mL) were added HATU (45 mg, 0.12 mmol, 1.5 equiv), 1-(2-aminoethyl)pyrrole-2,5-dione hydrochloride (Compound 77, 17 mg, 0.10 mmol, 1.20 equiv) and DIEA (31 mg, 0.24 mmol, 3.0 equiv) in portions at room temperature under nitrogen atmosphere. The resulting mixture was stirred for 4 h at room temperature under nitrogen atmosphere. LCMS indicated the reaction was completed. The residue was purified by Prep-HPLC (Column: XBridge Prep Phenyl OBD Column, 19×150 mm 5 um 13 nm; Mobile Phase A:water (0.05% TFA), Mobile Phase B:ACN; Flow rate: 25 mL/min; Gradient: 25 B to 43 B in 10 min; 220 nm; RT1:11.97 min). The collected fraction was lyophilized to afford 4-[[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)sulfamoyl]-N-[2-(2,5-dioxopyrrol-1-yl)ethyl]-3-nitrobenzamide (Compound (Ii), 27 mg, 36%) as a white solid. LCMS (ES, m/z): 879,881 [M+H]. ¹H NMR (300 MHz, DMSO-d₆) δ 11.00 (s, 1H), 9.01 (t, J=6.0 Hz, 1H), 8.82 (s, 1H), 8.20 (s, 1H), 8.11 (s, 2H), 7.71-7.67 (m, 2H), 7.52 (s, 1H), 7.44 (d, J=3.0 Hz, 1H), 7.21-7.12 (m, 2H), 7.02 (s, 2H), 6.84 (t, J=6.0 Hz, 1H), 5.14-5.08 (m, 1H), 4.48-4.28 (m, 4H), 3.62-3.50 (m, 6H), 3.40-3.28 (m, 2H), 2.95-2.85 (m, 4H), 2.80-2.73 (m, 2H), 2.65-2.60 (s, 1H), 2.41-2.27 (m, 1H), 2.05-1.95 (m, 1H).

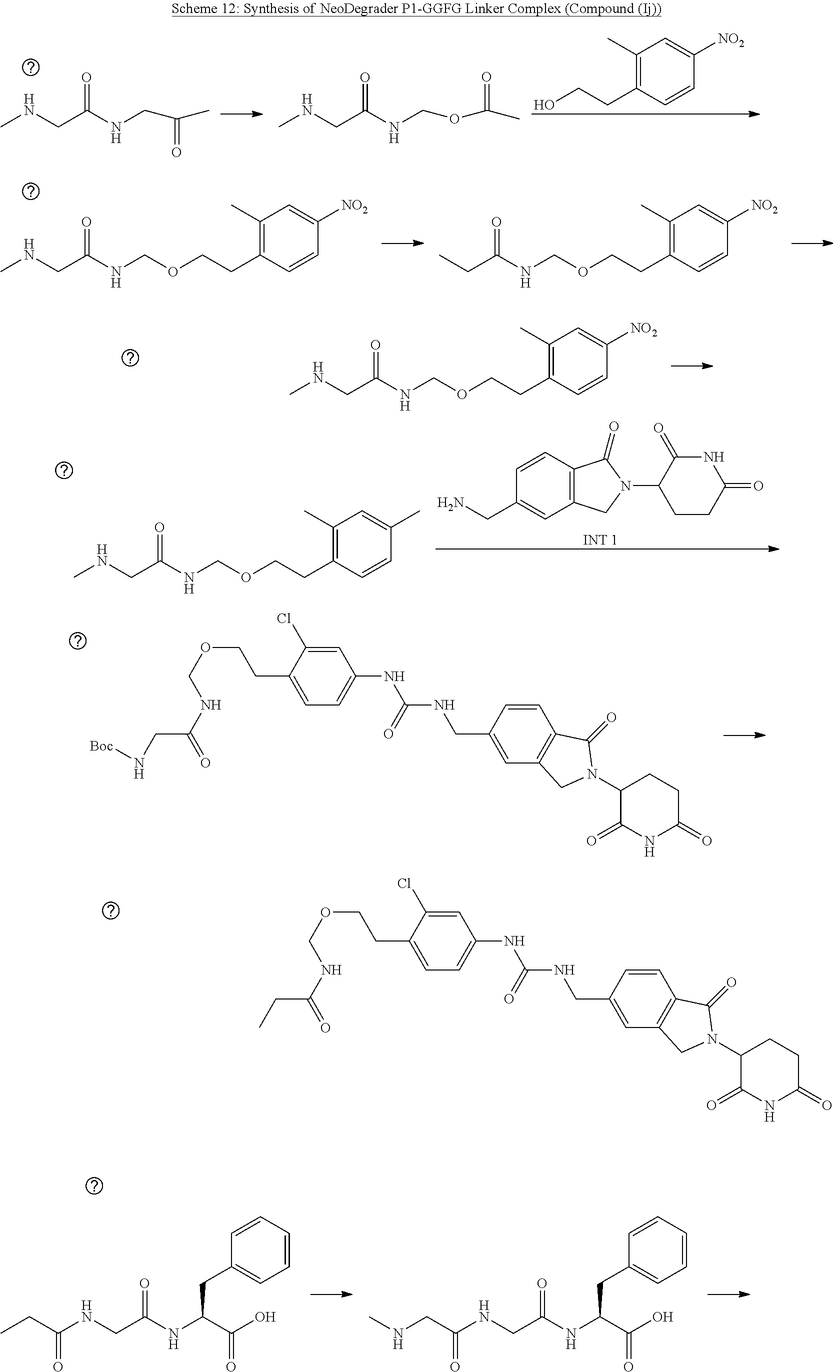

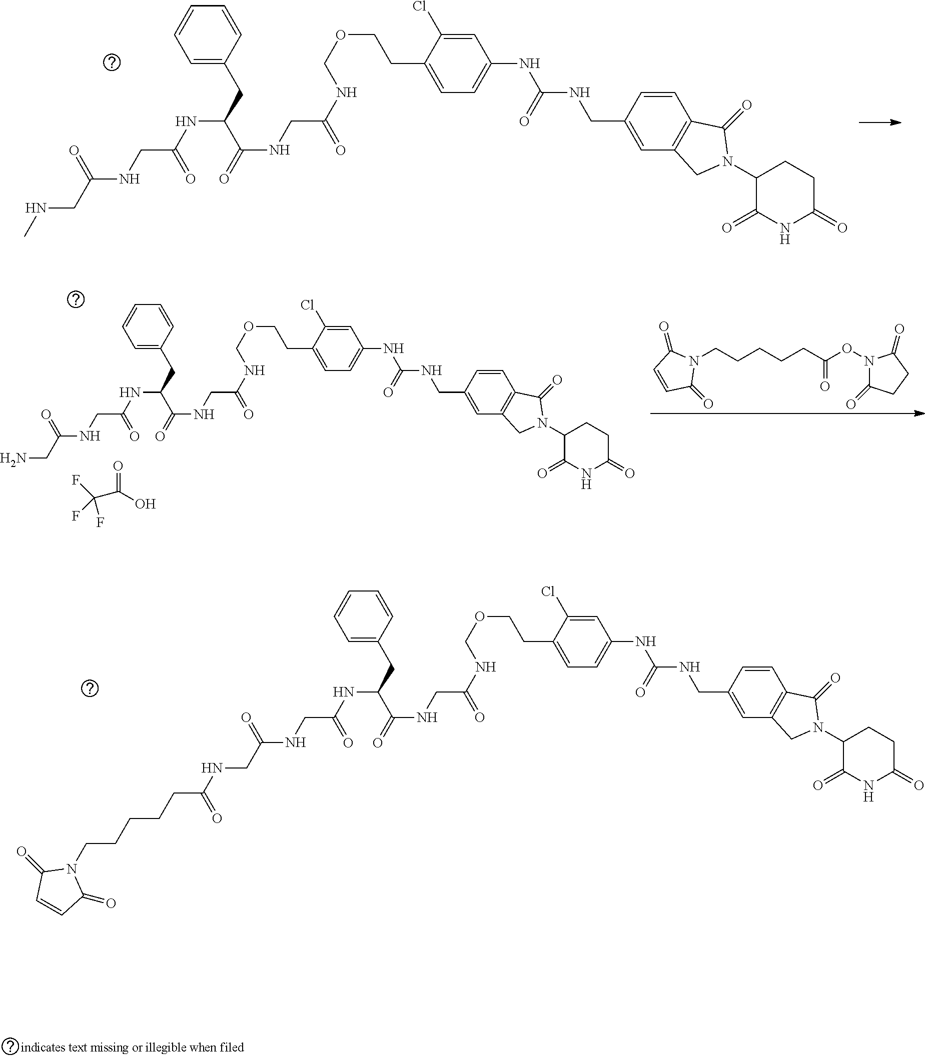

### Step 1. Synthesis of Compound 79

To a stirred mixture of (2-[[(9H-fluoren-9-ylmethoxy)carbonyl]amino]-acetamido)acetic acid (Compound 78, 10.00 g, 28.22 mmol, 1.00 equiv) and Pb(OAc)+(15.02 g, 33.86 mmol, 1.20 equiv) in THF (300 mL) and toluene (100 mL) were added pyridine (2.59 g, 32.74 mmol, 1.16 equiv) dropwise at room temperature under nitrogen atmosphere. The resulting mixture was stirred for overnight at 80° C. under nitrogen atmosphere. LCMS indicated the reaction was completed. The mixture was allowed to cool down to room temperature. The resulting mixture was filtered, and the filter cake was washed with ethyl acetate (20 mL). The filtrate was concentrated under reduced pressure. The residue was dissolved in ethyl acetate (20 mL), washed with water, brine, dried over anhydrous Na₂SO₄. After filtration, the filtrate was concentrated under reduced pressure. The residue was purified by silica gel column chromatography, eluted with PE/EtOAc (1:4) to afford (2-[[(9H-fluoren-9-ylmethoxy)carbonyl]amino]acetamido)methyl acetate (Compound 79, 6.5 g, 56%) as a white solid. LCMS (ESI, ms): 391[M+Na]⁺. ¹HNMR (300 MHz, CDCl₃) δ 7.80 (d, J=7.5 Hz, 2H), 7.62 (d, J=7.5 Hz, 2H), 7.45 (t, J=7.5 Hz, 2H), 7.36 (d, J=7.5 Hz, 2H), 7.18 (br s, 1H), 5.48 (br s, 1H), 5.28 (d, J=7.2 Hz, 2H), 4.48 (d, J=6.6 Hz, 2H), 4.26 (t, J=6.6 Hz, 1H), 3.93 (d, 5.4 Hz, 2H), 2.08 (s, 3H).

### Step 2. Synthesis of Compound 81

To a stirred mixture of (2-[[(9H-fluoren-9-ylmethoxy)carbonyl]-amino]acetamido)methyl acetate, 79 (2.00 g, 5.43 mmol, 1.00 equiv) and 2-(2-chloro-4-nitrophenyl)ethanol (Compound 3, 3.20 g, 15.85 mmol, 2.92 equiv) in DCM (40 mL) was added PPTS (400 mg, 1.59 mmol, 0.29 equiv) dropwise at 0° C. under nitrogen atmosphere. The resulting mixture was stirred for overnight at 45° C. under nitrogen atmosphere. 40% desired product could be detected by LCMS. The mixture was allowed to cool down to room temperature. The reaction was quenched with water/ice. The resulting mixture was extracted with EtOEt (3×20 mL). The combined organic layers were washed with brine (30 mL), dried over anhydrous Na₂SO₄. After filtration, the filtrate was concentrated under reduced pressure. The residue was purified by silica gel column chromatography, eluted with PE/EtOAc (1:9) to afford 9H-fluoren-9-ylmethyl N-[([[2-(2-chloro-4-nitrophenyl)ethoxy]methyl]carbamoyl)methyl]carbamate (Compound 81, 1.7 g, 55%) as a white solid. LCMS (ESI, ms): 510,512[M+H]⁺. ¹HNMR (300 MHz, DMSO-d₆): δ 8.58 (t, J=5.1 Hz, 1H), 8.22 (dd, J=12, 2.4 Hz, 1H), 7.89 (d, J=7.5 Hz, 1H), 7.71-7.54 (m, 4H), 7.43-7.29 (m, 4H), 4.56 (d, J=6.9 Hz, 2H), 4.30-4.16 (m, 3H), 3.70-3.61 (m, 4H), 3.04 (t, J=6.3 Hz, 2H).

### Step 3. Synthesis of Compound 82

To a stirred mixture of 9H-fluoren-9-ylmethyl N-[([[2-(2-chloro-4-nitrophenyl)ethoxy]methyl]carbamoyl)methyl]carbamate (Compound 81, 1.60 g, 3.14 mmol, 1.00 equiv) in DMF (5.0 mL) was added piperidine (1.0 mL) in portions at 0° C. under nitrogen atmosphere. The resulting mixture was stirred for 1 h at room temperature under nitrogen atmosphere. LCMS indicated the reaction was completed. The reaction mixture was purified by reverse flash chromatography with the following conditions: column, C18 silica gel; mobile phase, ACN in water (0.05% TFA), 0% to 50% gradient in 40 min; detector, UV 254 nm. This resulted in 2-amino-N-[[2-(2-chloro-4-nitrophenyl)ethoxy]methyl]acetamide (Compound 82, 750 mg, 76%) as a yellow oil. LCMS (ESI, ms) 288[M+H]⁺, 329[M+H+ACN]⁺

### Step 4. Synthesis of Compound 83

To a stirred mixture of 2-amino-N-[[2-(2-chloro-4-nitrophenyl)ethoxy]-methyl]acetamide (Compound 82, 750 mg, 2.61 mmol, 1.00 equiv) and Boc₂O (580 mg, 2.66 mmol, 1.02 equiv) in DMF (10.00 mL) was added NaHCO₃ (477 mg, 5.68 mmol, 2.18 equiv) in H₂O (10.00 mL) dropwise at 0° C. The resulting mixture was stirred for 3 h at room temperature. LCMS indicated the reaction was completed. The reaction was quenched by the addition of water (20 mL). The resulting mixture was extracted with EtOEt (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc (1:2) to afford tert-butyl N-[([[2-(2-chloro-4-nitrophenyl)ethoxy]methyl]carbamoyl)methyl]carbamate (Compound 83, 650 mg, 58%) as a yellow oil. LCMS(ESI, ms), 388[M+H]⁺, 332[M+H−56]⁺. ¹HNMR (400 MHz, CDCl₃) δ 8.21 (d, J=2.4 Hz, 1H), 8.04 (d, J=8.4 Hz, 2H), 7.46 (d, J=8.4 Hz, 1H), 7.05 (br s, 1H), 5.25 (br s, 1H), 4.73 (d, J=7.2 Hz, 2H), 3.81-3.73 (m, 4H), 3.34-3.32 (m, 2H), 3.08 (t, J=6.8 Hz, 2H), 1.42 (s, 9H).

### Step 5. Synthesis of Compound 84

To a stirred mixture of tert-butyl N-[([[2-(2-chloro-4-nitrophenyl)ethoxy]methyl]-carbamoyl)methyl]carbamate (Compound 83, 650 mg, 1.68 mmol, 1.00 equiv) and Fe(260 mg, 4.66 mmol, 2.78 equiv) in EtOH (9.00 mL) was added NH₄Cl (910 mg, 17.01 mmol, 10.1 equiv) in H₂O (3.00 mL) dropwise at room temperature. The resulting mixture was stirred for 4 h at 90° C. LCMS indicated the reaction was completed. The mixture was allowed to cool down to room temperature. The resulting mixture was concentrated under vacuum. The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc (1:1) to afford tert-butyl N-[([[2-(4-amino-2-chlorophenyl)ethoxy]methyl]-carbamoyl)methyl]carbamate (Compound 84, 500 mg, 83%) as a yellow solid. LCMS(ESI, ms): 358[M+H]⁺, 380[M+Na]⁺. ¹HNMR (300 MHz, CDCl₃) δ 7.02-6.96 (m, 2H), 6.68 (d, J=2.4 Hz, 1H), 6.52-6.49 (m, 1H), 5.29 (br s, 1H), 4.74 (d, J=6.9 Hz, 2H), 3.80-3.78 (m, 2H), 3.69-3.63 (m, 2H), 2.88 (t, J=7.2 Hz, 2H), 1.45 (s, 9H).

### Step 6. Synthesis of Compound 86

To a stirred mixture of 3-[5-(aminomethyl)-1-oxo-3H-isoindol-2-yl]piperidine-2,6-dione hydrochloride (Compound 85, 398 mg, 1.28 mmol, 0.92 equiv) and CDI (450 mg, 2.78 mmol, 1.99 equiv) in DMF (5.00 mL) was added TEA (300 mg, 2.96 mmol, 2.12 equiv) at 0° C. The resulting mixture was stirred for 2 h at room temperature. To the above mixture was added tert-butyl N-[([[2-(4-amino-2-chlorophenyl)ethoxy]methyl]carbamoyl)methyl]carbamate (Compound 84, 500 mg, 1.40 mmol, 1.00 equiv) and DMAP (550 mg, 4.50 mmol, 3.22 equiv) in portions. The resulting mixture was stirred for additional overnight at 60° C. LCMS indicated the reaction was completed. The mixture was allowed to cool down to room temperature. The reaction mixture was purified by reverse flash chromatography with the following conditions: column, C18 silica gel; mobile phase, ACN in water (0.1% FA), 0% to 50% gradient in 30 min; detector, UV 254 nm. This resulted in tert-butyl N-([[(2-[2-chloro-4-[([[2-(2,6-dioxopiperidin-3-yl)-1-oxo-3H-isoindol-5-yl]methyl]-carbamoyl)amino]phenyl]ethoxy)methyl]carbamoyl]methyl)carbamate (Compound 86, 550 mg, 60%) as a light brown solid. LCMS (ESI, ms): 657[M+H]⁺, 601[M+H−56]⁺, 557[M+H−100]⁺.

### Step 7. Synthesis of Compound 87

To a stirred mixture of tert-butyl N-([[(2-[2-chloro-4-[([[2-(2,6-dioxopiperidin-3-yl)-1-oxo-3H-isoindol-5-yl]methyl]carbamoyl)amino]phenyl]ethoxy)methyl]carbamoyl]methyl)-carbamate (Compound 86, 530 mg, 0.80 mmol, 1.00 equiv) in DCM (5.00 mL) was added TFA (1.00 mL) at 0° C. The resulting mixture was stirred for 30 min at 0° C. LCMS indicated the reaction was completed. The resulting mixture was concentrated under reduced pressure. This resulted in 2-amino-N-[(2-[2-chloro-4-[([[2-(2,6-dioxopiperidin-3-yl)-1-oxo-3H-isoindol-5-yl]methyl]carbamoyl)amino]phenyl]ethoxy)methyl]acetamide; trifluoroacetic acid (Compound 87, (510 mg, purity: 64%, yield: 60%) as an off-white solid. LCMS (ESI, ms): 557[M+H−TFA]⁺

### Step 8. Synthesis of Compound 89

To a stirred mixture of (2S)-2-[2-(2-aminoacetamido)acetamido]-3-phenylpropanoic acid (Compound 88, 2.00 g, 7.16 mmol, 1.00 equiv) and NaHCO₃ (1.80 g, 21.41 mmol, 3.00 equiv) in H₂O (40.00 mL) were added Boc₂O (1.86 g, 8.52 mmol, 1.20 equiv) in DMF (40.00 mL) dropwise at 0° C. The resulting mixture was stirred for overnight at room temperature. LCMS indicated the reaction was completed. The reaction was quenched with water at room temperature. The resulting mixture was extracted with EtOEt (3×50 mL). The combined organic layers were washed with brine (50 mL), dried over anhydrous Na₂SO₄. After filtration, the filtrate was concentrated under reduced pressure. The residue was purified by reverse flash chromatography with the following conditions: column, C18 silica gel; mobile phase, ACN water (0.05% TFA), 5% to 60% gradient in 30 min; detector, UV 220 nm. This resulted in (2S)-2-(2-[2-[(tert-butoxycarbonyl)amino]acetamido]acetamido)-3-phenylpropanoic acid (Compound 89, 1.8 g, 60%) as a white semi-solid. LCMS (ESI, ms): 380[M+H]⁺, 324[M+H−56]⁺. ¹HNMR: (300 MHz, DMSO-d₆) δ 8.17 (d, J=8.1 Hz, 1H), 7.93 (t, J=5.7 Hz, 1H), 7.31-7.20 (m, 5H), 7.00 (t, J=6.0 Hz, 1H), 4.46-4.39 (m, 1H), 3.78-3.67 (m, 2H), 3.56 (d, J=5.7 Hz, 2H), 3.09-3.02 (m, 1H), 2.92-2.73 (m, 1H), 1.39 (s, 9H).

### Step 9. Compound 90

To a stirred mixture of (2S)-2-(2-[2-[(tert-butoxycarbonyl)amino]acetamido]-acetamido)-3-phenylpropanoic acid (Compound 89, 340 mg, 0.90 mmol, 1.00 equiv) and HATU (340 mg, 0.90 mmol, 1.00 equiv) in DMF (5.00 mL) was added HOBT (102 mg, 0.75 mmol, 0.84 equiv) in portions at 0° C. The resulting mixture was stirred for 30 min at 0° C. To the above mixture was added 2-amino-N-[(2-[2-chloro-4-[([[2-(2,6-dioxopiperidin-3-yl)-1-oxo-3H-isoindol-5-yl]methyl]carbamoyl)amino]phenyl]ethoxy)methyl]acetamide; trifluoroacetic acid (Compound 87, 511 mg, purity: 64%, 0.48 mmol, 0.54 equiv) and DIEA (340 mg, 2.63 mmol, 2.94 equiv) at 0° C. The resulting mixture was stirred for additional 2 h at room temperature. LCMS indicated the reaction was completed. The reaction mixture was purified by reverse flash chromatography with the following conditions: column, C18 silica gel; mobile phase, ACN in water (0.1% FA), 0% to 50% gradient in 30 min; detector, UV 220 nm. The collected fraction was concentrated under vacuum. This resulted in tert-butyl N-[[([[(1S)-1-[([[(2-[2-chloro-4-[([[2-(2,6-dioxopiperidin-3-yl)-1-oxo-3H-isoindol-5-yl]methyl]carbamoyl)amino]phenyl]ethoxy)methyl]carbamoyl]methyl)carbamoyl]-2-phenylethyl]carbamoyl]methyl)carbamoyl]methyl]carbamate (Compound 90, 210 mg, 48%) as an off-white solid. LCMS (ESI, ms): 918[M+H]⁺, 818[M+H−100]⁺. ¹HNMR: (400 MHz, DMSO-d₆): δ 10.97 (s, 1H), 8.79 (s, 1H), 8.50 (t, J=6.4 Hz, 1H), 8.31 (t, J=4.4 Hz, 1H), 8.15 (d, J=9.6 Hz, 1H), 7.910 (t, J=8.0 Hz, 1H), 7.68-7.64 (m, 2H), 7.49 (s, 1H), 7.43 (d, J=9.6 Hz, 1H), 7.24-7.12 (m, 7H), 7.00-6.95 (m, 1H), 6.84 (t, J=6.4 Hz, 1H), 5.13-5.06 (m, 1H), 4.55-4.27 (m, 7H), 3.72-3.60 (m, 6H), 3.75-3.67 (m, 3H), 3.59-3.49 (m, 5H), 3.07-3.01 (m, 1H), 2.94-2.73 (m, 4H), 2.62-2.54 (m, 1H), 2.40-2.31 (m, 1H), 2.01-1.94 (m, 1H), 2.00-1.91 (m, 1H), 1.35 (s, 9H)

### Step 10. Synthesis of Compound 91

To a stirred mixture of tert-butyl N-[[([[(1S)-1-[([[(2-[2-chloro-4-[([[2-(2,6-dioxopiperidin-3-yl)-1-oxo-3H-isoindol-5-yl]methyl]carbamoyl)amino]phenyl]ethoxy)-methyl]carbamoyl]methyl)carbamoyl]-2-phenylethyl]carbamoyl]methyl)carbamoyl]-methyl]carbamate (Compound 90, 140 mg, 0.15 mmol, 1.00 equiv) in DCM (5.00 mL) was added TFA (1.00 mL) dropwise at 0° C. The resulting mixture was stirred for 30 min at 0° C. LCMS indicated the reaction was completed. The resulting mixture was concentrated under reduced pressure. This resulted in (2S)-2-[2-(2-aminoacetamido)acetamido]-N-([[(2-[2-chloro-4-[([[2-(2,6-dioxopiperidin-3-yl)-1-oxo-3H-isoindol-5-yl]methyl]carbamoyl)amino]phenyl]ethoxy)methyl]-carbamoyl]methyl)-3-phenylpropanamide; trifluoroacetic acid (Compound 91, 140 mg, 79%) as an off-white solid. LCMS(ESI, ms): 818[M+H−TFA]⁺.

### Step 1. Synthesis of Compound (Ij)

To a stirred mixture of (2S)-2-[2-(2-aminoacetamido)acetamido]-N-([[(2-[2-chloro-4-[([[2-(2,6-dioxopiperidin-3-yl)-1-oxo-3H-isoindol-5-yl]methyl]carbamoyl)amino]phenyl]-ethoxy)methyl]carbamoyl]methyl)-3-phenylpropanamide; trifluoroacetic acid (Compound 91, 140 mg, 0.15 mmol, 1.00 equiv) and DIEA (70 mg, 0.54 mmol, 3.61 equiv) in DMF (2.00 mL) was added 2,5-dioxopyrrolidin-1-yl 6-(2,5-dioxopyrrol-1-yl)hexanoate (Compound 92, 70 mg, 0.23 mmol, 1.50 equiv) in portions at 0° C. The resulting mixture was stirred for 2 h at room temperature. LCMS indicated the reaction was completed. The reaction mixture was directly purified by the following condition: Column: XSelect CSH Prep C18 OBD Column, 19×250 mm, 5 um; Mobile Phase A:Water(0.1% FA), Mobile Phase B:ACN; Flow rate: 25 mL/min; Gradient: 25 B to 50 B in 7 min; 254 nm; RT1:6.35 min; The collected fraction was lyophilized to give the crude product. The crude product was re-purified by the following condition:Column: Kinetex EVO C18 Column, 30×150.5 um; Mobile Phase A:Water(0.05% TFA), Mobile Phase B:ACN; Flow rate: 60 mL/min; Gradient: 20 B to 40 B in 7 min, 220 nm; RT1:6.77 min; The collected fraction was lyophilized to give the N-[[([[(1S)-1-[([[(2-[2-chloro-4-[([[2-(2,6-dioxopiperidin-3-yl)-1-oxo-3H-isoindol-5-yl]methyl]carbamoyl)amino]phenyl]ethoxy)methyl]carbamoyl]methyl)carbamoyl]-2-phenylethyl]carbamoyl]methyl)carbamoyl]methyl]-6-(2,5-dioxopyrrol-1-yl)hexanamide (Compound (Ik), 22.8 mg, 14%) as off-white solid. LCMS (ESI, ms): 1011[M+H]⁺. ¹HNMR: (400 MHz, DMSO-d₆): δ 10.95 (s, 1H), 8.79 (s, 1H), 8.51 (t, J=8.4 Hz, 1H), 8.29 (t, J=8.0 Hz, 1H), 8.12-8.01 (m, 3H), 7.70-7.66 (m, 2H), 7.44 (s, 1H), 7.42 (d, J=8.0 Hz, 1H), 7.23-7.16 (m, 7H), 6.99 (s, 2H), 6.82 (t, J=8.0 Hz, 1H), 5.13-5.09 (m, 1H), 4.55-4.28 (m, 7H), 3.72-3.60 (m, 6H), 3.55-3.51 (m, 2H), 3.36-3.34 (m, 2H), 3.05-3.00 (m, 1H), 2.94-2.72 (m, 4H), 2.62-2.54 (m, 1H), 2.40-2.32 (m, 1H), 2.12-2.05 (m, 2H), 2.00-1.91 (m, 1H), 1.50-1.38 (m, 4H), 1.19-1.10 (m, 2H)

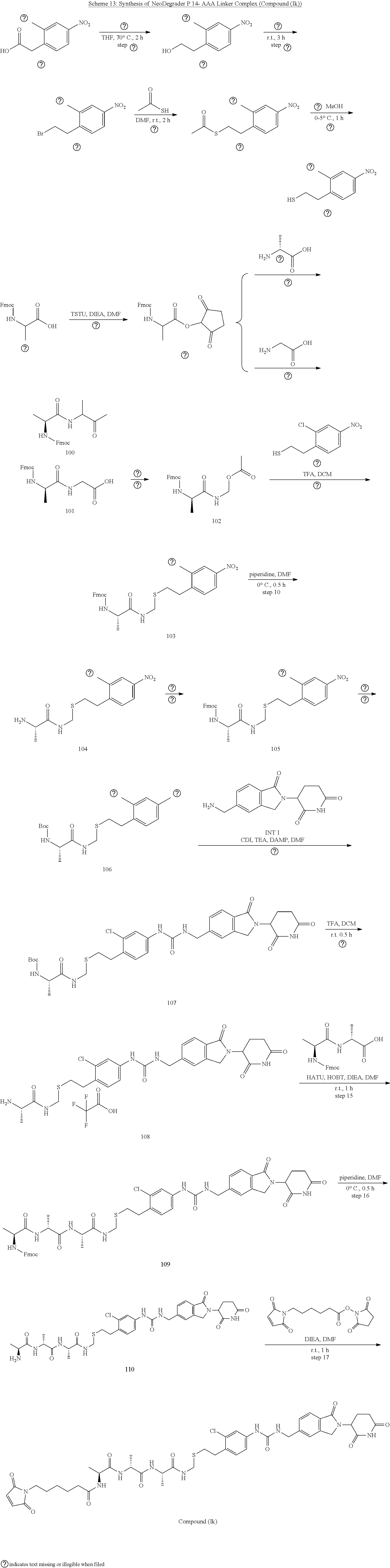

### Step 1. Synthesis of Compound 94

To a stirred solution of (2-chloro-4-nitrophenyl)acetic acid (Compound 93, 24.00 g, 111.32 mmol, 1.00 equiv) in THF (240.00 mL) were added BH₃-Me₂S (28.00 mL, 295.23 mmol, 2.65 equiv) dropwise under nitrogen atmosphere. The resulting mixture was stirred for 2 hours at 70° C. under nitrogen atmosphere. TLC (PE:EtOAc=3:1) indicated the reaction was completed. After cooled to room temperature, the resulting mixture was concentrated under vacuum. The residue was purified by silica gel column chromatography, eluted with PE/EtOAc (3:1) to afford 2-(2-chloro-4-nitrophenyl)ethanol (Compound 94, 18.00 g, 80%) as a light yellow solid. ¹H NMR (300 MHz, CDCl₃) δ 8.27 (s, 1H), 8.10-8.07 (m, 1H), 7.52 (d, J=3 Hz, 1H), 3.96 (t, J=6 Hz, 2H), 3.13 (t, J=6 Hz, 2H).

### Step 2. Synthesis of Compound 95

To a stirred solution of 2-(2-chloro-4-nitrophenyl)ethanol (Compound 94, 5.00 g, 24.80 mmol, 1.00 equiv) in DCM (100.00 mL) were added NBS (6.62 g, 1.50 equiv) and PPh₃ (9.76 g, 37.21 mmol, 1.50 equiv) in portions at room temperature under N₂. The resulting mixture was stirred overnight at room temperature under N₂. TLC (PE:EtOAc=10:1) indicated the reaction was completed. The reaction was concentrated to dryness under vacuum. The residue was purified by silica gel column chromatography, eluted with PE/EtOAc (4:1) to afford 1-(2-bromoethyl)-2-chloro-4-nitrobenzene (Compound 95, 5.10 g, 72%) as a red oil. ¹H NMR (400 MHz, DMSO-d₆) δ 8.28 (d, J=2.4 Hz, 1H), 8.18 (dd, J=8.4, 2.4 Hz, 1H), 7.73 (d, J=8.4 Hz, 1H), 3.79 4 (t, J=6.8 Hz, 2H), 3.38 (t, J=6.8 Hz, 2H).

### Step 3. Synthesis of Compound 96

To a solution of 1-(2-bromoethyl)-2-chloro-4-nitrobenzene (Compound 95, 5.00 g, 18.90 mmol, 1.00 equiv) in DMF (50.00 mL) was added potassium thioacetate (2.16 g, 18.90 mmol, 1.00 equiv) at room temperature under nitrogen atmosphere. The resulting mixture was stirred at room temperature for 2 hours. TLC (PE:EtOAc=10:1) indicated the reaction was completed. The reaction was diluted with water (600.00 mL), and extracted with EtOAc (2000 mL×3). The combined organic layer was washed with water (200.00 mL), brine (200.00 mL), dried over anhydrous Na₂SO₄ and concentrated to dryness under vacuum to afford 1-[[2-(2-chloro-4-nitrophenyl)ethyl]sulfanyl]ethenone (Compound 96, 4.50 g, 85%) as a red oil. ¹H NMR (400 MHz, CDCl₃) δ 8.24 (d, J=2.4 Hz, 1H), 8.07 (dd, J=8.4, 2.4 Hz, 1H), 7.45 (d, J=8.4 Hz, 1H), 3.20-3.05 (m, 4H), 2.34 (s, 3H).

### Step 4. Synthesis of Compound 97

To a stirred solution of 1-[[2-(2-chloro-4-nitrophenyl)ethyl]sulfanyl]ethenone (Compound 96, 2.00 g, 7.70 mmol, 1.00 equiv) in MeOH (300.00 mL) was added MeONa (6.93 mL, 37.33 mmol, 5.00 equiv, 30% in MeOH) at 0° C. under N₂. The resulting mixture was stirred at 0° C. under N₂ for 1 h. TLC (PE:EtOAc=10:1) indicated the reaction was completed. The reaction was quenched with AcOH to pH value to 3-4. The resulting mixture was concentrated to dryness under vacuum. The residue was diluted with DCM (50.00 mL) and filtered. The filtrate was purified with Prep-TLC (PE:EtOAc=10:1) to give 2-(2-chloro-4-nitrophenyl)ethanethiol (Compound 97, 1.35 g, 72%) as a light yellow oil. ¹H NMR (400 MHz, CDCl₃) δ 8.26 (d, J=2.4 Hz, 1H), 8.09 (dd, J=8.4, 2.4 Hz, 1H), 7.45 (d, J=8.4 Hz, 1H), 3.14 (t, J=8.0 Hz, 2H), 2.85 (dt, J=8.0, 7.2 Hz, 2H), 1.43 (t, J=7.2 Hz, 1H).

### Step 5. Synthesis Compound 99

To a stirred solution of (2S)-2-[[(9H-fluoren-9-ylmethoxy)carbonyl]amino]propanoic acid (Compound 98, 20.00 g, 64.24 mmol, 1.00 equiv) in DMF (200.00 mL) were added TSTU (25.18 g, 83.52 mmol, 1.30 equiv) and DIEA (16.60 g, 128.48 mmol, 2.00 equiv) at room temperature under air atmosphere. The resulting mixture was stirred for 1 h at room temperature. LCMS indicated the reaction was completed. The reaction was diluted with water (200.00 mL), was extracted with EtOAc (100.00 mL×3). The combined organic layer was washed with water (100.00 mL), brine (100.00 mL), dried over anhydrous Na₂SO₄ and concentrated to dryness in vacuum. The residue was purified by silica gel column chromatography, eluted with (PE:EtOAc=1:2) to give 2,5-dioxopyrrolidin-1-yl (2S)-2-[[(9H-fluoren-9-ylmethoxy)carbonyl]amino]propanoate (Compound 99, 25.00 g, 83%) t as a white solid. LCMS (ES, m/z): 431[M+Na]⁺.

### Step 6. Synthesis of Compound 100

To a solution of D-alanine (1.09 g, 0.012 mmol, 1.00 equiv) and NaHCO₃ (3.09 g, 0.04 mmol, 3.00 equiv) in water (50.00 mL) was added a solution of 2,5-dioxopyrrolidin-1-yl (2S)-2-[[(9H-fluoren-9-ylmethoxy)carbonyl]amino]propanoate (Compound 99, 5.00 g, 12.24 mmol, 1.00 equiv) in DMF (50.00 mL,). The resulting mixture was stirred at room temperature for 2 h. LCMS indicated the reaction was completed. The reaction was adjusted to pH value to 2-3 with 2 N HCl. The resulting mixture was extracted with EtOAc (100.00 mL×3), and the combined organic layer was washed with brine (100.00 mL×3), dried over anhydrous Na₂SO₄ and concentrated to dryness under vacuum to give (2R)-2-[(2S)-2-[[(9H-fluoren-9-ylmethoxy)carbonyl]-amino]propanamido]propanoic acid (Compound 100, 4.00 g, 71%) as a white solid. LCMS (ES, m/z): 383 [M+H]⁺

### Step 7. Synthesis of Compound 101

To a solution of glycine (3.68 g, 48.97 mmol, 1.00 equiv) and NaHCO₃ (12.34 g, 146.89 mmol, 3.00 equiv) in water (200.00 mL) was added solution of 2,5-dioxopyrrolidin-1-yl (2S)-2-[[(9H-fluoren-9-ylmethoxy)carbonyl]amino]propanoate (Compound 99, 20.00 g, 48.97 mmol, 1.00 equiv) in DMF (200.00 mL). The reaction was stirred at room temperature for 2 h. LCMS indicated the reaction was completed. The reaction was adjusted to pH value to 2-3 with 2 N HCl. The resulting mixture was extracted with EtOAc (500.00 mL×3), and the combined organic layer was washed with brine (500.00 mL), dried over anhydrous Na₂SO₄ and concentrated to dryness in vacuum to give [(2S)-2-[[(9H-fluoren-9-ylmethoxy)carbonyl]amino]propanamido]acetic acid (Compound 101, 15.00 g, 71%) as a white solid. LCMS (ES, m/z): 369 [M+H]⁺

### Step 8. Synthesis of Compound 102

A solution of [(2S)-2-[[(9H-fluoren-9-ylmethoxy)carbonyl]amino]propanamido]-acetic acid (Compound 101, 5.00 g, 13.57 mmol, 1.00 equiv), Pb(OAc)₄ (7.22 g, 16.28 mmol, 1.20 equiv) and pyridine (1.29 g, 16.31 mmol, 1.20 equiv) in THE (300.00 mL)/Toluene (100.00 mL) under N₂ was stirred at 80° C. for 16 h. LCMS indicated the reaction was completed. After cooled to room temperature, the reaction was filtered. The filter cake was washed with THF (100.00 mL). The combined organic layer was concentrated to dryness under vacuum. The residue was purified by silica gel column chromatography, eluted with (PE:EtOAc=1:2) to give [(2S)-2-[[(9H-fluoren-9-ylmethoxy)carbonyl]amino]propanamido]methyl acetate (Compound 102, 2.50 g, 45%) as a white solid. LCMS (ES, m/z): 405 [M+Na]⁺. ¹H NMR (400 MHz, Chloroform-d) δ 7.77 (t, J=7.6 Hz, 2H), 7.58 (d, J=7.6 Hz, 2H), 7.43-7.37 (m, 2H), 7.36-7.29 (m, 2H), 7.10 (s, 1H), 5.24 (d, J=7.6 Hz, 2H), 4.51-4.35 (m, 2H), 4.23-4.09 (m, 2H), 2.04 (s, 3H), 1.39 (d, J=6.8 Hz, 3H).

### Step 9. Synthesis of Compound 103

To a stirred solution of [(2S)-2-[[(9H-fluoren-9-ylmethoxy)carbonyl]amino]-propanamido]methyl acetate (Compound 102, 2.25 g, 5.88 mmol, 1.00 equiv) and 2-(2-chloro-4-nitrophenyl)ethanethiol (Compound 97, 1.28 g, 5.88 mmol, 1.00 equiv) in DCM (120 mL) was added TFA (0.27 mL, 2.37 mmol, 0.62 equiv) under N₂ at room temperature. The resulting mixture was stirred at room temperature for 16 hours. LCMS indicated the reaction was completed. The reaction was concentrated to dryness in vacuum. The residue was purified by silica gel column chromatography, eluted with (PE:EtOAc=1:4) to give to give 9H-fluoren-9-ylmethyl N-[(1S)-1-[([[2-(2-chloro-4-nitrophenyl)ethyl]sulfanyl]methyl)carbamoyl]ethyl]carbamate (Compound 103, 3.10 g, 90%) as a yellow solid. LCMS (ES, m/z): 540 [M+H]⁺

### Step 10. Synthesis of Compound 104

To a solution of 9H-fluoren-9-ylmethyl N-[(1S)-1-[([[2-(2-chloro-4-nitrophenyl)ethyl]sulfanyl]methyl)carbamoyl]ethyl]carbamate (Compound 103, 3.10 g, 5.74 mmol, 1.00 equiv) in DMF (155.00 mL) was added piperidine (31.00 mL) at 0° C. under N₂. The resulting mixture was stirred at 0° C. for 0.5 h under N₂. LCMS indicated the reaction was completed. The reaction was diluted with water (600.00 ml). The resulting mixture was extracted with EtOAc (200.00 mL×3). The combined organic layer was washed with brine (200.00 ml), dried over anhydrous Na₂SO₄ and concentrated to dryness under vacuum to give 3.00 g of the crude product. The crude product was re-purified by silica gel column chromatography, eluted with (DCM:MeOH=3:1) to give (2S)-2-amino-N-([[2-(2-chloro-4-nitrophenyl)ethyl]sulfanyl]methyl)propenamide, 104 (1.50 g, 78%) as a yellow oil. LCMS (ES, m/z): 318 [M+H]+.

### Step 11. Synthesis of Compound 105

To a solution of (2S)-2-amino-N-([[2-(2-chloro-4-nitrophenyl)ethyl]sulfanyl]methyl)-propenamide (Compound 104, 1.50 g, 4.72 mmol, 1.00 equiv) in DMF (75.00 mL) was added a solution of NaHCO₃ (0.59 g, 7.08 mmol, 1.50 equiv) in H₂O (10.00 mL) and Boc₂O (1.03 g, 4.72 mmol, 1.00 equiv) at room temperature. The reaction was stirred at room temperature for 1 h. LCMS indicated the reaction was completed. The reaction was diluted with water (500.00 mL), extracted with EtOAc (200.00 mL×3). The combined organic layer was washed with brine (200.00 mL×3), dried over anhydrous Na₂SO₄ and concentrated to dryness under vacuum to give tert-butyl N-[(1S)-1-[([[2-(2-chloro-4-nitrophenyl)ethyl]sulfanyl]methyl)carbamoyl]ethyl]carbamate (Compound 105, (1.82 g, 83) as a red oil. LCMS (ES, m/z): 418 [M+H]⁺, 318 [M+H−100]⁺

### Step 12. Synthesis of Compound 106

A slurry of tert-butyl N-[(1S)-1-[([[2-(2-chloro-4-nitrophenyl)ethyl]sulfanyl]-methyl)carbamoyl]ethyl]carbamate (Compound 105, 1.82 g, 4.36 mmol, 1.00 equiv), iron powder (2.43 g, 0.04 mmol, 10.00 equiv) and NH₄Cl (2.33 g, 0.04 mmol, 10.00 equiv) in EtOH (100.00 mL)/H₂O (50.00 mL) was stirred at 70° C. for 2 h. LCMS indicated the reaction was completed. The reaction was filtered. The filtrate was concentrated to dryness under vacuum. The residue was dissolved with DCM (50.00 mL) and filtered. The filtrate was concentrated to dryness and the residue was purified by silica gel column chromatography, eluted with (DCM:MeOH=13:1) to give tert-butyl N-[(1S)-1-[([[2-(4-amino-2-chlorophenyl)ethyl]sulfanyl]methyl)carbamoyl]ethyl]-carbamate (Compound 106, 1.20 g, 68%) as a yellow oil. LCMS (ES, m/z): 388 [M+H]⁺

### Step 13. Compound 107

To a stirred solution of 3-[5-(aminomethyl)-1-oxo-3H-isoindol-2-yl]piperidine-2,6-dione (INT 1, 352 mg, 1.29 mmol, 1.00 equiv) in DMF (5.00 mL) at 0° C. was added CDI (209.00 mg, 1.29 mmol, 1 equiv) and TEA (260 mg, 2.58 mmol, 2 equiv). The resulting mixture was stirred at 0° C. for 2 h. Then tert-butyl N-[(1S)-1-[([[2-(4-amino-2-chlorophenyl)ethyl]sulfanyl]-methyl)-carbamoyl]ethyl]carbamate (Compound 106, 500.00 mg, 1.29 mmol, 1.00 equiv) and DMAP (472 mg, 3.87 mmol, 3.00 equiv) were added. The resulting mixture was stirred at 60° C. for 24 h. LCMS indicated the reaction was completed. After cooled to room temperature, The reaction mixture was purified by reverse flash chromatography with the following conditions: column, C18 silica gel; mobile phase, ACN in water (0.1% FA), 0% to 60% gradient in 30 min; detector, UV 254 nm to give tert-butyl N-[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)ethyl]carbamate (Compound 107, 450.00 mg, 48%) t as a yellow solid. LCMS (ES, m/z): 687 [M+H]⁺

### Step 14. Compound 108

To a stirred solution of tert-butyl N-[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]-methyl]carbamoyl)ethyl]carbamate (Compound 107, 440.00 mg, 0.64 mmol, 1.00 equiv) in DCM (22.00 mL) was added TFA (2.20 mL) at room temperature. The resulting mixture was stirred at room temperature for 0.5 h. LCMS indicated the reaction was completed. The reaction was concentrated to dryness under vacuum to give (2S)-2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]-methyl]propanamide; trifluoroacetic acid (Compound 108, 400.00 mg, crude) as a red oil. The residue was used to next step without further purification. LCMS (ES, m/z): 587 [M+H−TFA]⁺

### Step 15. Synthesis of Compound 109

A solution of (2R)-2-[(2S)-2-[[(9H-fluoren-9-ylmethoxy)carbonyl]-amino]propanamido]propanoic acid (218 mg, 0.57 mmol, 1.00 equiv), HOBT (77 mg, 0.57 mmol, 1.00 equiv) and HATU (216 mg, 0.01 mmol, 1.00 equiv) was stirred at room temperature in air for 1 hour, then (2S)-2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]propanamide; trifluoroacetic acid (Compound 108, 400 mg, 0.57 mmol, 1.00 equiv) and DIEA (663 mg, 5.14 mmol, 9.00 equiv) was added at room temperature. The reaction was stirred at room temperature for 2 h. LCMS indicated the reaction was completed. The reaction mixture was purified by reverse flash chromatography with the following conditions: column, C18 silica gel; mobile phase, ACN in water (0.05% TFA), 0% to 50% gradient in 30 min; detector, UV 254 nm to give 9H-fluoren-9-ylmethyl N-[(1S)-1-[[(1R)-1-[[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)ethyl]carbamoyl]ethyl]carbamoyl]ethyl]carbamate (Compound 109, 480.00 mg, 75%) as a green solid. LCMS (ES, m/z): 951 [M+H]⁺

### Step 16. Compound 110

To a solution of 9H-fluoren-9-ylmethyl N-[(1S)-1-[[(1R)-1-[[(1S)-1-([[(2-[2-chloro-4-[([[2-(2,6-dioxopiperidin-3-yl)-1-oxo-3H-isoindol-5-yl]methyl]carbamoyl)amino]phenyl]-ethyl)sulfanyl]methyl]carbamoyl)ethyl]carbamoyl]ethyl]carbamoyl]ethyl]carbamate (Compound 109, 110.00 mg) in DMF (5.00 mL) was added piperidine (1.00 mL) at 0° C. The resulting mixture was stirred at 0° C. for 0.5 h. LCMS indicated the reaction was completed. The reaction mixture was purified by reverse flash chromatography with the following conditions: column, C18 silica gel; mobile phase, ACN in water (0.05% TFA), 0% to 60% gradient in 40 min; detector, UV 254 nm to give (2S)-2-[(2R)-2-[(2S)-2-aminopropanamido]propanamido]-N-[[(2-[2-chloro-4-[([[2-(2,6-dioxopiperidin-3-yl)-1-oxo-3H-isoindol-5-yl]methyl]carbamoyl)amino]-phenyl]ethyl)sulfanyl]methyl]propenamide (Compound 110, 80.00 mg, 60%) as a red solid. LCMS (ES, m/z): 729 [M+H]⁺. ¹H NMR (400 MHz, DMSO-d₆) δ 9.00 (br s, 1H), 8.53 br (s, 1H), 8.24 (d, J=7.6 Hz, 1H), 8.10 (br s, 1H), 7.69-7.62 (m, 2H), 7.49 (s, 1H), 7.42 (d, J=8.0 Hz, 1H), 7.26-7.13 (m, 3H), 7.00 (br s, 1H), 5.11-5.06 (m, 1H), 4.45-4.36 (m, 3H), 4.35-4.13 (m, 6H), 2.90-2.83 (m, 3H), 2.73-2.71 (m, 2H), 2.05-1.90 (m, 1H), 1.70-1.53 (m, 4H), 1.22-1.17 (m, 6H), 1.14-1.05 (m, 3H).

### Step 17. Synthesis of Compound (Ik)

To a solution of (2S)-2-[(2R)-2-[(2S)-2-aminopropanamido]propanamido]-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]propenamide (Compound 110, 63.00 mg, 0.09 mmol, 1.00 equiv) and 2,5-dioxopyrrolidin-1-yl 6-(2,5-dioxopyrrol-1-yl)hexanoate (26 mg, 0.09 mmol, 1.00 equiv) in DMF (1.50 mL, 19.38 mmol, 224.36 equiv) was added DIEA (22.33 mg, 0.17 mmol, 2.00 equiv) at room temperature in air. The reaction was stirred at room temperature for 1 h. The reaction mixture was purified by reverse flash chromatography with the following conditions: Column: Kinetex EVO C18 Column, 30×150.5 um; Mobile Phase A:water (0.05% TFA), Mobile Phase B:ACN; Flow rate: 60 mL/min; Gradient: 23 B to 43 B in 7 min, 254 nm; RT1:6.58). The collected fraction was lyophilized to give N-[(1S)-1-[[(1R)-1-[[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)ethyl]carbamoyl]ethyl]carbamoyl]ethyl]-6-(2,5-dioxopyrrol-1-yl)hexanamide (Compound (Ik), 16.10 mg, 20%) as a white solid. LCMS (ES, m/z): 922,924 [M+H]⁺. ¹HNMR (400 MHz, DMSO-d₆) δ 11.00 (s, 1H), 8.80 (s, 1H), 8.44-8.41 (m, 1H), 8.15 (d, J=7.2 Hz, 1H), 8.03-8.00 (m, 2H), 7.7-7.65 (m, 2H), 7.51 (s, 1H), 7.44 (d, J=8.0 Hz, 1H), 7.22-7.14 (m, 2H), 6.98 (s, 2H), 6.83-6.81 (m, 1H), 5.13-5.08 (m, 1H), 4.48-4.40 (m, 3H), 4.29-4.17 (m, 6H), 2.96-2.85 (m, 3H), 2.75-2.70 (m, 2H), 2.67-2.57 (m, 1H), 2.40-2.33 (m, 1H), 2.09-1.98 (m, 3H), 1.52-1.45 (m, 5H), 1.26-1.16 (m, 12H).

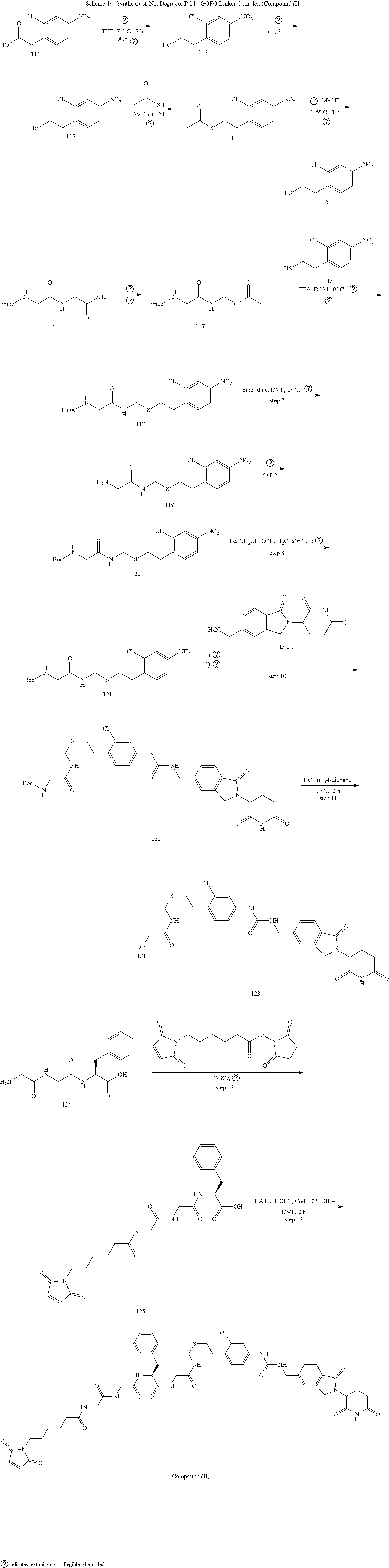

### Step 1. Synthesis of Compound 112

To a stirred solution of (2-chloro-4-nitrophenyl)acetic acid (Compound 111, 5.00 g, 23.19 mmol, 1.00 equiv) in THF (50 mL) were added BH₃-Me₂S (5.50 mL, 57.99 mmol, 2.50 equiv) in portions at room temperature under nitrogen atmosphere. The resulting mixture was stirred for 2 h at 70° C. under nitrogen atmosphere. TLC (PE:EtOAc=3:1) indicated the reaction was completed. The residue was purified by silica gel column chromatography, eluted with PE/EtOAc (2:1) to afford 2-(2-chloro-4-nitrophenyl)ethanol (Compound 112, 4.8 g, 92%) as a light yellow solid. ¹H NMR (400 MHz, Chloroform-d) δ 8.27 (d, J=2.4 Hz, 1H), 8.10 (dd, J=8.4, 2.4 Hz, 1H), 7.46 (s, 1H), 3.20-3.09 (m, 4H).

### Step 2. Synthesis of Compound 113

To a stirred solution of 2-(2-chloro-4-nitrophenyl)ethanol (Compound 112, 4.80 g, 23.81 mmol, 1.00 equiv) in DCM (100 mL) were added NBS (6.36 g, 35.71 mmol, 1.50 equiv) and PPh₃ (9.37 g, 35.72 mmol, 1.50 equiv) in portions at room temperature under air atmosphere. The resulting mixture was stirred for overnight at room temperature under air atmosphere. TLC (PE:EtOAc=10:1) indicated the reaction was completed. The reaction was concentrated to dryness under vacuum. The residue was purified by silica gel column chromatography, eluted with PE/EtOAc (4:1) to afford 1-(2-bromoethyl)-2-chloro-4-nitrobenzene (Compound 113, 3.9 g, 57%) as a red oil. ¹H NMR (400 MHz, Chloroform-d) δ8.29 (d, J=2.4 Hz, 1H), 8.13 (dd, J=8.4, 2.4 Hz, 1H), 7.50 (d, J=8.4 Hz, 1H), 3.67 (t, J=7.2 Hz, 2H), 3.42 (t, J=7.2 Hz, 2H).

### Step 3. Synthesis of Compound 114

To a solution of 1-(2-bromoethyl)-2-chloro-4-nitrobenzene (Compound 113, 3.90 g, 14.75 mmol, 1.00 equiv) in DMF (39 mL) was added potassium thioacetate (1.68 g, 14.75 mmol, 1.00 equiv) at room temperature. The resulting mixture was stirred at room temperature for 2 hours. TLC ((PE:EtOAc=10:1) indicated the reaction was completed. The reaction was diluted with water (600 mL). The resulting mixture was extracted with EA (200 mL*3). The combined organic layer was washed with water (200 mL), brine (200 mL), dried over anhydrous sodium sulfate and concentrated to dryness under vacuum to give 1-[[2-(2-chloro-4-nitrophenyl)ethyl]sulfanyl]ethenone (Compound 114, 3.7 g, 85%) as a red oil. ¹H NMR (400 MHz, Chloroform-d) δ 8.27 (d, J=2.4 Hz, 1H), 8.10 (dd, J=8.4, 2.4 Hz, 1H), 7.46 (s, 1H), 3.21-3.02 (m, 4H), 2.37 (s, 3H).

### Step 4. Synthesis of Compound 115

To a stirred solution of 1-[[2-(2-chloro-4-nitrophenyl)ethyl]sulfanyl]ethenone (Compound 114, 4.00 g, 15.40 mmol, 1.00 equiv) in MeOH (600 mL) was added MeONa (14.31 mL, 77.00 mmol, 5.00 equiv, 30%) at 0° C. N₂ for 1 h. The reaction mixture was stirred at 0° C. for 1 h. TLC indicate (PE:EA=10:1) the reaction was completed. The reaction was quenched with AcOH. The resulting mixture was concentrated to dryness under vacuum. The residue was diluted with DCM (100 mL) and filtered. The filtrate was purified by silica gel column chromatography, eluted with (PE:EtOAc=10:1) to give 2-(2-chloro-4-nitrophenyl)ethanethiol (Compound 115, 3 g, 80%) as a yellow oil. ¹H NMR (400 MHz, Chloroform-d) δ 8.28 (d, J=2.4 Hz, 1H), 8.11 (dd, J=8.4, 2.4 Hz, 1H), 7.48 (d, J=8.4 Hz, 1H), 3.17 (t, J=7.2 Hz, 2H), 2.87 (dt, J=8.0, 7.2 Hz, 2H), 1.46 (t, J=8.0 Hz, 1H).

### Step 5. Synthesis of Compound 117

To a stirred mixture of (2-[[(9H-fluoren-9-ylmethoxy)carbonyl]amino]-acetamido)acetic acid (Compound 116, 10 g, 28.22 mmol, 1.00 equiv) and Pb(OAc)₄ (15 g, 33.86 mmol, 1.20 equiv) in THF (300 mL) and toluene (100 mL) were added pyridine (2.59 g, 32.74 mmol, 1.16 equiv) dropwise at room temperature under nitrogen atmosphere. The resulting mixture was stirred for overnight at 80 degrees C. under nitrogen atmosphere. LCMS indicated the reaction was completed. The mixture was allowed to cool down to room temperature. The resulting mixture was filtered, the filter cake was washed with EA (20 mL). The filtrate was concentrated under vacuum. The residue was dissolved in EA (20 mL). The resulting mixture was washed with water, brine, dried over anhydrous Na₂SO₄. After filtration, the filtrate was concentrated under vacuum. The residue was purified by silica gel column chromatography, eluted with PE/EtOAc (1:4) to give (2-[[(9H-fluoren-9-ylmethoxy)carbonyl]amino]acetamido)methyl acetate (Compound 117, 6.5 g, 56%) as a white solid. ¹HNMR (300 MHz, CDCl₃) δ7.80 (d, J=7.5 Hz, 2H), 7.62 (d, J=7.5 Hz, 2H), 7.45 (t, d=7.5 Hz, 2H), 7.36 (d, d=7.5 Hz, 2H), 7.18 (br s, 1H), 5.48 (br s, 1H), 5.28 (d, J=7.2 Hz, 2H), 4.48 (d, J=6.6 Hz, 2H), 4.26 (t, J=6.6 Hz, 1H), 3.93 (d, 5.4 Hz, 2H), 2.08 (s, 3H). LCMS (ESI, ms): 391[M+Na]⁺

### Step 6. Synthesis of Compound 118

To a solution of (2-[[(9H-fluoren-9-ylmethoxy)carbonyl]amino]acetamido)methyl acetate (Compound 117, 3.00 g, 8.14 mmol, 1.00 equiv) and 2-(2-chloro-4-nitrophenyl)ethanethiol (Compound 115, 1.77 g, 8.13 mmol, 1.00 equiv)) in DCM (300 mL) was added TFA (0.56 g, 4.91 mmol, 0.60 equiv) at room temperature. The resulting mixture was stirred at 60° C. for 16 h. LCMS indicated the reaction was completed. The reaction was concentrated to dryness under vacuum. The residue was purified by silica gel column chromatography, eluted with (PE:EtOAc=2:3) to give 9H-fluoren-9-ylmethyl N-[[([[2-(2-chloro-4-nitrophenyl)ethyl]sulfanyl]methyl)carbamoyl]-methyl]carbamate (Compound 118, 3.7 g, 67%) as an off-white solid. LCMS (ES, m/z): 526,528 [M+H]⁺

### Step 7. Synthesis of Compound 119

To a solution of 9H-fluoren-9-ylmethyl N-[[([[2-(2-chloro-4-nitrophenyl)ethyl]sulfanyl]methyl)carbamoyl]methyl]carbamate (Compound 118, 3.70 g, 7.03 mmol, 1.00 equiv) in DMF (40 mL) was added piperidine (8 mL) at 0° C. The resulting mixture was stirred at 0° C. for 0.5 h. LCMS indicated the reaction was completed. The resulting mixture was diluted with water (400 mL), extracted with EA (200 mL×3). The combined organic layer was washed with water (200 mL), brine (200 mL), dried over anhydrous sodium sulfate and concentrated to dryness under vacuum. The residue was purified by silica gel column chromatography, eluted with (DCM:MeOH=10:1) to give 2-amino-N-([[2-(2-chloro-4-nitrophenyl)ethyl]sulfanyl]-methyl)acetamide (Compound 119, 1.01 g, 40%) as a yellow oil. LCMS (ES, m/z): 304,306 [M+H]⁺

### Step 8. Synthesis of Compound 120

To a solution of 2-amino-N-([[2-(2-chloro-4-nitrophenyl)ethyl]sulfanyl]methyl)-acetamide (Compound 119, 1.00 g, 3.29 mmol, 1.00 equiv) in DMF (50 mL) was added solution of NaHCO₃ (0.33 g, 3.92 mmol, 1.20 equiv) in water (10 mL), Boc₂O (0.72 g, 3.30 mmol, 1.00 equiv) at room temperature. The resulting mixture was stirred at room temperature for 1 h. LCMS indicated the reaction was completed. The reaction was diluted with water (500 mL), extracted with EtOAc (200 mL×3). The combined organic layer was washed with brine (200 mL), dried over anhydrous Na₂SO₄ and concentrated to dryness under vacuum. The residue was purified by silica gel column chromatography, eluted with (PE:EtOAc=1:3) to give tert-butyl N-[[([[2-(2-chloro-4-nitrophenyl)ethyl]sulfanyl]methyl)carbamoyl]methyl]carbamate (Compound 120, 810 mg, 54%) as a white solid. LCMS (ES, m/z): 404,406 [M+H]⁺, 304,306 [M+H−100]⁺

### Step 9. Synthesis of Compound 121

To a solution of tert-butyl N-[[([[2-(2-chloro-4-nitrophenyl)ethyl]sulfanyl]-methyl)carbamoyl]methyl]carbamate (Compound 120, 800.00 mg, 1.98 mmol, 1.00 equiv) in EtOH (40) was added iron powder (1106 mg, 19.81 mmol, 10.00 equiv) and solution of NH₄Cl (1059 mg, 19.81 mmol, 10.00 equiv) in water (10 mL) at room temperature. The resulting mixture was stirred at 70° C. for 2 h. LCMS indicated the reaction was completed. The reaction was filtered. The filtrate was concentrated to dryness under vacuum. The residue was dissolved with DCM (50.00 mL) and filtered. The filtrate was purified by silica gel column chromatography, eluted with (DCM:MeOH=13:1) to give tert-butyl N-[[([[2-(4-amino-2-chlorophenyl)ethyl]sulfanyl]methyl)-carbamoyl]methyl]carbamate (Compound 121, 610 mg, 74%) as a yellow oil. LCMS (ES, m/z): 374,376 [M+H]⁺, 374,376 [M+H−100]⁺

### Step 10. Synthesis of Compound 122

To a solution of 3-[5-(aminomethyl)-1-oxo-3H-isoindol-2-yl]piperidine-2,6-dione (INT 1, 219 mg, 0.80 mmol, 1.00 equiv) in DMF (10 mL) was added CDI (130 mg, 0.80 mmol, 1.00 equiv) and TEA (81 mg, 0.80 mmol, 1.00 equiv) at 0° C. in air. The resulting mixture was stirred at room temperature for 2 h. Then tert-butyl N-[[([[2-(4-amino-2-chlorophenyl)ethyl]sulfanyl]-methyl)carbamoyl]methyl]carbamate (Compound 121, 300 mg, 0.80 mmol, 1.00 equiv) and DMAP (294 mg, 2.41 mmol, 3.00 equiv) was added at room temperature in air. The resulting mixture was stirred at 60° C. for 48 h. LCMS indicated the reaction was completed. The resulting mixture purified by reverse flash chromatography with the following conditions: column, C18 silica gel; mobile phase, ACN in water (0.05% TFA), 0% to 60% gradient in 30 min; detector, UV 254 nm to give tert-butyl N-[([[(2-[2-chloro-4-[([[2-(2,6-dioxopiperidin-3-yl)-1-oxo-3H-isoindol-5-yl]methyl]-carbamoyl)amino]phenyl]ethyl)sulfanyl]methyl]carbamoyl)methyl]carbamate (Compound 122, 270 mg, 49%) as a yellow solid. LCMS (ES, m/z): 673,675 [M+H]⁺, 573,575 [M+H−100]⁺

### Step 11. Synthesis of Compound-123

To a solution of tert-butyl N-[([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)methyl]-carbamate (Compound 122, 250 mg, 0.37 mmol) in 1,4-dioxane (12 mL) was added HCl (4N in 1,4-dioxane, 6 mL) at 0° C. under N₂. The reaction was stirred at room temperature for 2 h. LCMS indicated the reaction was completed. The reaction mixture was concentrated to dryness under vacuum to give 2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]acetamide (Compound 123, 260 mg, crude) as a brown solid. LCMS (ES, m/z): 573,575 [M+H−HCl]⁺

### Step 12. Synthesis of Compound-125

A solution of (2S)-2-[2-(2-aminoacetamido)acetamido]-3-phenylpropanoic acid (Compound 124, 500 mg, 1.79 mmol, 1.00 equiv) and 2,5-dioxopyrrolidin-1-yl 6-(2,5-dioxopyrrol-1-yl)hexanoate (552 mg, 1.79 mmol, 1.00 equiv) in DMSO (5.00 mL) was stirred at room temperature in air for 16 h. LCMS indicated the reaction was completed. The reaction mixture was purified by reverse flash chromatography with the following conditions: column, C18 silica gel; mobile phase, ACN in water (0.1% FA), 0% to 60% gradient in 30 min; detector, UV 220 nm to give (2S)-2-(2-[2-[6-(2,5-dioxopyrrol-1-yl)hexanamido]acetamido]acetamido)-3-phenylpropanoic acid (Compound 125, 760 mg, 83%) as a white solid. LCMS (ES, m/z): 473 [M+H]⁺

### Step 13. Synthesis of Compound (II)

To a solution of (2S)-2-(2-[2-[6-(2,5-dioxopyrrol-1-yl)hexanamido]acetamido]-acetamido)-3-phenylpropanoic acid (Compound 125, 175 mg, 0.37 mmol, 1.00 equiv) in DMF (5.00 mL) were added HATU (141 mg, 0.37 mmol, 1.00 equiv) and HOBT (50 mg, 0.37 mmol, 1.00 equiv) at room temperature in air. The resulting mixture was stirred at room temperature for 1 h. Then 2-amino-N-[[(2-[2-chloro-4-[([[2-(2,6-dioxopiperidin-3-yl)-1-oxo-3H-isoindol-5-yl]methyl]-carbamoyl)amino]phenyl]ethyl)sulfanyl]methyl]acetamide (Compound 123, 250 mg, 0.37 mmol, 1.00 equiv, 85%) and DIEA (240 mg, 1.85 mmol, 5.00 equiv) were added. The resulting mixture was stirred at room temperature for 1 h. LCMS indicated the reaction was completed. The reaction mixture was purified by the following condition: Column: XSelect CSH Prep C18 OBD Column, 19*250 mm, 5 um; Mobile Phase A:water (0.05% FA), Mobile Phase B:ACN; Flow rate: 25 mL/min; Gradient: 30 B to 60 B in 7 min, 254 nm; RT1:6.67 min to give 75 mg of the crude product. The crude product was re-purified by reverse flash chromatography with the following conditions: Column: XBridge Shield RP18 OBD Column, 19*250 mm, 10 um; Mobile Phase A:water (0.1% FA), Mobile Phase B:ACN; Flow rate: 25 mL/min; Gradient: 25 B to 44 B in 10 min; 254 nm; RT1:10.52 min. The collected fraction was lyophilized to give Compound (Il) (41.6 mg, 10%) as a white solid. ¹HNMR (400 MHz, DMSO-d₆) δ10.99 (s, 1H), 8.79 (s, 1H), 8.38 (t, J=6.0 Hz, 1H), 8.31 (t, J=6.0 Hz, 1H), 8.12 (d, J=8.4 Hz, 1H), 8.06 (t, J=5.6 Hz, 1H), 8.01 (t, J=6.0 Hz, 1H), 7.70-7.66 (m, 2H), 7.51 (s, 1H), 7.44 (d, J=8.0 Hz, 1H), 7.25-7.21 (m, 5H), 7.19-7.14 (m, 2H), 6.99 (s, 2H), 6.82 (t, J=6.0 Hz, 1H), 5.13-5.08 (m, 1H), 4.47-4.40 (m, 4H), 4.33-4.29 (m, 3H), 3.76-3.70 (m, 3H), 3.67-3.55 (m, 3H), 3.38-3.36 (m, 2H), 3.06-3.02 (m, 1H), 2.91-2.86 (m, 3H), 2.82-2.70 (m, 3H), 2.62-2.57 (m, 1H), 2.50-2.45 (m, 1H), 2.10 (m, 2H), 2.05-1.95 (m, 1H), 1.50-1.44 (m, 4H), 1.20-1.16 (m, 2H). LCMS (ES, m/z): 1027,1029 [M+H]⁺

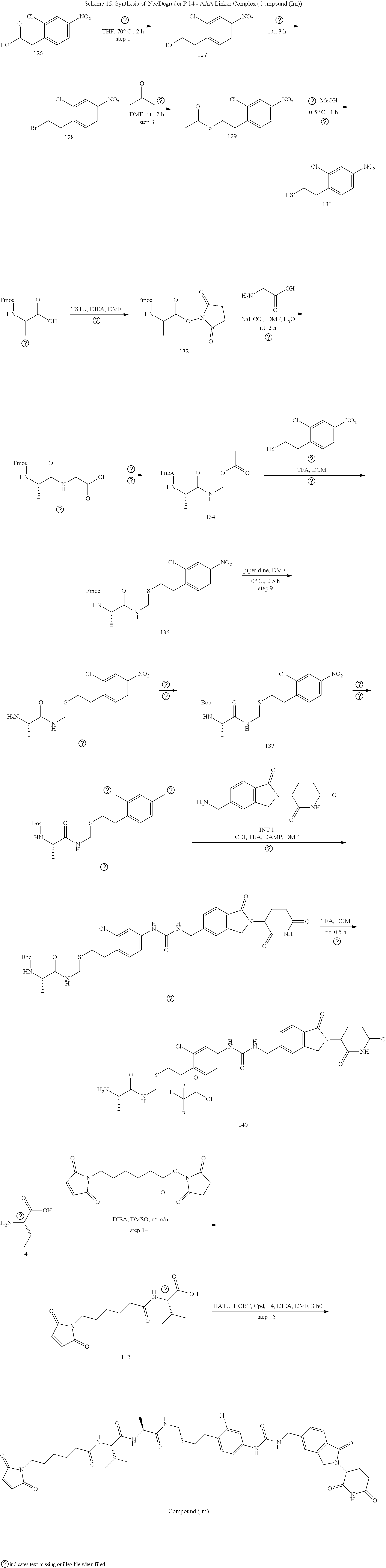

### Step 1. Synthesis of Compound 127

To a stirred solution of (2-chloro-4-nitrophenyl)acetic acid (Compound 126, 24.00 g, 111.32 mmol, 1.00 equiv) in THE (240.00 mL) were added BH₃-Me₂S (28.00 mL, 295.23 mmol, 2.65 equiv) dropwise under nitrogen atmosphere. The resulting mixture was stirred for 2 hours at 70° C. under nitrogen atmosphere. TLC (PE:EtOAc=3:1) indicated the reaction was completed. After cooled to room temperature, the resulting mixture was concentrated under vacuum. The residue was purified by silica gel column chromatography, eluted with PE/EtOAc (3:1) to afford 2-(2-chloro-4-nitrophenyl)ethanol (Compound 127, 18.00 g, 80%) as a light yellow solid. ¹H NMR (300 MHz, CD₃Cl) δ8.27 (s, 1H), 8.10-8.07 (m, 1H), 7.52 (d, J=3 Hz, 1H), 3.96 (t, J=6 Hz, 2H), 3.13 (t, J=6 Hz, 2H).

### Step 2. Synthesis of Compound 128

To a stirred solution of 2-(2-chloro-4-nitrophenyl)ethanol (Compound 127, 5.00 g, 24.80 mmol, 1.00 equiv) in DCM (100.00 mL) were added NBS (6.62 g, 1.50 equiv) and PPh₃ (9.76 g, 37.21 mmol, 1.50 equiv) in portions at room temperature under N₂. The resulting mixture was stirred overnight at room temperature under N₂. TLC (PE:EtOAc=10:1) indicated the reaction was completed. The reaction was concentrated to dryness under vacuum. The residue was purified by silica gel column chromatography, eluted with PE/EtOAc (4:1) to afford 1-(2-bromoethyl)-2-chloro-4-nitrobenzene (Compound 128, 5.10 g, 72.31%) as a red oil. ¹H NMR (400 MHz, DMSO-d₆) δ 8.28 (d, J=2.4 Hz, 1H), 8.18 (dd, J=8.4, 2.4 Hz, 1H), 7.73 (d, J=8.4 Hz, 1H), 3.79 (t, J=7.2 Hz, 2H), 3.38 (t, J=7.2 Hz, 2H).

### Step 3. Synthesis of Compound 129

To a solution of 1-(2-bromoethyl)-2-chloro-4-nitrobenzene (Compound 128, 5.00 g, 18.90 mmol, 1.00 equiv) in DMF (50.00 mL) was added potassium thioacetate (2.16 g, 18.91 mmol, 1.00 equiv) at room temperature under nitrogen atmosphere. The resulting mixture was stirred at room temperature for 2 hours. TLC (PE:EtOAc=10:1) indicated the reaction was completed. The reaction was diluted with water (600.00 mL). The resulting mixture was extracted with EtOAc (200.00 mL*3). The combined organic layer was washed with water (200.00 mL), brine (200.00 mL*3), dried over anhydrous Na₂SO₄ and concentrated to dryness under vacuum to afford 1-[[2-(2-chloro-4-nitrophenyl)ethyl]sulfanyl]ethenone (Compound 129, 4.50 g, 85%) as a red oil. ¹H NMR (400 MHz, CDCl₃) δ 8.24 (d, J=2.4 Hz, 1H), 8.07 (dd, J=8.4, 2.4 Hz, 1H), 7.45 (d, J=8.4 Hz, 1H), 3.20-3.05 (m, 4H), 2.34 (s, 3H).

### Step 4. Synthesis of Compound 130

To a stirred solution of 1-[[2-(2-chloro-4-nitrophenyl)ethyl]sulfanyl]ethenone (Compound 129, 2.00 g, 7.70 mmol, 1.00 equiv) in MeOH (300.00 mL) was added MeONa (6.93 mL, 37.33 mmol, 5.00 equiv, 30%) at 0° C. under N₂. The resulting mixture was stirred at 0° C. under N₂ for 1 h. TLC (PE:EtOAc=10:1) indicated the reaction was completed. The reaction was quenched with AcOH to pH value to 3-4. The resulting mixture was concentrated to dryness under vacuum. The residue was diluted with DCM (50.00 mL) and filtered. The filtrate was purified with Prep-TLC (PE:EtOAc=10:1) to give 2-(2-chloro-4-nitrophenyl)ethanethiol (Compound 130, 1.35 g, 72%) as a light yellow oil. ¹H NMR (400 MHz, CDCl₃) δ 8.26 (d, J=2.4 Hz, 1H), 8.09 (dd, J=8.4, 2.4 Hz, 1H), 7.45 (d, J=8.4 Hz, 1H), 3.14 (dd, J=8.0, 6.8 Hz, 2H), 2.85 (dt, J=8.0, 7.2 Hz, 2H), 1.43 (t, J=8.0 Hz, 1H).

### Step 5. Synthesis of Compound 132

To a stirred solution of (2S)-2-[[(9H-fluoren-9-ylmethoxy)carbonyl]amino]propanoic acid (Compound 131, 20.00 g, 64.24 mmol, 1.00 equiv) in DMF (200.00 mL) were added TSTU (25.18 g, 83.52 mmol, 1.30 equiv) and DIEA (16.60 g, 128.48 mmol, 2.00 equiv) at room temperature under air atmosphere. The resulting mixture was stirred for 1 h at room temperature. LCMS indicated the reaction was completed. The reaction was diluted with water (200.00 mL), the resulting mixture was extracted with ETOAC (100.00 mL*3). The combined organic layer was washed with water (100.00 mL), brine (100.00 mL), dried over anhydrous Na₂SO₄ and concentrated to dryness in vacuum. The residue was purified by silica gel column chromatography, eluted with (PE:EtOAc=1:2) to give 2,5-dioxopyrrolidin-1-yl (2S)-2-[[(9H-fluoren-9-ylmethoxy)carbonyl]amino]propanoate (Compound 132, 25.00 g, 83%) t as a white solid. LCMS (ES, m/z): 431[M+Na]⁺

### Step 6. Synthesis of Compound 133

To a solution of glycine (3.68 g, 48.97 mmol, 1.00 equiv) and NaHCO₃ (12.34 g, 146.89 mmol, 3.00 equiv) in water (200.00 mL) was added solution of 2,5-dioxopyrrolidin-1-yl (2S)-2-[[(9H-fluoren-9-ylmethoxy)carbonyl]amino]propanoate (Compound 132, 20.00 g, 48.97 mmol, 1.00 equiv) in DMF (200.00 mL). The reaction was stirred at room temperature for 2 h. LCMS indicated the reaction was completed. The reaction was adjusted to pH value to 2-3 with 2 N HCl. The resulting mixture was extracted with EtOAc (500.00 mL*3), the combined organic layer was washed with brine (500.00 mL), dried over anhydrous Na₂SO₄ and concentrated to dryness in vacuum to give [(2S)-2-[[(9H-fluoren-9-ylmethoxy)carbonyl]amino]propanamido]acetic acid (Compound 133, 15.00 g, 71%) as a white solid. LCMS (ES, m/z): 369 [M+H]⁺

### Step 7. Synthesis of Compound 134

A solution of [(2S)-2-[[(9H-fluoren-9-ylmethoxy)carbonyl]amino]-propanamido]acetic acid (Compound 133, 5.00 g, 13.57 mmol, 1.00 equiv), Pb(OAc)₄ (7.22 g, 16.28 mmol, 1.20 equiv) and pyridine (1.29 g, 16.31 mmol, 1.20 equiv) in THE (300.00 mL)/Toluene (100.00 mL) under N₂ was stirred at 80° C. for 16 h. LCMS indicated the reaction was completed. After cooled to room temperature, the reaction was filtered. The filter cake was washed with THE (100.00 mL). The combined organic layer was concentrated to dryness under vacuum. The residue was purified by silica gel column chromatography, eluted with (PE:ETOAC=1:2) to give [(2S)-2-[[(9H-fluoren-9-ylmethoxy)carbonyl]amino]propanamido]methyl acetate (Compound 134, 2.50 g, 45%) as a white solid. LCMS (ES, m/z): 405 [M+Na]. ¹H NMR (400 MHz, Chloroform-d) δ 7.77-7.73 (m, 2H), 7.58 (d, J=7.6 Hz, 2H), 7.43-7.37 (m, 2H), 7.36-7.29 (m, 2H), 7.10 (s, 1H), 5.24 (d, J=7.6 Hz, 2H), 4.51-4.35 (m, 2H), 4.22 (t, J=6.8 Hz, 2H), 2.04 (s, 3H), 1.39 (d, J=6.8 Hz, 3H).

### Step 8. Synthesis of Compound 135

To a stirred solution of [(2S)-2-[[(9H-fluoren-9-ylmethoxy)carbonyl]amino]-propanamido]methyl acetate (Compound 134, 2.25 g, 5.88 mmol, 1.00 equiv) and 2-(2-chloro-4-nitrophenyl)ethanethiol (Compound 500, 1.28 g, 5.88 mmol, 1.00 equiv) in DCM (120 mL) was added TFA (0.27 mL, 2.376 mmol, 0.62 equiv) under N₂ at room temperature. The resulting mixture was stirred at 40° C. for 16 hours. LCMS indicated the reaction was completed. The reaction was concentrated to dryness in vacuum to give 9H-fluoren-9-ylmethyl N-[(1S)-1-[([[2-(2-chloro-4-nitrophenyl)ethyl]sulfanyl]methyl)carbamoyl]ethyl]carbamate (Compound 135, 3.10 g, 90%) as a yellow solid. LCMS (ES, m/z): 540,542 [M+H]⁺.

### Step 9. Synthesis of Compound 136

To a solution of 9H-fluoren-9-ylmethyl N-[(1S)-1-[([[2-(2-chloro-4-nitrophenyl)ethyl]sulfanyl]methyl)carbamoyl]ethyl]carbamate (Compound 135, 3.10 g, 5.74 mmol, 1.00 equiv) in DMF (155.00 mL) was added piperidine (31.00 mL) at 0° C. under N₂. The resulting mixture was stirred at 0° C. for 0.5 h under N₂. LCMS indicated the reaction was completed. The reaction was diluted with water (600.00 ml). The resulting mixture was extracted with EA (200.00 mL×3). The combined organic layer was washed with brine (200.00 ml), dried over anhydrous Na₂SO₄ and concentrated to dryness under vacuum to give 3.00 g of the crude product. The crude product was re-purified by silica gel column chromatography, eluted with (DCM:MeOH=3:1) to give (2S)-2-amino-N-([[2-(2-chloro-4-nitrophenyl)ethyl]sulfanyl]methyl)propenamide (Compound 136, 1.50 g, 78%) as a yellow oil. LCMS (ES, m/z): 318,320 [M+H]⁺.

### Step 10. Synthesis of Compound 137

To a solution of (2S)-2-amino-N-([[2-(2-chloro-4-nitrophenyl)ethyl]sulfanyl]-methyl)propenamide (Compound 136, 1.50 g, 4.72 mmol, 1.00 equiv) in DMF (75.00 mL) was added a solution of NaHCO₃ (0.59 g, 7.08 mmol, 1.50 equiv) in H₂O (10.00 mL) and Boc₂O (1.03 g, 4.72 mmol, 1.00 equiv) at room temperature in air. The reaction was stirred at room temperature for 1 h. LCMS indicated the reaction was completed. The reaction was diluted with water (500.00 mL), extracted with EtOAc (200.00 mL×3). The combined organic layer was washed with brine (200.00 mL*3), dried over anhydrous Na₂SO₄ and concentrated to dryness under vacuum to give tert-butyl N-[(1S)-1-[([[2-(2-chloro-4-nitrophenyl)ethyl]sulfanyl]methyl)-carbamoyl]ethyl]carbamate (Compound 137, 1.82 g, 83%) as a red oil. LCMS (ES, m/z): 418,420 [M+H]⁺, 318,320 [M+H−100]⁺

### Step 11. Synthesis of Compound 138

A slurry of tert-butyl N-[(1S)-1-[([[2-(2-chloro-4-nitrophenyl)ethyl]-sulfanyl]methyl)carbamoyl]ethyl]carbamate (Compound 137, 1.82 g, 4.36 mmol, 1.00 equiv), iron powder (2.43 g, 0.04 mmol, 10.00 equiv) and NH₄Cl (2.33 g, 0.04 mmol, 10.00 equiv) in EtOH (100.00 mL)/H₂O (50.00 mL) was stirred at 70° C. for 2 h. LCMS indicated the reaction was completed. The reaction was filtered. The filtrate was concentrated to dryness under vacuum. The residue was dissolved with DCM (50.00 mL) and filtered. The filtrate was purified by silica gel column chromatography, eluted with (DCM:MeOH=13:1) to give tert-butyl N-[(1S)-1-[([[2-(4-amino-2-chlorophenyl)ethyl]sulfanyl]methyl)carbamoyl]ethyl]carbamate (Compound 138, 1.20 g, 68%) as a yellow oil. LCMS (ES, m/z): 388,390 [M+H]⁺, 288,290 [M+H−100]₊

### Step 12. Synthesis of Compound 139

To a stirred solution of 3-[5-(aminomethyl)-1-oxo-3H-isoindol-2-yl]piperidine-2,6-dione (INT 1, 352 mg, 1.29 mmol, 1.00 equiv) in DMF (5.00 mL) at 0° C. was added CDI (209.00 mg, 1.29 mmol, 1 equiv) and TEA (260 mg, 2.58 mmol, 2 equiv). The resulting mixture was stirred at 0° C. for 2 h. Then tert-butyl N-[(1S)-1-[([[2-(4-amino-2-chlorophenyl)ethyl]sulfanyl]-methyl)carbamoyl]-ethyl]carbamate (Compound 138, 500.00 mg, 1.29 mmol, 1.00 equiv) and DMAP (472 mg, 3.87 mmol, 3.00 equiv) were added. The resulting mixture was stirred at 60° C. for 24 h. LCMS indicated the reaction was completed. After cooled to room temperature, The reaction mixture was purified by reverse flash chromatography with the following conditions: column, C18 silica gel; mobile phase, ACN in water (0.1% FA), 0% to 60% gradient in 30 min; detector, UV 254 nm to give tert-butyl N-[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]carbamoyl)ethyl]carbamate, (Compound 139, 450.00 mg, 48%) t as a yellow solid. LCMS (ES, m/z): 687,689 [M+H]⁺, 587,589 [M+H−100]⁺

### Step 13. Synthesis of Compound 140

To a stirred solution of tert-butyl N-[(1S)-1-([[(2-[2-chloro-4-[([[2-(2,6-dioxopiperidin-3-yl)-1-oxo-3H-isoindol-5-yl]methyl]carbamoyl)amino]phenyl]ethyl)sulfanyl]-methyl]carbamoyl)ethyl]carbamate (Compound 139, 440.00 mg, 0.64 mmol, 1.00 equiv) in DCM (22.00 mL) was added TFA (2.20 mL) at room temperature. The resulting mixture was stirred at room temperature for 0.5 h. LCMS indicated the reaction was completed. The reaction was concentrated to dryness under vacuum to give (2S)-2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]propanamide; trifluoroacetic acid (Compound 140, 400.00 mg) as a red oil. LCMS (ES, m/z): 578,589 [M+H−TFA]⁺

### Step 14. Synthesis of Compound 142

To a slurry of L-valine (Compound 141, 0.50 g, 4.27 mmol, 1.00 equiv) in DMSO (10 mL) was added 2,5-dioxopyrrolidin-1-yl 6-(2,5-dioxopyrrol-1-yl)hexanoate (1.32 g, 4.28 mmol, 1.00 equiv) and DIEA (1103 mg, 8.54 mmol, 2.00 equiv). The resulting mixture was stirred at room temperature for 4 h. LCMS indicated the reaction was completed. The reaction mixture was purified by reverse flash chromatography with the following conditions: column, C18 silica gel; mobile phase, ACN in water (0.1% FA), 0% to 60% gradient in 30 min; detector, UV 220 nm to give (2S)-2-[6-(2,5-dioxopyrrol-1-yl)hexanamido]-3-methylbutanoic acid (Compound 142, 1.2 g, 72%) as a brown solid. LCMS (ES, m/z): 311 [M+H]⁺

### Step 15. Synthesis of Compound (Im)

A solution of (2S)-2-[6-(2,5-dioxopyrrol-1-yl)hexanamido]-3-methylbutanoic acid, (Compound 142, 59 mg, 0.19 mmol, 1.00 equiv), HOBT (26 mg, 0.19 mmol, 1.00 equiv) and HATU (72 mg, 0.19 mmol, 1.00 equiv) in DMF (2 mL) was stirred at room temperature in air for 1 hour. Then (2S)-2-amino-N-[[(2-[2-chloro-4-[([[2-(2,6-dioxopiperidin-3-yl)-1-oxo-3H-isoindol-5-yl]methyl]carbamoyl)amino]phenyl]ethyl)sulfanyl]methyl]propanamide trifluoroacetic acid (Compound 140, 200 mg, 0.19 mmol, 1.00 equiv, 66.70%) and DIEA (197 mg, 1.52 mmol, 8.00 equiv) was added at room temperature. The reaction mixture was stirred at room temperature for 2 h. LCMS indicated the reaction was completed. The resulting mixture was purified by reverse flash chromatography with the following conditions: Column: YMC-Actus Triart C18, 30 mm×150 mm, 5 um; Mobile Phase A:Water (0.1% FA), Mobile Phase B:ACN; Flow rate: 60 mL/min; Gradient: 28 B to 45 B in 10 min, 254 nm; RT1:9.67 min. The collected fraction was lyophilized to give N-[(1S)-1-[[(1S)-1-([[(2-[2-chloro-4-[([[2-(2,6-dioxopiperidin-3-yl)-1-oxo-3H-isoindol-5-yl]methyl]carbamoyl)amino]-phenyl]ethyl)sulfanyl]methyl]carbamoyl)ethyl]carbamoyl]-2-methylpropyl]-6-(2,5-dioxopyrrol-1-yl)hexanamide (Compound (Im), 27.8 mg, 16%) as a white solid. LCMS (ES, m/z): 879,881 [M+H]⁺. ¹H NMR (400 MHz, DMSO-d₆) δ10.99 (s, 1H), 8.80 (s, 1H), 8.47 (t, J=6.0 Hz, 1H), 8.03 (d, J=7.2 Hz, 1H), 7.78 (d, J=8.8 Hz, 1H), 7.70-7.66 (m, 2H), 7.51 (s, 1H), 7.44 (d, J=8.0 Hz, 1H), 7.21-7.14 (m, 2H), 6.99 (s, 2H), 6.82 (t, J=6.0 Hz, 1H), 5.13-5.10 (m, 1H), 4.47-4.40 (m, 3H), 4.33-4.29 (m, 3H), 4.24 (t, J=7.2 Hz, 1H), 4.14 (t, J=6.8 Hz, 1H), 3.38-3.36 (m, 1H), 2.97-2.90 (m, 1H), 2.86 (t, J=7.6 Hz, 2H), 2.73-2.67 (m, 2H), 2.62-2.57 (m, 1H), 2.40-2.35 (m, 1H), 2.20-2.05 (m, 2H), 2.02-1.96 (m, 1H), 1.95-1.88 (m, 1H), 1.48-1.46 (m, 4H), 1.23-1.16 (m, 6H), 0.83-0.78 (m, 6H).

### Example 4: Preparation and Characterization of NeoDegrader Conjugates

A solution of antibody was treated with 30 equivalents of tris-(2-carboxyethyl)phosphine (TCEP) and incubated at 37° C. for 1 hour to reduce the interchain disulfides. The reduced antibody was purified into 50 mM EPPS, 5 mM EDTA pH 7.0 buffer using illustra NAP columns (GE Healthcare).

Conjugation was effected by treatment of a solution of reduced anti-CD33 antibody comprising a heavy chain having SEQ ID NO: 9 and a light chain having SEQ ID NO: 10 (“CD33AB”) at 2-5 mg/mL in 50 mM EPPS, 5 mM EDTA pH 7.0 with 12 equivalents of linker-neoDegrader added as a stock solution in N,N-dimethylacetamide (DMA) such that the final concentration of DMA was 15% (v/v). The resulting reaction mixture was left overnight at 4° C. The resulting neoDegrader conjugate was purified into 20 mM succinate, 8% sucrose, 0.01% Tween-20 pH 5.5 using illustra NAP columns (GE Healthcare) and concentrated using Amicon Ultra centrifugal concentrators with 50 kD molecular weight cutoff (Millipore).

Concentration and monomer were determined by size exclusion chromatography using a 7.8×300 mM TSKGel 3000SWXL column with 5 μm particles (Tosoh Bioscience), eluting isocratically with 400 mM sodium perchlorate, 50 mM sodium phosphate, 5% (v/v) isopropanol mobile phase running at 0.5 mg/mL for 30 min. NeoDegrader conjugates were quantitated from antibody standard curves, detecting at 214 nm.

Drug to antibody ratio (DAR) was determined by hydrophobic interaction chromatography using a 4.6×35 mm TSKgel Butyl-NPR column with 2.5 μm particles. Mobile phase A was 1.5 M ammonium sulfate, 25 mM sodium phosphate pH 7.0. Mobile phase B was 25 mM sodium phosphate pH 7.0, 25% (v/v) isopropanol. Analytes were eluted with a linear gradient of 0-100% B in 12 min. at a flow rate of 0.6 mL/min. Detection was at 214 nm.

Free linker-payload was determined by mixed-mode chromatography using a 4.6×250 mm HISEP column with 2.5 μm particles (Supelco). Mobile phase A was 100 mM ammonium acetate. Mobile phase B was 100% acetonitrile. Analytes were eluted with a gradient of 25-40% B in 25 min., then 40-100% B in 2 min at a flow rate of 0.7 mL/min. Column temperature was 35° C. Free linker-payload was quantitated using an external standard curve, detecting at 254 nm.

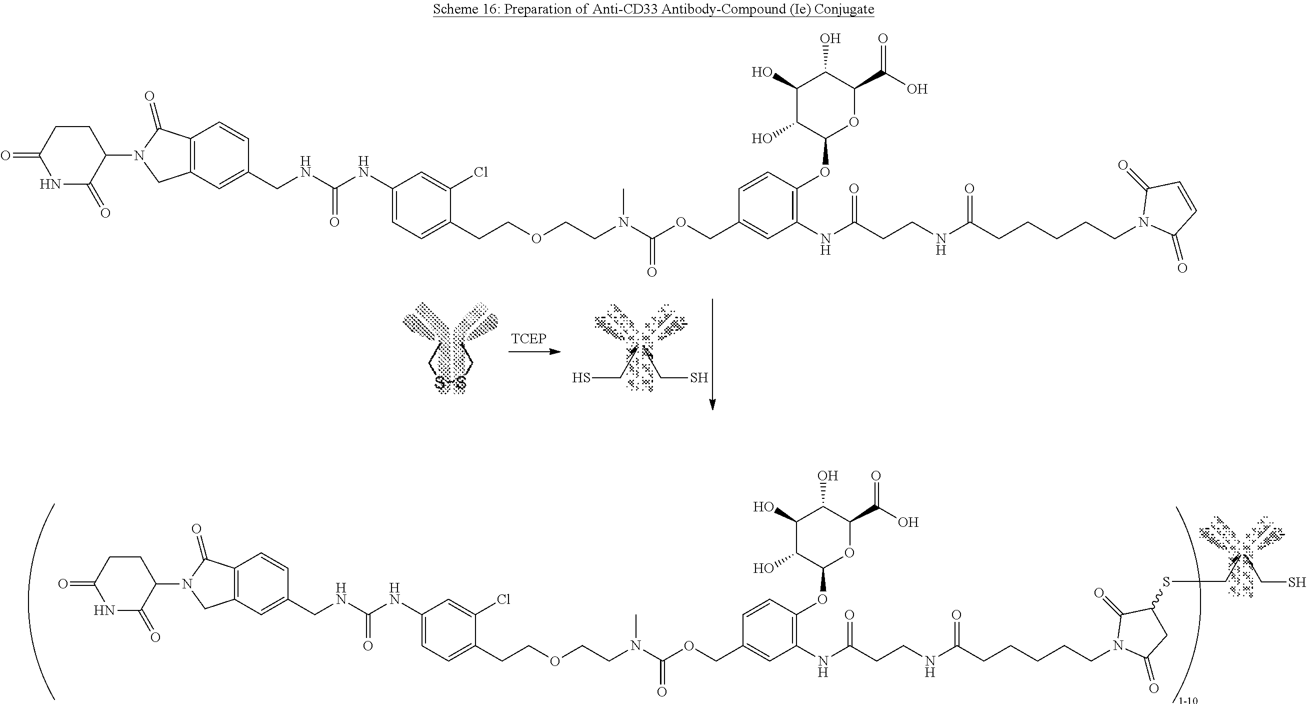

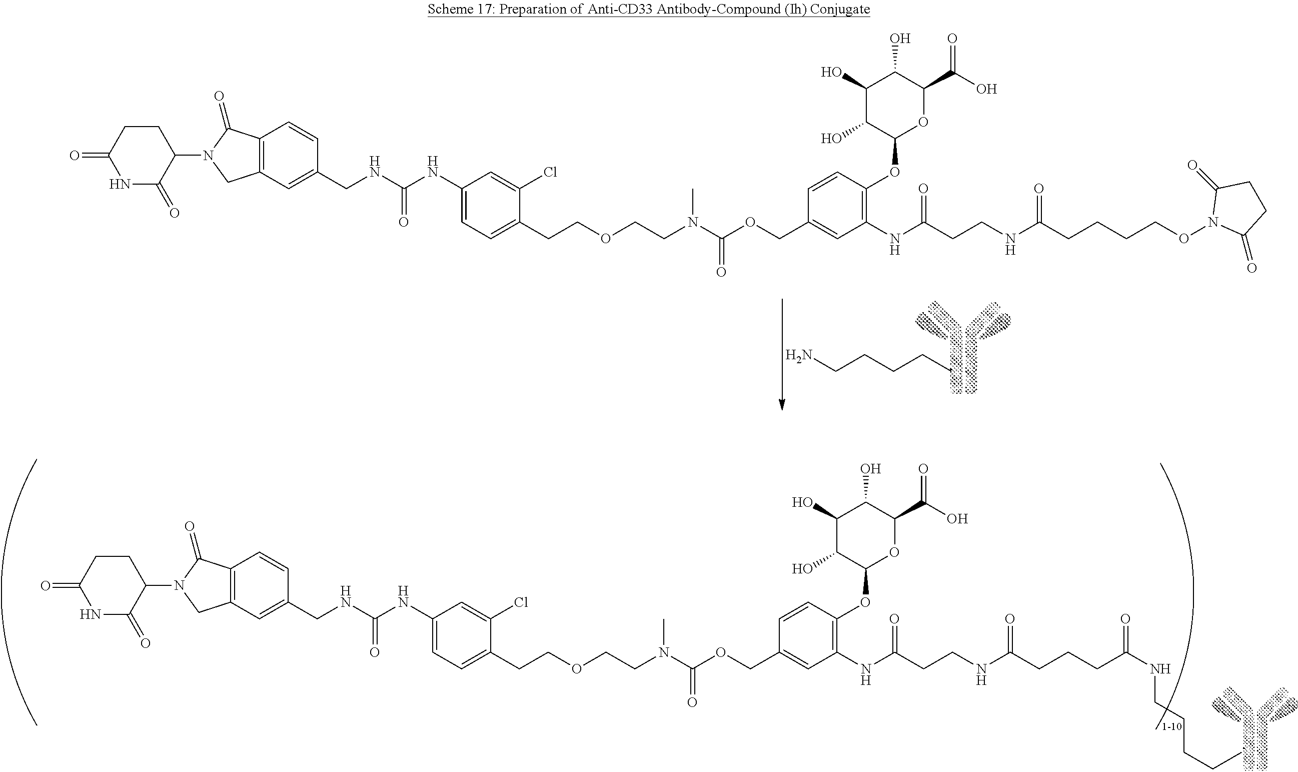

Other conjugates can be prepared in a similar fashion using the appropriate antibody.

### Example 5: Treatment of Acute Myeloid Leukemia (AML) with Anti-CD33 Antibody-neoDegrader Conjugate

Anti-CD33 antibody (CD33AB)-neoDegrader compounds were tested in athymic nude mice (Crl:NU(NCr)-Foxn1^{nu}, Charles River). 1×10⁷ MV411 human acute monocytic leukemia cells (ATCC© CRL-5991™) in 50% Matrigel were injected subcutaneously in the flank of the mice (0.1 mL/mouse). The mice were dosed with anti-CD33 antibody-neoDegrader conjugates, non-targeting neoDegrader conjugates, and vehicle control once tumors reached an average size of 100-150 mm³.

The stock solutions of CD33AB-Compound (Ia), CD33AB—Compound (Ie), and CD33AB-Compound (Ih) were diluted with vehicle to obtain 0.3, 0.283, and 0.299 mg/mL dosing solutions, which provided 3, 2.83, and 2.99 mg/kg in a dosing volume of 10 mL/kg (0.2 mL per 20 g mouse), adjusted to the body weight of each animal. This dosing strategy ensured the delivery of the same amount of payloads to each testing group. Mylotarg was diluted in 0.9% sodium chloride solution to 0.01 mg/mL, which provided 3 mg/kg in a dosing volume of 10 mL/kg (0.2 mL per 20 g mouse). Venetoclax was formulated in solvent composed of 60% PG, 30% PEG400, 10% ethanol via ultrasonication to obtain a dosing suspension of 5 mg/mL, which delivered 50 mg/kg when administered in a volume of 10 mL/kg. CC-90009 was centrifuged to collect the powder at the bottom; then N-methyl-2-pyrrolidinone (NMP), PEG400 and saline were added and mixed well one by one to obtain a 0.5 mg/mL dosing solution in 5% NMP, 45% PEG400 and 50% saline, which delivered 5 mg/kg when administered in a volume of 10 mL/kg.

Mice were divided into 7 treatment groups (N=9/group), as follows: 1) vehicle; 2) CD33AB-Compound (Ia) (3 mg/kg, iv, qd×1); 3) CD33AB-Compound (Ie) (2.83 mg/kg, iv, qd×1); 4) CD33AB-Compound (Ih) (2.99 mg/kg, iv, qd×1); 5) Mylotarg (0.1 mg/kg, iv, qd×1); 6) Venetoclax (50 mg/kg, po, qd×21); 7) CC-90009 (5 mg/kg, ip, bid×10). Test articles for groups 1-5 were administered intravenously (i.v.) as a single dose (qd×1) in volumes adjusted for body weight (0.200 mL/20 g mouse). Venetoclax was administered orally (po) while CC-90009 was administered intraperitoneally (ip) in a dosing volume of 10 mL/kg (0.2 mL per 20 g mouse) scaled to the BW of each animal.

Tumors were measured using calipers twice per week, and each animal was euthanized when its tumor reached the endpoint volume (2,000 mm³) or on the last day (Day 45) of the study, whichever came first. The MTV(n) was defined as the median tumor volume on the last day of the study in the number of animals remaining (n) whose tumors had not attained the endpoint volume.

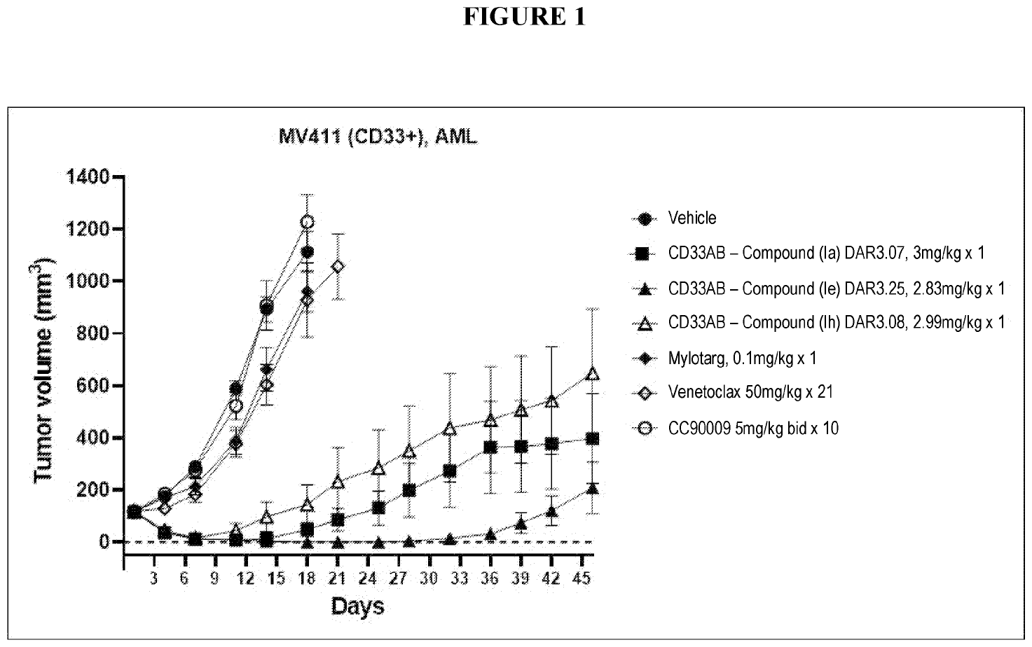

As shown in FIG. 1, all of the neoDegrader conjugates provided slower tumor growth over time compared to the vehicle.

### Example 6: Activity of AB1—Compound (Ia) Conjugate Against Human Leukemia Models

In vitro cytotoxicity was measured using a panel of CD33 positive acute myeloid leukemia cell lines and a panel of non-AML CD33-negative cells. The cells, at a predetermined concentration, were plated into 96 well plates, and, after overnight incubation at 37° C./5CO₂, serial dilutions of each test article (TA) were added to the cells. Cells were incubated with test articles for 72 hours, and viability was detected with CellTiter-Glo® reagent (Promega). The luminescent values were normalized for each cell line, and the IC50s were calculated using Prizm software.

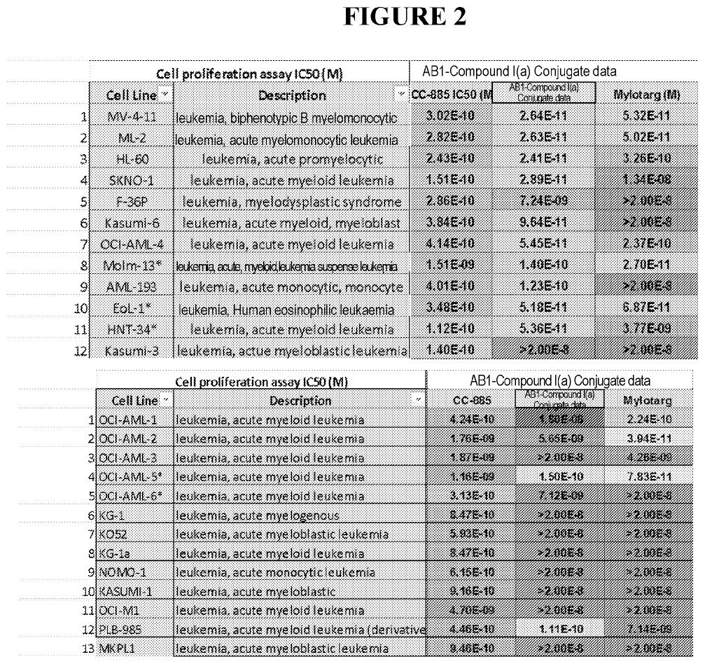

Results indicated that a huMy9-6 derived antibody, AB1, conjugated to Compound Ia (“AB—Compound (Ia)”) exhibited comparable overall in vitro efficacy as CC-885 (a known GSPT1 degrader) or Mylotarg on CD33-positive AML cells—with some cases of superior efficacy. (See FIG. 2.) Additionally, consistent with the hypothesis of targeted CD33-mediated delivery of the GSPT1 degrader payload, the AB1—Compound (Ia) was inactive in CD33-negative cell models.

### Example 7: Treatment of Acute Myeloid Leukemia (AML) with Antibody-neoDegrader Conjugates

Subcutaneous tumor model MV4-11 human acute myelocytic leukemia cells (1×10⁶ cells in 0.1 mL) were subcutaneously inoculated into the right flank of female athymic nude mice. Mice were treated with test article either by intravenous injection into a lateral tail vein, intraperitoneal injection, oral gavage, or combinations thereof starting when tumors reached 150 mm³ in size. Tumor size and mouse body weight were measured twice per week.

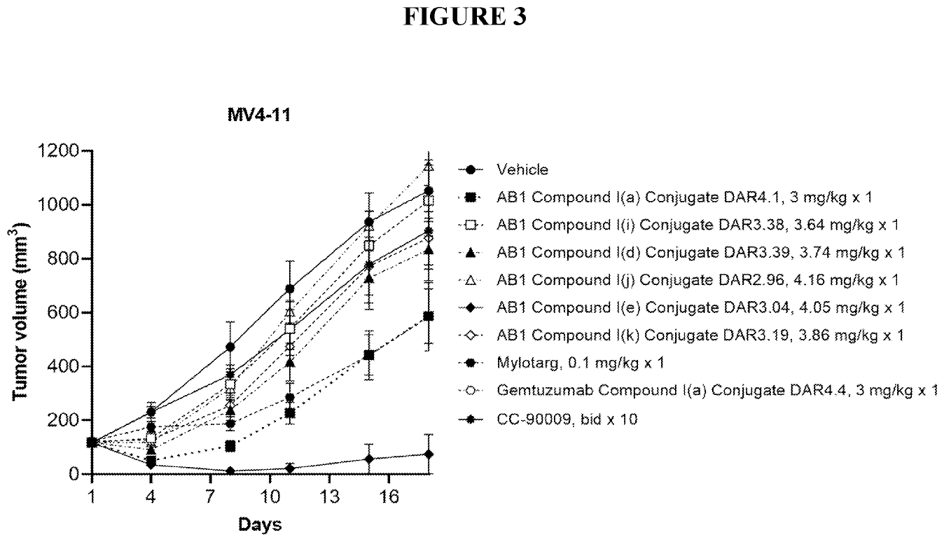

Consistent with the observations in vitro, in vivo treatment of a CD33-positive AML model tumor (MV4-11) with several AB1-based conjugates releasing neoDegrader P1 resulted in tumor regressions, with the most robust effects seen with a conjugate containing a beta-glucuronide release trigger and native cysteine conjugation. (See FIG. 3.)

### Example 8: Stability of CD33AB—Compound (Ia) and Gemtuzumab-Compound (Ia) Conjugates

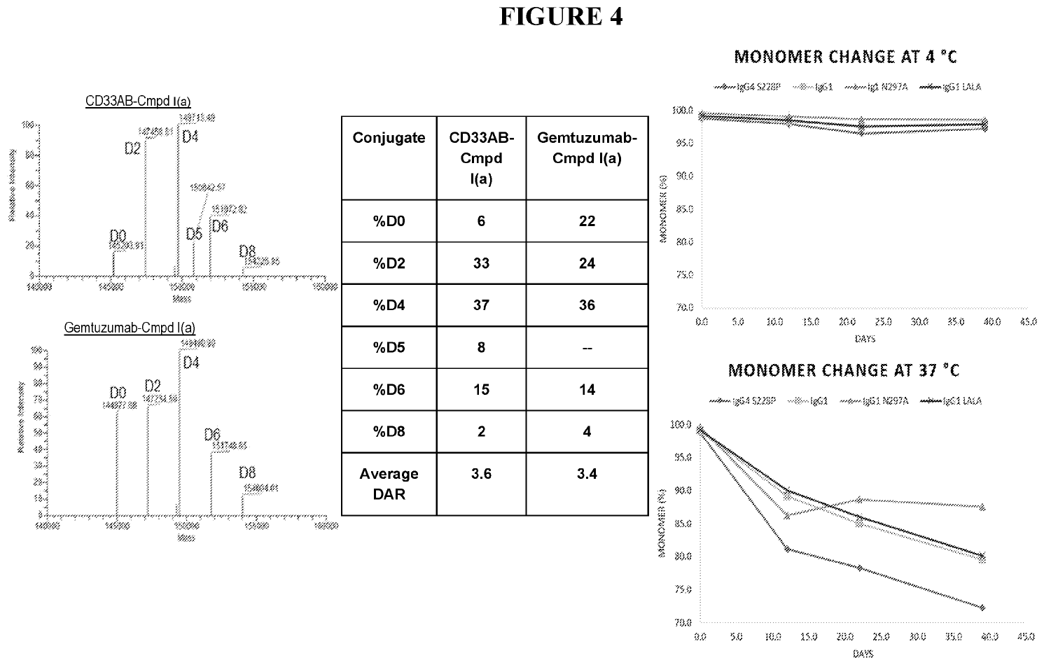

Stability assays of antiCD33—Compound (Ia) conjugates (˜8 DAR) were carried out over 40 days using Gemtuzumab, IgG1, CD33AB, and IgG1 L234A/L235A “LALA” Ab formats at 2.5 mg/mL in 20 mM succinate, 8% sucrose, 0.01% Tween-20 pH 5.5 and assayed by size exclusion chromatrophaphy (SEC). At 4° C., no significant change in concentration or monomer was observed for any of the conjugates. (FIG. 4.) In contrast at 37° C., native Gemtuzumab-Compound (Ia) as well as the Compound (Ia) conjugates using IgG1 and IgG1 LALA backbone showed significant aggregation over 39 days (15-28% increase from 20-39 days). However, CD33AB-Compound (Ia) (using IgG1 N297A backbone) maintains monomeric state above 88% over 39 days at 37° C. (FIG. 4.) Maintaining high monomer as in CD33AB-Compound (Ia) over several days is generally a desirable property for ADCs as aggregation in blood circulation can lead to rapid clearance and increased toxicity, narrowing the therapeutic index. In addition, by native SEC-MS analysis, native Gemtuzumab-Compound (Ia) shows much higher unconjugated antibody than the CD33AB derived conjugate made with the same molar equivalents of payload-linker. Generally, having low levels of unconjugated antibody in an ADC, as in CD33AB-Compound (Ia), is a desirable quality attribute. Also, more TCEP was needed to reduce Gemtuzumab than CD33AB (4.5 vs 2.5 molar equivalents). Using less reducing agent, as in CD33AB-Compound (Ia), to manufacture ADCs is desirable to lower cost of goods and simplify the purification process.

### Example 9: Activity of antiCD33 neoDegrader Conjugates Against Human Leukemia Models

Subcutaneous tumor model MV4-11 human acute myelocytic leukemia cells (1×10⁶ cells in 0.1 mL) were subcutaneously inoculated into the right flank of female athymic nude mice. Mice were treated with test article either by intravenous injection into a lateral tail vein, intraperitoneal injection, oral gavage, or combinations thereof starting when tumors reached 150 mm³ in size. Tumor size and mouse body weight were measured twice per week.

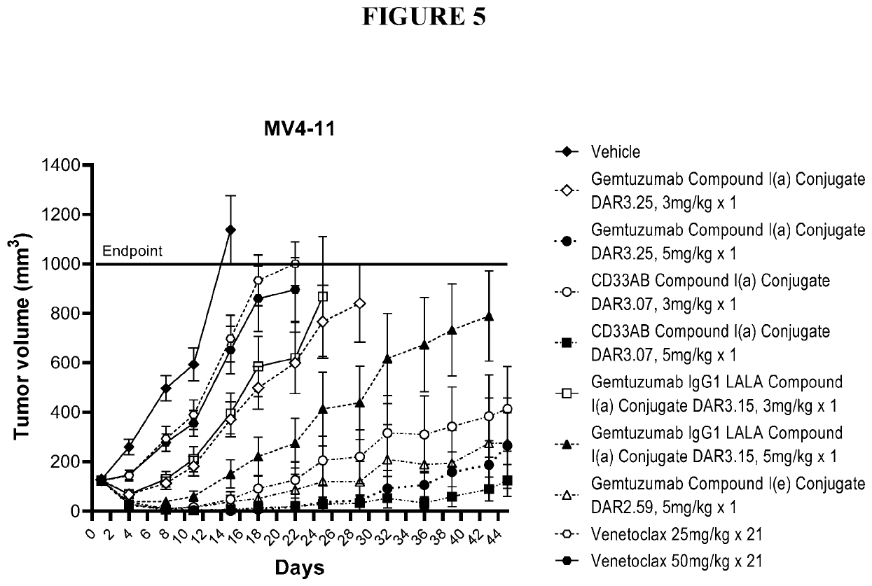

Consistent with the observations in vitro, in vivo treatment of a CD33-positive AML model tumor (MV4-11) with the various CD33-based conjugates, releasing neoDegrader P1 resulted in tumor regressions, with the most stable conjugate (CD33AB-Compound (Ia)) exhibiting robust anti-tumor efficacy. (FIG. 5.)

### Example 10: Activity of antiCD33 neoDegradader Conjugates Against Mylotarg-Insensitive Cell Lines

The cytotoxicity of test articles (TA) were measured using a panel of CD33 positive acute myeloid leukemia cell lines known to be Mylotarg insensitive (AML-193 and Kasumi-6). The cells, at a predetermined concentration, were plated into 96 well plates, and, after overnight incubation at 37° C./5CO₂, serial dilutions of each test article (TA) were added to the cells. Cells were incubated with test articles for 72 hours, and viability was detected with CellTiter-Glo® reagent (Promega). The luminescent values were normalized for each cell line, and the IC50s were calculated using Prizm software.

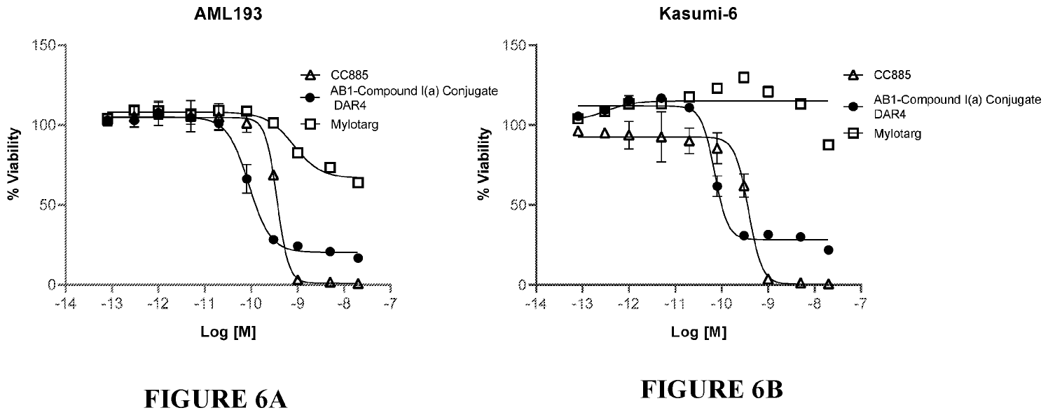

As shown in FIGS. 6A and 6B, the anti-CD33 neo Degrader conjugate had good activity against both cell lines.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A conjugate of formula (I): 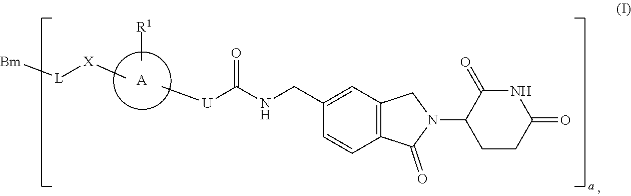
or a pharmaceutically acceptable salt thereof, wherein:
a is an integer from 1 to 10;
A is phenyl or a C₄-C₁₀cycloalkyl ring;
U is selected from NH and CF₂;
R¹ is independently selected from hydrogen and halo;
X is selected from —NR²—, ═C(CH₃)—, -Q-(CH₂)ₙ—, and -Q(CH₂)ₘQ′(CH₂)ₙ—; wherein
Q and Q′ are each independently O, S, or N(R²)ᵥ;
v is 1 or 2;
each R² is independently hydrogen or C₁-C₆alkyl;
n is an integer from 1 to 6; and
m is an integer from 2 to 6;
wherein the left side of each group is attached to L and the right side is attached to A;
provided that when X is NH or -Q-(CH₂)ₙ—, R¹ is halo;
L is a cleavable linker or non-cleavable linker; and
Bm is an anti-CD33 antibody or antigen-binding portion thereof comprising a heavy chain variable region (VH) complementarity determining region (CDR) 1 (VH-CDR1) comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a light chain variable region (VL) CDR1 (VL-CDR1) comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7.

2. The conjugate of claim 1, wherein the anti-CD33 antibody or antigen-binding portion thereof comprises a VH comprising the amino acid sequence set forth in SEQ ID NO: 4 and a VL comprising the amino acid sequence as set forth in SEQ ID NO: 8.

3. The conjugate of claim 1 or 2, wherein the anti-CD33 antibody or antigen-binding portion thereof comprises a constant region, wherein the constant region comprises at least one amino acid different from Gemtuzumab.

4. The conjugate of any one of claims 1 to 3, wherein the anti-CD33 antibody or antigen-binding portion thereof is an IgG1 antibody or antigen-binding portion thereof.

5. The conjugate of any one of claims 1 to 4, wherein the anti-CD33 antibody or antigen-binding portion thereof comprises alanine at amino acid 297 corresponding to the constant region.

6. The conjugate of any one of claims 1 to 5, wherein the anti-CD33 antibody comprises a heavy chain as set forth in SEQ ID NO: 9 and a light chain as set forth in SEQ ID NO: 10.

7. The conjugate of any one of claims 1 to 6, or a pharmaceutically acceptable salt thereof, wherein a is an integer from 2 to 8.

8. The conjugate of any one of claims 1 to 7, or a pharmaceutically acceptable salt thereof, wherein L is a non-cleavable linker.

9. The conjugate of claim 8, or a pharmaceutically acceptable salt thereof, wherein L is selected from the group consisting of 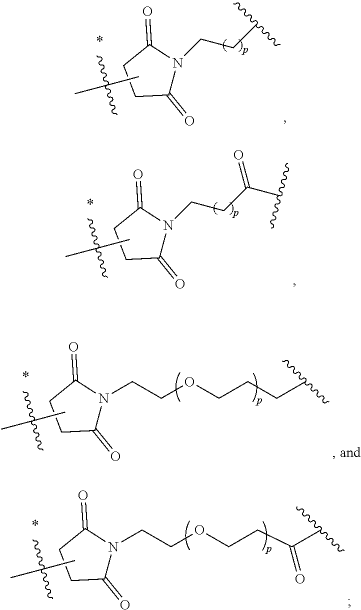
wherein:
p is an integer from 1 to 10; [symbol]
is the point of attachment to X; and [symbol]
is the point attachment to the anti-CD33 antibody or antigen-binding portion thereof.

10. The conjugate of claim 9, or a pharmaceutically acceptable salt thereof, wherein L is

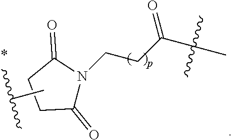

11. The conjugate of claim 9 or 10, or a pharmaceutically acceptable salt thereof, wherein p is 5.

12. The conjugate of any one of claims 1 to 7, or a pharmaceutically acceptable salt thereof, wherein L is a cleavable linker.

13. The conjugate of claim 12, or a pharmaceutically acceptable salt thereof, wherein the cleavable linker is cleavable by a protease.

14. The conjugate of claim 12 or 13, or a pharmaceutically acceptable salt thereof, wherein L is selected from the group consisting of 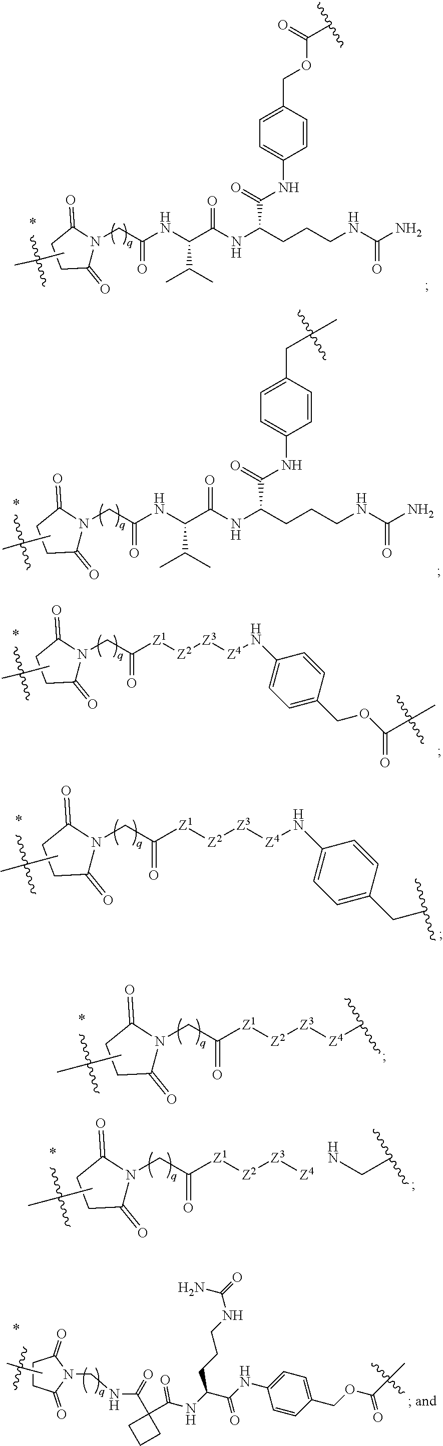 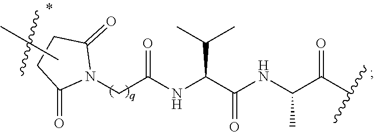
wherein:
q is an integer from 2 to 10;
Z¹, Z², Z³, and Z⁴ are each independently absent or a naturally-occurring amino acid residue in the L- or D-configuration, provided that at least two of Z, Z², Z³, and Z⁴ are amino acid residues; [symbol]
is the point of attachment to X; and [symbol]
is the point of attachment to the anti-CD33 antibody or antigen-binding portion thereof.

15. The conjugate of claim 14, or a pharmaceutically acceptable salt thereof, wherein Z¹, Z², Z³, and Z⁴ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴ are amino acid residues.

16. The conjugate of claim 15, or a pharmaceutically acceptable salt thereof, wherein:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine; and
Z⁴ is selected from the group consisting of L-alanine, D-alanine, L-citrulline, D-citrulline, L-asparagine, D-asparagine, L-lysine, D-lysine, L-phenylalamine, D-phenylalanine, and glycine.

17. The conjugate of claim 14, or a pharmaceutically acceptable salt thereof, wherein L is

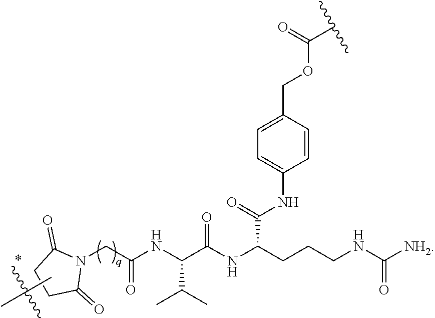

18. The conjugate of claim 17, or a pharmaceutically acceptable salt thereof, wherein q is 5.

19. The conjugate of claim 12, or a pharmaceutically acceptable salt thereof, wherein L is a bioreducible linker.

20. The conjugate of claim 12 or 19, wherein L is selected from the group consisting of 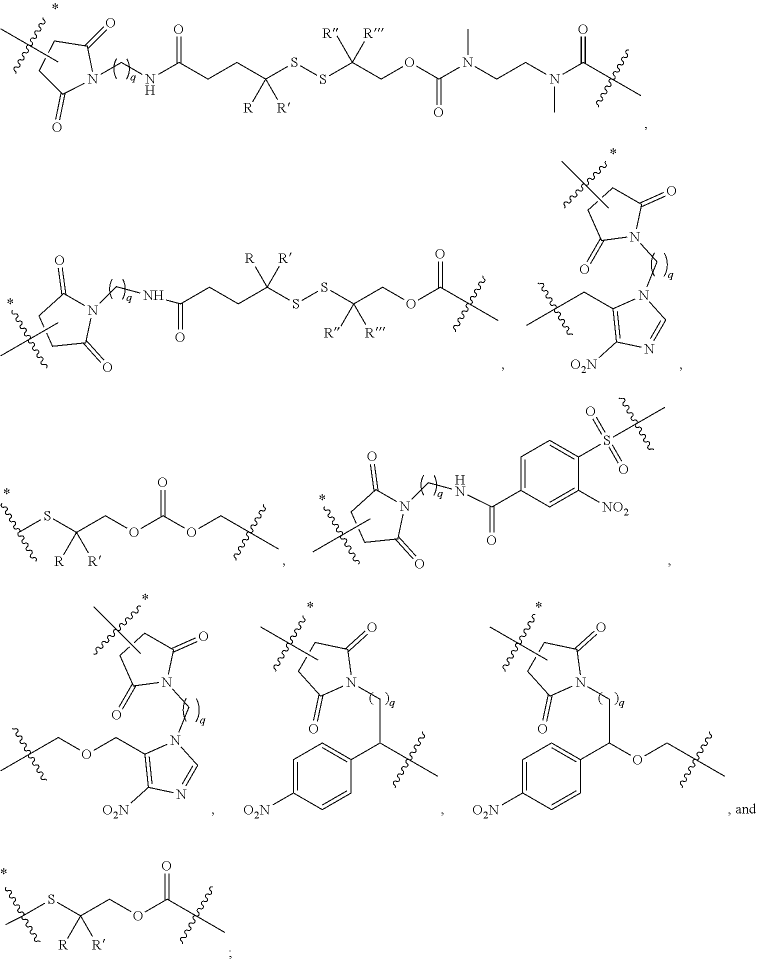
wherein:
q is an integer from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring; [symbol]
is the point of attachment to X; and [symbol]
is the point of attachment to the anti-CD33 antibody or antigen-binding portion thereof.

21. The conjugate of claim 12, or a pharmaceutically acceptable salt thereof, wherein L is an acid cleavable linker.

22. The conjugate of claim 12 or 21, or a pharmaceutically acceptable salt thereof, wherein L is selected from the group consisting of 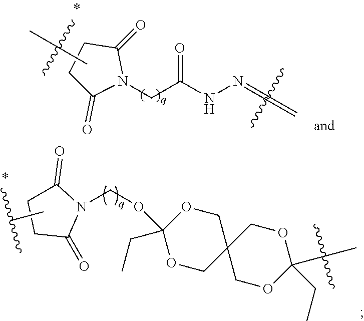
wherein:
q is an integer from 2 to 10; [symbol]
is the point of attachment to X; and [symbol]
is the point of attachment to the anti-CD33 antibody or antigen-binding portion thereof.

23. The conjugate of claim 12, or a pharmaceutically acceptable salt thereof, wherein L is a click-to-release linker.

24. The conjugate of claim 12 or 23, or a pharmaceutically acceptable salt thereof, wherein L is selected from 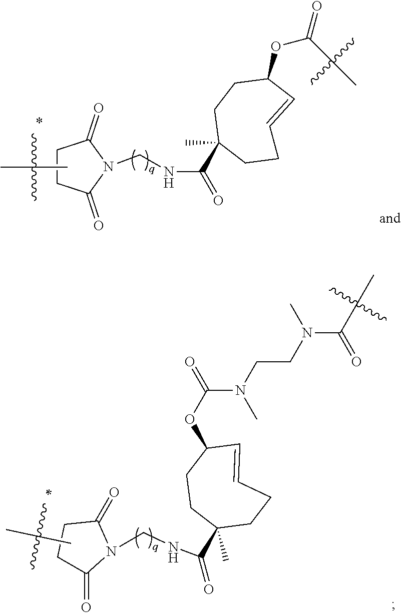
wherein:
q is an integer from 2 to 10; [symbol]
is the point of attachment to X; and [symbol]
the point of attachment to the anti-CD33 antibody or antigen-binding portion thereof.

25. The conjugate of claim 12, or a pharmaceutically acceptable salt thereof, wherein L is a pyrophosphatase cleavable linker.

26. The conjugate of claim 25, or a pharmaceutically acceptable salt thereof, wherein L is 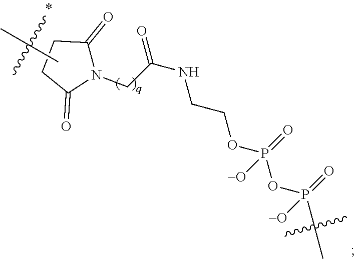
wherein:
q is an integer from 2 to 10; [symbol]
is the point of attachment to X; and [symbol]
is the point of attachment to the anti-CD33 antibody or antigen-binding portion thereof.

27. The conjugate of claim 12, or a pharmaceutically acceptable salt thereof, wherein L is a beta-glucuronidase cleavable linker.

28. The conjugate of claim 12 or 27, or a pharmaceutically acceptable salt thereof, wherein L is selected from 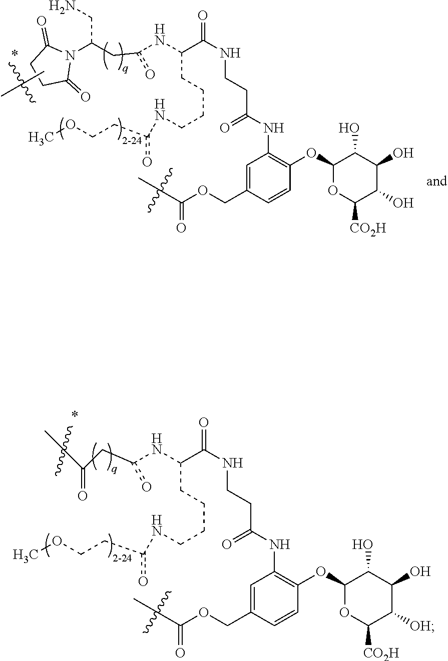
wherein:
q is an integer from 2 to 10;
---- is absent or a bond; [symbol]
is the point of attachment to X; and [symbol]
is the point of attachment to the anti-CD33 antibody or antigen-binding portion thereof.

29. The conjugate of any one of claims 1 to 28, or a pharmaceutically acceptable salt thereof, wherein:
A is phenyl;
U is NH;
R¹ is halo; and
X is —N(R²)ᵥ(CH₂)ₘO(CH₂)ₙ—; wherein:
v is 1;
m and n are 2; and
R² is methyl.

30. The conjugate of any one of claims 1 to 28, wherein:
A is phenyl;
U is NH;
R¹ is halo; and
X is —N(R²)ᵥ(CH₂)ₘO(CH₂)ₙ—; wherein:
v is 2;
m and n are 2; and
each R² is methyl.

31. The conjugate of any one of claims 1 to 28, wherein:
A is phenyl;
U is NH;
R¹ is halo; and
X is —O(CH₂)ₙ—; wherein:
n is 2.

32. The conjugate of any one of claims 1 to 28, wherein:
A is phenyl;
U is NH;
R¹ is halo; and
X is —S(CH₂)ₙ—; wherein:
n is 2.

33. The conjugate of any one of claims 1 to 28, wherein:
A is phenyl;
U is NH;
R¹ is hydrogen; and
X is —NR²—; wherein:
R² is methyl.

34. The conjugate of any one of claims 1 to 28, wherein:
A is phenyl;
U is NH;
R¹ is halo; and
X is —NR²—; wherein:
R² is hydrogen.

35. The conjugate of any one of claims 1 to 28, wherein:
A is phenyl;
U is NH;
R¹ is hydrogen; and
X is —C(CH₃)═.

36. The conjugate of any one of claims 1 to 28, wherein:
A is a C₄-C₁₀cycloalkyl ring;
U is NH;
R¹ is hydrogen; and
X is —N(R²)(CH₂)ₘO(CH₂)ₙ—; wherein:
n is 1;
m is 2; and
R² is methyl.

37. A conjugate of formula (V): 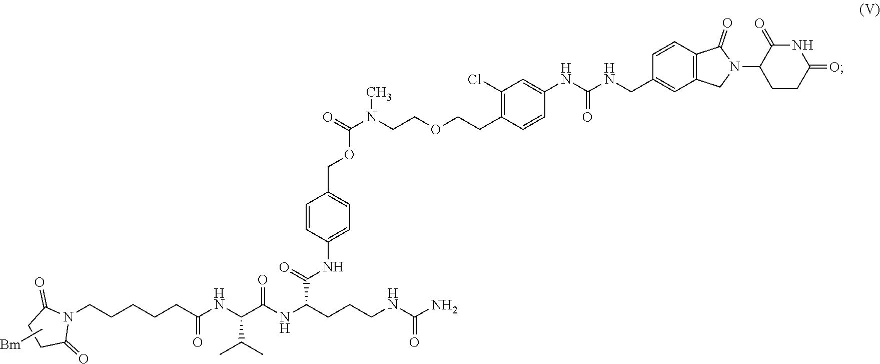
or a pharmaceutically acceptable salt thereof, wherein Bm is an anti-CD33 antibody or antigen-binding portion thereof comprising a VH-CDR1 comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a VL-CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7.

38. A conjugate of formula (VI): 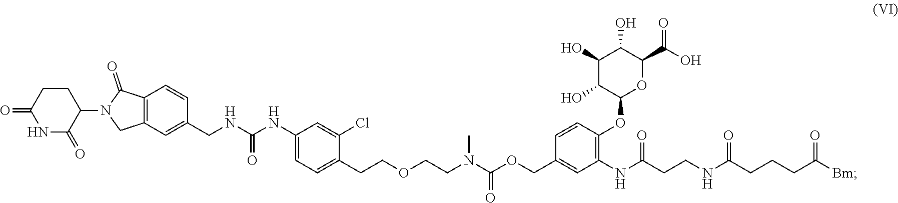
or a pharmaceutically acceptable salt thereof, wherein Bm is an anti-CD33 antibody or antigen-binding portion thereof comprising a VH-CDR1 comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a VL-CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7.

39. A conjugate of formula (VII): 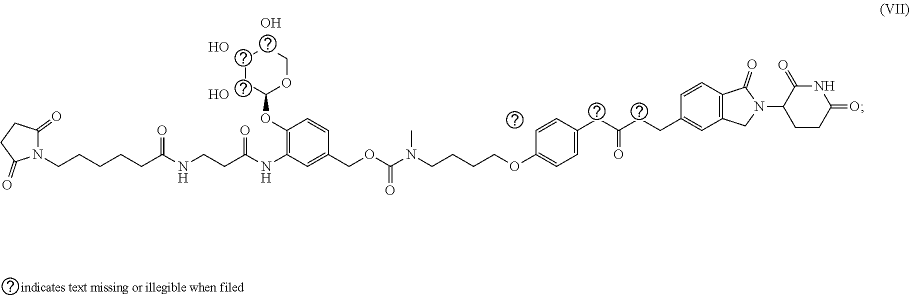
or a pharmaceutically acceptable salt thereof, wherein Bm is an anti-CD33 antibody or antigen-binding portion thereof comprising a VH-CDR1 comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a VL-CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7.

40. The conjugate of any one of claims 37-39 or a pharmaceutically acceptable salt thereof, wherein the anti-CD33 antibody or antigen-binding portion thereof comprises (i) a VH comprising the amino acid sequence as set forth in SEQ ID NO: 4 and a VL comprising the amino acid sequence as set forth in SEQ ID NO: 8 or (ii) a heavy chain comprising the amino acid sequence as set forth in SEQ ID NO: 9 and a light chain comprising the amino acid sequence as set forth in SEQ ID NO: 10.

41. A pharmaceutical composition comprising a conjugate or compound of any one of claims 1 to 40, or a pharmaceutically acceptable salt thereof, and one or more pharmaceutically acceptable carriers.

42. A method of treating cancer in a subject in need thereof, the method comprising administering to the subject a pharmaceutically acceptable amount of a conjugate or composition of any of claims 1 to 41, or a pharmaceutically acceptable salt thereof.

43. The method of claim 42, wherein the cancer is a hematological/blood cancer.

44. The method of claim 42, wherein the cancer is a multiple myeloma, leukemia, malignant lymphoma, Hodgkin's disease, or chronic myeloproliferative disease.

45. The method of claim 42, wherein the cancer is acute myeloid leukemia or lymphoma.

46. The method of claim 42, wherein the cancer is acute myeloid leukemia.

47. The method of any one of claims 42-46, wherein the cancer is resistant or refractory to Mylotarg.

48. A method of treating myelodysplastic syndrome in a subject in need thereof, the method comprising administering to the subject a pharmaceutically acceptable amount of a conjugate or composition of any of claims 1 to 41, or a pharmaceutically acceptable salt thereof.

49. The method of any one of claims 42 to 48, further comprising administering to the subject a pharmaceutically acceptable amount of an additional agent prior to, after, or simultaneously with the conjugate of any one of claims 1 to 40, or a pharmaceutically acceptable salt thereof.

50. The method of claim 49, wherein the additional agent is a cytotoxic agent or an immune response modifier.

51. The method of claim 50, wherein the immune response modifier is a checkpoint inhibitor.

52. The method of claim 51 wherein the checkpoint inhibitor comprises a PD-1 inhibitor, a PD-L1 inhibitor, a CTLA-4 inhibitor, a TIM3 inhibitor, and/or a LAG-3 inhibitor.

53. A method of preparing the conjugate of claim 1, or a pharmaceutically acceptable salt thereof, the process comprising reacting an anti-CD33 antibody or antigen-binding portion thereof with a compound of formula (I-1): 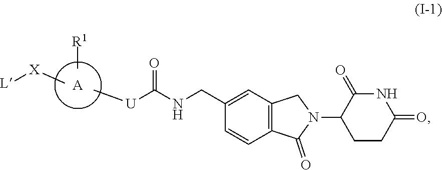
or a pharmaceutically acceptable salt thereof, wherein:
a is an integer from 1 to 10;
A is phenyl or a C₄-C₁₀cycloalkyl ring;
R¹ is independently selected from hydrogen and halo;
U is selected from NH and CF₂;
X is selected from —N(R²)ᵥ—, ═C(CH₃)—, -Q-(CH₂)ₙ—, and -Q(CH₂)ₘQ′(CH₂)ₙ—; wherein
v is 1 or 2;
Q and Q′ are each independently O, S, or NR²;
each R² is independently hydrogen or C₁-C₆alkyl;
n is an integer from 1 to 6; and
m is an integer from 2 to 6;
wherein the left side of each group is attached to L′ and the right side is attached to A;
provided that when X is NH or -Q-(CH₂)ₙ—, R¹ is halo;
L′ is a cleavable or non-cleavable linker precursor that conjugates to the anti-CD33 antibody or antigen-binding portion thereof, wherein the anti-CD33 antibody or antigen-binding portion thereof comprises a VH-CDR1 comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a light chain variable region (VL) CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7.

54. The method of claim 53, wherein the anti-CD33 antibody or antigen-binding portion thereof comprises a VH comprising the amino acid sequence as set forth in SEQ ID NO: 4 and a VL comprising the amino acid sequence as set forth in SEQ ID NO: 8.

55. The method of claim 53 or 54, wherein the anti-CD33 antibody or antigen-binding portion thereof comprises a constant region, wherein the constant region comprises at least one amino acid different from Gemtuzumab.

56. The method of any one of claims 53 to 55, wherein the anti-CD33 antibody or antigen-binding portion thereof is an IgG1 antibody or antigen-binding portion thereof.

57. The method of any one of claims 53 to 56, wherein the anti-CD33 antibody or antigen-binding portion thereof comprises alanine at amino acid 297 corresponding to the constant region.

58. The method of any one of claim 53 or 57, wherein the anti-CD33 antibody comprises a heavy chain as set forth in SEQ ID NO: 9 and a light chain as set forth in SEQ ID NO: 10.

59. The method of any one of claims 53 to 58, further comprising reducing the anti-CD33 antibody or antigen-binding portion thereof prior to reacting with the compound of formula (I-1).

60. The method of any one of claims 53 to 59, wherein a is an integer from 2 to 8.

61. The method of any one of claims 53 to 60, wherein L′ is a non-cleavable linker precursor, a cleavable linker precursor, a bioreducible linker precursor, an acid cleavable linker precursor, a click-to-release linker precursor, a pyrophosphatase cleavable linker precursor, a beta-glucoronidase cleavable linker precursor, or any combination thereof.


SEQUENCE LISTING (28 sequences)

SEQ ID NO: 1 - protein, 5 aa, Artificial Sequence
  Heavy Chain CDR1
DSNIH

SEQ ID NO: 2 - protein, 17 aa, Artificial Sequence
  Heavy Chain CDR2
YIYPYNGGTDYNQKFKN

SEQ ID NO: 3 - protein, 7 aa, Artificial Sequence
  Heavy Chain CDR3
GNPWLAY

SEQ ID NO: 4 - protein, 116 aa, Artificial Sequence [sequence omitted]

SEQ ID NO: 5 - protein, 15 aa, Artificial Sequence
  Light Chain CDR1
RASESLDNYGIRFLT

SEQ ID NO: 6 - protein, 8 aa, Artificial Sequence
  Light Chain CDR2
AASNQGSG

SEQ ID NO: 7 - protein, 9 aa, Artificial Sequence
  Light Chain CDR3
QQTKEVPWS

SEQ ID NO: 8 - protein, 111 aa, Artificial Sequence [sequence omitted]

SEQ ID NO: 9 - protein, 446 aa, Artificial Sequence
  CD33AB Heavy Chain
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, Artificial Sequence
  CD33AB Light Chain
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 443 aa, Artificial Sequence
  Gemtuzumab Heavy Chain
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 218 aa, Artificial Sequence; identical to SEQ ID NO: 10
  Gemtuzumab Light Chain

SEQ ID NO: 13 - protein, 364 aa, Artificial Sequence
  Canonical CD33
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 14 - protein, 237 aa, Artificial Sequence
  CD33 Human Isoform 2
MPLLLLLPLLWADLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 15 - protein, 310 aa, Artificial Sequence
  CD33 Human Isoform 3
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPVR

SEQ ID NO: 16 - protein, 418 aa, Artificial Sequence
  CD33 Human Isoform X1
MDLGEAATRARPAVISPGVNSCAQKSTSEWKDFRHGVRMSQMALKEALEAASSDMPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 17 - protein, 402 aa, Artificial Sequence
  CD33 Human Isoform X2
MDLGEAATRARPAVISPGVNSCAQKSTSEWKDFRHGVRMSQMALKEALEAASSDMPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTWKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 18 - protein, 291 aa, Artificial Sequence
  CD33 Human Isoform X4
MDLGEAATRARPAVISPGVNSCAQKSTSEWKDFRHGVRMSQMALKEALEAASSDMPLLLLLPLLWADLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 19 - protein, 5 aa, Artificial Sequence
  huMy9-6 Heavy Chain CDR1
SYYIH

SEQ ID NO: 20 - protein, 17 aa, Artificial Sequence
  huMy9-6 Heavy Chain CDR2
VIYPGNDDISYNQKFQG

SEQ ID NO: 21 - protein, 9 aa, Artificial Sequence
  huMy9-6 Heavy Chain CDR3
EVRLRYFDV

SEQ ID NO: 22 - protein, 17 aa, Artificial Sequence
  huMy9-6 Light Chain CDR1
KSSQSVFFSSSQKNYLA

SEQ ID NO: 23 - protein, 7 aa, Artificial Sequence
  huMy9-6 Light Chain CDR2
WASTRES

SEQ ID NO: 24 - protein, 8 aa, Artificial Sequence
  huMy9-6 Light Chain CDR3
HQYLSSRT

SEQ ID NO: 25 - protein, 445 aa, Artificial Sequence
  huMy9-6-IgG4-S228P Anti-CD33 Heavy Chain
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 219 aa, Artificial Sequence
  huMy9-6-IgG4-S228P Anti-CD33 Light Chain
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 118 aa, Artificial Sequence
  huMy9-6 Heavy Chain Variable Region
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 28 - protein, 112 aa, Artificial Sequence
  huMy9-6 Light Chain Variable Region
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIK